(19) United States
(12) Patent Application Publication
(10) Pub. No.: US 2024/0024273 A1
(43) Pub. Date: Jan. 25, 2024

(54) TOPICAL ADMINISTRATION OF 2-(DIETHYLAMINO)ETHYL 2-(4-ISOBUTYLPHENYL)PROPRIONATE FOR TREATMENT OF DISEASES

(71) Applicants: Chongxi Yu, Princeton, NJ (US); TECHFIELDS PHARMA CO., LTD., Suzhou, Jiangsu (CN)

(72) Inventors: Chongxi Yu, Princeton, NJ (US); Lina Xu, Suzhou, Jiangsu (CN)

(21) Appl. No.: 18/251,336

(22) PCT Filed: Jun. 4, 2021
(86) PCT No.: PCT/CN2021/098323

§ 371 Date: May 1, 2023

(30) Foreign Application Priority Data
Jun. 5, 2020 (WO) PCT/CN2020/094560

(51) Int. Cl. A61K 31/222; A61K 9/00; A61P 19/02; A61P 29/00

(52) U.S. Cl. CPC A61K 31/222; A61K 9/0014; A61P 19/02; A61P 29/00

(57) ABSTRACT

Provided herein is 2-(diethylamino) ethyl 2-(4-isobutylphenyl) propionate or pharmaceutically acceptable salts thereof. Furthermore, for use and methods in the treatment of various pains and inflammation, in particular osteoarthritis with pharmaceutical compositions, treatment kits and devices comprising 2-(diethylamino) ethyl 2-(4-isobutylphenyl) propionate and/or pharmaceutically acceptable salts thereof, as well as dosage forms, dosages, and methods of use thereof through topical administration, for treatment of various pains and inflammation, in particular osteoarthritis, are disclosed.

### TECHNICAL FIELD OF THE DISCLOSURE

The present disclosure relates to the field of medical application, in particular use of 2-(diethylamino)ethyl 2-(4-isobutylphenyl)propionate, and pharmaceutically acceptable salts thereof, for the treatment of pains and/or inflammation.

### BACKGROUND OF THE DISCLOSURE

Ibuprofen, i.e., 2-(4-isobutylphenyl)propionic acid, a non-steroidal anti-inflammatory drug (NSAID), has been used in humans for more than fifty years. Ibuprofen is a known medicine with analgesic, antiphlogistic and antipyretic properties, that is used widely for treatment of symptoms in relation with various diseases, for example, inflammatory diseases and pains, such as rheumatic diseases, headaches, migraines, toothaches, back aches, muscle pain, post-operative pain, and the like.

Ibuprofen is usually administered through oral administration to reach the action site of a condition or disease. As disclosed in U.S. Pat. No. 9,872,846, with an administration dose up to about 50 mg/kg or even 200 mg/kg, ibuprofen was administered orally for treating the patients.

### SUMMARY OF THE DISCLOSURE

The present disclosure relates to use of 2-(diethylamino)ethyl 2-(4-isobutylphenyl)propionate or pharmaceutically acceptable salts thereof for treatment of symptoms that can be treated with Ibuprofen. In one aspect, the present disclosure relates to a method of treatment including administrating 2-(diethylamino)ethyl 2-(4-isobutylphenyl)propionate and/or pharmaceutically acceptable salts thereof. The present disclosure relates to 2-(diethylamino)ethyl 2-(4-isobutylphenyl)propionate or pharmaceutically acceptable salts thereof for use in treatment of osteoarthritis. The present disclosure relates to use of 2-(diethylamino)ethyl 2-(4-isobutylphenyl)propionate or pharmaceutically acceptable salts thereof in the manufacture of a medicament. The present disclosure relates to a kit including 2-(diethylamino)ethyl 2-(4-isobutylphenyl)propionate and/or pharmaceutically acceptable salts thereof. The present disclosure relates to a therapeutic system including a composition including 2-(diethylamino)ethyl 2-(4-isobutylphenyl)propionate and/or pharmaceutically acceptable salts thereof. In some embodiments, the present disclosure relates to a dosage form in which a certain concentration of 2-(diethylamino)ethyl 2-(4-isobutylphenyl)propionate and/or pharmaceutically acceptable salts thereof. In some embodiments, the present disclosure relates to a device capable of administering a certain unit dose. In one aspect, the present disclosure relates to topical administration of 2-(diethylamino)ethyl 2-(4-isobutylphenyl)propionate or pharmaceutically acceptable salts thereof. Specifically, the present disclosure relates to a certain optimum dosage forms and/or dosages of 2-(diethylamino)ethyl 2-(4-isobutylphenyl)propionate or pharmaceutically acceptable salts thereof.

In some embodiments, the present disclosure relates to a method of treatment of a subject, including topical administrating 2-(diethylamino)ethyl 2-(4-isobutylphenyl)propionate and/or pharmaceutically acceptable salts thereof to the subject, in particular to one or more sites of the subject, in an amount of about 1 mg to about 80 mg, in particular 1 mg to 80 mg, per day, in particular per day per site.

In some embodiments, the present disclosure relates to 2-(diethylamino)ethyl 2-(4-isobutylphenyl)propionate or pharmaceutically acceptable salts thereof for use in treatment of a subject, wherein 2-(diethylamino)ethyl 2-(4-isobutylphenyl)propionate and/or pharmaceutically acceptable salts thereof is topically administered to the subject, in particular to one or more sites of the subject, in an amount of about 1 mg to about 80 mg, in particular 1 mg to 80 mg, per day, in particular per day per site.

In some embodiments, the present disclosure relates to use of 2-(diethylamino)ethyl 2-(4-isobutylphenyl)propionate or pharmaceutically acceptable salts thereof in the manufacture of a medicament, wherein 2-(diethylamino)ethyl 2-(4-isobutylphenyl)propionate or pharmaceutically acceptable salts thereof is topically administered, to a subject, in particular to one or more sites of a subject, in an amount of about 1 mg to about 80 mg, in particular 1 mg to 80 mg, per day, in particular per day per site.

In some embodiments, the present disclosure relates to a kit for treatment of a subject, including 2-(diethylamino)ethyl 2-(4-isobutylphenyl)propionate and/or pharmaceutically acceptable salts thereof for being topically administered to the subject, in particular to one or more sites of the subject, in an amount of about 1 mg to about 80 mg, in particular 1 mg to 80 mg, per day, in particular per day per site.

In some embodiments, the present disclosure relates to a therapeutic system for treatment of a subject, including a composition of which 2-(diethylamino)ethyl 2-(4-isobutylphenyl)propionate is an active ingredient, 2-(diethylamino)ethyl 2-(4-isobutylphenyl)propionate present as a free base or as a pharmaceutically acceptable salt, wherein in the system, 2-(diethylamino)ethyl 2-(4-isobutylphenyl)propionate is topically administered to the subject, in particular to one or more sites of the subject, in an amount of about 1 mg to about 80 mg, in particular 1 mg to 80 mg, per day, in particular per day per site.

In some embodiments, the present disclosure relates to a method of treatment of a subject, including topical administrating 2-(diethylamino)ethyl 2-(4-isobutylphenyl)propionate and/or pharmaceutically acceptable salts thereof to the subject, in particular to one or more sites of the subject, in an amount of about 0.1 mg to about 40 mg, in particular 0.1 mg to 40 mg, per dose, in particular per dose per site.

In some embodiments, the present disclosure relates to 2-(diethylamino)ethyl 2-(4-isobutylphenyl)propionate or pharmaceutically acceptable salts thereof for use in treatment of a subject, wherein 2-(diethylamino)ethyl 2-(4-isobutylphenyl)propionate and/or pharmaceutically acceptable salts thereof is topically administered to the subject, in particular to one or more sites of the subject, in an amount of about 0.1 mg to about 40 mg, in particular 0.1 mg to 40 mg, per dose, in particular per dose per site.

In some embodiments, the present disclosure relates to use of 2-(diethylamino)ethyl 2-(4-isobutylphenyl)propionate or pharmaceutically acceptable salts thereof in the manufacture of a medicament, wherein 2-(diethylamino)ethyl 2-(4-isobutylphenyl)propionate or pharmaceutically acceptable salts thereof is topically administered, to a subject, in particular to one or more sites of a subject, in an amount of about 0.1 mg to about 40 mg, in particular 0.1 mg to 40 mg, per dose, in particular per dose per site.

In some embodiments, the present disclosure relates to a kit for treatment of a subject, including 2-(diethylamino)ethyl 2-(4-isobutylphenyl)propionate and/or pharmaceutically acceptable salts thereof for being topically administered to the subject, in particular to one or more sites of the subject, in an amount of about 0.1 mg to about 40 mg, in particular 0.1 mg to 40 mg, per dose, in particular per dose per site.

In some embodiments, the present disclosure relates to a therapeutic system for treatment of a subject, including a composition of which 2-(diethylamino)ethyl 2-(4-isobutylphenyl)propionate is an active ingredient, 2-(diethylamino)ethyl 2-(4-isobutylphenyl)propionate present as a free base or as a pharmaceutically acceptable salt, wherein in the system, 2-(diethylamino)ethyl 2-(4-isobutylphenyl)propionate is topically administered to the subject, in particular to one or more sites of the subject, in an amount of about 0.1 mg to about 40 mg, in particular 0.1 mg to 40 mg, per dose, in particular per dose per site.

In some embodiments, the present disclosure relates to a method of treatment of a subject, including topical administrating 2-(diethylamino)ethyl 2-(4-isobutylphenyl)propionate and/or pharmaceutically acceptable salts thereof to the subject, in particular to one or more sites of the subject, in an amount of about 5 μg/cm² to about 2 mg/cm², in particular 5 μg/cm² to 2 mg/cm², per dose, in particular per dose per site.

In some embodiments, the present disclosure relates to 2-(diethylamino)ethyl 2-(4-isobutylphenyl)propionate or pharmaceutically acceptable salts thereof for use in treatment of a subject, wherein 2-(diethylamino)ethyl 2-(4-isobutylphenyl)propionate and/or pharmaceutically acceptable salts thereof is topically administered to the subject, in particular to one or more sites of the subject, in an amount of about 5 μg/cm² to about 2 mg/cm², in particular 5 μg/cm² to 2 mg/cm², per dose, in particular per dose per site.

In some embodiments, the present disclosure relates to use of 2-(diethylamino)ethyl 2-(4-isobutylphenyl)propionate or pharmaceutically acceptable salts thereof in the manufacture of a medicament, wherein 2-(diethylamino)ethyl 2-(4-isobutylphenyl)propionate or pharmaceutically acceptable salts thereof is topically administered, to a subject, in particular to one or more sites of a subject, in an amount of about 5 μg/cm² to about 2 mg/cm², in particular 5 μg/cm² to 2 mg/cm², per dose, in particular per dose per site.

In some embodiments, the present disclosure relates to a kit for treatment of a subject, including 2-(diethylamino)ethyl 2-(4-isobutylphenyl)propionate and/or pharmaceutically acceptable salts thereof for being topically administered to the subject, in particular to one or more sites of the subject, in an amount of about 5 μg/cm² to about 2 mg/cm², in particular 5 μg/cm² to 2 mg/cm², per dose, in particular per dose per site.

In some embodiments, the present disclosure relates to a therapeutic system for treatment of a subject, including a composition of which 2-(diethylamino)ethyl 2-(4-isobutylphenyl)propionate is an active ingredient, 2-(diethylamino)ethyl 2-(4-isobutylphenyl)propionate present as a free base or as a pharmaceutically acceptable salt, wherein in the system, 2-(diethylamino)ethyl 2-(4-isobutylphenyl)propionate is topically administered to the subject, in particular to one or more sites of the subject, in an amount of about 5 μg/cm² to about 2 mg/cm², in particular 5 μg/cm² to 2 mg/cm², per dose, in particular per dose per site.

In some embodiments, the present disclosure relates to a dosage form, wherein a concentration of 2-(diethylamino)ethyl 2-(4-isobutylphenyl)propionate and/or pharmaceutically acceptable salts thereof in the dosage form is about 10 mg/mL to about 200 mg/mL, in particular 10 mg/mL to 200 mg/mL, or about 10 mg/g to about 200 mg/g, in particular 10 mg/g to 200 g.

In some embodiments, the present disclosure relates to a device capable of administering a unit dose of about 0.5 mg to about 30 mg, in particular 0.5 mg to 30 mg, of 2-(diethylamino)ethyl 2-(4-isobutylphenyl)propionate and/or pharmaceutically acceptable salts thereof.

In some embodiments, the present disclosure relates to a spray capable of spraying a unit dose of about 0.5 mg to about 30 mg, in particular 0.5 mg to 30 mg, of 2-(diethylamino)ethyl 2-(4-isobutylphenyl)propionate and/or pharmaceutically acceptable salts thereof.

In one aspect, the present disclosure is intended to assess the efficacy and safety of 2-(diethylamino)ethyl 2-(4-isobutylphenyl)propionate when administered to the knee as a topical spray in subjects with OA of the knee.

### BRIEF DESCRIPTION OF THE DRAWINGS

FIG. 1 is a study schema of the Phase 1 clinical study in some embodiments of the present disclosure.

FIG. 2 is a line chart of mean (SD) 2-(diethylamino)ethyl 2-(4-isobutylphenyl)propionate plasma concentration versus time by treatment on Day 1 (linear scale) for all randomized subjects in Phase 1 Clinical Study in some embodiments of the present disclosure.

FIG. 3 is a line chart of mean (SD) 2-(diethylamino)ethyl 2-(4-isobutylphenyl)propionate plasma concentration versus time by treatment on Day 12 (linear scale) for all randomized subjects in Phase 1 Clinical Study in some embodiments of the present disclosure.

FIG. 4 is a line chart of mean (SD) 2-(diethylamino)ethyl 2-(4-isobutylphenyl)propionate plasma concentration versus time by treatment on Day 1 (semi-log scale) for all randomized subjects in Phase 1 Clinical Study in some embodiments of the present disclosure.

FIG. 5 is a line chart of mean (SD) 2-(diethylamino)ethyl 2-(4-isobutylphenyl)propionate plasma concentration versus time by treatment on Day 12 (semi-log scale) for all randomized subjects in Phase 1 Clinical Study in some embodiments of the present disclosure.

FIG. 6 is a line chart of mean (SD) 2-(diethylamino)ethyl 2-(4-isobutylphenyl)propionate plasma concentration versus time by treatment on Day 1 from time 0 to 24 hours (linear scale) for all randomized subjects in Phase 1 Clinical Study in some embodiments of the present disclosure.

FIG. 7 is a line chart of mean (SD) 2-(diethylamino)ethyl 2-(4-isobutylphenyl)propionate plasma concentration versus time by treatment on Day 12 from time 0 to 48 hours (linear scale) for all randomized subjects in Phase 1 Clinical Study in some embodiments of the present disclosure.

FIG. 8 is a line chart of mean (SD) ibuprofen plasma concentration versus time by treatment on Day 1 (linear scale) for all randomized subjects in Phase 1 Clinical Study in some embodiments of the present disclosure.

FIG. 9 is a line chart of mean (SD) ibuprofen plasma concentration versus time by treatment on Day 12 (linear scale) for all randomized subjects in Phase 1 Clinical Study in some embodiments of the present disclosure.

FIG. 10 is a line chart of mean (SD) ibuprofen plasma concentration versus time by treatment on Day 1 (semi-log scale) for all randomized subjects in Phase 1 Clinical Study in some embodiments of the present disclosure.

FIG. 11 is a line chart of mean (SD) ibuprofen plasma concentration versus time by treatment on Day 12 (semi-log scale) for all randomized subjects in Phase 1 Clinical Study in some embodiments of the present disclosure.

FIG. 12 is a line chart of mean (SD) ibuprofen plasma concentration versus time by treatment on Day 1 from time 0 to 48 hours (linear scale) for all randomized subjects in Phase 1 Clinical Study in some embodiments of the present disclosure.

FIG. 13 is a line chart of mean (SD) ibuprofen plasma concentration versus time by treatment on Day 12 from time 0 to 48 hours (linear scale) for all randomized subjects in Phase 1 Clinical Study in some embodiments of the present disclosure.

FIG. 14 is a line chart of changes in WOMAC Pain Subscale Scores (mm) for transdermal 2-(diethylamino)ethyl 2-(4-isobutylphenyl)propionate 8.75, 17.5 and 35 mg/knee b.i.d. at week 2, 4, 8 and 12, and follow up (7 days after the treatment was stopped) in the Phase 2 clinical study in some embodiments of the present disclosure.

FIG. 15 is a line chart of changes in WOMAC Pain Subscale Scores (%) for transdermal 2-(diethylamino)ethyl 2-(4-isobutylphenyl)propionate 8.75, 17.5 and 35 mg/knee b.i.d. at week 2, 4, 8 and 12, and follow up (7 days after the treatment was stopped) in the Phase 2 clinical study in some embodiments of the present disclosure.

FIG. 16 is a line chart of changes in WOMAC Pain Scores (mm) for transdermal 2-(diethylamino)ethyl 2-(4-isobutylphenyl)propionate 8.75, 17.5 and 35 mg/knee b.i.d. at week 2, 4, 8 and 12, and follow up (7 days after the treatment was stopped) (adjusted by 58.3% of the testing drug amount) in the Phase 2 clinical study in some embodiments of the present disclosure.

FIG. 17 is a line chart of changes in WOMAC Pain Scores (%) for transdermal 2-(diethylamino)ethyl 2-(4-isobutylphenyl)propionate 8.75, 17.5 and 35 mg/knee b.i.d. at week 2, 4, 8 and 12, and follow up (7 days after the treatment was stopped) (adjusted by 58.3% of the testing drug amount) in the Phase 2 clinical study in some embodiments of the present disclosure.

FIG. 18 is a line chart of changes in WOMAC Joint Stiffness Subscale Scores (mm) for transdermal 2-(diethylamino)ethyl 2-(4-isobutylphenyl)propionate 8.75, 17.5 and 35 mg/knee b.i.d. at week 2, 4, 8 and 12, and follow up (7 days after the treatment was stopped) in the Phase 2 clinical study in some embodiments of the present disclosure.

FIG. 19 is a line chart of changes in WOMAC Joint Stiffness Subscale Scores (%) for transdermal 2-(diethylamino)ethyl 2-(4-isobutylphenyl)propionate 8.75, 17.5 and 35 mg/knee b.i.d. at week 2, 4, 8 and 12, and follow up (7 days after the treatment was stopped) in the Phase 2 clinical study in some embodiments of the present disclosure.

FIG. 20 is a line chart of changes in WOMAC Joint Stiffness Subscale Scores (mm) for transdermal 2-(diethylamino)ethyl 2-(4-isobutylphenyl)propionate 8.75, 17.5 and 35 mg/knee b.i.d. at week 2, 4, 8 and 12, and follow up (7 days after the treatment was stopped) (adjusted by 58.3% of the testing drug amount) in the Phase 2 clinical study in some embodiments of the present disclosure.

FIG. 21 is a line chart of changes in WOMAC Joint Stiffness Subscale Scores (%) for transdermal 2-(diethylamino)ethyl 2-(4-isobutylphenyl)propionate 8.75, 17.5 and 35 mg/knee b.i.d. at week 2, 4, 8 and 12, and follow up (7 days after the treatment was stopped) (adjusted by 58.3% of the testing drug amount) in the Phase 2 clinical study in some embodiments of the present disclosure.

FIG. 22 is a line chart of changes in WOMAC Difficulty Performing Daily Activities Subscale Scores (mm) for transdermal 2-(diethylamino)ethyl 2-(4-isobutylphenyl)propionate 8.75, 17.5 and 35 mg/knee b.i.d. at week 2, 4, 8 and 12, and follow up (7 days after the treatment was stopped) in the Phase 2 clinical study in some embodiments of the present disclosure.

FIG. 23 is a line chart of changes in WOMAC Difficulty Performing Daily Activities Subscale Scores (%) for transdermal 2-(diethylamino)ethyl 2-(4-isobutylphenyl)propionate 8.75, 17.5 and 35 mg/knee b.i.d. at week 2, 4, 8 and 12, and follow up (7 days after the treatment was stopped) in the Phase 2 clinical study in some embodiments of the present disclosure.

FIG. 24 is a line chart of changes in WOMAC Difficulty Performing Daily Activities Subscale Scores (mm) for transdermal 2-(diethylamino)ethyl 2-(4-isobutylphenyl)propionate 8.75, 17.5 and 35 mg/knee b.i.d. at week 2, 4, 8 and 12, and follow up (7 days after the treatment was stopped) (adjusted by 58.3% of the testing drug amount) in the Phase 2 clinical study in some embodiments of the present disclosure.

FIG. 25 is a line chart of changes in WOMAC Difficulty Performing Daily Activities Subscale Scores (%) for transdermal 2-(diethylamino)ethyl 2-(4-isobutylphenyl)propionate 8.75, 17.5 and 35 mg/knee b.i.d. at week 2, 4, 8 and 12, and follow up (7 days after the treatment was stopped) (adjusted by 58.3% of the testing drug amount) in the Phase 2 clinical study in some embodiments of the present disclosure.

FIG. 26 is a line chart of mean (SD) Ibuprofen Plasma Concentration vs. Time by Treatment at Week 8 (linear scale) (n=18-20) in the second Phase 2 clinical study in some embodiments of the present disclosure.

FIG. 27 is a line chart of mean (SD) Ibuprofen Plasma Concentration vs. Time by Treatment at Week 12 (linear scale) (n=18-20) in the second Phase 2 clinical study in some embodiments of the present disclosure.

### DETAILED DESCRIPTION OF THE DISCLOSURE

Unless defined otherwise, all technical and scientific terms used herein have the same meaning as commonly understood by one of ordinary skill in the art to which this invention belongs.

All publications, patents and other references mentioned herein are incorporated by reference in their entireties for all purposes.

In some embodiments, the present disclosure relates to a pharmaceutical composition capable of penetrating cartilage and methods of using the pharmaceutical composition for treating pains, especially osteoarthritis of humans and animals.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The terms “a” and “an” and “the” and similar references in the context of describing the invention (especially in the context of the following claims) are to be construed to cover both the singular and the plural, unless otherwise indicated herein or clearly contradicted by context. The use of the word “a” or “an” when used in conjunction with the term “comprising” in the claims and/or the specification may mean “one”, but it is also consistent with the meaning of “one or more”, “at least one”, and “one or more than one”.

Where the plural form is used for compounds, salts, and the like, this is taken to mean also a single compound, salt, or the like.

The term “and/or” as used herein refers to and encompasses any and all possible combinations of one or more of the associated listed items. When used in a list of two or more items, the term “and/or” means that any one of the listed items can be employed by itself, or any combination of two or more of the listed items can be employed. For example, if a composition, a combination, a constitution, a juxtaposition, or a group is described as including (or comprising) components A, B, C, and/or D, the composition can contain A alone; B alone; C alone; D alone; A and B in combination; A and C in combination; A and D in combination; B and C in combination; B and D in combination; C and D in combination; A, B, and C in combination; A, B, and D in combination; A, C, and D in combination; B, C, and D in combination; or A, B, C, and D in combination.

Throughout this application, the term “about” or “approximately” is used to indicate that a value includes the inherent variation of error for the device, the method being employed to determine the value, or the variation that exists among the study subjects. In one aspect, the terms “about” or “approximately” usually mean within 10%, in particular within 9%, in particular within 8%, in particular within 7%, in particular within 6%, in particular within 5%, in particular within 4%, in particular within 3%, in particular within 2%, in particular within 1%, of a given value or range.

The term “treat”, “treating” or “treatment” as used herein comprises treatment or therapeutic regimen relieving, reducing or alleviating at least one symptom in a patient or effecting a delay of progression of a proliferative disorder. For example, treatment can be the diminishment of one or several symptoms of a disorder or complete eradication of a disorder, such as osteoarthritis. Within the meaning of the present disclosure, the term “treat” also denotes to arrest, delay the onset (i.e., the period prior to clinical manifestation of a disorder) and/or reduce the risk of developing or worsening a disorder.

In some embodiments, the term “dose”, as used herein, means a drug or active component taken each time by an individual subject, in particular the total amount of a drug or active component taken each time by an individual subject, for one site.

In some embodiments, the term “dosage form”, as used herein, means a unit of administration of an active agent. Examples of dosage forms include tablets, capsules, injections, suspensions, liquids, emulsions, creams, ointments, suppositories, inhalable forms, transdermal forms, and the like.

In some embodiments, the term “unit dose” or “dosage unit” refers to a dosage form that is configured to deliver a specified quantity or dosage of composition or component thereof. Examples of dosage forms for topical administration include, but are not limited to, transdermal patch, cream, foam, gel, lotion, ointment, paste, powder, shake lotion, solid, sponge, tape, tinkture, vapor, injection, drops, rinces, spray, and solution. A “unit dose” or “dosage unit” may be configured to provide a full unit dose or fraction thereof (e.g., ½, ⅓, or ¼ of a dose). A predetermined quantity in each unit dose can depend on factors that include, but are not limited to, the unique characteristics of the active compound and the particular therapeutic effect to be achieved, and the limitations inherent in the art of creating and administering such unit doses. For instance, a unit dose may be, a transdermal patch, a spray, i.e., once spray in the spray application, a droplet of the dropping application, a certain length of the tape, rice-sized or bean-sized ointment, or a scoop or a spoon of ointment. Unit dose measuring devices, such as a cup, scoop, syringe, dropper, spoon, or colonic irrigation device, may hold the dosage form, for instance cream, foam, gel, lotion, ointment, paste, powder, shake lotion and solid, a measured quantity of composition equaling a full unit dose or fraction thereof (e.g., ½, ⅓, or ¼ of a dose). There may be a single unit dose, or multiple unit doses, in a single dose of administration. The kit may include instructions regarding the size of the unit dose, or fraction thereof.

The term “pharmaceutically acceptable” is defined herein to refer to those compounds, materials, compositions and/or dosage forms, which are, within the scope of sound medical judgment, suitable for contact with the tissues a patient without excessive toxicity, irritation allergic response and other problem complications commensurate with a reasonable benefit/risk ratio.

The term “pharmaceutical composition” is defined herein to refer to a substance or a mixture or solution containing at least one therapeutic agent to be administered to a patient, in order to prevent or treat, in particular to treat a particular disease or condition affecting the patient.

It is understood that the therapeutic agent may be administered each day in a single unit dose or multiple unit doses and/or administered each day in a single dose (once per day, q.d.) or divided doses (more than once per day, e.g., twice per day, b.i.d.).

The term “day” as used herein refers to either one calendar day in any time zone or one 24-hour period.

The terms “patient” or “subject” is intended to include animals, including warm-blooded animals. Examples of patients include mammals, e.g., humans, dogs, cows, horses, pigs, sheep, goats, cats, mice, rabbits, rats, and transgenic non-human animals. In some embodiments, the patient is a human, e.g., a human suffering from, at risk of suffering from, or potentially capable of suffering from a disease, for instance suffering from osteoarthritis.

In some embodiments, the term “transdermal administration” means administration of a transdermal dose, unit dose or dosage form; the term “transdermally administering” means administering a transdermal dose, unit dose or dosage form; and the term “transdermally administered” means administered by a transdermal dose, unit dose or dosage form. That a patient and/or subject is “transdermally administered” is equivalent to that a patient and/or subject is subjected to a “transdermal administration”. “Transdermally administering” to a patient and/or subject is equivalent to subjecting a “transdermal administration” to a patient and/or subject.

In some embodiments, the term “site” (of the subject) is the region/location of a human body where a symptom was found, for example, joint, muscle, bone and/or cartilage, etc., itself having the symptom, particular the pain, the inflammation and/or the disease, particularly the symptom, pain, inflammation and/or disease of joint, muscle, bone and/or cartilage, etc., more particularly osteoarthritis; and/or the joint, muscle, bone and/or cartilage, etc., where a cause of the symptom, particular the pain, the inflammation and/or the disease of joint, muscle, bone and/or cartilage etc., more particularly osteoarthritis, is located.

In some embodiments, correspondingly, the term “administrating to the site” (of the subject) means administrating to: (a) the place on the skin and/or body surface which is in correspondence with, or, close to the “site”; and/or (b) the place on the skin and/or body surface that provides an accessible route to the “site”.

For instance, the site could be the joint itself suffering from, at risk of suffering from, or potentially capable of suffering from the symptom, particular the pain, the inflammation and/or disease of joint, muscle, bone and/or cartilage, etc., more particularly osteoarthritis, while administrating to the site could be administrating to the skin and/or body surface near, in particular within about 1 cm to about 15 cm, in particular about 3 cm to about 10 cm, in particular a distance selected from about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, from the joint, muscle, bone or cartilage etc., and/or an environment about 1 cm to about 15 cm, in particular about 3 cm to about 10 cm, in particular a distance selected from about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, around all directions of the joint, muscle, bone or cartilage etc.

In some embodiments, the term “close” or “close to” means within about 1 cm to about 15 cm, in particular about 3 cm to about 10 cm, in particular a distance selected from about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, from the center of the site, i.e. joint, muscle, bone or cartilage etc., and/or an environment about 1 cm to about 15 cm, in particular about 3 cm to about 10 cm, in particular a distance selected from about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, around all directions of the center of the site, i.e. joint, muscle, bone or cartilage, etc.

In some embodiments, the term “symptom” refers to any symptoms such as diseases, inflammation, pain, fever, gout, dysmenorrhea, joint swelling, morning stiffness, rheumatoid disorders, or injury. In particular, the symptom could be pain or inflammation related to musculoskeletal system, for instance arthritis, in particular osteoarthritis or rheumatoid arthritis.

In some embodiments, the term “pain” refers to any pain such as acute pain, bone pain, joint pain, muscle pain, cartilage pain, migraine pain, headache, cluster headache, menstrual cramps, neuropathic pain, post-operative pain, chronic lower back pain, herpes neuralgia, phantom limb pain, central pain, dental pain, neuropathic pain, opioid-resistant pain, visceral pain, surgical pain, injury pain, pain during labor and delivery, pain resulting from burns, sunburn, gout, lupus, fibromyalgia, post-partum pain, angina pain, cystitis, inflammation, arthritis pain, septic arthritis pain, osteoarthritis pain, juvenile rheumatoid arthritis pain, ankylosing spondylitis and dysmenorrhea.

In some embodiments, 2-(diethylamino)ethyl 2-(4-isobutylphenyl)propionate or pharmaceutically acceptable salts thereof is administered in an amount of about 1 mg to about 64 mg, in particular 1 mg to 64 mg, per day, in particular per day per site. In some embodiments, 2-(diethylamino)ethyl 2-(4-isobutylphenyl)propionate or pharmaceutically acceptable salts thereof is administered in an amount of about 2 mg to about 56 mg, in particular 2 mg to 56 mg, per day, in particular per day per site. In some embodiments, 2-(diethylamino)ethyl 2-(4-isobutylphenyl)propionate or pharmaceutically acceptable salts thereof is administered in an amount of about 3 mg to about 48 mg, in particular 3 mg to 48 mg, per day, in particular per day per site. In some embodiments, 2-(diethylamino)ethyl 2-(4-isobutylphenyl)propionate or pharmaceutically acceptable salts thereof is administered in an amount of about 4 mg to about 40 mg, in particular 4 mg to 40 mg, per day, in particular per day per site. In some embodiments, 2-(diethylamino)ethyl 2-(4-isobutylphenyl)propionate or pharmaceutically acceptable salts thereof is administered in an amount of about 4 mg to about 32 mg, in particular 4 mg to 32 mg, per day, in particular per day per site. In some embodiments, 2-(diethylamino)ethyl 2-(4-isobutylphenyl)propionate or pharmaceutically acceptable salts thereof is administered in an amount of about 8 mg to about 32 mg, in particular 8 mg to 32 mg, per day, in particular per day per site. In some embodiments, 2-(diethylamino)ethyl 2-(4-isobutylphenyl)propionate or pharmaceutically acceptable salts thereof is administered in an amount of about 8 mg to about 18 mg, in particular 8 mg to 18 mg, per day, in particular per day per site. In some embodiments, 2-(diethylamino)ethyl 2-(4-isobutylphenyl)propionate or pharmaceutically acceptable salts thereof is administered in an amount of about 4 mg to about 8 mg, in particular 4 mg to 8 mg, per day, in particular per day per site. In some embodiments, 2-(diethylamino)ethyl 2-(4-isobutylphenyl)propionate or pharmaceutically acceptable salts thereof is administered in an amount of about 8 mg to about 16 mg, in particular 8 mg to 16 mg, per day, in particular per day per site. In some embodiments, 2-(diethylamino)ethyl 2-(4-isobutylphenyl)propionate or pharmaceutically acceptable salts thereof is administered in an amount of about 16 mg to about 32 mg, in particular 16 mg to 32 mg, per day, in particular per day per site. In some embodiments, 2-(diethylamino)ethyl 2-(4-isobutylphenyl)propionate or pharmaceutically acceptable salts thereof is administered in an amount selected from a group consisting of 4.5 mg, 9 mg, 13.5 mg, 18 mg, 22.5 mg, 27 mg, 31.5 mg, 36 mg, 40.5 mg, 45 mg, 49.5 mg, 54 mg, 58.5 mg, 63 mg, 67.5 mg and 72 mg, per day, in particular per day per site.

In some embodiments, 2-(diethylamino)ethyl 2-(4-isobutylphenyl)propionate or pharmaceutically acceptable salts thereof is administered in an amount selected from a group consisting of 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, 5 mg, 5.1 mg, 5.2 mg, 5.3 mg, 5.4 mg, 5.5 mg, 5.6 mg, 5.7 mg, 5.8 mg, 5.9 mg, 6 mg, 6.1 mg, 6.2 mg, 6.3 mg, 6.4 mg, 6.5 mg, 6.6 mg, 6.7 mg, 6.8 mg, 6.9 mg, 7 mg, 7.1 mg, 7.2 mg, 7.3 mg, 7.4 mg, 7.5 mg, 7.6 mg, 7.7 mg, 7.8 mg, 7.9 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, 10 mg, 10.1 mg, 10.2 mg, 10.3 mg, 10.4 mg, 10.5 mg, 10.6 mg, 10.7 mg, 10.8 mg, 10.9 mg, 11 mg, 11.1 mg, 11.2 mg, 11.3 mg, 11.4 mg, 11.5 mg, 11.6 mg, 11.7 mg, 11.8 mg, 11.9 mg, 12 mg, 12.1 mg, 12.2 mg, 12.3 mg, 12.4 mg, 12.5 mg, 12.6 mg, 12.7 mg, 12.8 mg, 12.9 mg, 13 mg, 13.1 mg, 13.2 mg, 13.3 mg, 13.4 mg, 13.5 mg, 13.6 mg, 13.7 mg, 13.8 mg, 13.9 mg, 14 mg, 14.1 mg, 14.2 mg, 14.3 mg, 14.4 mg, 14.5 mg, 14.6 mg, 14.7 mg, 14.8 mg, 14.9 mg, 15 mg, 15.1 mg, 15.2 mg, 15.3 mg, 15.4 mg, 15.5 mg, 15.6 mg, 15.7 mg, 15.8 mg, 15.9 mg, 16 mg, 16.1 mg, 16.2 mg, 16.3 mg, 16.4 mg, 16.5 mg, 16.6 mg, 16.7 mg, 16.8 mg, 16.9 mg, 17 mg, 17.1 mg, 17.2 mg, 17.3 mg, 17.4 mg, 17.5 mg, 17.6 mg, 17.7 mg, 17.8 mg, 17.9 mg, 18 mg, 18.1 mg, 18.2 mg, 18.3 mg, 18.4 mg, 18.5 mg, 18.6 mg, 18.7 mg, 18.8 mg, 18.9 mg, 19 mg, 19.1 mg, 19.2 mg, 19.3 mg, 19.4 mg, 19.5 mg, 19.6 mg, 19.7 mg, 19.8 mg, 19.9 mg, 20 mg, 20.1 mg, 20.2 mg, 20.3 mg, 20.4 mg, 20.5 mg, 20.6 mg, 20.7 mg, 20.8 mg, 20.9 mg, 21 mg, 21.1 mg, 21.2 mg, 21.3 mg, 21.4 mg, 21.5 mg, 21.6 mg, 21.7 mg, 21.8 mg, 21.9 mg, 22 mg, 22.1 mg, 22.2 mg, 22.3 mg, 22.4 mg, 22.5 mg, 22.6 mg, 22.7 mg, 22.8 mg, 22.9 mg, 23 mg, 23.1 mg, 23.2 mg, 23.3 mg, 23.4 mg, 23.5 mg, 23.6 mg, 23.7 mg, 23.8 mg, 23.9 mg, 24 mg, 24.1 mg, 24.2 mg, 24.3 mg, 24.4 mg, 24.5 mg, 24.6 mg, 24.7 mg, 24.8 mg, 24.9 mg, 25 mg, 25.1 mg, 25.2 mg, 25.3 mg, 25.4 mg, 25.5 mg, 25.6 mg, 25.7 mg, 25.8 mg, 25.9 mg, 26 mg, 26.1 mg, 26.2 mg, 26.3 mg, 26.4 mg, 26.5 mg, 26.6 mg, 26.7 mg, 26.8 mg, 26.9 mg, 27 mg, 27.1 mg, 27.2 mg, 27.3 mg, 27.4 mg, 27.5 mg, 27.6 mg, 27.7 mg, 27.8 mg, 27.9 mg, 28 mg, 28.1 mg, 28.2 mg, 28.3 mg, 28.4 mg, 28.5 mg, 28.6 mg, 28.7 mg, 28.8 mg, 28.9 mg, 29 mg, 29.1 mg, 29.2 mg, 29.3 mg, 29.4 mg, 29.5 mg, 29.6 mg, 29.7 mg, 29.8 mg, 29.9 mg, 30 mg, 30.1 mg, 30.2 mg, 30.3 mg, 30.4 mg, 30.5 mg, 30.6 mg, 30.7 mg, 30.8 mg, 30.9 mg, 31 mg, 31.1 mg, 31.2 mg, 31.3 mg, 31.4 mg, 31.5 mg, 31.6 mg, 31.7 mg, 31.8 mg, 31.9 mg, 32 mg, 32.1 mg, 32.2 mg, 32.3 mg, 32.4 mg, 32.5 mg, 32.6 mg, 32.7 mg, 32.8 mg, 32.9 mg, 33 mg, 33.1 mg, 33.2 mg, 33.3 mg, 33.4 mg, 33.5 mg, 33.6 mg, 33.7 mg, 33.8 mg, 33.9 mg, 34 mg, 34.1 mg, 34.2 mg, 34.3 mg, 34.4 mg, 34.5 mg, 34.6 mg, 34.7 mg, 34.8 mg, 34.9 mg, 35 mg, 35.1 mg, 35.2 mg, 35.3 mg, 35.4 mg, 35.5 mg, 35.6 mg, 35.7 mg, 35.8 mg, 35.9 mg, 36 mg, 36.1 mg, 36.2 mg, 36.3 mg, 36.4 mg, 36.5 mg, 36.6 mg, 36.7 mg, 36.8 mg, 36.9 mg, 37 mg, 37.1 mg, 37.2 mg, 37.3 mg, 37.4 mg, 37.5 mg, 37.6 mg, 37.7 mg, 37.8 mg, 37.9 mg, 38 mg, 38.1 mg, 38.2 mg, 38.3 mg, 38.4 mg, 38.5 mg, 38.6 mg, 38.7 mg, 38.8 mg, 38.9 mg, 39 mg, 39.1 mg, 39.2 mg, 39.3 mg, 39.4 mg, 39.5 mg, 39.6 mg, 39.7 mg, 39.8 mg, 39.9 mg, 40 mg, 40.1 mg, 40.2 mg, 40.3 mg, 40.4 mg, 40.5 mg, 40.6 mg, 40.7 mg, 40.8 mg, 40.9 mg, 41 mg, 41.1 mg, 41.2 mg, 41.3 mg, 41.4 mg, 41.5 mg, 41.6 mg, 41.7 mg, 41.8 mg, 41.9 mg, 42 mg, 42.1 mg, 42.2 mg, 42.3 mg, 42.4 mg, 42.5 mg, 42.6 mg, 42.7 mg, 42.8 mg, 42.9 mg, 43 mg, 43.1 mg, 43.2 mg, 43.3 mg, 43.4 mg, 43.5 mg, 43.6 mg, 43.7 mg, 43.8 mg, 43.9 mg, 44 mg, 44.1 mg, 44.2 mg, 44.3 mg, 44.4 mg, 44.5 mg, 44.6 mg, 44.7 mg, 44.8 mg, 44.9 mg, 45 mg, 45.1 mg, 45.2 mg, 45.3 mg, 45.4 mg, 45.5 mg, 45.6 mg, 45.7 mg, 45.8 mg, 45.9 mg, 46 mg, 46.1 mg, 46.2 mg, 46.3 mg, 46.4 mg, 46.5 mg, 46.6 mg, 46.7 mg, 46.8 mg, 46.9 mg, 47 mg, 47.1 mg, 47.2 mg, 47.3 mg, 47.4 mg, 47.5 mg, 47.6 mg, 47.7 mg, 47.8 mg, 47.9 mg, 48 mg, 48.1 mg, 48.2 mg, 48.3 mg, 48.4 mg, 48.5 mg, 48.6 mg, 48.7 mg, 48.8 mg, 48.9 mg, 49 mg, 49.1 mg, 49.2 mg, 49.3 mg, 49.4 mg, 49.5 mg, 49.6 mg, 49.7 mg, 49.8 mg, 49.9 mg, 50 mg, 50.1 mg, 50.2 mg, 50.3 mg, 50.4 mg, 50.5 mg, 50.6 mg, 50.7 mg, 50.8 mg, 50.9 mg, 51 mg, 51.1 mg, 51.2 mg, 51.3 mg, 51.4 mg, 51.5 mg, 51.6 mg, 51.7 mg, 51.8 mg, 51.9 mg, 52 mg, 52.1 mg, 52.2 mg, 52.3 mg, 52.4 mg, 52.5 mg, 52.6 mg, 52.7 mg, 52.8 mg, 52.9 mg, 53 mg, 53.1 mg, 53.2 mg, 53.3 mg, 53.4 mg, 53.5 mg, 53.6 mg, 53.7 mg, 53.8 mg, 53.9 mg, 54 mg, 54.1 mg, 54.2 mg, 54.3 mg, 54.4 mg, 54.5 mg, 54.6 mg, 54.7 mg, 54.8 mg, 54.9 mg, 55 mg, 55.1 mg, 55.2 mg, 55.3 mg, 55.4 mg, 55.5 mg, 55.6 mg, 55.7 mg, 55.8 mg, 55.9 mg, 56 mg, 56.1 mg, 56.2 mg, 56.3 mg, 56.4 mg, 56.5 mg, 56.6 mg, 56.7 mg, 56.8 mg, 56.9 mg, 57 mg, 57.1 mg, 57.2 mg, 57.3 mg, 57.4 mg, 57.5 mg, 57.6 mg, 57.7 mg, 57.8 mg, 57.9 mg, 58 mg, 58.1 mg, 58.2 mg, 58.3 mg, 58.4 mg, 58.5 mg, 58.6 mg, 58.7 mg, 58.8 mg, 58.9 mg, 59 mg, 59.1 mg, 59.2 mg, 59.3 mg, 59.4 mg, 59.5 mg, 59.6 mg, 59.7 mg, 59.8 mg, 59.9 mg, 60 mg, 60.1 mg, 60.2 mg, 60.3 mg, 60.4 mg, 60.5 mg, 60.6 mg, 60.7 mg, 60.8 mg, 60.9 mg, 61 mg, 61.1 mg, 61.2 mg, 61.3 mg, 61.4 mg, 61.5 mg, 61.6 mg, 61.7 mg, 61.8 mg, 61.9 mg, 62 mg, 62.1 mg, 62.2 mg, 62.3 mg, 62.4 mg, 62.5 mg, 62.6 mg, 62.7 mg, 62.8 mg, 62.9 mg, 63 mg, 63.1 mg, 63.2 mg, 63.3 mg, 63.4 mg, 63.5 mg, 63.6 mg, 63.7 mg, 63.8 mg, 63.9 mg, 64 mg, 64.1 mg, 64.2 mg, 64.3 mg, 64.4 mg, 64.5 mg, 64.6 mg, 64.7 mg, 64.8 mg, 64.9 mg, 65 mg, 65.1 mg, 65.2 mg, 65.3 mg, 65.4 mg, 65.5 mg, 65.6 mg, 65.7 mg, 65.8 mg, 65.9 mg, 66 mg, 66.1 mg, 66.2 mg, 66.3 mg, 66.4 mg, 66.5 mg, 66.6 mg, 66.7 mg, 66.8 mg, 66.9 mg, 67 mg, 67.1 mg, 67.2 mg, 67.3 mg, 67.4 mg, 67.5 mg, 67.6 mg, 67.7 mg, 67.8 mg, 67.9 mg, 68 mg, 68.1 mg, 68.2 mg, 68.3 mg, 68.4 mg, 68.5 mg, 68.6 mg, 68.7 mg, 68.8 mg, 68.9 mg, 69 mg, 69.1 mg, 69.2 mg, 69.3 mg, 69.4 mg, 69.5 mg, 69.6 mg, 69.7 mg, 69.8 mg, 69.9 mg, 70 mg, 70.1 mg, 70.2 mg, 70.3 mg, 70.4 mg, 70.5 mg, 70.6 mg, 70.7 mg, 70.8 mg, 70.9 mg, 71 mg, 71.1 mg, 71.2 mg, 71.3 mg, 71.4 mg, 71.5 mg, 71.6 mg, 71.7 mg, 71.8 mg, 71.9 mg, 72 mg, 72.1 mg, 72.2 mg, 72.3 mg, 72.4 mg, 72.5 mg, 72.6 mg, 72.7 mg, 72.8 mg, 72.9 mg, 73 mg, 73.1 mg, 73.2 mg, 73.3 mg, 73.4 mg, 73.5 mg, 73.6 mg, 73.7 mg, 73.8 mg, 73.9 mg, 74 mg, 74.1 mg, 74.2 mg, 74.3 mg, 74.4 mg, 74.5 mg, 74.6 mg, 74.7 mg, 74.8 mg, 74.9 mg, 75 mg, 75.1 mg, 75.2 mg, 75.3 mg, 75.4 mg, 75.5 mg, 75.6 mg, 75.7 mg, 75.8 mg, 75.9 mg, 76 mg, 76.1 mg, 76.2 mg, 76.3 mg, 76.4 mg, 76.5 mg, 76.6 mg, 76.7 mg, 76.8 mg, 76.9 mg, 77 mg, 77.1 mg, 77.2 mg, 77.3 mg, 77.4 mg, 77.5 mg, 77.6 mg, 77.7 mg, 77.8 mg, 77.9 mg, 78 mg, 78.1 mg, 78.2 mg, 78.3 mg, 78.4 mg, 78.5 mg, 78.6 mg, 78.7 mg, 78.8 mg, 78.9 mg, 79 mg, 79.1 mg, 79.2 mg, 79.3 mg, 79.4 mg, 79.5 mg, 79.6 mg, 79.7 mg, 79.8 mg, 79.9 mg, and 80 mg, per day, in particular per day per site.

In some embodiments, 2-(diethylamino)ethyl 2-(4-isobutylphenyl)propionate or pharmaceutically acceptable salts thereof is administered in an amount selected from a group consisting of 4.375 mg, 8.75 mg, 13.125 mg, 17.5 mg, 21.875 mg, 26.25 mg, 30.625 mg, 35 mg, 39.375 mg, 43.75 mg, 48.125 mg, 52.5 mg, 56.875 mg, 61.25 mg, 65.625 mg, and 70 mg, per day, in particular per day per site.

In some embodiments, 2-(diethylamino)ethyl 2-(4-isobutylphenyl)propionate or pharmaceutically acceptable salts thereof is administered in an amount of about 0.1 mg to about 40 mg, in particular 0.1 mg to 40 mg per dose, in particular per dose per site. In some embodiments, 2-(diethylamino)ethyl 2-(4-isobutylphenyl)propionate or pharmaceutically acceptable salts thereof is administered in an amount of about 1 mg to about 32 mg, in particular 1 mg to 32 mg, per dose, in particular per dose per site. In some embodiments, 2-(diethylamino)ethyl 2-(4-isobutylphenyl)propionate or pharmaceutically acceptable salts thereof is administered in an amount of about 1.5 mg to about 24 mg, in particular 1.5 mg to 24 mg, per dose, in particular per dose per site. In some embodiments, 2-(diethylamino)ethyl 2-(4-isobutylphenyl)propionate or pharmaceutically acceptable salts thereof is administered in an amount of about 2 mg to about 20 mg, in particular 2 mg to 20 mg, per dose, in particular per dose per site. In some embodiments, 2-(diethylamino)ethyl 2-(4-isobutylphenyl)propionate or pharmaceutically acceptable salts thereof is administered in an amount of about 4 mg to about 18 mg, in particular 4 mg to 18 mg, per dose, in particular per dose per site. In some embodiments, 2-(diethylamino)ethyl 2-(4-isobutylphenyl)propionate or pharmaceutically acceptable salts thereof is administered in an amount of about 4 mg to about 8 mg, in particular 4 mg to 8 mg, per dose, in particular per dose per site. In some embodiments, 2-(diethylamino)ethyl 2-(4-isobutylphenyl)propionate or pharmaceutically acceptable salts thereof is administered in an amount of about 8 mg to about 16 mg, in particular 8 mg to 16 mg, per dose, in particular per dose per site. In some embodiments, 2-(diethylamino)ethyl 2-(4-isobutylphenyl)propionate or pharmaceutically acceptable salts thereof is administered in an amount of about 16 mg to about 32 mg, in particular 16 mg to 32 mg, per dose, in particular per dose per site. In some embodiments, 2-(diethylamino)ethyl 2-(4-isobutylphenyl)propionate or pharmaceutically acceptable salts thereof is administered in an amount selected from a group consisting of 1 mg, 2.25 mg, 4.5 mg, 6.75 mg, 9 mg, 11.25 mg, 13.5 mg, 15.75 mg, 18 mg, 20.25 mg, 22.5 mg, 24.75 mg, 27 mg, 29.25 mg, 31.5 mg, 33.75 mg, and 36 mg, per dose, in particular per dose per site.

In some embodiments, 2-(diethylamino)ethyl 2-(4-isobutylphenyl)propionate or pharmaceutically acceptable salts thereof is administered in an amount selected from a group consisting of 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.55 mg, 1.6 mg, 1.65 mg, 1.7 mg, 1.75 mg, 1.8 mg, 1.85 mg, 1.9 mg, 1.95 mg, 2 mg, 2.05 mg, 2.1 mg, 2.15 mg, 2.2 mg, 2.25 mg, 2.3 mg, 2.35 mg, 2.4 mg, 2.45 mg, 2.5 mg, 2.55 mg, 2.6 mg, 2.65 mg, 2.7 mg, 2.75 mg, 2.8 mg, 2.85 mg, 2.9 mg, 2.95 mg, 3 mg, 3.05 mg, 3.1 mg, 3.15 mg, 3.2 mg, 3.25 mg, 3.3 mg, 3.35 mg, 3.4 mg, 3.45 mg, 3.5 mg, 3.55 mg, 3.6 mg, 3.65 mg, 3.7 mg, 3.75 mg, 3.8 mg, 3.85 mg, 3.9 mg, 3.95 mg, 4 mg, 4.05 mg, 4.1 mg, 4.15 mg, 4.2 mg, 4.25 mg, 4.3 mg, 4.35 mg, 4.4 mg, 4.45 mg, 4.5 mg, 4.55 mg, 4.6 mg, 4.65 mg, 4.7 mg, 4.75 mg, 4.8 mg, 4.85 mg, 4.9 mg, 4.95 mg, 5 mg, 5.05 mg, 5.1 mg, 5.15 mg, 5.2 mg, 5.25 mg, 5.3 mg, 5.35 mg, 5.4 mg, 5.45 mg, 5.5 mg, 5.55 mg, 5.6 mg, 5.65 mg, 5.7 mg, 5.75 mg, 5.8 mg, 5.85 mg, 5.9 mg, 5.95 mg, 6 mg, 6.05 mg, 6.1 mg, 6.15 mg, 6.2 mg, 6.25 mg, 6.3 mg, 6.35 mg, 6.4 mg, 6.45 mg, 6.5 mg, 6.55 mg, 6.6 mg, 6.65 mg, 6.7 mg, 6.75 mg, 6.8 mg, 6.85 mg, 6.9 mg, 6.95 mg, 7 mg, 7.05 mg, 7.1 mg, 7.15 mg, 7.2 mg, 7.25 mg, 7.3 mg, 7.35 mg, 7.4 mg, 7.45 mg, 7.5 mg, 7.55 mg, 7.6 mg, 7.65 mg, 7.7 mg, 7.75 mg, 7.8 mg, 7.85 mg, 7.9 mg, 7.95 mg, 8 mg, 8.05 mg, 8.1 mg, 8.15 mg, 8.2 mg, 8.25 mg, 8.3 mg, 8.35 mg, 8.4 mg, 8.45 mg, 8.5 mg, 8.55 mg, 8.6 mg, 8.65 mg, 8.7 mg, 8.75 mg, 8.8 mg, 8.85 mg, 8.9 mg, 8.95 mg, 9 mg, 9.05 mg, 9.1 mg, 9.15 mg, 9.2 mg, 9.25 mg, 9.3 mg, 9.35 mg, 9.4 mg, 9.45 mg, 9.5 mg, 9.55 mg, 9.6 mg, 9.65 mg, 9.7 mg, 9.75 mg, 9.8 mg, 9.85 mg, 9.9 mg, 9.95 mg, 10 mg, 10.05 mg, 10.1 mg, 10.15 mg, 10.2 mg, 10.25 mg, 10.3 mg, 10.35 mg, 10.4 mg, 10.45 mg, 10.5 mg, 10.55 mg, 10.6 mg, 10.65 mg, 10.7 mg, 10.75 mg, 10.8 mg, 10.85 mg, 10.9 mg, 10.95 mg, 11 mg, 11.05 mg, 11.1 mg, 11.15 mg, 11.2 mg, 11.25 mg, 11.3 mg, 11.35 mg, 11.4 mg, 11.45 mg, 11.5 mg, 11.55 mg, 11.6 mg, 11.65 mg, 11.7 mg, 11.75 mg, 11.8 mg, 11.85 mg, 11.9 mg, 11.95 mg, 12 mg, 12.05 mg, 12.1 mg, 12.15 mg, 12.2 mg, 12.25 mg, 12.3 mg, 12.35 mg, 12.4 mg, 12.45 mg, 12.5 mg, 12.55 mg, 12.6 mg, 12.65 mg, 12.7 mg, 12.75 mg, 12.8 mg, 12.85 mg, 12.9 mg, 12.95 mg, 13 mg, 13.05 mg, 13.1 mg, 13.15 mg, 13.2 mg, 13.25 mg, 13.3 mg, 13.35 mg, 13.4 mg, 13.45 mg, 13.5 mg, 13.55 mg, 13.6 mg, 13.65 mg, 13.7 mg, 13.75 mg, 13.8 mg, 13.85 mg, 13.9 mg, 13.95 mg, 14 mg, 14.05 mg, 14.1 mg, 14.15 mg, 14.2 mg, 14.25 mg, 14.3 mg, 14.35 mg, 14.4 mg, 14.45 mg, 14.5 mg, 14.55 mg, 14.6 mg, 14.65 mg, 14.7 mg, 14.75 mg, 14.8 mg, 14.85 mg, 14.9 mg, 14.95 mg, 15 mg, 15.05 mg, 15.1 mg, 15.15 mg, 15.2 mg, 15.25 mg, 15.3 mg, 15.35 mg, 15.4 mg, 15.45 mg, 15.5 mg, 15.55 mg, 15.6 mg, 15.65 mg, 15.7 mg, 15.75 mg, 15.8 mg, 15.85 mg, 15.9 mg, 15.95 mg, 16 mg, 16.05 mg, 16.1 mg, 16.15 mg, 16.2 mg, 16.25 mg, 16.3 mg, 16.35 mg, 16.4 mg, 16.45 mg, 16.5 mg, 16.55 mg, 16.6 mg, 16.65 mg, 16.7 mg, 16.75 mg, 16.8 mg, 16.85 mg, 16.9 mg, 16.95 mg, 17 mg, 17.05 mg, 17.1 mg, 17.15 mg, 17.2 mg, 17.25 mg, 17.3 mg, 17.35 mg, 17.4 mg, 17.45 mg, 17.5 mg, 17.55 mg, 17.6 mg, 17.65 mg, 17.7 mg, 17.75 mg, 17.8 mg, 17.85 mg, 17.9 mg, 17.95 mg, 18 mg, 18.05 mg, 18.1 mg, 18.15 mg, 18.2 mg, 18.25 mg, 18.3 mg, 18.35 mg, 18.4 mg, 18.45 mg, 18.5 mg, 18.55 mg, 18.6 mg, 18.65 mg, 18.7 mg, 18.75 mg, 18.8 mg, 18.85 mg, 18.9 mg, 18.95 mg, 19 mg, 19.05 mg, 19.1 mg, 19.15 mg, 19.2 mg, 19.25 mg, 19.3 mg, 19.35 mg, 19.4 mg, 19.45 mg, 19.5 mg, 19.55 mg, 19.6 mg, 19.65 mg, 19.7 mg, 19.75 mg, 19.8 mg, 19.85 mg, 19.9 mg, 19.95 mg, 20 mg, 20.05 mg, 20.1 mg, 20.15 mg, 20.2 mg, 20.25 mg, 20.3 mg, 20.35 mg, 20.4 mg, 20.45 mg, 20.5 mg, 20.55 mg, 20.6 mg, 20.65 mg, 20.7 mg, 20.75 mg, 20.8 mg, 20.85 mg, 20.9 mg, 20.95 mg, 21 mg, 21.05 mg, 21.1 mg, 21.15 mg, 21.2 mg, 21.25 mg, 21.3 mg, 21.35 mg, 21.4 mg, 21.45 mg, 21.5 mg, 21.55 mg, 21.6 mg, 21.65 mg, 21.7 mg, 21.75 mg, 21.8 mg, 21.85 mg, 21.9 mg, 21.95 mg, 22 mg, 22.05 mg, 22.1 mg, 22.15 mg, 22.2 mg, 22.25 mg, 22.3 mg, 22.35 mg, 22.4 mg, 22.45 mg, and 22.5 mg, 22.55 mg, 22.6 mg, 22.65 mg, 22.7 mg, 22.75 mg, 22.8 mg, 22.85 mg, 22.9 mg, 22.95 mg, 23 mg, 23.05 mg, 23.1 mg, 23.15 mg, 23.2 mg, 23.25 mg, 23.3 mg, 23.35 mg, 23.4 mg, 23.45 mg, 23.5 mg, 23.55 mg, 23.6 mg, 23.65 mg, 23.7 mg, 23.75 mg, 23.8 mg, 23.85 mg, 23.9 mg, 23.95 mg, 24 mg, 24.05 mg, 24.1 mg, 24.15 mg, 24.2 mg, 24.25 mg, 24.3 mg, 24.35 mg, 24.4 mg, 24.45 mg, 24.5 mg, 24.55 mg, 24.6 mg, 24.65 mg, 24.7 mg, 24.75 mg, 24.8 mg, 24.85 mg, 24.9 mg, 24.95 mg, 25 mg, 25.05 mg, 25.1 mg, 25.15 mg, 25.2 mg, 25.25 mg, 25.3 mg, 25.35 mg, 25.4 mg, 25.45 mg, 25.5 mg, 25.55 mg, 25.6 mg, 25.65 mg, 25.7 mg, 25.75 mg, 25.8 mg, 25.85 mg, 25.9 mg, 25.95 mg, 26 mg, 26.05 mg, 26.1 mg, 26.15 mg, 26.2 mg, 26.25 mg, 26.3 mg, 26.35 mg, 26.4 mg, 26.45 mg, 26.5 mg, 26.55 mg, 26.6 mg, 26.65 mg, 26.7 mg, 26.75 mg, 26.8 mg, 26.85 mg, 26.9 mg, 26.95 mg, 27 mg, 27.05 mg, 27.1 mg, 27.15 mg, 27.2 mg, 27.25 mg, 27.3 mg, 27.35 mg, 27.4 mg, 27.45 mg, 27.5 mg, 27.55 mg, 27.6 mg, 27.65 mg, 27.7 mg, 27.75 mg, 27.8 mg, 27.85 mg, 27.9 mg, 27.95 mg, 28 mg, 28.05 mg, 28.1 mg, 28.15 mg, 28.2 mg, 28.25 mg, 28.3 mg, 28.35 mg, 28.4 mg, 28.45 mg, 28.5 mg, 28.55 mg, 28.6 mg, 28.65 mg, 28.7 mg, 28.75 mg, 28.8 mg, 28.85 mg, 28.9 mg, 28.95 mg, 29 mg, 29.05 mg, 29.1 mg, 29.15 mg, 29.2 mg, 29.25 mg, 29.3 mg, 29.35 mg, 29.4 mg, 29.45 mg, 29.5 mg, 29.55 mg, 29.6 mg, 29.65 mg, 29.7 mg, 29.75 mg, 29.8 mg, 29.85 mg, 29.9 mg, 29.95 mg, 30 mg, 30.05 mg, 30.1 mg, 30.15 mg, 30.2 mg, 30.25 mg, 30.3 mg, 30.35 mg, 30.4 mg, 30.45 mg, 30.5 mg, 30.55 mg, 30.6 mg, 30.65 mg, 30.7 mg, 30.75 mg, 30.8 mg, 30.85 mg, 30.9 mg, 30.95 mg, 31 mg, 31.05 mg, 31.1 mg, 31.15 mg, 31.2 mg, 31.25 mg, 31.3 mg, 31.35 mg, 31.4 mg, 31.45 mg, 31.5 mg, 31.55 mg, 31.6 mg, 31.65 mg, 31.7 mg, 31.75 mg, 31.8 mg, 31.85 mg, 31.9 mg, 31.95 mg, 32 mg, 32.05 mg, 32.1 mg, 32.15 mg, 32.2 mg, 32.25 mg, 32.3 mg, 32.35 mg, 32.4 mg, 32.45 mg, 32.5 mg, 32.55 mg, 32.6 mg, 32.65 mg, 32.7 mg, 32.75 mg, 32.8 mg, 32.85 mg, 32.9 mg, 32.95 mg, 33 mg, 33.05 mg, 33.1 mg, 33.15 mg, 33.2 mg, 33.25 mg, 33.3 mg, 33.35 mg, 33.4 mg, 33.45 mg, 33.5 mg, 33.55 mg, 33.6 mg, 33.65 mg, 33.7 mg, 33.75 mg, 33.8 mg, 33.85 mg, 33.9 mg, 33.95 mg, 34 mg, 34.05 mg, 34.1 mg, 34.15 mg, 34.2 mg, 34.25 mg, 34.3 mg, 34.35 mg, 34.4 mg, 34.45 mg, 34.5 mg, 34.55 mg, 34.6 mg, 34.65 mg, 34.7 mg, 34.75 mg, 34.8 mg, 34.85 mg, 34.9 mg, 34.95 mg, 35 mg, 35.05 mg, 35.1 mg, 35.15 mg, 35.2 mg, 35.25 mg, 35.3 mg, 35.35 mg, 35.4 mg, 35.45 mg, 35.5 mg, 35.55 mg, 35.6 mg, 35.65 mg, 35.7 mg, 35.75 mg, 35.8 mg, 35.85 mg, 35.9 mg, 35.95 mg, 36 mg, 36.05 mg, 36.1 mg, 36.15 mg, 36.2 mg, 36.25 mg, 36.3 mg, 36.35 mg, 36.4 mg, 36.45 mg, 36.5 mg, 36.55 mg, 36.6 mg, 36.65 mg, 36.7 mg, 36.75 mg, 36.8 mg, 36.85 mg, 36.9 mg, 36.95 mg, 37 mg, 37.05 mg, 37.1 mg, 37.15 mg, 37.2 mg, 37.25 mg, 37.3 mg, 37.35 mg, 37.4 mg, 37.45 mg, 37.5 mg, 37.55 mg, 37.6 mg, 37.65 mg, 37.7 mg, 37.75 mg, 37.8 mg, 37.85 mg, 37.9 mg, 37.95 mg, 38 mg, 38.05 mg, 38.1 mg, 38.15 mg, 38.2 mg, 38.25 mg, 38.3 mg, 38.35 mg, 38.4 mg, 38.45 mg, 38.5 mg, 38.55 mg, 38.6 mg, 38.65 mg, 38.7 mg, 38.75 mg, 38.8 mg, 38.85 mg, 38.9 mg, 38.95 mg, 39 mg, 39.05 mg, 39.1 mg, 39.15 mg, 39.2 mg, 39.25 mg, 39.3 mg, 39.35 mg, 39.4 mg, 39.45 mg, 39.5 mg, 39.55 mg, 39.6 mg, 39.65 mg, 39.7 mg, 39.75 mg, 39.8 mg, 39.85 mg, 39.9 mg, 39.95 mg, and 40 mg, per dose, in particular per dose per site.

In some embodiments, 2-(diethylamino)ethyl 2-(4-isobutylphenyl)propionate or pharmaceutically acceptable salts thereof is administered in an amount selected from a group consisting of 2.1875 mg, 4.375 mg, 6.5625 mg, 8.75 mg, 10.9375 mg, 13.125 mg, 15.3125 mg, 17.5 mg, 19.6875 mg, 21.875 mg, 24.0625 mg, 26.25 mg, 28.4375 mg, 30.625 mg, 32.8125 mg, and 35 mg, per dose, in particular per dose per site.

In some embodiments, 2-(diethylamino)ethyl 2-(4-isobutylphenyl)propionate or pharmaceutically acceptable salts thereof is administered in an amount of about 5 μg/cm² to about 4 mg/cm² per dose. In some embodiments, 2-(diethylamino)ethyl 2-(4-isobutylphenyl)propionate or pharmaceutically acceptable salts thereof is administered in an amount of about 5 μg/cm² to about 2 mg/cm² per dose. In some embodiments, 2-(diethylamino)ethyl 2-(4-isobutylphenyl)propionate or pharmaceutically acceptable salts thereof is administered in an amount of about 10 μg/cm² to about 2 mg/cm² per dose. In some embodiments, 2-(diethylamino)ethyl 2-(4-isobutylphenyl)propionate or pharmaceutically acceptable salts thereof is administered in an amount of about 30 μg/cm² to about 2 mg/cm² per dose. In some embodiments, 2-(diethylamino)ethyl 2-(4-isobutylphenyl)propionate or pharmaceutically acceptable salts thereof is administered in an amount of about 35 μg/cm² to about 1.5 mg/cm² per dose. In some embodiments, 2-(diethylamino)ethyl 2-(4-isobutylphenyl)propionate or pharmaceutically acceptable salts thereof is administered in an amount of about 40 μg/cm² to about 1 mg/cm² per dose. In some embodiments, 2-(diethylamino)ethyl 2-(4-isobutylphenyl)propionate or pharmaceutically acceptable salts thereof is administered in an amount of about 45 μg/cm² to about 750 μg/cm² per dose. In some embodiments, 2-(diethylamino)ethyl 2-(4-isobutylphenyl)propionate or pharmaceutically acceptable salts thereof is administered in an amount of about 48 μg/cm² to about 600 μg/cm² per dose. In some embodiments, 2-(diethylamino)ethyl 2-(4-isobutylphenyl)propionate or pharmaceutically acceptable salts thereof is administered in an amount of about 50 μg/cm² to about 500 μg/cm² per dose. In some embodiments, 2-(diethylamino)ethyl 2-(4-isobutylphenyl)propionate or pharmaceutically acceptable salts thereof is administered in an amount of about 60 μg/cm² to about 2 mg/cm² per dose. In some embodiments, 2-(diethylamino)ethyl 2-(4-isobutylphenyl)propionate or pharmaceutically acceptable salts thereof is administered in an amount of about 80 μg/cm² to about 2 mg/cm² per dose. In some embodiments, 2-(diethylamino)ethyl 2-(4-isobutylphenyl)propionate or pharmaceutically acceptable salts thereof is administered in an amount of about 90 μg/cm² to about 1.5 mg/cm² per dose. In some embodiments, 2-(diethylamino)ethyl 2-(4-isobutylphenyl)propionate or pharmaceutically acceptable salts thereof is administered in an amount of about 96 μg/cm² to about 1.2 mg/cm² per dose. In some embodiments, 2-(diethylamino)ethyl 2-(4-isobutylphenyl)propionate or pharmaceutically acceptable salts thereof is administered in an amount of about 100 μg/cm² to about 1 mg/cm² per dose. In some embodiments, 2-(diethylamino)ethyl 2-(4-isobutylphenyl)propionate or pharmaceutically acceptable salts thereof is administered in an amount of about 45 μg/cm² to about 90 μg/cm² per dose. In some embodiments, 2-(diethylamino)ethyl 2-(4-isobutylphenyl)propionate or pharmaceutically acceptable salts thereof is administered in an amount of about 60 μg/cm² to about 120 μg/cm² per dose. In some embodiments, 2-(diethylamino)ethyl 2-(4-isobutylphenyl)propionate or pharmaceutically acceptable salts thereof is administered in an amount of about 90 μg/cm² to about 180 μg/cm² per dose. In some embodiments, 2-(diethylamino)ethyl 2-(4-isobutylphenyl)propionate or pharmaceutically acceptable salts thereof is administered in an amount of about 120 μg/cm² to about 240 μg/cm² per dose. In some embodiments, 2-(diethylamino)ethyl 2-(4-isobutylphenyl)propionate or pharmaceutically acceptable salts thereof is administered in an amount of about 180 μg/cm² to about 360 μg/cm² per dose. In some embodiments, 2-(diethylamino)ethyl 2-(4-isobutylphenyl)propionate or pharmaceutically acceptable salts thereof is administered in an amount of about 240 μg/cm² to about 480 μg/cm² per dose. In some embodiments, 2-(diethylamino)ethyl 2-(4-isobutylphenyl)propionate or pharmaceutically acceptable salts thereof is administered in an amount of about 360 μg/cm² to about 720 μg/cm² per dose. In some embodiments, 2-(diethylamino)ethyl 2-(4-isobutylphenyl)propionate or pharmaceutically acceptable salts thereof is administered in an amount of about 480 μg/cm² to about 960 μg/cm² per dose. In some embodiments, 2-(diethylamino)ethyl 2-(4-isobutylphenyl)propionate or pharmaceutically acceptable salts thereof is administered in an amount of about 720 μg/cm² to about 1.44 mg/cm² per dose. In some embodiments, 2-(diethylamino)ethyl 2-(4-isobutylphenyl)propionate or pharmaceutically acceptable salts thereof is administered in an amount of about 960 μg/cm² to about 1.92 mg/cm² per dose.

In some embodiments, 2-(diethylamino)ethyl 2-(4-isobutylphenyl)propionate or pharmaceutically acceptable salts thereof is administered in an amount selected from a group consisting of 5 μg/cm², 5.5 μg/cm², 6 μg/cm², 6.5 μg/cm², 7 μg/cm², 7.5 μg/cm², 8 μg/cm², 8.5 μg/cm², 9 μg/cm², 9.5 μg/cm², 10 μg/cm², 11 μg/cm², 12 μg/cm², 13 μg/cm², 14 μg/cm², 15 μg/cm², 16 μg/cm², 17 μg/cm², 18 μg/cm², 19 μg/cm², 20 μg/cm², 21 μg/cm², 22 μg/cm², 23 μg/cm², 24 μg/cm², 25 μg/cm², 26 μg/cm², 27 μg/cm², 28 μg/cm², 29 μg/cm², 30 μg/cm², 31 μg/cm², 32 μg/cm², 33 μg/cm², 34 μg/cm², 35 μg/cm², 36 μg/cm², 37 μg/cm², 38 μg/cm², 39 μg/cm², 40 μg/cm², 41 μg/cm², 42 μg/cm², 43 μg/cm², 44 μg/cm², 45 μg/cm², 46 μg/cm², 47 μg/cm², 48 μg/cm², 49 μg/cm², 50 μg/cm², 51 μg/cm², 52 μg/cm², 53 μg/cm², 54 μg/cm², 55 μg/cm², 56 μg/cm², 57 μg/cm², 58 μg/cm², 59 μg/cm², 60 μg/cm², 62 μg/cm², 64 μg/cm², 66 μg/cm², 68 μg/cm², 70 μg/cm², 72 μg/cm², 74 μg/cm², 76 μg/cm², 78 μg/cm², 80 μg/cm², 82 μg/cm², 84 μg/cm², 86 μg/cm², 88 μg/cm², 90 μg/cm², 92 μg/cm², 94 μg/cm², 96 μg/cm², 98 μg/cm², 100 μg/cm², 1.02 μg/cm², 104 μg/cm², 106 μg/cm², 108 μg/cm², 110 μg/cm², 112 μg/cm², 114 μg/cm², 116 μg/cm², 118 μg/cm², 120 μg/cm², 125 μg/cm², 130 μg/cm², 135 μg/cm², 140 μg/cm², 145 μg/cm², 150 μg/cm², 155 μg/cm², 160 μg/cm², 165 μg/cm², 170 μg/cm², 175 μg/cm², 180 μg/cm², 185 μg/cm², 190 μg/cm², 195 μg/cm², 200 μg/cm², 205 μg/cm², 210 μg/cm², 215 μg/cm², 220 μg/cm², 225 μg/cm², 230 μg/cm², 235 μg/cm², 240 μg/cm², 250 μg/cm², 260 μg/cm², 270 μg/cm², 280 μg/cm², 290 μg/cm², 300 μg/cm², 310 μg/cm², 320 μg/cm², 330 μg/cm², 340 μg/cm², 350 μg/cm², 360 μg/cm², 370 μg/cm², 380 μg/cm², 390 μg/cm², 400 μg/cm², 410 μg/cm², 420 μg/cm², 430 μg/cm², 440 μg/cm², 450 μg/cm², 460 μg/cm², 470 μg/cm², 480 μg/cm², 490 μg/cm², 500 μg/cm², 510 μg/cm², 520 μg/cm², 530 μg/cm², 540 μg/cm², 550 μg/cm², 560 μg/cm², 570 μg/cm², 580 μg/cm², 590 μg/cm² 600 μg/cm², 620 μg/cm², 640 μg/cm², 660 μg/cm², 680 μg/cm², 700 μg/cm², 720 μg/cm², 740 μg/cm², 760 μg/cm², 780 μg/cm², 800 μg/cm², 820 μg/cm², 840 μg/cm², 860 μg/cm², 880 μg/cm², 900 μg/cm², 920 μg/cm², 940 μg/cm², 960 μg/cm², 980 μg/cm², 1 mg/cm², 1.02 mg/cm², 1.04 mg/cm², 1.06 mg/cm², 1.08 mg/cm², 1.1 mg/cm², 1.12 mg/cm², 1.14 mg/cm², 1.16 mg/cm², 1.18 mg/cm², 1.2 mg/cm², 1.22 mg/cm², 1.24 mg/cm², 1.26 mg/cm², 1.28 mg/cm², 1.3 mg/cm², 1.32 mg/cm², 1.34 mg/cm², 1.36 mg/cm², 1.38 mg/cm², 1.4 mg/cm², 1.42 mg/cm², 1.44 mg/cm², 1.46 mg/cm², 1.48 mg/cm², 1.5 mg/cm², 1.52 mg/cm², 1.54 mg/cm², 1.56 mg/cm², 1.58 mg/cm², 1.6 mg/cm², 1.62 mg/cm², 1.64 mg/cm², 1.66 mg/cm², 1.68 mg/cm², 1.7 mg/cm², 1.72 mg/cm², 1.74 mg/cm², 1.76 mg/cm², 1.78 mg/cm², 1.8 mg/cm², 1.82 mg/cm², 1.84 mg/cm², 1.86 mg/cm², 1.88 mg/cm², 1.9 mg/cm², 1.92 mg/cm², 1.94 mg/cm², 1.96 mg/cm², 1.98 mg/cm², 2 mg/cm², 2.05 mg/cm², 2.1 mg/cm², 2.15 mg/cm², 2.2 mg/cm², 2.25 mg/cm², 2.3 mg/cm², 2.35 mg/cm², 2.4 mg/cm², 2.45 mg/cm², 2.5 mg/cm², 2.55 mg/cm², 2.6 mg/cm², 2.65 mg/cm², 2.7 mg/cm², 2.75 mg/cm², 2.8 mg/cm², 2.85 mg/cm², 2.9 mg/cm², 2.95 mg/cm², 3 mg/cm², 3.05 mg/cm², 3.1 mg/cm², 3.15 mg/cm², 3.2 mg/cm², 3.25 mg/cm², 3.3 mg/cm², 3.35 mg/cm², 3.4 mg/cm², 3.45 mg/cm², 3.5 mg/cm², 3.55 mg/cm², 3.6 mg/cm², 3.65 mg/cm², 3.7 mg/cm², 3.75 mg/cm², 3.8 mg/cm², 3.85 mg/cm², 3.9 mg/cm², 3.95 mg/cm², and 4 mg/cm² per dose.

In some embodiments, 2-(diethylamino)ethyl 2-(4-isobutylphenyl)propionate or pharmaceutically acceptable salts thereof is administered in an amount selected from a group consisting of 37.5 μg/cm², 75 μg/cm², 112.5 μg/cm², 150 μg/cm², 187.5 μg/cm², 225 μg/cm², 262.5 μg/cm², 300 μg/cm², 337.5 μg/cm², and 375 μg/cm² per dose.

In some embodiments, the subject is a warm-blooded animal. In some embodiments, the subject is a mammal. In some embodiments, the subject is a primate. In some embodiments, the subject is a human. In some embodiments, the subject is a human adult. In some embodiments, the age of the adult is more than or equal to an age selected from a group consisting of 15, 16, 17, 18, 19, 20, 21, 22, 23, 24, 25, 26, 27, 28, 29 and 30.

In some embodiments, the subject is, and/or the medicament is for, a subject suffering from, at risk of suffering from, or potentially capable of suffering from symptoms. In some embodiments, the subject is, and/or the medicament is for, a subject suffering from, at risk of suffering from, or potentially capable of suffering from symptoms, in particular a subject suffering from, at risk of suffering from, or potentially capable of suffering from diseases, inflammation, pain, fever, gout, dysmenorrhea, joint swelling, morning stiffness, rheumatoid disorders, or injury. In some embodiments, the symptoms are diseases, inflammation, pain, fever, gout, dysmenorrhea, joint swelling, morning stiffness, rheumatoid disorders, or injury. In some embodiments, the subject is, and/or the medicament is for, a subject suffering from, at risk of suffering from, or potentially capable of suffering from symptoms, in particular a subject suffering from, at risk of suffering from, or potentially capable of suffering from diseases, inflammation or pain.

In some embodiments, the subject is, and/or the medicament is for, a subject suffering from, at risk of suffering from, or potentially capable of suffering from pain.

In some embodiments, the subject is, and/or the medicament is for, subject suffering from, at risk of suffering from, or potentially capable of suffering from joint pain, bone pain, cartilage pain, muscle pain, dental pain, headache, dysmenorrhea or menstrual cramps. In some embodiments, the pain is joint pain. In some embodiments, the pain is bone pain. In some embodiments, the pain is cartilage pain. In some embodiments, the pain is muscle pain. In some embodiments, the pain is dental pain. In some embodiments, the pain is headache. In some embodiments, the pain is dysmenorrhea. In some embodiments, the pain is menstrual cramps. In some embodiments, the pain is arthritis pain. In some embodiments, the pain is osteoarthritis pain. In some embodiments, the pain is hurt. In some embodiments, the pain is postoperative pain. In some embodiments, the pain is gout pain. In some embodiments, the pain is lupus pain. In some embodiments, the pain is fibromyalgia. In some embodiments, the pain is dysmenorrhea. In some embodiments, the pain is joint swelling. In some embodiments, the pain is morning stiffness. In some embodiments, the pain is rheumatoid disorders. In some embodiments, the pain is minor injury.

In some embodiments, the pain is a joint pain of one or both knees, one or both ankles, one or both elbows, one or both wrists, one or more neck spines, one or more back spines, one or both shoulders, one side or both sides of hips, one or more of fingers, and/or one or more of toes. In some embodiments, the pain is knee joint pain. In some embodiments, the pain is ankle joint pain. In some embodiments, the pain is elbow joint pain. In some embodiments, the pain is wrist joint pain. In some embodiments, the pain is neck spine joint pain. In some embodiments, the pain is back spine joint pain. In some embodiments, the pain is shoulder joint pain. In some embodiments, the pain is hip joint pain. In some embodiments, the pain is finger joint pain. In some embodiments, the pain is toe joint pain.

In some embodiments, the pain is a cartilage pain of one or both knees, one or both ankles, one or both elbows, one or both wrists, one or more neck spines, one or more back spines, one or both shoulders, one side or both sides of hips, one or more of fingers, and/or one or more of toes. In some embodiments, the pain is knee cartilage pain. In some embodiments, the pain is ankle cartilage pain. In some embodiments, the pain is elbow cartilage pain. In some embodiments, the pain is wrist cartilage pain. In some embodiments, the pain is neck spine cartilage pain. In some embodiments, the pain is back spine cartilage pain. In some embodiments, the pain is shoulder cartilage pain. In some embodiments, the pain is hip cartilage pain. In some embodiments, the pain is finger cartilage pain. In some embodiments, the pain is toe cartilage pain.

In some embodiments, the pain is bone pain of head, neck spine, shoulder, upper arm, forearm, palm, finger, shoulder blade, rib, back spine, hip, thigh, calf, sole, or toe bones.

In some embodiments, the pain is head bone pain. In some embodiments, the pain is neck spine pain. In some embodiments, the pain is shoulder bone pain. In some embodiments, the pain is upper arm bone pain. In some embodiments, the pain is forearm bone pain. In some embodiments, the pain is palm bone pain. In some embodiments, the pain is finger bone pain. In some embodiments, the pain is shoulder blade pain. In some embodiments, the pain is rib pain. In some embodiments, the pain is back spine pain. In some embodiments, the pain is hip bone pain. In some embodiments, the pain is thigh bone pain. In some embodiments, the pain is calf bone pain. In some embodiments, the pain is sole bone pain. In some embodiments, the pain is toe bone pain.

In some embodiments, the pain is muscle pain of head, neck, shoulder, upper arm, forearm, palm, finger, chest, abdomen, back, hip, thigh, calf, sole, or toe muscles.

In some embodiments, the pain is head muscle pain. In some embodiments, the pain is neck muscle pain. In some embodiments, the pain is shoulder muscle pain. In some embodiments, the pain is upper arm muscle pain. In some embodiments, the pain is forearm muscle pain. In some embodiments, the pain is palm muscle pain. In some embodiments, the pain is finger muscle pain. In some embodiments, the pain is chest muscle pain. In some embodiments, the pain is abdomen muscle pain. In some embodiments, the pain is back muscle pain. In some embodiments, the pain is hip muscle pain. In some embodiments, the pain is thigh muscle pain. In some embodiments, the pain is calf muscle pain. In some embodiments, the pain is sole muscle pain. In some embodiments, the pain is toe muscle pain.

In some embodiments, the subject is, and/or the medicament is for, a subject suffering from, at risk of suffering from, or potentially capable of suffering from inflammation.

In some embodiments, the subject is, and/or the medicament is for, subject suffering from, at risk of suffering from, or potentially capable of suffering from joint inflammation, bone inflammation, cartilage inflammation, muscle inflammation, or dental inflammation. In some embodiments, the inflammation is joint inflammation. In some embodiments, the inflammation is bone inflammation. In some embodiments, the inflammation is cartilage inflammation. In some embodiments, the inflammation is muscle inflammation. In some embodiments, the inflammation is dental inflammation.

In some embodiments, the inflammation is a joint inflammation of one or both knees, one or both ankles, one or both elbows, one or both wrists, one or more neck spines, one or more back spines, one or both shoulders, one side or both sides of hips, one or more of fingers, and/or one or more of toes. In some embodiments, the inflammation is knee joint inflammation. In some embodiments, the inflammation is ankle joint inflammation. In some embodiments, the inflammation is elbow joint inflammation. In some embodiments, the inflammation is wrist joint inflammation. In some embodiments, the inflammation is neck spine joint inflammation. In some embodiments, the inflammation is back spine joint inflammation. In some embodiments, the inflammation is shoulder joint inflammation. In some embodiments, the inflammation is hip joint inflammation. In some embodiments, the inflammation is finger joint inflammation. In some embodiments, the inflammation is toe joint inflammation.

In some embodiments, the inflammation is a cartilage inflammation of one or both knees, one or both ankles, one or both elbows, one or both wrists, one or more neck spines, one or more back spines, one or both shoulders, one side or both sides of hips, one or more of fingers, and/or one or more of toes. In some embodiments, the inflammation is knee cartilage inflammation. In some embodiments, the inflammation is ankle cartilage inflammation. In some embodiments, the inflammation is elbow cartilage inflammation. In some embodiments, the inflammation is wrist cartilage inflammation. In some embodiments, the inflammation is neck spine cartilage inflammation. In some embodiments, the inflammation is back spine cartilage inflammation. In some embodiments, the inflammation is shoulder cartilage inflammation. In some embodiments, the inflammation is hip cartilage inflammation. In some embodiments, the inflammation is finger cartilage inflammation. In some embodiments, the inflammation is toe cartilage inflammation.

In some embodiments, the inflammation is bone inflammation of head, neck spine, shoulder, upper arm, forearm, palm, finger, shoulder blade, rib, back spine, hip, thigh, calf, sole, or toe bones.

In some embodiments, the inflammation is head bone inflammation. In some embodiments, the inflammation is neck spine inflammation. In some embodiments, the inflammation is shoulder bone inflammation. In some embodiments, the inflammation is upper arm bone inflammation. In some embodiments, the inflammation is forearm bone inflammation. In some embodiments, the inflammation is palm bone inflammation. In some embodiments, the inflammation is finger bone inflammation. In some embodiments, the inflammation is shoulder blade inflammation. In some embodiments, the inflammation is rib inflammation. In some embodiments, the inflammation is back spine inflammation. In some embodiments, the inflammation is hip bone inflammation. In some embodiments, the inflammation is thigh bone inflammation. In some embodiments, the inflammation is calf bone inflammation. In some embodiments, the inflammation is sole bone inflammation. In some embodiments, the inflammation is toe bone inflammation.

In some embodiments, the inflammation is muscle inflammation of head, neck, shoulder, upper arm, forearm, palm, finger, chest, abdomen, back, hip, thigh, calf, sole, or toe muscles.

In some embodiments, the inflammation is head muscle inflammation. In some embodiments, the inflammation is neck muscle inflammation. In some embodiments, the inflammation is shoulder muscle inflammation. In some embodiments, the inflammation is upper arm muscle inflammation. In some embodiments, the inflammation is forearm muscle inflammation. In some embodiments, the inflammation is palm muscle inflammation. In some embodiments, the inflammation is finger muscle inflammation. In some embodiments, the inflammation is chest muscle inflammation. In some embodiments, the inflammation is abdomen muscle inflammation. In some embodiments, the inflammation is back muscle inflammation. In some embodiments, the inflammation is hip muscle inflammation. In some embodiments, the inflammation is thigh muscle inflammation. In some embodiments, the inflammation is calf muscle inflammation. In some embodiments, the inflammation is sole muscle inflammation. In some embodiments, the inflammation is toe muscle inflammation.

In some embodiments, the subject is, and/or the medicament is for, a subject suffering from, at risk of suffering from, or potentially capable of suffering from a disease. In some embodiments, the disease is an inflammation. In some embodiments, the disease is a joint disease. In some embodiments, the disease is a muscle disease. In some embodiments, the disease is an arthritis. In some embodiments, the disease is selected from a group consisting of osteoarthritis, rheumatoid arthritis, gout, lupus, fibromyalgia, and septic arthritis. In some embodiments, the disease is osteoarthritis. In some embodiments, the disease is rheumatoid arthritis. In some embodiments, the disease is a hurting disease.

In some embodiments, the disease is a muscle disease, a cartilage disease, or a bone disease. In some embodiments, the disease is a myositis.

In some embodiments, the joint of the joint disease, arthritis or osteoarthritis is a knee, an ankle, an elbow, a wrist, a neck spine, a back spine, a shoulder, a hip, a finger, and/or a toe. In some embodiments, the joint of the joint disease, arthritis or osteoarthritis is a knee. In some embodiments, the joint of the joint disease or osteoarthritis is an ankle. In some embodiments, the joint of the joint disease, arthritis or osteoarthritis is an elbow. In some embodiments, the joint of the joint disease, arthritis or osteoarthritis is a wrist. In some embodiments, the joint of the joint disease, arthritis or osteoarthritis is one or more of back spine joints. In some embodiments, the joint of the joint disease, arthritis or osteoarthritis is one or more of back spine joints. In some embodiments, the joint of the joint disease, arthritis or osteoarthritis is a shoulder. In some embodiments, the joint of the joint disease, arthritis or is osteoarthritis is a hip. In some embodiments, the joint of the joint disease, arthritis or osteoarthritis is a finger. In some embodiments, the joint of the joint disease, arthritis or osteoarthritis is a toe.

In some embodiments, the osteoarthritis is a knee osteoarthritis, an ankle osteoarthritis, an elbow osteoarthritis, a wrist osteoarthritis, a neck osteoarthritis, aback osteoarthritis, a shoulder osteoarthritis, a hip osteoarthritis, a finger osteoarthritis, and/or a toe osteoarthritis. In some embodiments, the osteoarthritis is a knee osteoarthritis. In some embodiments, the osteoarthritis is an ankle osteoarthritis. In some embodiments, the osteoarthritis is an elbow osteoarthritis. In some embodiments, the osteoarthritis is a wrist osteoarthritis. In some embodiments, the osteoarthritis is a neck spine osteoarthritis. In some embodiments, the osteoarthritis is a back spine osteoarthritis. In some embodiments, the osteoarthritis is a shoulder osteoarthritis. In some embodiments, the osteoarthritis is a hip osteoarthritis. In some embodiments, the osteoarthritis is a finger osteoarthritis. In some embodiments, the osteoarthritis is a toe osteoarthritis.

In some embodiments, the rheumatoid arthritis is a knee rheumatoid arthritis, an ankle rheumatoid arthritis, an elbow rheumatoid arthritis, a wrist rheumatoid arthritis, a neck rheumatoid arthritis, a back rheumatoid arthritis, a shoulder rheumatoid arthritis, a hip rheumatoid arthritis, a finger rheumatoid arthritis, and/or a toe rheumatoid arthritis. In some embodiments, the rheumatoid arthritis is a knee rheumatoid arthritis. In some embodiments, the rheumatoid arthritis is an ankle rheumatoid arthritis. In some embodiments, the rheumatoid arthritis is an elbow rheumatoid arthritis. In some embodiments, the rheumatoid arthritis is a wrist rheumatoid arthritis. In some embodiments, the rheumatoid arthritis is a neck spine rheumatoid arthritis. In some embodiments, the rheumatoid arthritis is aback spine rheumatoid arthritis. In some embodiments, the rheumatoid arthritis is a shoulder rheumatoid arthritis. In some embodiments, the rheumatoid arthritis is a hip rheumatoid arthritis. In some embodiments, the rheumatoid arthritis is a finger rheumatoid arthritis. In some embodiments, the rheumatoid arthritis is a toe rheumatoid arthritis.

In some embodiments, the subject is, and/or the medicament is for, a subject suffering from, at risk of suffering from, or potentially capable of suffering from fever.

In some embodiments, the subject is, and/or the medicament is for, a subject suffering from, at risk of suffering from, or potentially capable of suffering from gout seizures.

In some embodiments, the gout seizure is a knee gout seizure, an ankle gout seizure, an elbow gout seizure, a wrist gout seizure, a neck gout seizure, a back gout seizure, a shoulder gout seizure, a hip gout seizure, a finger gout seizure, and/or a toe gout seizure. In some embodiments, the gout seizure is a knee gout seizure. In some embodiments, the gout seizure is an ankle gout seizure. In some embodiments, the gout seizure is an elbow gout seizure. In some embodiments, the gout seizure is a wrist gout seizure. In some embodiments, the gout seizure is a neck spine gout seizure. In some embodiments, the gout seizure is a back spine gout seizure. In some embodiments, the gout seizure is a shoulder gout seizure. In some embodiments, the gout seizure is a hip gout seizure. In some embodiments, the gout seizure is a finger gout seizure. In some embodiments, the gout seizure is a toe gout seizure.

In some embodiments, 2-(diethylamino)ethyl 2-(4-isobutylphenyl)propionate or pharmaceutically acceptable salts thereof is topically administered to a joint suffering from, at risk of suffering from, or potentially capable of suffering from joint disease. In some embodiments, 2-(diethylamino)ethyl 2-(4-isobutylphenyl)propionate or pharmaceutically acceptable salts thereof is topically administered to a joint suffering from, at risk of suffering from, or potentially capable of suffering from arthritis. In some embodiments, 2-(diethylamino)ethyl 2-(4-isobutylphenyl)propionate or pharmaceutically acceptable salts thereof is topically administered to a joint suffering from, at risk of suffering from, or potentially capable of suffering from osteoarthritis.

In some embodiments, 2-(diethylamino)ethyl 2-(4-isobutylphenyl)propionate or pharmaceutically acceptable salts thereof is topically administered to one or more joints suffering from, at risk of suffering from, or potentially capable of suffering from osteoarthritis.

In some embodiments, 2-(diethylamino)ethyl 2-(4-isobutylphenyl)propionate or pharmaceutically acceptable salts thereof is topically administered to one or both knees, one or both ankles, one or both elbows, one or both wrists, one or more neck spines, one or more back spines, one or both shoulders, one side or both sides of hips, one or more of fingers, and/or one or more of toes.

In some embodiments, the one or more sites of the subject includes a head, one or both knees, one or both ankles, one or more thighs, one or more calves, one or more soles, one or both elbows, one or both wrists, one or both upper arms, one or both forearms, one or both palms, a neck, a back, one or both shoulders, one side or both sides of chest, an abdomen, one side or both sides of hips, one or more of fingers, and/or one or more of toes.

In some embodiments, the one or more sites of the subject includes one knee or two knees. In some embodiments, the one or more sites of the subject includes one knees. In some embodiments, the one or more sites of the subject includes two knees. In some embodiments, the one or more sites of the subject includes one ankle or two ankles. In some embodiments, the one or more sites of the subject includes one thigh or two thighs. In some embodiments, the one or more sites of the subject includes one calf or two calves. In some embodiments, the one or more sites of the subject includes one sole or two soles. In some embodiments, the one or more sites of the subject includes one elbow or two elbows. In some embodiments, the one or more sites of the subject includes one wrist or two wrists. In some embodiments, the one or more sites of the subject includes one upper arm or two upper arms. In some embodiments, the one or more sites of the subject includes one forearm or two forearms. In some embodiments, the one or more sites of the subject includes one palm or two palms. In some embodiments, the one or more sites of the subject includes a neck. In some embodiments, the one or more sites of the subject includes a back.

In some embodiments, the one or more sites of the subject includes one shoulder or two shoulders. In some embodiments, the one or more sites of the subject includes one side or two sides of chest. In some embodiments, the one or more sites of the subject includes an abdomen. In some embodiments, the one or more sites of the subject includes one side or two side of hips. In some embodiments, the one or more sites of the subject includes 1, 2, 3, 4, 5, 6, 7, 8, 9 or 10 fingers. In some embodiments, the one or more sites of the subject includes 1, 2, 3, 4, 5, 6, 7, 8, 9 or 10 toes.

In some embodiments, the one or more sites of the subject includes one or more joints, one or more muscles, one or more bones, one or more cartilages, and/or one or more soft tissues. In some embodiments, the one or more sites of the subject includes one or more joints. In some embodiments, the one or more sites of the subject includes one or more muscles. In some embodiments, the one or more sites of the subject includes one or more bones. In some embodiments, the one or more sites of the subject includes or one or more cartilages. In some embodiments, the one or more sites of the subject includes or one or more parts of soft tissues.

In some embodiments, the one or more sites of the subject includes one or more joints, one or more muscles, one or more bones, one or more cartilages, and/or one or more parts of soft tissues suffering from, at risk of suffering from, or potentially capable of suffering from pain. In some embodiments, the one or more sites of the subject includes one or more joints suffering from, at risk of suffering from, or potentially capable of suffering from joint pain. In some embodiments, the one or more sites of the subject includes one or more muscles suffering from, at risk of suffering from, or potentially capable of suffering from muscle pain. In some embodiments, the one or more sites of the subject includes one or more bones suffering from, at risk of suffering from, or potentially capable of suffering from bone pain. In some embodiments, the one or more sites of the subject includes or one or more cartilages suffering from, at risk of suffering from, or potentially capable of suffering from cartilage pain. In some embodiments, the one or more sites of the subject includes or one or more parts of soft tissues suffering from, at risk of suffering from, or potentially capable of suffering from soft tissue pain.

In some embodiments, the one or more sites of the subject includes one or more joints, one or more muscles, one or more bones, one or more cartilages, and/or one or more parts of soft tissues suffering from, at risk of suffering from, or potentially capable of suffering from inflammation. In some embodiments, the one or more sites of the subject includes one or more joints suffering from, at risk of suffering from, or potentially capable of suffering from arthritis. In some embodiments, the one or more sites of the subject includes one or more joints suffering from, at risk of suffering from, or potentially capable of suffering from osteoarthritis.

In some embodiments, the one or more sites of the subject includes one or both knee joints, one or both ankle joints, one or both elbow joints, one or both wrist joints, one or more neck spine joints, one or more back spine joints, one or both shoulder joints, one or both of hip joints, one or more of finger joints, and/or one or more of toe joints.

In some embodiments, the one or more sites of the subject includes one or both knee joints. In some embodiments, the one or more sites of the subject includes one or both ankle joints. In some embodiments, the one or more sites of the subject includes one or both elbow joints. In some embodiments, the one or more sites of the subject includes one or both wrist joints. In some embodiments, the one or more sites of the subject includes one or more neck spine joints. In some embodiments, the one or more sites of the subject includes one or more back spine joints. In some embodiments, the one or more sites of the subject includes one or both shoulder joints. In some embodiments, the one or more sites of the subject includes one or both of hip joints. In some embodiments, the one or more sites of the subject includes one or more of finger joints. In some embodiments, the one or more sites of the subject includes one or more of toe joints.

In some embodiments, the one or more sites of the subject includes one or more head muscles, one or more neck muscles, one or more shoulder muscles, one or more upper arm muscles, one or more forearm muscles, one or more palm muscles, one or more finger muscles, one or more chest muscles, one or more abdomen muscles, one or more back muscles, one or more hip muscles, one or more thigh muscles, one or more calf muscles, one or more sole muscles, and/or one or more toe muscles.

In some embodiments, the one or more sites of the subject includes one or more head muscles. In some embodiments, the one or more sites of the subject includes one or more neck muscles. In some embodiments, the one or more sites of the subject includes one or more shoulder muscles. In some embodiments, the one or more sites of the subject includes one or more upper arm muscles. In some embodiments, the one or more sites of the subject includes one or more forearm muscles. In some embodiments, the one or more sites of the subject includes one or more palm muscles. In some embodiments, the one or more sites of the subject includes one or more finger muscles. In some embodiments, the one or more sites of the subject includes one or more chest muscles. In some embodiments, the one or more sites of the subject includes one or more abdomen muscles. In some embodiments, the one or more sites of the subject includes one or more back muscles. In some embodiments, the one or more sites of the subject includes one or more hip muscles. In some embodiments, the one or more sites of the subject includes one or more thigh muscles. In some embodiments, the one or more sites of the subject includes one or more calf muscles. In some embodiments, the one or more sites of the subject includes one or more sole muscles. In some embodiments, the one or more sites of the subject includes one or more toe muscles.

In some embodiments, the one or more sites of the subject includes one or more head bones, one or more neck spine bones, one or more shoulder bones, one or more upper arm bones, one or more forearm bones, one or more palm bones, one or more finger bones, one or more shoulder blade bones, one or more rib bones, one or more back spine bones, one or more hip bones, one or more thigh bones, one or more calf bones, one or more sole bones, and/or one or more toe bones.

In some embodiments, the one or more sites of the subject includes one or more head bones. In some embodiments, the one or more sites of the subject includes one or more neck spine bones. In some embodiments, the one or more sites of the subject includes one or more shoulder bones. In some embodiments, the one or more sites of the subject includes one or more upper arm bones. In some embodiments, the one or more sites of the subject includes one or more forearm bones. In some embodiments, the one or more sites of the subject includes one or more palm bones. In some embodiments, the one or more sites of the subject includes one or more finger bones. In some embodiments, the one or more sites of the subject includes one or more shoulder blade bones. In some embodiments, the one or more sites of the subject includes one or more rib bones. In some embodiments, the one or more sites of the subject includes one or more back spine bones. In some embodiments, the one or more sites of the subject includes one or more hip bones. In some embodiments, the one or more sites of the subject includes one or more thigh bones. In some embodiments, the one or more sites of the subject includes one or more calf bones. In some embodiments, the one or more sites of the subject includes one or more sole bones. In some embodiments, the one or more sites of the subject includes one or more toe bones.

In some embodiments, the one or more sites of the subject includes one or more knee cartilages, one or more ankle cartilages, one or more elbow cartilages, one or more wrist cartilages, one or more neck spine cartilages, one or more back spine cartilages, one or more shoulder cartilages, one or more of hip cartilages, one or more of finger cartilages, and/or one or more of toe cartilages.

In some embodiments, the one or more sites of the subject includes one or more knee cartilages. In some embodiments, the one or more sites of the subject includes one or more ankle cartilages. In some embodiments, the one or more sites of the subject includes one or more elbow cartilages. In some embodiments, the one or more sites of the subject includes one or more wrist cartilages. In some embodiments, the one or more sites of the subject includes one or more neck spine cartilages. In some embodiments, the one or more sites of the subject includes one or more back spine cartilages. In some embodiments, the one or more sites of the subject includes one or more shoulder cartilages. In some embodiments, the one or more sites of the subject includes one or more of hip cartilages. In some embodiments, the one or more sites of the subject includes one or more of finger cartilages. In some embodiments, the one or more sites of the subject includes one or more of toe cartilages.

In some embodiments, one of the one or more sites of the subject is one joint, one muscle, one bone, one cartilage, or one area of soft tissues. In some embodiments, one of the one or more sites of the subject is one joint. In some embodiments, one of the one or more sites of the subject is one muscle. In some embodiments, one of the one or more sites of the subject is one bone. In some embodiments, one of the one or more sites of the subject is one cartilage. In some embodiments, one of the one or more sites of the subject is one area of soft tissues.

In some embodiments, one of the one or more sites of the subject is one joint, one muscle, one bone, one cartilage, or one area of soft tissues suffering from, at risk of suffering from, or potentially capable of suffering from pain. In some embodiments, one of the one or more sites of the subject is one joint suffering from, at risk of suffering from, or potentially capable of suffering from joint pain. In some embodiments, one of the one or more sites of the subject is one muscles suffering from, at risk of suffering from, or potentially capable of suffering from muscle pain. In some embodiments, one of the one or more sites of the subject is one bone suffering from, at risk of suffering from, or potentially capable of suffering from bone pain. In some embodiments, one of the one or more sites of the subject is one cartilage suffering from, at risk of suffering from, or potentially capable of suffering from cartilage pain. In some embodiments, one of the one or more sites of the subject is one area of soft tissues suffering from, at risk of suffering from, or potentially capable of suffering from soft tissue pain.

In some embodiments, one of the one or more sites of the subject is one joint, one muscle, one bone, one cartilage, or one area of soft tissues suffering from, at risk of suffering from, or potentially capable of suffering from inflammation. In some embodiments, one of the one or more sites of the subject is one joint suffering from, at risk of suffering from, or potentially capable of suffering from arthritis. In some embodiments, one of the one or more sites of the subject is one joint suffering from, at risk of suffering from, or potentially capable of suffering from osteoarthritis.

In some embodiments, each of the sites of the subject is any one joint suffering from, at risk of suffering from, or potentially capable of suffering from osteoarthritis.

In some embodiments, any one of the sites of the subject are selected from any one in a group consisting of knees, ankles, elbows, wrists, shoulders, hips, fingers, and toes. In some embodiments, each of the sites of the subject are selected from a group consisting of knees, ankles, thighs, calves, soles, elbows, wrists, upper arms, forearms, palms, neck, back, shoulders, hips, chest, abdomen, hips, fingers, and toes.

In some embodiments, one of the one or more sites of the subject is one head, one knee, one ankle, one thigh, one calf, one sole, one elbow, one wrist, one upper arm, one forearm, one palm, one neck, one back, one shoulder, one side of chest, one abdomen, one side of hips, one finger, or one toe.

In some embodiments, one of the one or more sites of the subject is one head. In some embodiments, one of the one or more sites of the subject is one knee. In some embodiments, one of the one or more sites of the subject is one ankle. In some embodiments, one of the one or more sites of the subject is one thigh. In some embodiments, one of the one or more sites of the subject is one calf. In some embodiments, one of the one or more sites of the subject is one sole. In some embodiments, one of the one or more sites of the subject is one elbow. In some embodiments, one of the one or more sites of the subject is one wrist. In some embodiments, one of the one or more sites of the subject is one upper arm. In some embodiments, one of the one or more sites of the subject is one forearm. In some embodiments, one of the one or more sites of the subject is one palm. In some embodiments, one of the one or more sites of the subject is one neck. In some embodiments, one of the one or more sites of the subject is one back. In some embodiments, one of the one or more sites of the subject is one shoulder. In some embodiments, one of the one or more sites of the subject is one side of chest. In some embodiments, one of the one or more sites of the subject is one abdomen. In some embodiments, one of the one or more sites of the subject is one side of hips. In some embodiments, one of the one or more sites of the subject is one finger. In some embodiments, one of the one or more sites of the subject is one toe.

In some embodiments, one of the one or more sites of the subject is one knee joint, one ankle joint, one elbow joint, one wrist joint, one neck spine joint, one back spine joint, one shoulder joint, one hip joint, one finger joint, or one toe joint.

In some embodiments, one of the one or more sites of the subject is one head muscle, one neck muscle, one shoulder muscle, one upper arm muscle, one forearm muscle, one palm muscle, one finger muscle, one chest muscle, one abdomen muscle, one back muscle, one hip muscle, one thigh muscle, one calf muscle, one sole muscle, or one toe muscle.

In some embodiments, one of the one or more sites of the subject is one head muscle. In some embodiments, one of the one or more sites of the subject is one neck muscle. In some embodiments, one of the one or more sites of the subject is one shoulder muscle. In some embodiments, one of the one or more sites of the subject is one upper arm muscle. In some embodiments, one of the one or more sites of the subject is one forearm muscle. In some embodiments, one of the one or more sites of the subject is one palm muscle. In some embodiments, one of the one or more sites of the subject is one finger muscle. In some embodiments, one of the one or more sites of the subject is one chest muscle. In some embodiments, one of the one or more sites of the subject is one abdomen muscle. In some embodiments, one of the one or more sites of the subject is one back muscle. In some embodiments, one of the one or more sites of the subject is one hip muscle. In some embodiments, one of the one or more sites of the subject is one thigh muscle. In some embodiments, one of the one or more sites of the subject is one calf muscle. In some embodiments, one of the one or more sites of the subject is one sole muscle. In some embodiments, one of the one or more sites of the subject is one toe muscle.

In some embodiments, one of the one or more sites of the subject is one head bone, one neck spine bone, one shoulder bone, one upper arm bone, one forearm bone, one palm bone, one finger bone, one shoulder blade bone, one rib bone, one back spine bone, one hip bone, one thigh bone, one calf bone, one sole bone, or one toe bone.

In some embodiments, one of the one or more sites of the subject is one head bone. In some embodiments, one of the one or more sites of the subject is one neck spine bone. In some embodiments, one of the one or more sites of the subject is one shoulder bone. In some embodiments, one of the one or more sites of the subject is one upper arm bone. In some embodiments, one of the one or more sites of the subject is one forearm bone. In some embodiments, one of the one or more sites of the subject is one palm bone. In some embodiments, one of the one or more sites of the subject is one finger bone. In some embodiments, one of the one or more sites of the subject is one shoulder blade bone. In some embodiments, one of the one or more sites of the subject is one rib bone. In some embodiments, one of the one or more sites of the subject is one back spine bone. In some embodiments, one of the one or more sites of the subject is one hip bone. In some embodiments, one of the one or more sites of the subject is one thigh bone. In some embodiments, one of the one or more sites of the subject is one calf bone. In some embodiments, one of the one or more sites of the subject is one sole bone. In some embodiments, one of the one or more sites of the subject is or one toe bone.

In some embodiments, one of the one or more sites of the subject is one knee cartilage, one ankle cartilage, one elbow cartilage, one wrist cartilage, one neck spine cartilage, one back spine cartilage, one shoulder cartilage, one hips cartilage, one finger cartilage, or one toe cartilage.

In some embodiments, one of the one or more sites of the subject is one knee cartilage. In some embodiments, one of the one or more sites of the subject is one ankle cartilage. In some embodiments, one of the one or more sites of the subject is one elbow cartilage. In some embodiments, one of the one or more sites of the subject is one wrist cartilage. In some embodiments, one of the one or more sites of the subject is one neck spine cartilage. In some embodiments, one of the one or more sites of the subject is one back spine cartilage. In some embodiments, one of the one or more sites of the subject is one shoulder cartilage. In some embodiments, one of the one or more sites of the subject is one hips cartilage. In some embodiments, one of the one or more sites of the subject is one finger cartilage. In some embodiments, one of the one or more sites of the subject is or one toe cartilage.

In some embodiments, 2-(diethylamino)ethyl 2-(4-isobutylphenyl)propionate or pharmaceutically acceptable salts thereof is topically administered to one or both knees, one or both ankles, one or both elbows, one or both wrists, one or more neck spines, one or more back spines, one or both shoulders, a side of hips, one or more of fingers, one or more of toes, and one or more areas of soft tissues.

In some embodiments, a site of the subject includes one or more surfaces. In some embodiments, a site of the subject includes the medial surface, the lateral surface, the front surface and/or the back surface thereof. In some embodiments, the site includes the medial surface thereof. In some embodiments, the site includes the lateral surface thereof. In some embodiments, the site includes the front surface thereof. In some embodiments, the site includes the back surface thereof. In some embodiments, the site includes the medial surface and the lateral surface thereof. In some embodiments, the site includes the front surface and the back surface thereof. In some embodiments, the site includes the medial surface, the front surface and the back surface thereof. In some embodiments, the site includes the lateral surface, the front surface and the back surface thereof. In some embodiments, the site includes the medial surface, the front surface and the lateral surface thereof. In some embodiments, the site includes the lateral surface, the front surface and the medial surface thereof. In some embodiments, the site includes the medial surface, the lateral surface, the front surface and the back surface thereof.

In some embodiments, a site of the subject includes one or more surfaces of the joint, or around, or close to the joint. In some embodiments, a site of the subject includes one or more surfaces of the joint. In some embodiments, a site of the subject includes one or more surfaces around or close to the muscle. In some embodiments, a site of the subject includes one or more surfaces around or close to the bone. In some embodiments, a site of the subject includes one or more surfaces around or close to the cartilage.

In some embodiments, 2-(diethylamino)ethyl 2-(4-isobutylphenyl)propionate or pharmaceutically acceptable salts thereof is topically administered to one or more surfaces of the knee or knees. In some embodiments, 2-(diethylamino)ethyl 2-(4-isobutylphenyl)propionate or pharmaceutically acceptable salts thereof is topically administered to the medial surface, the lateral surface, the front surface and/or the back surface of the knee or knees. In some embodiments, 2-(diethylamino)ethyl 2-(4-isobutylphenyl)propionate or pharmaceutically acceptable salts thereof is topically administered to the medial surface of the knee or knees. In some embodiments, 2-(diethylamino)ethyl 2-(4-isobutylphenyl)propionate or pharmaceutically acceptable salts thereof is topically administered to the lateral surface of the knee or knees. In some embodiments, 2-(diethylamino)ethyl 2-(4-isobutylphenyl)propionate or pharmaceutically acceptable salts thereof is topically administered to the front surface of the knee or knees. In some embodiments, 2-(diethylamino)ethyl 2-(4-isobutylphenyl)propionate or pharmaceutically acceptable salts thereof is topically administered to the back surface of the knee or knees. In some embodiments, 2-(diethylamino)ethyl 2-(4-isobutylphenyl)propionate or pharmaceutically acceptable salts thereof is topically administered to the medial surface and the lateral surface of the knee or knees. In some embodiments, 2-(diethylamino)ethyl 2-(4-isobutylphenyl)propionate or pharmaceutically acceptable salts thereof is topically administered to the front surface and the back surface of the knee or knees. In some embodiments, 2-(diethylamino)ethyl 2-(4-isobutylphenyl)propionate or pharmaceutically acceptable salts thereof is topically administered to the medial surface, the front surface and the back surface of the knee or knees. In some embodiments, 2-(diethylamino)ethyl 2-(4-isobutylphenyl)propionate or pharmaceutically acceptable salts thereof is topically administered to the lateral surface, the front surface and the back surface of the knee or knees. In some embodiments, 2-(diethylamino)ethyl 2-(4-isobutylphenyl)propionate or pharmaceutically acceptable salts thereof is topically administered to the medial surface, the front surface and the lateral surface of the knee or knees. In some embodiments, 2-(diethylamino)ethyl 2-(4-isobutylphenyl)propionate or pharmaceutically acceptable salts thereof is topically administered to the lateral surface, the front surface and the medial surface of the knee or knees. In some embodiments, 2-(diethylamino)ethyl 2-(4-isobutylphenyl)propionate or pharmaceutically acceptable salts thereof is topically administered to the medial surface, the lateral surface, the front surface and the back surface of the knee or knees.

In some embodiments, 2-(diethylamino)ethyl 2-(4-isobutylphenyl)propionate or pharmaceutically acceptable salts thereof is topically administered to the surface around and/or close to one or both elbow joint. In some embodiments, 2-(diethylamino)ethyl 2-(4-isobutylphenyl)propionate or pharmaceutically acceptable salts thereof is topically administered to the surface around and/or close to one or both wrist joints. In some embodiments, 2-(diethylamino)ethyl 2-(4-isobutylphenyl)propionate or pharmaceutically acceptable salts thereof is topically administered to the surface around and/or close to one or more of neck spine joints. In some embodiments, 2-(diethylamino)ethyl 2-(4-isobutylphenyl)propionate or pharmaceutically acceptable salts thereof is topically administered to the surface close to one or more back spine joints. In some embodiments, 2-(diethylamino)ethyl 2-(4-isobutylphenyl)propionate or pharmaceutically acceptable salts thereof is topically administered to the surface around and/or close to one or both shoulder joints. In some embodiments, 2-(diethylamino)ethyl 2-(4-isobutylphenyl)propionate or pharmaceutically acceptable salts thereof is topically administered to the surface around and/or close to one or both hip joints. In some embodiments, 2-(diethylamino)ethyl 2-(4-isobutylphenyl)propionate or pharmaceutically acceptable salts thereof is topically administered to the surface around and/or close to the joints of 1, 2, 3, 4, 5, 6, 7, 8, 9 or 10 fingers. In some embodiments, 2-(diethylamino)ethyl 2-(4-isobutylphenyl)propionate or pharmaceutically acceptable salts thereof is topically administered to the surface around and/or close to the joints of 1, 2, 3, 4, 5, 6, 7, 8, 9 or 10 toes.

In some embodiments, 2-(diethylamino)ethyl 2-(4-isobutylphenyl)propionate or pharmaceutically acceptable salts thereof is topically administered to one or more surfaces around and/or close to one or both elbow joint. In some embodiments, 2-(diethylamino)ethyl 2-(4-isobutylphenyl)propionate or pharmaceutically acceptable salts thereof is topically administered to one or more surfaces around and/or close to one or both wrist joints. In some embodiments, 2-(diethylamino)ethyl 2-(4-isobutylphenyl)propionate or pharmaceutically acceptable salts thereof is topically administered to one or more surfaces around and/or close to one or more neck spine joints. In some embodiments, 2-(diethylamino)ethyl 2-(4-isobutylphenyl)propionate or pharmaceutically acceptable salts thereof is topically administered to one or more surfaces close to one or more back spine joints. In some embodiments, 2-(diethylamino)ethyl 2-(4-isobutylphenyl)propionate or pharmaceutically acceptable salts thereof is topically administered to one or more surfaces around and/or close to one or both shoulder joints. In some embodiments, 2-(diethylamino)ethyl 2-(4-isobutylphenyl)propionate or pharmaceutically acceptable salts thereof is topically administered to one or more surfaces around and/or close to one or both hip joints. In some embodiments, 2-(diethylamino)ethyl 2-(4-isobutylphenyl)propionate or pharmaceutically acceptable salts thereof is topically administered to one or more surfaces around and/or close to the joints of 1, 2, 3, 4, 5, 6, 7, 8, 9 or 10 fingers. In some embodiments, 2-(diethylamino)ethyl 2-(4-isobutylphenyl)propionate or pharmaceutically acceptable salts thereof is topically administered to one or more surfaces around and/or close to the joints of 1, 2, 3, 4, 5, 6, 7, 8, 9 or 10 toes.

In some embodiments, 2-(diethylamino)ethyl 2-(4-isobutylphenyl)propionate or pharmaceutically acceptable salts thereof is topically administered until no pain on the administered area. In some embodiments, 2-(diethylamino)ethyl 2-(4-isobutylphenyl)propionate or pharmaceutically acceptable salts thereof is topically administered until no pain on one or both knees, one or both ankles, one or both elbows, one or both wrists, neck, back, one or both shoulders, one side or both sides of hips, one or more of fingers, and/or one or more of toes.

In some embodiments, 2-(diethylamino)ethyl 2-(4-isobutylphenyl)propionate or pharmaceutically acceptable salts thereof is topically administered until no pain on one or both knees. In some embodiments, 2-(diethylamino)ethyl 2-(4-isobutylphenyl)propionate or pharmaceutically acceptable salts thereof is topically administered until no pain on one or both ankles. In some embodiments, 2-(diethylamino)ethyl 2-(4-isobutylphenyl)propionate or pharmaceutically acceptable salts thereof is topically administered until no pain on one or both elbows. In some embodiments, 2-(diethylamino)ethyl 2-(4-isobutylphenyl)propionate or pharmaceutically acceptable salts thereof is topically administered until no pain on one or both wrists. In some embodiments, 2-(diethylamino)ethyl 2-(4-isobutylphenyl)propionate or pharmaceutically acceptable salts thereof is topically administered until no pain the neck. In some embodiments, 2-(diethylamino)ethyl 2-(4-isobutylphenyl)propionate or pharmaceutically acceptable salts thereof is topically administered until no pain on the back. In some embodiments, 2-(diethylamino)ethyl 2-(4-isobutylphenyl)propionate or pharmaceutically acceptable salts thereof is topically administered until no pain on one or both shoulders. In some embodiments, 2-(diethylamino)ethyl 2-(4-isobutylphenyl)propionate or pharmaceutically acceptable salts thereof is topically administered until no pain on one or both side of hips. In some embodiments, 2-(diethylamino)ethyl 2-(4-isobutylphenyl)propionate or pharmaceutically acceptable salts thereof is topically administered until no pain on one or more of fingers. In some embodiments, 2-(diethylamino)ethyl 2-(4-isobutylphenyl)propionate or pharmaceutically acceptable salts thereof is topically administered until no pain on one or more of toes.

In some embodiments, 2-(diethylamino)ethyl 2-(4-isobutylphenyl)propionate is topically administered by a transdermal administration. In some embodiments, 2-(4-isobutylphenyl)propionate or pharmaceutically acceptable salts thereof is topically administered by a dosage form selected from one or more of transdermal patch, cream, foam, gel, lotion, ointment, paste, powder, shake lotion, solid, sponge, tape, tinkture, vapor, injection, drops, rinces, spray, and solution. In some embodiments, 2-(4-isobutylphenyl)propionate or pharmaceutically acceptable salts thereof is topically administered by a dosage form selected from one or more of one or more of transdermal drops, rinses and spray. In some embodiments, 2-(diethylamino)ethyl 2-(4-isobutylphenyl)propionate is topically administered by a spray. In some embodiments, 2-(diethylamino)ethyl 2-(4-isobutylphenyl)propionate is topically administered by a spray for joint suffering from osteoarthritis. In some embodiments, 2-(diethylamino)ethyl 2-(4-isobutylphenyl)propionate is topically administered by a drop. In some embodiments, 2-(diethylamino)ethyl 2-(4-isobutylphenyl)propionate is topically administered by a drop for joint suffering from osteoarthritis.

In some embodiments, 2-(4-isobutylphenyl)propionate or pharmaceutically acceptable salts thereof is topically administered by a dosage form including one or more unit doses. In some embodiments, the dosage from is selected from one or more of transdermal patch, cream, foam, gel, lotion, ointment, paste, powder, shake lotion, solid, sponge, tape, tinkture, vapor, injection, drops, rinses, spray, and solution, and the dosage form including one or more unit doses.

In some embodiments, the dosage form is spray application. In some embodiments, the dosage form is a spray for joint suffering from osteoarthritis. In some embodiments, the dosage form is a plurality times of sprays, and the each one of the unit doses is once spray in the plurality of sprays. In some embodiments, the dosage form is a plurality of patches, and the each one of the unit doses is a patch in the plurality of patches. In some embodiments, the dosage form is drop application. In some embodiments, the dosage form is a drop for joint suffering from osteoarthritis. In some embodiments, the dosage form is a plurality times of drops, and the each one of the unit doses is once drop in the plurality of drops.

In some embodiments, a composition comprising 2-(diethylamino)ethyl 2-(4-isobutylphenyl)propionate is topically administered to the subject. In some embodiments, the unit dose comprising a composition comprising 2-(diethylamino)ethyl 2-(4-isobutylphenyl)propionate is topically administered to the subject.

In some embodiments, 2-(diethylamino)ethyl 2-(4-isobutylphenyl)propionate is topically administered as dissolving in a solution. In some embodiments, the composition is a solution. In some embodiments, the composition is an alcohol solution. In some embodiments, the composition is an acetone solution. In some embodiments, the composition is a dimethyl sulfoxide solution. In some embodiments, the composition is an alcohol water solution. In some embodiments, the composition is an acetone water solution. In some embodiments, the composition is a dimethyl sulfoxide water solution. In some embodiments, the composition is a solution including water and alcohol, wherein said alcohol is at least one, two or more selected from a group consisting of methanol, ethanol, propanol, isopropanol, n-butanol, isobutanol, tert-butanol, n-amyl alcohol, isoamyl alcohol, active amyl alcohol, tert-amyl alcohol, neopentyl alcohol, methyl n-propyl carbinol, methyl isopropyl carbinol and 3-pentanol. In some embodiments, the composition is a solution including water, and ethanol and/or isopropanol.

In some embodiments, the composition is an ethanol water solution. In some embodiments, the composition is 0% to 50% (v/v) ethanol water solution. In some embodiments, the composition is 10%, 15%, 20%, 25%, 30%, 35%, 40%, 45% or 50% (v/v) ethanol water solution. In some embodiments, the composition is 25% (v/v) ethanol water solution.

In some embodiments, in the composition, the concentration of 2-(diethylamino)ethyl 2-(4-isobutylphenyl)propionate and/or pharmaceutically acceptable salts thereof is about 10 mg/mL to about 200 mg/mL, in particular 10 mg/mL to 200 mg/mL. In some embodiments, in the composition, the concentration of 2-(diethylamino)ethyl 2-(4-isobutylphenyl)propionate and/or pharmaceutically acceptable salts thereof is about 30 mg/mL to about 100 mg/mL, in particular 30 mg/mL to 100 mg/mL. In some embodiments, in the composition, the concentration of 2-(diethylamino)ethyl 2-(4-isobutylphenyl)propionate and/or pharmaceutically acceptable salts thereof is about 50 mg/mL to about 80 mg/mL, in particular 50 mg/mL to 80 mg/mL. In some embodiments, in the composition, the concentration of 2-(diethylamino)ethyl 2-(4-isobutylphenyl)propionate and/or pharmaceutically acceptable salts thereof is about 60 mg/mL to about 75 mg/mL, in particular 60 mg/mL to 75 mg/mL. In some embodiments, in the composition, the concentration of 2-(diethylamino)ethyl 2-(4-isobutylphenyl)propionate and/or pharmaceutically acceptable salts thereof is about 70 mg/mL to about 72 mg/mL, in particular 70 mg/mL to 72 mg/mL. In some embodiments, in the composition, the concentration of 2-(diethylamino)ethyl 2-(4-isobutylphenyl)propionate and/or pharmaceutically acceptable salts thereof is about 70 mg/mL, in particular 70 mg/mL. In some embodiments, in the composition, the concentration of 2-(diethylamino)ethyl 2-(4-isobutylphenyl)propionate and/or pharmaceutically acceptable salts thereof is about 72 mg/mL, in particular 72 mg/mL. In some embodiments, in the composition, the concentration of 2-(diethylamino)ethyl 2-(4-isobutylphenyl)propionate and/or pharmaceutically acceptable salts thereof is selected from a group consisting of 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 85 mg/mL, 90 mg/mL, 95 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, and 200 mg/mL.

In some embodiments, in the composition, the concentration of 2-(diethylamino)ethyl 2-(4-isobutylphenyl)propionate and/or pharmaceutically acceptable salts thereof is about 10 mg/g to about 200 mg/g, in particular 10 mg/g to 200 mg/g. In some embodiments, in the composition, the concentration of 2-(diethylamino)ethyl 2-(4-isobutylphenyl)propionate and/or pharmaceutically acceptable salts thereof is about 30 mg/g to about 100 mg/g, in particular 30 mg/g to 100 mg/g. In some embodiments, in the composition, the concentration of 2-(diethylamino)ethyl 2-(4-isobutylphenyl)propionate and/or pharmaceutically acceptable salts thereof is about 50 mg/g to about 80 mg/g, in particular 50 mg/g to 80 mg/g. In some embodiments, in the composition, the concentration of 2-(diethylamino)ethyl 2-(4-isobutylphenyl)propionate and/or pharmaceutically acceptable salts thereof is about 60 mg/g to about 75 mg/g, in particular 60 mg/g to 75 mg/g. In some embodiments, in the composition, the concentration of 2-(diethylamino)ethyl 2-(4-isobutylphenyl)propionate and/or pharmaceutically acceptable salts thereof is about 70 mg/g to about 72 mg/g, in particular 70 mg/g to 72 mg/g. In some embodiments, in the composition, the concentration of 2-(diethylamino)ethyl 2-(4-isobutylphenyl)propionate and/or pharmaceutically acceptable salts thereof is about 70 mg/g, in particular 70 mg/g. In some embodiments, in the composition, the concentration of 2-(diethylamino)ethyl 2-(4-isobutylphenyl)propionate and/or pharmaceutically acceptable salts thereof is about 72 mg/g, in particular 72 mg/g. In some embodiments, in the composition, the concentration of 2-(diethylamino)ethyl 2-(4-isobutylphenyl)propionate and/or pharmaceutically acceptable salts thereof is selected from a group consisting of 5 mg/g, 10 mg/g, 15 mg/g, 20 mg/g, 25 mg/g, 30 mg/g, 35 mg/g, 40 mg/g, 45 mg/g, 50 mg/g, 55 mg/g, 60 mg/g, 61 mg/g, 62 mg/g, 63 mg/g, 64 mg/g, 65 mg/g, 66 mg/g, 67 mg/g, 68 mg/g, 69 mg/g, 70 mg/g, 71 mg/g, 72 mg/g, 73 mg/g, 74 mg/g, 75 mg/g, 76 mg/g, 77 mg/g, 78 mg/g, 79 mg/g, 80 mg/g, 85 mg/g, 90 mg/g, 95 mg/g, 100 mg/g, 110 mg/g, 120 mg/g, 130 mg/g, 140 mg/g, 150 mg/g, 160 mg/g, 170 mg/g, 180 mg/g, 190 mg/g, and 200 mg/g.

In some embodiments, the volume of the composition in the unit dose is about 0.01 mL to about 1 mL, in particular 0.01 mL to 1 mL. In some embodiments, the volume of the composition in the unit dose is about 0.03 mL to about 0.3 mL, in particular 0.03 mL to 0.3 mL. In some embodiments, the volume of the composition in the unit dose is about 0.05 mL to about 0.2 mL, in particular 0.05 mL to 0.2 mL. In some embodiments, the volume of the composition in the unit dose is about 0.05 mL to about 0.1 mL, in particular 0.05 mL to 0.1 mL. In some embodiments, the volume of the composition in the unit dose is about 0.07 mL, in particular 0.07 mL. In some embodiments, the volume of the composition in the unit dose is selected from a group consisting of 0.01 mL, 0.015 mL, 0.02 mL, 0.025 mL, 0.03 mL, 0.035 mL, 0.04 mL, 0.045 mL, 0.05 mL, 0.0525 mL, 0.055 mL, 0.0575 mL, 0.06 mL, 0.0625 mL, 0.065 mL, 0.0675 mL, 0.07 mL, 0.0725 mL, 0.075 mL, 0.08 ML, 0.085 ML, 0.09 ML, 0.095 ML, 0.1 mL, 0.11 mL, 0.12 mL, 0.13 mL, 0.14 mL, 0.15 mL, 0.16 mL, 0.18 mL, 0.2 mL, 0.25 mL, 0.3 mL, 0.35 mL, 0.4 mL, 0.45 mL, 0.5 mL, 0.6 mL, 0.7 mL, 0.8 mL, 0.9 mL, and 1 mL.

In some embodiments, 2-(diethylamino)ethyl 2-(4-isobutylphenyl)propionate or pharmaceutically acceptable salts thereof is administered in an amount of about 0.1 mg to about 20 mg, in particular 0.1 mg to 20 mg per unit dose. In some embodiments, 2-(diethylamino)ethyl 2-(4-isobutylphenyl)propionate or pharmaceutically acceptable salts thereof is administered in an amount of about 0.2 mg to about 18 mg, in particular 0.2 mg to 18 mg per unit dose. In some embodiments, 2-(diethylamino)ethyl 2-(4-isobutylphenyl)propionate or pharmaceutically acceptable salts thereof is administered in an amount of about 0.4 mg to about 16 mg, in particular 0.4 mg to 16 mg per unit dose. In some embodiments, 2-(diethylamino)ethyl 2-(4-isobutylphenyl)propionate or pharmaceutically acceptable salts thereof is administered in an amount of about 0.6 mg to about 14 mg, in particular 0.6 mg to 14 mg per unit dose. In some embodiments, 2-(diethylamino)ethyl 2-(4-isobutylphenyl)propionate or pharmaceutically acceptable salts thereof is administered in an amount of about 0.8 mg to about 12 mg, in particular 0.8 mg to 12 mg per unit dose. In some embodiments, 2-(diethylamino)ethyl 2-(4-isobutylphenyl)propionate or pharmaceutically acceptable salts thereof is administered in an amount of about 1 mg to about 10 mg, in particular 1 mg to 10 mg per unit dose. In some embodiments, 2-(diethylamino)ethyl 2-(4-isobutylphenyl)propionate or pharmaceutically acceptable salts thereof is administered in an amount of about 1.2 mg to about 9 mg, in particular 1.2 mg to 9 mg per unit dose. In some embodiments, 2-(diethylamino)ethyl 2-(4-isobutylphenyl)propionate or pharmaceutically acceptable salts thereof is administered in an amount of about 1.5 mg to about 8 mg, in particular 1.5 mg to 8 mg per unit dose. In some embodiments, 2-(diethylamino)ethyl 2-(4-isobutylphenyl)propionate or pharmaceutically acceptable salts thereof is administered in an amount of about 1.8 mg to about 7 mg, in particular 1.8 mg to 7 mg per unit dose. In some embodiments, 2-(diethylamino)ethyl 2-(4-isobutylphenyl)propionate or pharmaceutically acceptable salts thereof is administered in an amount of about 2 mg to about 6 mg, in particular 2 mg to 6 mg per unit dose. In some embodiments, 2-(diethylamino)ethyl 2-(4-isobutylphenyl)propionate or pharmaceutically acceptable salts thereof is administered in an amount of about 3 mg to about 5 mg, in particular 3 mg to 5 mg per unit dose. In some embodiments, 2-(diethylamino)ethyl 2-(4-isobutylphenyl)propionate or pharmaceutically acceptable salts thereof is administered in an amount of about 4 mg to about 4.75 mg, in particular 4 mg to 4.75 mg per unit dose. In some embodiments, 2-(diethylamino)ethyl 2-(4-isobutylphenyl)propionate or pharmaceutically acceptable salts thereof is administered in an amount of about 4.5 mg, in particular 4.5 mg per unit dose.

In some embodiments, one or more of the unit doses is topically administered to the subject in a single dose, per site, wherein the one or more of unit doses are selected from a group consisting of 1 unit dose, 2 unit doses, 3 unit doses, 4 unit doses, 5 unit doses, 6 unit doses, 7 unit doses, 8 unit doses, 9 unit doses, 10 unit doses, 11 unit doses, 12 unit doses, 13 unit doses, 14 unit doses, 15 unit doses, 16 unit doses, 17 unit doses, 18 unit doses, 19 unit doses, and 20 unit doses. In some embodiments, 1-10 unit doses is topically administered to the subject in a single dose, per site. In some embodiments, 1-8 unit doses is topically administered to the subject in a single dose, per site. In some embodiments, 2-8 unit doses is topically administered to the subject in a single dose, per site. In some embodiments, 2-4 unit doses is topically administered to the subject in a single dose, per site. In some embodiments, 4-8 unit doses is topically administered to the subject in a single dose, per site. In some embodiments, 1 unit dose is topically administered to the subject in a single dose, per site. In some embodiments, 2 unit doses is topically administered to the subject in a single dose, per site. In some embodiments, 3 unit doses is topically administered to the subject in a single dose, per site. In some embodiments, 4 unit doses is topically administered to the subject in a single dose, per site. In some embodiments, 5 unit doses is topically administered to the subject in a single dose, per site. In some embodiments, 6 unit doses is topically administered to the subject in a single dose, per site. In some embodiments, 7 unit doses is topically administered to the subject in a single dose, per site. In some embodiments, 8 unit doses is topically administered to the subject in a single dose, per site. In some embodiments, 9 unit doses is topically administered to the subject in a single dose, per site. In some embodiments, 10 unit doses is topically administered to the subject in a single dose, per site.

In some embodiments, 2-(diethylamino)ethyl 2-(4-isobutylphenyl)propionate or pharmaceutically acceptable salts thereof is administered by a spray capable of spraying about 0.1 mg to about 20 mg, in particular 0.1 mg to 20 mg, of 2-(diethylamino)ethyl 2-(4-isobutylphenyl)propionate and/or pharmaceutically acceptable salts thereof, per spray. In some embodiments, 2-(diethylamino)ethyl 2-(4-isobutylphenyl)propionate or pharmaceutically acceptable salts thereof is administered by a spray capable of spraying about 0.2 mg to about 18 mg, in particular 0.2 mg to 18 mg per spray. In some embodiments, 2-(diethylamino)ethyl 2-(4-isobutylphenyl)propionate or pharmaceutically acceptable salts thereof is administered by a spray capable of spraying about 0.4 mg to about 16 mg, in particular 0.4 mg to 16 mg per spray. In some embodiments, 2-(diethylamino)ethyl 2-(4-isobutylphenyl)propionate or pharmaceutically acceptable salts thereof is administered by a spray capable of spraying about 0.6 mg to about 14 mg, in particular 0.6 mg to 14 mg per spray. In some embodiments, 2-(diethylamino)ethyl 2-(4-isobutylphenyl)propionate or pharmaceutically acceptable salts thereof is administered by a spray capable of spraying about 0.8 mg to about 12 mg, in particular 0.8 mg to 12 mg per spray. In some embodiments, 2-(diethylamino)ethyl 2-(4-isobutylphenyl)propionate or pharmaceutically acceptable salts thereof is administered by a spray capable of spraying about 1 mg to about 10 mg, in particular 1 mg to 10 mg per spray. In some embodiments, 2-(diethylamino)ethyl 2-(4-isobutylphenyl)propionate or pharmaceutically acceptable salts thereof is administered by a spray capable of spraying about 1.2 mg to about 9 mg, in particular 1.2 mg to 9 mg per spray. In some embodiments, 2-(diethylamino)ethyl 2-(4-isobutylphenyl)propionate or pharmaceutically acceptable salts thereof is administered by a spray capable of spraying about 1.5 mg to about 8 mg, in particular 1.5 mg to 8 mg per spray. In some embodiments, 2-(diethylamino)ethyl 2-(4-isobutylphenyl)propionate or pharmaceutically acceptable salts thereof is administered by a spray capable of spraying about 1.8 mg to about 7 mg, in particular 1.8 mg to 7 mg per spray. In some embodiments, 2-(diethylamino)ethyl 2-(4-isobutylphenyl)propionate or pharmaceutically acceptable salts thereof is administered by a spray capable of spraying about 2 mg to about 6 mg, in particular 2 mg to 6 mg per spray. In some embodiments, 2-(diethylamino)ethyl 2-(4-isobutylphenyl)propionate or pharmaceutically acceptable salts thereof is administered by a spray capable of spraying about 3 mg to about 5 mg, in particular 3 mg to 5 mg per spray. In some embodiments, 2-(diethylamino)ethyl 2-(4-isobutylphenyl)propionate or pharmaceutically acceptable salts thereof is administered by a spray capable of spraying about 4 mg to about 4.75 mg, in particular 4 mg to 4.75 mg per spray. In some embodiments, 2-(diethylamino)ethyl 2-(4-isobutylphenyl)propionate or pharmaceutically acceptable salts thereof is administered by a spray capable of spraying about 4.5 mg, in particular 4.5 mg per spray.

In some embodiments, the dosage form is a spray. In some embodiments, the volume of the composition per spray is about 0.01 mL to about 1 mL, in particular 0.01 mL to 1 mL. In some embodiments, the volume of the composition per spray is about 0.03 mL to about 0.3 mL, in particular 0.03 mL to 0.3 mL. In some embodiments, the volume of the composition per spray is about 0.05 mL to about 0.2 mL, in particular 0.05 mL to 0.2 mL. In some embodiments, the volume of the composition per spray is about 0.05 mL to about 0.1 mL, in particular 0.05 mL to 0.1 mL. In some embodiments, the volume of the composition per spray is about 0.0625 mL, in particular 0.0625 mL. In some embodiments, the volume of the composition per spray is selected from a group consisting of 0.01 mL, 0.015 mL, 0.02 mL, 0.025 mL, 0.03 mL, 0.035 mL, 0.04 mL, 0.045 mL, 0.05 mL, 0.0525 mL, 0.055 mL, 0.0575 mL, 0.06 mL, 0.0625 mL, 0.065 mL, 0.0675 mL, 0.07 mL, 0.0725 mL, 0.075 mL, 0.08 ML, 0.085 ML, 0.09 ML, 0.095 mL, 0.1 mL, 0.11 mL, 0.12 mL, 0.13 mL, 0.14 mL, 0.15 mL, 0.16 ML, 0.18 mL, 0.2 mL, 0.25 ML, 0.3 ML, 0.35 mL, 0.4 mL, 0.45 mL, 0.5 mL, 0.6 mL, 0.7 mL, 0.8 mL, 0.9 mL, and 1 mL.

In some embodiments, the dosage form is a spray. In some embodiments, 2-(diethylamino)ethyl 2-(4-isobutylphenyl)propionate or pharmaceutically acceptable salts thereof is administered in an amount of about 0.1 mg to about 20 mg, in particular 0.1 mg to 20 mg per spray. In some embodiments, 2-(diethylamino)ethyl 2-(4-isobutylphenyl)propionate or pharmaceutically acceptable salts thereof is administered in an amount of about 0.2 mg to about 18 mg, in particular 0.2 mg to 18 mg per spray. In some embodiments, 2-(diethylamino)ethyl 2-(4-isobutylphenyl)propionate or pharmaceutically acceptable salts thereof is administered in an amount of about 0.4 mg to about 16 mg, in particular 0.4 mg to 16 mg per spray. In some embodiments, 2-(diethylamino)ethyl 2-(4-isobutylphenyl)propionate or pharmaceutically acceptable salts thereof is administered in an amount of about 0.6 mg to about 14 mg, in particular 0.6 mg to 14 mg per spray. In some embodiments, 2-(diethylamino)ethyl 2-(4-isobutylphenyl)propionate or pharmaceutically acceptable salts thereof is administered in an amount of about 0.8 mg to about 12 mg, in particular 0.8 mg to 12 mg per spray. In some embodiments, 2-(diethylamino)ethyl 2-(4-isobutylphenyl)propionate or pharmaceutically acceptable salts thereof is administered in an amount of about 1 mg to about 10 mg, in particular 1 mg to 10 mg per spray. In some embodiments, 2-(diethylamino)ethyl 2-(4-isobutylphenyl)propionate or pharmaceutically acceptable salts thereof is administered in an amount of about 1.2 mg to about 9 mg, in particular 1.2 mg to 9 mg per spray. In some embodiments, 2-(diethylamino)ethyl 2-(4-isobutylphenyl)propionate or pharmaceutically acceptable salts thereof is administered in an amount of about 1.5 mg to about 8 mg, in particular 1.5 mg to 8 mg per spray. In some embodiments, 2-(diethylamino)ethyl 2-(4-isobutylphenyl)propionate or pharmaceutically acceptable salts thereof is administered in an amount of about 1.8 mg to about 7 mg, in particular 1.8 mg to 7 mg per spray. In some embodiments, 2-(diethylamino)ethyl 2-(4-isobutylphenyl)propionate or pharmaceutically acceptable salts thereof is administered in an amount of about 2 mg to about 6 mg, in particular 2 mg to 6 mg per spray. In some embodiments, 2-(diethylamino)ethyl 2-(4-isobutylphenyl)propionate or pharmaceutically acceptable salts thereof is administered in an amount of about 3 mg to about 5 mg, in particular 3 mg to 5 mg per spray. In some embodiments, 2-(diethylamino)ethyl 2-(4-isobutylphenyl)propionate or pharmaceutically acceptable salts thereof is administered in an amount of about 4 mg to about 4.75 mg, in particular 4 mg to 4.75 mg per spray. In some embodiments, 2-(diethylamino)ethyl 2-(4-isobutylphenyl)propionate or pharmaceutically acceptable salts thereof is administered in an amount of about 4.5 mg, in particular 4.5 mg per spray.

In some embodiments, the drug strength per spray is between 0.1 mg to 75 mg of free base of 2-(diethylamino)ethyl 2-(4-isobutylphenyl)propionate. In some embodiments, the drug strength per spray is about 1 mg to about 20 mg, in particular 1 mg to 20 mg, of free base of 2-(diethylamino)ethyl 2-(4-isobutylphenyl)propionate. In some embodiments, the drug strength per spray is about 1.2 mg to about 10 mg, in particular 1.2 mg to 10 mg, of free base of 2-(diethylamino)ethyl 2-(4-isobutylphenyl)propionate. In some embodiments, the drug strength per spray is about 0.1 mg to about 20 mg, in particular 0.1 mg to 20 mg, of free base of 2-(diethylamino)ethyl 2-(4-isobutylphenyl)propionate. In some embodiments, the drug strength per spray is about 2 mg to about 6 mg, in particular 2 mg to 6 mg, of free base of 2-(diethylamino)ethyl 2-(4-isobutylphenyl)propionate. In some embodiments, the drug strength per spray is about 3 mg to about 5 mg, in particular 3 mg to 5 mg, of free base of 2-(diethylamino)ethyl 2-(4-isobutylphenyl)propionate. In some embodiments, the drug strength per spray is about 4 mg to about 4.5 mg, in particular 4 mg to 4.5 mg, of free base of 2-(diethylamino)ethyl 2-(4-isobutylphenyl)propionate.

In some embodiments, 2-(diethylamino)ethyl 2-(4-isobutylphenyl)propionate or pharmaceutically acceptable salts thereof is administered by a drop capable of dropping about 0.1 mg to about 20 mg, in particular 0.1 mg to 20 mg, of 2-(diethylamino)ethyl 2-(4-isobutylphenyl)propionate and/or pharmaceutically acceptable salts thereof, per drop. In some embodiments, 2-(diethylamino)ethyl 2-(4-isobutylphenyl)propionate or pharmaceutically acceptable salts thereof is administered by a drop capable of dropping about 0.2 mg to about 18 mg, in particular 0.2 mg to 18 mg per drop. In some embodiments, 2-(diethylamino)ethyl 2-(4-isobutylphenyl)propionate or pharmaceutically acceptable salts thereof is administered by a drop capable of dropping about 0.4 mg to about 16 mg, in particular 0.4 mg to 16 mg per drop. In some embodiments, 2-(diethylamino)ethyl 2-(4-isobutylphenyl)propionate or pharmaceutically acceptable salts thereof is administered by a drop capable of dropping about 0.6 mg to about 14 mg, in particular 0.6 mg to 14 mg per drop. In some embodiments, 2-(diethylamino)ethyl 2-(4-isobutylphenyl)propionate or pharmaceutically acceptable salts thereof is administered by a drop capable of dropping about 0.8 mg to about 12 mg, in particular 0.8 mg to 12 mg per drop. In some embodiments, 2-(diethylamino)ethyl 2-(4-isobutylphenyl)propionate or pharmaceutically acceptable salts thereof is administered by a drop capable of dropping about 1 mg to about 10 mg, in particular 1 mg to 10 mg per drop. In some embodiments, 2-(diethylamino)ethyl 2-(4-isobutylphenyl)propionate or pharmaceutically acceptable salts thereof is administered by a drop capable of dropping about 1.2 mg to about 9 mg, in particular 1.2 mg to 9 mg per drop. In some embodiments, 2-(diethylamino)ethyl 2-(4-isobutylphenyl)propionate or pharmaceutically acceptable salts thereof is administered by a drop capable of dropping about 1.5 mg to about 8 mg, in particular 1.5 mg to 8 mg per drop. In some embodiments, 2-(diethylamino)ethyl 2-(4-isobutylphenyl)propionate or pharmaceutically acceptable salts thereof is administered by a drop capable of dropping about 1.8 mg to about 7 mg, in particular 1.8 mg to 7 mg per drop. In some embodiments, 2-(diethylamino)ethyl 2-(4-isobutylphenyl)propionate or pharmaceutically acceptable salts thereof is administered by a drop capable of dropping about 2 mg to about 6 mg, in particular 2 mg to 6 mg per drop. In some embodiments, 2-(diethylamino)ethyl 2-(4-isobutylphenyl)propionate or pharmaceutically acceptable salts thereof is administered by a drop capable of dropping about 3 mg to about 5 mg, in particular 3 mg to 5 mg per drop. In some embodiments, 2-(diethylamino)ethyl 2-(4-isobutylphenyl)propionate or pharmaceutically acceptable salts thereof is administered by a drop capable of dropping about 4 mg to about 4.75 mg, in particular 4 mg to 4.75 mg per drop. In some embodiments, 2-(diethylamino)ethyl 2-(4-isobutylphenyl)propionate or pharmaceutically acceptable salts thereof is administered by a drop capable of dropping about 4.5 mg, in particular 4.5 mg per drop.

In some embodiments, the dosage form is a drop. In some embodiments, the volume of the composition per drop is about 0.01 mL to about 1 mL, in particular 0.01 mL to 1 mL. In some embodiments, the volume of the composition per drop is about 0.03 mL to about 0.3 mL, in particular 0.03 mL to 0.3 mL. In some embodiments, the volume of the composition per drop is about 0.05 mL to about 0.2 mL, in particular 0.05 mL to 0.2 mL. In some embodiments, the volume of the composition per drop is about 0.05 mL to about 0.1 mL, in particular 0.05 mL to 0.1 mL. In some embodiments, the volume of the composition per drop is about 0.0625 mL, in particular 0.0625 mL. In some embodiments, the volume of the composition per drop is selected from a group consisting of 0.01 mL, 0.015 mL, 0.02 mL, 0.025 mL, 0.03 mL, 0.035 mL, 0.04 mL, 0.045 mL, 0.05 mL, 0.0525 mL, 0.055 mL, 0.0575 mL, 0.06 mL, 0.0625 mL, 0.065 mL, 0.0675 mL, 0.07 mL, 0.0725 mL, 0.075 mL, 0.08 mL, 0.085 mL, 0.09 mL, 0.095 mL, 0.1 mL, 0.11 mL, 0.12 mL, 0.13 mL, 0.14 mL, 0.15 mL, 0.16 mL, 0.18 mL, 0.2 mL, 0.25 mL, 0.3 mL, 0.35 mL, 0.4 mL, 0.45 mL, 0.5 mL, 0.6 mL, 0.7 mL, 0.8 mL, 0.9 mL, and 1 mL.

In some embodiments, the dosage form is a drop. In some embodiments, 2-(diethylamino)ethyl 2-(4-isobutylphenyl)propionate or pharmaceutically acceptable salts thereof is administered in an amount of about 0.1 mg to about 20 mg, in particular 0.1 mg to 20 mg per drop. In some embodiments, 2-(diethylamino)ethyl 2-(4-isobutylphenyl)propionate or pharmaceutically acceptable salts thereof is administered in an amount of about 0.2 mg to about 18 mg, in particular 0.2 mg to 18 mg per drop. In some embodiments, 2-(diethylamino)ethyl 2-(4-isobutylphenyl)propionate or pharmaceutically acceptable salts thereof is administered in an amount of about 0.4 mg to about 16 mg, in particular 0.4 mg to 16 mg per drop. In some embodiments, 2-(diethylamino)ethyl 2-(4-isobutylphenyl)propionate or pharmaceutically acceptable salts thereof is administered in an amount of about 0.6 mg to about 14 mg, in particular 0.6 mg to 14 mg per drop. In some embodiments, 2-(diethylamino)ethyl 2-(4-isobutylphenyl)propionate or pharmaceutically acceptable salts thereof is administered in an amount of about 0.8 mg to about 12 mg, in particular 0.8 mg to 12 mg per drop. In some embodiments, 2-(diethylamino)ethyl 2-(4-isobutylphenyl)propionate or pharmaceutically acceptable salts thereof is administered in an amount of about 1 mg to about 10 mg, in particular 1 mg to 10 mg per drop. In some embodiments, 2-(diethylamino)ethyl 2-(4-isobutylphenyl)propionate or pharmaceutically acceptable salts thereof is administered in an amount of about 1.2 mg to about 9 mg, in particular 1.2 mg to 9 mg per drop. In some embodiments, 2-(diethylamino)ethyl 2-(4-isobutylphenyl)propionate or pharmaceutically acceptable salts thereof is administered in an amount of about 1.5 mg to about 8 mg, in particular 1.5 mg to 8 mg per drop. In some embodiments, 2-(diethylamino)ethyl 2-(4-isobutylphenyl)propionate or pharmaceutically acceptable salts thereof is administered in an amount of about 1.8 mg to about 7 mg, in particular 1.8 mg to 7 mg per drop. In some embodiments, 2-(diethylamino)ethyl 2-(4-isobutylphenyl)propionate or pharmaceutically acceptable salts thereof is administered in an amount of about 2 mg to about 6 mg, in particular 2 mg to 6 mg per drop. In some embodiments, 2-(diethylamino)ethyl 2-(4-isobutylphenyl)propionate or pharmaceutically acceptable salts thereof is administered in an amount of about 3 mg to about 5 mg, in particular 3 mg to 5 mg per drop. In some embodiments, 2-(diethylamino)ethyl 2-(4-isobutylphenyl)propionate or pharmaceutically acceptable salts thereof is administered in an amount of about 4 mg to about 4.75 mg, in particular 4 mg to 4.75 mg per drop. In some embodiments, 2-(diethylamino)ethyl 2-(4-isobutylphenyl)propionate or pharmaceutically acceptable salts thereof is administered in an amount of about 4.5 mg, in particular 4.5 mg per drop.

In some embodiments, the drug strength per drop is between 0.1 mg to 75 mg of free base of 2-(diethylamino)ethyl 2-(4-isobutylphenyl)propionate. In some embodiments, the drug strength per drop is about 1 mg to about 20 mg, in particular 1 mg to 20 mg, of free base of 2-(diethylamino)ethyl 2-(4-isobutylphenyl)propionate. In some embodiments, the drug strength per drop is about 1.2 mg to about 10 mg, in particular 1.2 mg to 10 mg, of free base of 2-(diethylamino)ethyl 2-(4-isobutylphenyl)propionate. In some embodiments, the drug strength per drop is about 0.1 mg to about 20 mg, in particular 0.1 mg to 20 mg, of free base of 2-(diethylamino)ethyl 2-(4-isobutylphenyl)propionate. In some embodiments, the drug strength per drop is about 2 mg to about 6 mg, in particular 2 mg to 6 mg, of free base of 2-(diethylamino)ethyl 2-(4-isobutylphenyl)propionate. In some embodiments, the drug strength per drop is about 3 mg to about 5 mg, in particular 3 mg to 5 mg, of free base of 2-(diethylamino)ethyl 2-(4-isobutylphenyl)propionate. In some embodiments, the drug strength per drop is about 4 mg to about 4.5 mg, in particular 4 mg to 4.5 mg, of free base of 2-(diethylamino)ethyl 2-(4-isobutylphenyl)propionate.

In some embodiments, 2-(diethylamino)ethyl 2-(4-isobutylphenyl)propionate or pharmaceutically acceptable salts thereof is administered by a patch capable of administering about 0.1 mg to about 20 mg, in particular 0.1 mg to 20 mg, of 2-(diethylamino)ethyl 2-(4-isobutylphenyl)propionate and/or pharmaceutically acceptable salts thereof, per patch. In some embodiments, 2-(diethylamino)ethyl 2-(4-isobutylphenyl)propionate or pharmaceutically acceptable salts thereof is administered by a patch capable of administering about 0.2 mg to about 18 mg, in particular 0.2 mg to 18 mg per patch. In some embodiments, 2-(diethylamino)ethyl 2-(4-isobutylphenyl)propionate or pharmaceutically acceptable salts thereof is administered by a patch capable of administering about 0.4 mg to about 16 mg, in particular 0.4 mg to 16 mg per patch. In some embodiments, 2-(diethylamino)ethyl 2-(4-isobutylphenyl)propionate or pharmaceutically acceptable salts thereof is administered by a patch capable of administering about 0.6 mg to about 14 mg, in particular 0.6 mg to 14 mg per patch. In some embodiments, 2-(diethylamino)ethyl 2-(4-isobutylphenyl)propionate or pharmaceutically acceptable salts thereof is administered by a patch capable of administering about 0.8 mg to about 12 mg, in particular 0.8 mg to 12 mg per patch. In some embodiments, 2-(diethylamino)ethyl 2-(4-isobutylphenyl)propionate or pharmaceutically acceptable salts thereof is administered by a patch capable of administering about 1 mg to about 10 mg, in particular 1 mg to 10 mg per patch. In some embodiments, 2-(diethylamino)ethyl 2-(4-isobutylphenyl)propionate or pharmaceutically acceptable salts thereof is administered by a patch capable of administering about 1.2 mg to about 9 mg, in particular 1.2 mg to 9 mg per patch. In some embodiments, 2-(diethylamino)ethyl 2-(4-isobutylphenyl)propionate or pharmaceutically acceptable salts thereof is administered by a patch capable of administering about 1.5 mg to about 8 mg, in particular 1.5 mg to 8 mg per patch. In some embodiments, 2-(diethylamino)ethyl 2-(4-isobutylphenyl)propionate or pharmaceutically acceptable salts thereof is administered by a patch capable of administering about 1.8 mg to about 7 mg, in particular 1.8 mg to 7 mg per patch. In some embodiments, 2-(diethylamino)ethyl 2-(4-isobutylphenyl)propionate or pharmaceutically acceptable salts thereof is administered by a patch capable of administering about 2 mg to about 6 mg, in particular 2 mg to 6 mg per patch. In some embodiments, 2-(diethylamino)ethyl 2-(4-isobutylphenyl)propionate or pharmaceutically acceptable salts thereof is administered by a patch capable of administering about 3 mg to about 5 mg, in particular 3 mg to 5 mg per patch. In some embodiments, 2-(diethylamino)ethyl 2-(4-isobutylphenyl)propionate or pharmaceutically acceptable salts thereof is administered by a patch capable of administering about 4 mg to about 4.75 mg, in particular 4 mg to 4.75 mg per patch. In some embodiments, 2-(diethylamino)ethyl 2-(4-isobutylphenyl)propionate or pharmaceutically acceptable salts thereof is administered by a patch capable of administering about 4.5 mg, in particular 4.5 mg per patch.

In some embodiments, the dosage form is a patch. In some embodiments, the volume of the composition per patch is about 0.01 mL to about 1 mL, in particular 0.01 mL to 1 mL. In some embodiments, the volume of the composition per patch is about 0.03 mL to about 0.3 mL, in particular 0.03 mL to 0.3 mL. In some embodiments, the volume of the composition per patch is about 0.05 mL to about 0.2 mL, in particular 0.05 mL to 0.2 mL. In some embodiments, the volume of the composition per patch is about 0.05 mL to about 0.1 mL, in particular 0.05 mL to 0.1 mL. In some embodiments, the volume of the composition per patch is about 0.0625 mL, in particular 0.0625 mL. In some embodiments, the volume of the composition per patch is selected from a group consisting of 0.01 mL, 0.015 mL, 0.02 mL, 0.025 mL, 0.03 mL, 0.035 mL, 0.04 mL, 0.045 mL, 0.05 mL, 0.0525 mL, 0.055 mL, 0.0575 mL, 0.06 mL, 0.0625 mL, 0.065 mL, 0.0675 mL, 0.07 mL, 0.0725 mL, 0.075 mL, 0.08 ML, 0.085 ML, 0.09 ML, 0.095 mL, 0.1 mL, 0.11 mL, 0.12 mL, 0.13 mL, 0.14 mL, 0.15 mL, 0.16 ML, 0.18 mL, 0.2 mL, 0.25 ML, 0.3 ML, 0.35 mL, 0.4 mL, 0.45 mL, 0.5 mL, 0.6 mL, 0.7 mL, 0.8 mL, 0.9 mL, and 1 mL.

In some embodiments, the dosage form is a patch. In some embodiments, 2-(diethylamino)ethyl 2-(4-isobutylphenyl)propionate or pharmaceutically acceptable salts thereof is administered in an amount of about 0.1 mg to about 20 mg, in particular 0.1 mg to 20 mg per patch. In some embodiments, 2-(diethylamino)ethyl 2-(4-isobutylphenyl)propionate or pharmaceutically acceptable salts thereof is administered in an amount of about 0.2 mg to about 18 mg, in particular 0.2 mg to 18 mg per patch. In some embodiments, 2-(diethylamino)ethyl 2-(4-isobutylphenyl)propionate or pharmaceutically acceptable salts thereof is administered in an amount of about 0.4 mg to about 16 mg, in particular 0.4 mg to 16 mg per patch. In some embodiments, 2-(diethylamino)ethyl 2-(4-isobutylphenyl)propionate or pharmaceutically acceptable salts thereof is administered in an amount of about 0.6 mg to about 14 mg, in particular 0.6 mg to 14 mg per patch. In some embodiments, 2-(diethylamino)ethyl 2-(4-isobutylphenyl)propionate or pharmaceutically acceptable salts thereof is administered in an amount of about 0.8 mg to about 12 mg, in particular 0.8 mg to 12 mg per patch. In some embodiments, 2-(diethylamino)ethyl 2-(4-isobutylphenyl)propionate or pharmaceutically acceptable salts thereof is administered in an amount of about 1 mg to about 10 mg, in particular 1 mg to 10 mg per patch. In some embodiments, 2-(diethylamino)ethyl 2-(4-isobutylphenyl)propionate or pharmaceutically acceptable salts thereof is administered in an amount of about 1.2 mg to about 9 mg, in particular 1.2 mg to 9 mg per patch. In some embodiments, 2-(diethylamino)ethyl 2-(4-isobutylphenyl)propionate or pharmaceutically acceptable salts thereof is administered in an amount of about 1.5 mg to about 8 mg, in particular 1.5 mg to 8 mg per patch. In some embodiments, 2-(diethylamino)ethyl 2-(4-isobutylphenyl)propionate or pharmaceutically acceptable salts thereof is administered in an amount of about 1.8 mg to about 7 mg, in particular 1.8 mg to 7 mg per patch. In some embodiments, 2-(diethylamino)ethyl 2-(4-isobutylphenyl)propionate or pharmaceutically acceptable salts thereof is administered in an amount of about 2 mg to about 6 mg, in particular 2 mg to 6 mg per patch. In some embodiments, 2-(diethylamino)ethyl 2-(4-isobutylphenyl)propionate or pharmaceutically acceptable salts thereof is administered in an amount of about 3 mg to about 5 mg, in particular 3 mg to 5 mg per patch. In some embodiments, 2-(diethylamino)ethyl 2-(4-isobutylphenyl)propionate or pharmaceutically acceptable salts thereof is administered in an amount of about 4 mg to about 4.75 mg, in particular 4 mg to 4.75 mg per patch. In some embodiments, 2-(diethylamino)ethyl 2-(4-isobutylphenyl)propionate or pharmaceutically acceptable salts thereof is administered in an amount of about 4.5 mg, in particular 4.5 mg per patch.

In some embodiments, 2-(diethylamino)ethyl 2-(4-isobutylphenyl)propionate or pharmaceutically acceptable salts thereof is administered in an amount, administered by a spray in an amount, administered by a spray capable of administering in an amount, administered by a drop in an amount, administered by a drop capable of administering in an amount, administered by a patch in an amount, and/or administered a patch capable of administering in an amount, selected from a group consisting of 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.55 mg, 1.6 mg, 1.65 mg, 1.7 mg, 1.75 mg, 1.8 mg, 1.85 mg, 1.9 mg, 1.95 mg, 2 mg, 2.05 mg, 2.1 mg, 2.15 mg, 2.2 mg, 2.25 mg, 2.3 mg, 2.35 mg, 2.4 mg, 2.45 mg, 2.5 mg, 2.55 mg, 2.6 mg, 2.65 mg, 2.7 mg, 2.75 mg, 2.8 mg, 2.85 mg, 2.9 mg, 2.95 mg, 3 mg, 3.05 mg, 3.1 mg, 3.15 mg, 3.2 mg, 3.25 mg, 3.3 mg, 3.35 mg, 3.4 mg, 3.45 mg, 3.5 mg, 3.55 mg, 3.6 mg, 3.65 mg, 3.7 mg, 3.75 mg, 3.8 mg, 3.85 mg, 3.9 mg, 3.95 mg, 4 mg, 4.025 mg, 4.05 mg, 4.075 mg, 4.1 mg, 4.125 mg, 4.15 mg, 4.175 mg, 4.2 mg, 4.225 mg, 4.25 mg, 4.275 mg, 4.3 mg, 4.325 mg, 4.35 mg, 4.375 mg, 4.4 mg, 4.425 mg, 4.45 mg, 4.475 mg, 4.5 mg, 4.525 mg, 4.55 mg, 4.575 mg, 4.6 mg, 4.625 mg, 4.65 mg, 4.675 mg, 4.7 mg, 4.725 mg, 4.75 mg, 4.775 mg, 4.8 mg, 4.825 mg, 4.85 mg, 4.875 mg, 4.9 mg, 4.925 mg, 4.95 mg, 4.975 mg, 5 mg, 5.05 mg, 5.1 mg, 5.15 mg, 5.2 mg, 5.25 mg, 5.3 mg, 5.35 mg, 5.4 mg, 5.45 mg, 5.5 mg, 5.55 mg, 5.6 mg, 5.65 mg, 5.7 mg, 5.75 mg, 5.8 mg, 5.85 mg, 5.9 mg, 5.95 mg, 6 mg, 6.05 mg, 6.1 mg, 6.15 mg, 6.2 mg, 6.25 mg, 6.3 mg, 6.35 mg, 6.4 mg, 6.45 mg, 6.5 mg, 6.55 mg, 6.6 mg, 6.65 mg, 6.7 mg, 6.75 mg, 6.8 mg, 6.85 mg, 6.9 mg, 6.95 mg, 7 mg, 7.05 mg, 7.1 mg, 7.15 mg, 7.2 mg, 7.25 mg, 7.3 mg, 7.35 mg, 7.4 mg, 7.45 mg, 7.5 mg, 7.55 mg, 7.6 mg, 7.65 mg, 7.7 mg, 7.75 mg, 7.8 mg, 7.85 mg, 7.9 mg, 7.95 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, 10 mg, 10.1 mg, 10.2 mg, 10.3 mg, 10.4 mg, 10.5 mg, 10.6 mg, 10.7 mg, 10.8 mg, 10.9 mg, 11 mg, 11.1 mg, 11.2 mg, 11.3 mg, 11.4 mg, 11.5 mg, 11.6 mg, 11.7 mg, 11.8 mg, 11.9 mg, 12 mg, 12.1 mg, 12.2 mg, 12.3 mg, 12.4 mg, 12.5 mg, 12.6 mg, 12.7 mg, 12.8 mg, 12.9 mg, 13 mg, 13.1 mg, 13.2 mg, 13.3 mg, 13.4 mg, 13.5 mg, 13.6 mg, 13.7 mg, 13.8 mg, 13.9 mg, 14 mg, 14.1 mg, 14.2 mg, 14.3 mg, 14.4 mg, 14.5 mg, 14.6 mg, 14.7 mg, 14.8 mg, 14.9 mg, 15 mg, 15.1 mg, 15.2 mg, 15.3 mg, 15.4 mg, 15.5 mg, 15.6 mg, 15.7 mg, 15.8 mg, 15.9 mg, 16 mg, 16.1 mg, 16.2 mg, 16.3 mg, 16.4 mg, 16.5 mg, 16.6 mg, 16.7 mg, 16.8 mg, 16.9 mg, 17 mg, 17.1 mg, 17.2 mg, 17.3 mg, 17.4 mg, 17.5 mg, 17.6 mg, 17.7 mg, 17.8 mg, 17.9 mg, 18 mg, 18.1 mg, 18.2 mg, 18.3 mg, 18.4 mg, 18.5 mg, 18.6 mg, 18.7 mg, 18.8 mg, 18.9 mg, 19 mg, 19.1 mg, 19.2 mg, 19.3 mg, 19.4 mg, 19.5 mg, 19.6 mg, 19.7 mg, 19.8 mg, 19.9 mg and 20 mg, per unit dose, per spray, per drop, and/or per patch.

In some embodiments, the drug strength per patch is between 0.1 mg to 75 mg of free base of 2-(diethylamino)ethyl 2-(4-isobutylphenyl)propionate. In some embodiments, the drug strength per patch is about 1 mg to about 20 mg, in particular 1 mg to 20 mg, of free base of 2-(diethylamino)ethyl 2-(4-isobutylphenyl)propionate. In some embodiments, the drug strength per patch is about 1.2 mg to about 10 mg, in particular 1.2 mg to 10 mg, of free base of 2-(diethylamino)ethyl 2-(4-isobutylphenyl)propionate. In some embodiments, the drug strength per patch is about 0.1 mg to about 20 mg, in particular 0.1 mg to 20 mg, of free base of 2-(diethylamino)ethyl 2-(4-isobutylphenyl)propionate. In some embodiments, the drug strength per patch is about 2 mg to about 6 mg, in particular 2 mg to 6 mg, of free base of 2-(diethylamino)ethyl 2-(4-isobutylphenyl)propionate. In some embodiments, the drug strength per patch is about 3 mg to about 5 mg, in particular 3 mg to 5 mg, of free base of 2-(diethylamino)ethyl 2-(4-isobutylphenyl)propionate. In some embodiments, the drug strength per patch is about 4 mg to about 4.5 mg, in particular 4 mg to 4.5 mg, of free base of 2-(diethylamino)ethyl 2-(4-isobutylphenyl)propionate.

In some embodiments, the drug strength per spray, per drop and/or per patch is selected from a group consisting of 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.55 mg, 1.6 mg, 1.65 mg, 1.7 mg, 1.75 mg, 1.8 mg, 1.85 mg, 1.9 mg, 1.95 mg, 2 mg, 2.05 mg, 2.1 mg, 2.15 mg, 2.2 mg, 2.25 mg, 2.3 mg, 2.35 mg, 2.4 mg, 2.45 mg, 2.5 mg, 2.55 mg, 2.6 mg, 2.65 mg, 2.7 mg, 2.75 mg, 2.8 mg, 2.85 mg, 2.9 mg, 2.95 mg, 3 mg, 3.05 mg, 3.1 mg, 3.15 mg, 3.2 mg, 3.25 mg, 3.3 mg, 3.35 mg, 3.4 mg, 3.45 mg, 3.5 mg, 3.55 mg, 3.6 mg, 3.65 mg, 3.7 mg, 3.75 mg, 3.8 mg, 3.85 mg, 3.9 mg, 3.95 mg, 4 mg, 4.025 mg, 4.05 mg, 4.075 mg, 4.1 mg, 4.125 mg, 4.15 mg, 4.175 mg, 4.2 mg, 4.225 mg, 4.25 mg, 4.275 mg, 4.3 mg, 4.325 mg, 4.35 mg, 4.375 mg, 4.4 mg, 4.425 mg, 4.45 mg, 4.475 mg, 4.5 mg, 4.525 mg, 4.55 mg, 4.575 mg, 4.6 mg, 4.625 mg, 4.65 mg, 4.675 mg, 4.7 mg, 4.725 mg, 4.75 mg, 4.775 mg, 4.8 mg, 4.825 mg, 4.85 mg, 4.875 mg, 4.9 mg, 4.925 mg, 4.95 mg, 4.975 mg, 5 mg, 5.05 mg, 5.1 mg, 5.15 mg, 5.2 mg, 5.25 mg, 5.3 mg, 5.35 mg, 5.4 mg, 5.45 mg, 5.5 mg, 5.55 mg, 5.6 mg, 5.65 mg, 5.7 mg, 5.75 mg, 5.8 mg, 5.85 mg, 5.9 mg, 5.95 mg, 6 mg, 6.05 mg, 6.1 mg, 6.15 mg, 6.2 mg, 6.25 mg, 6.3 mg, 6.35 mg, 6.4 mg, 6.45 mg, 6.5 mg, 6.55 mg, 6.6 mg, 6.65 mg, 6.7 mg, 6.75 mg, 6.8 mg, 6.85 mg, 6.9 mg, 6.95 mg, 7 mg, 7.05 mg, 7.1 mg, 7.15 mg, 7.2 mg, 7.25 mg, 7.3 mg, 7.35 mg, 7.4 mg, 7.45 mg, 7.5 mg, 7.55 mg, 7.6 mg, 7.65 mg, 7.7 mg, 7.75 mg, 7.8 mg, 7.85 mg, 7.9 mg, 7.95 mg, 8 mg, of free base of 2-(diethylamino)ethyl 2-(4-isobutylphenyl)propionate.

In some embodiments, 2-(diethylamino)ethyl 2-(4-isobutylphenyl)propionate or pharmaceutically acceptable salts thereof is administered once, twice, three times, four times, five times or six times a day, or once every one, two, three, four, five, six, or seven days.

In some embodiments, 2-(diethylamino)ethyl 2-(4-isobutylphenyl)propionate or pharmaceutically acceptable salts thereof is administered once, twice, three times, four times, five times, six times, seven times or eight times a day. In some embodiments, 2-(diethylamino)ethyl 2-(4-isobutylphenyl)propionate or pharmaceutically acceptable salts thereof is administered once a day. In some embodiments, 2-(diethylamino)ethyl 2-(4-isobutylphenyl)propionate or pharmaceutically acceptable salts thereof is administered twice a day. In some embodiments, 2-(diethylamino)ethyl 2-(4-isobutylphenyl)propionate or pharmaceutically acceptable salts thereof is administered three times a day. In some embodiments, 2-(diethylamino)ethyl 2-(4-isobutylphenyl)propionate or pharmaceutically acceptable salts thereof is administered four a day. In some embodiments, 2-(diethylamino)ethyl 2-(4-isobutylphenyl)propionate or pharmaceutically acceptable salts thereof is administered five times a day. In some embodiments, 2-(diethylamino)ethyl 2-(4-isobutylphenyl)propionate or pharmaceutically acceptable salts thereof is administered six times a day. In some embodiments, 2-(diethylamino)ethyl 2-(4-isobutylphenyl)propionate or pharmaceutically acceptable salts thereof is administered seven times a day. In some embodiments, 2-(diethylamino)ethyl 2-(4-isobutylphenyl)propionate or pharmaceutically acceptable salts thereof is administered eight times a day.

In some embodiments, 2-(diethylamino)ethyl 2-(4-isobutylphenyl)propionate or pharmaceutically acceptable salts thereof is administered once every hour or once every 4 to 16 hours. In some embodiments, 2-(diethylamino)ethyl 2-(4-isobutylphenyl)propionate or pharmaceutically acceptable salts thereof is administered once every hour or once every 8 to 12 hours. In some embodiments, 2-(diethylamino)ethyl 2-(4-isobutylphenyl)propionate or pharmaceutically acceptable salts thereof is administered once every hour or once every 2, 3, 4, 5, 6, 7, 8, 9, 10, 11, 12, 13, 14, 15, 16, 17, 18, 19, 20, 21, 22 or 23 hours. In some embodiments, 2-(diethylamino)ethyl 2-(4-isobutylphenyl)propionate or pharmaceutically acceptable salts thereof is administered once every hour or once every 12 hours.

In some embodiments, 2-(diethylamino)ethyl 2-(4-isobutylphenyl)propionate or pharmaceutically acceptable salts thereof is administered once every hour. In some embodiments, 2-(diethylamino)ethyl 2-(4-isobutylphenyl)propionate or pharmaceutically acceptable salts thereof is administered once 2 hours. In some embodiments, 2-(diethylamino)ethyl 2-(4-isobutylphenyl)propionate or pharmaceutically acceptable salts thereof is administered once 3 hours. In some embodiments, 2-(diethylamino)ethyl 2-(4-isobutylphenyl)propionate or pharmaceutically acceptable salts thereof is administered once 4 hours. In some embodiments, 2-(diethylamino)ethyl 2-(4-isobutylphenyl)propionate or pharmaceutically acceptable salts thereof is administered once 5 hours. In some embodiments, 2-(diethylamino)ethyl 2-(4-isobutylphenyl)propionate or pharmaceutically acceptable salts thereof is administered once 6 hours. In some embodiments, 2-(diethylamino)ethyl 2-(4-isobutylphenyl)propionate or pharmaceutically acceptable salts thereof is administered once 7 hours. In some embodiments, 2-(diethylamino)ethyl 2-(4-isobutylphenyl)propionate or pharmaceutically acceptable salts thereof is administered once 8 hours. In some embodiments, 2-(diethylamino)ethyl 2-(4-isobutylphenyl)propionate or pharmaceutically acceptable salts thereof is administered once 9 hours. In some embodiments, 2-(diethylamino)ethyl 2-(4-isobutylphenyl)propionate or pharmaceutically acceptable salts thereof is administered once 10 hours. In some embodiments, 2-(diethylamino)ethyl 2-(4-isobutylphenyl)propionate or pharmaceutically acceptable salts thereof is administered once 11 hours. In some embodiments, 2-(diethylamino)ethyl 2-(4-isobutylphenyl)propionate or pharmaceutically acceptable salts thereof is administered once 12 hours. In some embodiments, 2-(diethylamino)ethyl 2-(4-isobutylphenyl)propionate or pharmaceutically acceptable salts thereof is administered once 13 hours. In some embodiments, 2-(diethylamino)ethyl 2-(4-isobutylphenyl)propionate or pharmaceutically acceptable salts thereof is administered once 14 hours. In some embodiments, 2-(diethylamino)ethyl 2-(4-isobutylphenyl)propionate or pharmaceutically acceptable salts thereof is administered once 15 hours. In some embodiments, 2-(diethylamino)ethyl 2-(4-isobutylphenyl)propionate or pharmaceutically acceptable salts thereof is administered once 16 hours. In some embodiments, 2-(diethylamino)ethyl 2-(4-isobutylphenyl)propionate or pharmaceutically acceptable salts thereof is administered once 17 hours. In some embodiments, 2-(diethylamino)ethyl 2-(4-isobutylphenyl)propionate or pharmaceutically acceptable salts thereof is administered once 18 hours. In some embodiments, 2-(diethylamino)ethyl 2-(4-isobutylphenyl)propionate or pharmaceutically acceptable salts thereof is administered once 19 hours. In some embodiments, 2-(diethylamino)ethyl 2-(4-isobutylphenyl)propionate or pharmaceutically acceptable salts thereof is administered once 20 hours. In some embodiments, 2-(diethylamino)ethyl 2-(4-isobutylphenyl)propionate or pharmaceutically acceptable salts thereof is administered once 2 hours. In some embodiments, 2-(diethylamino)ethyl 2-(4-isobutylphenyl)propionate or pharmaceutically acceptable salts thereof is administered once 21 hours. In some embodiments, 2-(diethylamino)ethyl 2-(4-isobutylphenyl)propionate or pharmaceutically acceptable salts thereof is administered once 22 hours. In some embodiments, 2-(diethylamino)ethyl 2-(4-isobutylphenyl)propionate or pharmaceutically acceptable salts thereof is administered once 23 hours.

In some embodiments, 2-(diethylamino)ethyl 2-(4-isobutylphenyl)propionate or pharmaceutically acceptable salts thereof is administered once every one, two, three, four, five, six, or seven days. In some embodiments, 2-(diethylamino)ethyl 2-(4-isobutylphenyl)propionate or pharmaceutically acceptable salts thereof is administered daily. In some embodiments, 2-(diethylamino)ethyl 2-(4-isobutylphenyl)propionate or pharmaceutically acceptable salts thereof is administered intermittently. In some embodiments, 2-(diethylamino)ethyl 2-(4-isobutylphenyl)propionate or pharmaceutically acceptable salts thereof is administered once every two days. In some embodiments, 2-(diethylamino)ethyl 2-(4-isobutylphenyl)propionate or pharmaceutically acceptable salts thereof is administered once every three days. In some embodiments, 2-(diethylamino)ethyl 2-(4-isobutylphenyl)propionate or pharmaceutically acceptable salts thereof is administered once every four days. In some embodiments, 2-(diethylamino)ethyl 2-(4-isobutylphenyl)propionate or pharmaceutically acceptable salts thereof is administered once every five days. In some embodiments, 2-(diethylamino)ethyl 2-(4-isobutylphenyl)propionate or pharmaceutically acceptable salts thereof is administered once every six days. In some embodiments, 2-(diethylamino)ethyl 2-(4-isobutylphenyl)propionate or pharmaceutically acceptable salts thereof is administered once every week.

In some embodiments, the topical administration is administered for 1 day to lifetime. In some embodiments, the topical administration is administered for 7 to 365 consecutive or non-consecutive days. In some embodiments, the topical administration is administered for 7 to 98 consecutive or non-consecutive days. In some embodiments, the topical administration is administered for 14 to 91 consecutive or non-consecutive days. In some embodiments, the topical administration is administered for 14 to 84 consecutive or non-consecutive days. In some embodiments, the topical administration is administered for 28 to 84 consecutive or non-consecutive days. In some embodiments, the topical administration is administered for 56 to 84 consecutive or non-consecutive days.

In some embodiments, the topical administration is administered for 7 to 98 consecutive days. In some embodiments, the topical administration is administered for 14 to 91 consecutive days. In some embodiments, the topical administration is administered for 14 to 84 consecutive days. In some embodiments, the topical administration is administered for 28 to 84 consecutive days. In some embodiments, the topical administration is administered for 56 to 84 consecutive days.

In some embodiments, the topical administration is administered for at least one or more consecutive days or non-consecutive days, particularly a number of said one or more consecutive or non-consecutive days is selected from a group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, and 140.

In some embodiments, the device of the present disclosure includes a dosage form selected from one or more of transdermal patch, cream, foam, gel, lotion, ointment, paste, powder, shake lotion, solid, sponge, tape, tinkture, vapor, injection, drops, rinses, spray, and solution. In some embodiments, the device of the present disclosure includes a dosage form selected from transdermal solutions, including one or more of transdermal drops, rinses and spray.

In some embodiments, the device of the present disclosure is a device capable of administrating about 0.1 mg to about 20 mg, in particular 0.1 mg to 20 mg, of 2-(diethylamino)ethyl 2-(4-isobutylphenyl)propionate and/or pharmaceutically acceptable salts thereof in each dose. In some embodiments, the device of the present disclosure is a device capable of administrating about 0.2 mg to about 18 mg, in particular 0.2 mg to 18 mg, of 2-(diethylamino)ethyl 2-(4-isobutylphenyl)propionate and/or pharmaceutically acceptable salts thereof in each dose. In some embodiments, the device of the present disclosure is a device capable of administrating about 0.4 mg to about 16 mg, in particular 0.4 mg to 16 mg, of 2-(diethylamino)ethyl 2-(4-isobutylphenyl)propionate and/or pharmaceutically acceptable salts thereof in each dose. In some embodiments, the device of the present disclosure is a device capable of administrating about 0.6 mg to about 14 mg, in particular 0.6 mg to 14 mg, of 2-(diethylamino)ethyl 2-(4-isobutylphenyl)propionate and/or pharmaceutically acceptable salts thereof in each dose. In some embodiments, the device of the present disclosure is a device capable of administrating about 0.8 mg to about 12 mg, in particular 0.8 mg to 12 mg, of 2-(diethylamino)ethyl 2-(4-isobutylphenyl)propionate and/or pharmaceutically acceptable salts thereof in each dose. In some embodiments, the device of the present disclosure is a device capable of administrating about 1 mg to about 10 mg, in particular 1 mg to 10 mg, of 2-(diethylamino)ethyl 2-(4-isobutylphenyl)propionate and/or pharmaceutically acceptable salts thereof in each dose. In some embodiments, the device of the present disclosure is a device capable of administrating about 1.2 mg to about 9 mg, in particular 1.2 mg to 9 mg, of 2-(diethylamino)ethyl 2-(4-isobutylphenyl)propionate and/or pharmaceutically acceptable salts thereof in each dose. In some embodiments, the device of the present disclosure is a device capable of administrating about 1.5 mg to about 8 mg, in particular 1.5 mg to 8 mg, of 2-(diethylamino)ethyl 2-(4-isobutylphenyl)propionate and/or pharmaceutically acceptable salts thereof in each dose. In some embodiments, the device of the present disclosure is a device capable of administrating about 1.8 mg to about 7 mg, in particular 1.8 mg to 7 mg, of 2-(diethylamino)ethyl 2-(4-isobutylphenyl)propionate and/or pharmaceutically acceptable salts thereof in each dose. In some embodiments, the device is a device capable of administrating about 2 mg to about 6 mg, in particular 2 mg to 6 mg, of 2-(diethylamino)ethyl 2-(4-isobutylphenyl)propionate and/or pharmaceutically acceptable salts thereof in each dose. In some embodiments, the device is a device capable of administrating about 3 mg to about 5 mg, in particular 3 mg to 5 mg, of 2-(diethylamino)ethyl 2-(4-isobutylphenyl)propionate and/or pharmaceutically acceptable salts thereof in each dose. In some embodiments, the device is a device capable of administrating about 4 mg to about 4.75 mg, in particular 4 mg to 4.75 mg, of 2-(diethylamino)ethyl 2-(4-isobutylphenyl)propionate and/or pharmaceutically acceptable salts thereof in each dose. In some embodiments, the device is a device capable of administrating about 4.375 mg to about 4.5 mg, in particular 4.375 mg to 4.5 mg, of 2-(diethylamino)ethyl 2-(4-isobutylphenyl)propionate and/or pharmaceutically acceptable salts thereof in each dose.

In some embodiments, the device is a spray capable of spraying about 0.1 mg to about 20 mg, in particular 0.1 mg to 20 mg, of 2-(diethylamino)ethyl 2-(4-isobutylphenyl)propionate and/or pharmaceutically acceptable salts thereof in each spray. In some embodiments, the device is a spray capable of spraying about 0.2 mg to about 18 mg, in particular 0.2 mg to 18 mg, of 2-(diethylamino)ethyl 2-(4-isobutylphenyl)propionate and/or pharmaceutically acceptable salts thereof in each spray. In some embodiments, the device is a spray capable of spraying about 0.4 mg to about 16 mg, in particular 0.4 mg to 16 mg, of 2-(diethylamino)ethyl 2-(4-isobutylphenyl)propionate and/or pharmaceutically acceptable salts thereof in each spray. In some embodiments, the device is a spray capable of spraying about 0.6 mg to about 14 mg, in particular 0.6 mg to 14 mg, of 2-(diethylamino)ethyl 2-(4-isobutylphenyl)propionate and/or pharmaceutically acceptable salts thereof in each spray. In some embodiments, the device is a spray capable of spraying about 0.8 mg to about 12 mg, in particular 0.7 mg to 12 mg, of 2-(diethylamino)ethyl 2-(4-isobutylphenyl)propionate and/or pharmaceutically acceptable salts thereof in each spray. In some embodiments, the device is a spray capable of spraying about 1 mg to about 10 mg, in particular 0.1 mg to 10 mg, of 2-(diethylamino)ethyl 2-(4-isobutylphenyl)propionate and/or pharmaceutically acceptable salts thereof in each spray. In some embodiments, the device is a spray capable of spraying about 1.2 mg to about 9 mg, in particular 1.2 mg to 9 mg, of 2-(diethylamino)ethyl 2-(4-isobutylphenyl)propionate and/or pharmaceutically acceptable salts thereof in each spray. In some embodiments, the device is a spray capable of spraying about 1.5 mg to about 8 mg, in particular 1.5 mg to 8 mg, of 2-(diethylamino)ethyl 2-(4-isobutylphenyl)propionate and/or pharmaceutically acceptable salts thereof in each spray. In some embodiments, the device is a spray capable of spraying about 1.8 mg to about 7 mg, in particular 1.8 mg to 7 mg, of 2-(diethylamino)ethyl 2-(4-isobutylphenyl)propionate and/or pharmaceutically acceptable salts thereof in each spray. In some embodiments, the device is a spray capable of spraying about 2 mg to about 6 mg, in particular 2 mg to 6 mg, of 2-(diethylamino)ethyl 2-(4-isobutylphenyl)propionate and/or pharmaceutically acceptable salts thereof in each spray. In some embodiments, the device is a spray capable of spraying about 3 mg to about 5 mg, in particular 3 mg to 5 mg, of 2-(diethylamino)ethyl 2-(4-isobutylphenyl)propionate and/or pharmaceutically acceptable salts thereof in each spray. In some embodiments, the device is a spray capable of spraying about 4 mg to about 4.75 mg, in particular 4 mg to 4.75 mg, of 2-(diethylamino)ethyl 2-(4-isobutylphenyl)propionate and/or pharmaceutically acceptable salts thereof in each spray. In some embodiments, the device is a spray capable of spraying about 4.375 mg to about 4.5 mg, in particular 4.375 mg to 4.5 mg, of 2-(diethylamino)ethyl 2-(4-isobutylphenyl)propionate and/or pharmaceutically acceptable salts thereof in each spray.

In some embodiments, the device is a spray including a nozzle, the nozzle sprays about 0.1 mg to about 20 mg, in particular 0.1 mg to 20 mg, of 2-(diethylamino)ethyl 2-(4-isobutylphenyl)propionate and/or pharmaceutically acceptable salts thereof in each time of pressing the nozzle. In some embodiments, the device is a spray including a nozzle, the nozzle sprays about 0.2 mg to about 18 mg, in particular 0.2 mg to 18 mg, of 2-(diethylamino)ethyl 2-(4-isobutylphenyl)propionate and/or pharmaceutically acceptable salts thereof in each time of pressing the nozzle. In some embodiments, the device is a spray including a nozzle, the nozzle sprays about 0.4 mg to about 16 mg, in particular 0.4 mg to 16 mg, of 2-(diethylamino)ethyl 2-(4-isobutylphenyl)propionate and/or pharmaceutically acceptable salts thereof in each time of pressing the nozzle. In some embodiments, the device is a spray including a nozzle, the nozzle sprays about 0.6 mg to about 14 mg, in particular 0.6 mg to 14 mg, of 2-(diethylamino)ethyl 2-(4-isobutylphenyl)propionate and/or pharmaceutically acceptable salts thereof in each time of pressing the nozzle. In some embodiments, the device is a spray including a nozzle, the nozzle sprays about 0.8 mg to about 12 mg, in particular 0.8 mg to 12 mg, of 2-(diethylamino)ethyl 2-(4-isobutylphenyl)propionate and/or pharmaceutically acceptable salts thereof in each time of pressing the nozzle. In some embodiments, the device is a spray including a nozzle, the nozzle sprays about 1 mg to about 10 mg, in particular 1 mg to 10 mg, of 2-(diethylamino)ethyl 2-(4-isobutylphenyl)propionate and/or pharmaceutically acceptable salts thereof in each time of pressing the nozzle. In some embodiments, the device is a spray including a nozzle, the nozzle sprays about 1.2 mg to about 9 mg, in particular 1.2 mg to 9 mg, of 2-(diethylamino)ethyl 2-(4-isobutylphenyl)propionate and/or pharmaceutically acceptable salts thereof in each time of pressing the nozzle. In some embodiments, the device is a spray including a nozzle, the nozzle sprays about 1.5 mg to about 8 mg, in particular 1.5 mg to 8 mg, of 2-(diethylamino)ethyl 2-(4-isobutylphenyl)propionate and/or pharmaceutically acceptable salts thereof in each time of pressing the nozzle. In some embodiments, the device is a spray including a nozzle, the nozzle sprays about 1.8 mg to about 7 mg, in particular 1.8 mg to 7 mg, of 2-(diethylamino)ethyl 2-(4-isobutylphenyl)propionate and/or pharmaceutically acceptable salts thereof in each time of pressing the nozzle. In some embodiments, the device is a spray including a nozzle, the nozzle sprays about 4 mg to about 8 mg, in particular 4 mg to 8 mg, of 2-(diethylamino)ethyl 2-(4-isobutylphenyl)propionate and/or pharmaceutically acceptable salts thereof in each time of pressing the nozzle. In some embodiments, the device is a spray including a nozzle, the nozzle sprays about 2 mg to about 6 mg, in particular 2 mg to 6 mg, of 2-(diethylamino)ethyl 2-(4-isobutylphenyl)propionate and/or pharmaceutically acceptable salts thereof in each time of pressing the nozzle. In some embodiments, the device is a spray including a nozzle, the nozzle sprays about 3 mg to about 5 mg, in particular 3 mg to 5 mg, of 2-(diethylamino)ethyl 2-(4-isobutylphenyl)propionate and/or pharmaceutically acceptable salts thereof in each time of pressing the nozzle. In some embodiments, the spray of the present disclosure is a spray including a nozzle, the nozzle sprays about 4 mg to about 4.75 mg, in particular 4 mg to 4.75 mg, of 2-(diethylamino)ethyl 2-(4-isobutylphenyl)propionate and/or pharmaceutically acceptable salts thereof in each time of pressing the nozzle.

In some embodiments, the device is a drop capable of dropping about 0.1 mg to about 20 mg, in particular 0.1 mg to 20 mg, of 2-(diethylamino)ethyl 2-(4-isobutylphenyl)propionate and/or pharmaceutically acceptable salts thereof in each drop. In some embodiments, the device is a drop capable of dropping about 0.2 mg to about 18 mg, in particular 0.2 mg to 18 mg, of 2-(diethylamino)ethyl 2-(4-isobutylphenyl)propionate and/or pharmaceutically acceptable salts thereof in each drop. In some embodiments, the device is a drop capable of dropping about 0.4 mg to about 16 mg, in particular 0.4 mg to 16 mg, of 2-(diethylamino)ethyl 2-(4-isobutylphenyl)propionate and/or pharmaceutically acceptable salts thereof in each drop. In some embodiments, the device is a drop capable of dropping about 0.6 mg to about 14 mg, in particular 0.6 mg to 14 mg, of 2-(diethylamino)ethyl 2-(4-isobutylphenyl)propionate and/or pharmaceutically acceptable salts thereof in each drop. In some embodiments, the device is a drop capable of dropping about 0.8 mg to about 12 mg, in particular 0.7 mg to 12 mg, of 2-(diethylamino)ethyl 2-(4-isobutylphenyl)propionate and/or pharmaceutically acceptable salts thereof in each drop. In some embodiments, the device is a drop capable of dropping about 1 mg to about 10 mg, in particular 0.1 mg to 10 mg, of 2-(diethylamino)ethyl 2-(4-isobutylphenyl)propionate and/or pharmaceutically acceptable salts thereof in each drop. In some embodiments, the device is a drop capable of dropping about 1.2 mg to about 9 mg, in particular 1.2 mg to 9 mg, of 2-(diethylamino)ethyl 2-(4-isobutylphenyl)propionate and/or pharmaceutically acceptable salts thereof in each drop. In some embodiments, the device is a drop capable of dropping about 1.5 mg to about 8 mg, in particular 1.5 mg to 8 mg, of 2-(diethylamino)ethyl 2-(4-isobutylphenyl)propionate and/or pharmaceutically acceptable salts thereof in each drop. In some embodiments, the device is a drop capable of dropping about 1.8 mg to about 7 mg, in particular 1.8 mg to 7 mg, of 2-(diethylamino)ethyl 2-(4-isobutylphenyl)propionate and/or pharmaceutically acceptable salts thereof in each drop. In some embodiments, the device is a drop capable of dropping about 2 mg to about 6 mg, in particular 2 mg to 6 mg, of 2-(diethylamino)ethyl 2-(4-isobutylphenyl)propionate and/or pharmaceutically acceptable salts thereof in each drop. In some embodiments, the device is a drop capable of dropping about 3 mg to about 5 mg, in particular 3 mg to 5 mg, of 2-(diethylamino)ethyl 2-(4-isobutylphenyl)propionate and/or pharmaceutically acceptable salts thereof in each drop. In some embodiments, the device is a drop capable of dropping about 4 mg to about 4.75 mg, in particular 4 mg to 4.75 mg, of 2-(diethylamino)ethyl 2-(4-isobutylphenyl)propionate and/or pharmaceutically acceptable salts thereof in each drop. In some embodiments, the device is a drop capable of dropping about 4.375 mg to about 4.5 mg, in particular 4.375 mg to 4.5 mg, of 2-(diethylamino)ethyl 2-(4-isobutylphenyl)propionate and/or pharmaceutically acceptable salts thereof in each drop.

In some embodiments, the device is a device capable of administrating in an amount, a spray including an amount, a spray including a nozzle spraying in an amount, or a drop including an amount, selected from a group consisting of 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.55 mg, 1.6 mg, 1.65 mg, 1.7 mg, 1.75 mg, 1.8 mg, 1.85 mg, 1.9 mg, 1.95 mg, 2 mg, 2.05 mg, 2.1 mg, 2.15 mg, 2.2 mg, 2.25 mg, 2.3 mg, 2.35 mg, 2.4 mg, 2.45 mg, 2.5 mg, 2.55 mg, 2.6 mg, 2.65 mg, 2.7 mg, 2.75 mg, 2.8 mg, 2.85 mg, 2.9 mg, 2.95 mg, 3 mg, 3.05 mg, 3.1 mg, 3.15 mg, 3.2 mg, 3.25 mg, 3.3 mg, 3.35 mg, 3.4 mg, 3.45 mg, 3.5 mg, 3.55 mg, 3.6 mg, 3.65 mg, 3.7 mg, 3.75 mg, 3.8 mg, 3.85 mg, 3.9 mg, 3.95 mg, 4 mg, 4.025 mg, 4.05 mg, 4.075 mg, 4.1 mg, 4.125 mg, 4.15 mg, 4.175 mg, 4.2 mg, 4.225 mg, 4.25 mg, 4.275 mg, 4.3 mg, 4.325 mg, 4.35 mg, 4.375 mg, 4.4 mg, 4.425 mg, 4.45 mg, 4.475 mg, 4.5 mg, 4.525 mg, 4.55 mg, 4.575 mg, 4.6 mg, 4.625 mg, 4.65 mg, 4.675 mg, 4.7 mg, 4.725 mg, 4.75 mg, 4.775 mg, 4.8 mg, 4.825 mg, 4.85 mg, 4.875 mg, 4.9 mg, 4.925 mg, 4.95 mg, 4.975 mg, 5 mg, 5.05 mg, 5.1 mg, 5.15 mg, 5.2 mg, 5.25 mg, 5.3 mg, 5.35 mg, 5.4 mg, 5.45 mg, 5.5 mg, 5.55 mg, 5.6 mg, 5.65 mg, 5.7 mg, 5.75 mg, 5.8 mg, 5.85 mg, 5.9 mg, 5.95 mg, 6 mg, 6.05 mg, 6.1 mg, 6.15 mg, 6.2 mg, 6.25 mg, 6.3 mg, 6.35 mg, 6.4 mg, 6.45 mg, 6.5 mg, 6.55 mg, 6.6 mg, 6.65 mg, 6.7 mg, 6.75 mg, 6.8 mg, 6.85 mg, 6.9 mg, 6.95 mg, 7 mg, 7.05 mg, 7.1 mg, 7.15 mg, 7.2 mg, 7.25 mg, 7.3 mg, 7.35 mg, 7.4 mg, 7.45 mg, 7.5 mg, 7.55 mg, 7.6 mg, 7.65 mg, 7.7 mg, 7.75 mg, 7.8 mg, 7.85 mg, 7.9 mg, 7.95 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, 10 mg, 10.1 mg, 10.2 mg, 10.3 mg, 10.4 mg, 10.5 mg, 10.6 mg, 10.7 mg, 10.8 mg, 10.9 mg, 11 mg, 11.1 mg, 11.2 mg, 11.3 mg, 11.4 mg, 11.5 mg, 11.6 mg, 11.7 mg, 11.8 mg, 11.9 mg, 12 mg, 12.1 mg, 12.2 mg, 12.3 mg, 12.4 mg, 12.5 mg, 12.6 mg, 12.7 mg, 12.8 mg, 12.9 mg, 13 mg, 13.1 mg, 13.2 mg, 13.3 mg, 13.4 mg, 13.5 mg, 13.6 mg, 13.7 mg, 13.8 mg, 13.9 mg, 14 mg, 14.1 mg, 14.2 mg, 14.3 mg, 14.4 mg, 14.5 mg, 14.6 mg, 14.7 mg, 14.8 mg, 14.9 mg, 15 mg, 15.1 mg, 15.2 mg, 15.3 mg, 15.4 mg, 15.5 mg, 15.6 mg, 15.7 mg, 15.8 mg, 15.9 mg, 16 mg, 16.1 mg, 16.2 mg, 16.3 mg, 16.4 mg, 16.5 mg, 16.6 mg, 16.7 mg, 16.8 mg, 16.9 mg, 17 mg, 17.1 mg, 17.2 mg, 17.3 mg, 17.4 mg, 17.5 mg, 17.6 mg, 17.7 mg, 17.8 mg, 17.9 mg, 18 mg, 18.1 mg, 18.2 mg, 18.3 mg, 18.4 mg, 18.5 mg, 18.6 mg, 18.7 mg, 18.8 mg, 18.9 mg, 19 mg, 19.1 mg, 19.2 mg, 19.3 mg, 19.4 mg, 19.5 mg, 19.6 mg, 19.7 mg, 19.8 mg, 19.9 mg and 20 mg, of 2-(diethylamino)ethyl 2-(4-isobutylphenyl)propionate and/or pharmaceutically acceptable salts thereof, in each dose.

In some embodiments, the device is a patch capable of administering about 0.1 mg to about 20 mg, in particular 0.1 mg to 20 mg, of 2-(diethylamino)ethyl 2-(4-isobutylphenyl)propionate and/or pharmaceutically acceptable salts thereof in each patch. In some embodiments, the device is a patch capable of administering about 0.2 mg to about 18 mg, in particular 0.2 mg to 18 mg, of 2-(diethylamino)ethyl 2-(4-isobutylphenyl)propionate and/or pharmaceutically acceptable salts thereof in each patch. In some embodiments, the device is a patch capable of administering about 0.4 mg to about 16 mg, in particular 0.4 mg to 16 mg, of 2-(diethylamino)ethyl 2-(4-isobutylphenyl)propionate and/or pharmaceutically acceptable salts thereof in each patch. In some embodiments, the device is a patch capable of administering about 0.6 mg to about 14 mg, in particular 0.6 mg to 14 mg, of 2-(diethylamino)ethyl 2-(4-isobutylphenyl)propionate and/or pharmaceutically acceptable salts thereof in each patch. In some embodiments, the device is a patch capable of administering about 0.8 mg to about 12 mg, in particular 0.7 mg to 12 mg, of 2-(diethylamino)ethyl 2-(4-isobutylphenyl)propionate and/or pharmaceutically acceptable salts thereof in each patch. In some embodiments, the device is a patch capable of administering about 1 mg to about 10 mg, in particular 0.1 mg to 10 mg, of 2-(diethylamino)ethyl 2-(4-isobutylphenyl)propionate and/or pharmaceutically acceptable salts thereof in each patch. In some embodiments, the device is a patch capable of administering about 1.2 mg to about 9 mg, in particular 1.2 mg to 9 mg, of 2-(diethylamino)ethyl 2-(4-isobutylphenyl)propionate and/or pharmaceutically acceptable salts thereof in each patch. In some embodiments, the device is a patch capable of administering about 1.5 mg to about 8 mg, in particular 1.5 mg to 8 mg, of 2-(diethylamino)ethyl 2-(4-isobutylphenyl)propionate and/or pharmaceutically acceptable salts thereof in each patch. In some embodiments, the device is a patch capable of administering about 1.8 mg to about 7 mg, in particular 1.8 mg to 7 mg, of 2-(diethylamino)ethyl 2-(4-isobutylphenyl)propionate and/or pharmaceutically acceptable salts thereof in each patch. In some embodiments, the device is a patch capable of administering about 2 mg to about 6 mg, in particular 2 mg to 6 mg, of 2-(diethylamino)ethyl 2-(4-isobutylphenyl)propionate and/or pharmaceutically acceptable salts thereof in each patch. In some embodiments, the device is a patch capable of administering about 3 mg to about 5 mg, in particular 3 mg to 5 mg, of 2-(diethylamino)ethyl 2-(4-isobutylphenyl)propionate and/or pharmaceutically acceptable salts thereof in each patch. In some embodiments, the device is a patch capable of administering about 4 mg to about 4.75 mg, in particular 4 mg to 4.75 mg, of 2-(diethylamino)ethyl 2-(4-isobutylphenyl)propionate and/or pharmaceutically acceptable salts thereof in each patch. In some embodiments, the device is a patch capable of administering about 4.375 mg to about 4.5 mg, in particular 4.375 mg to 4.5 mg, of 2-(diethylamino)ethyl 2-(4-isobutylphenyl)propionate and/or pharmaceutically acceptable salts thereof in each patch.

In some embodiments, the device is a patch including an amount selected from a group consisting of 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.55 mg, 1.6 mg, 1.65 mg, 1.7 mg, 1.75 mg, 1.8 mg, 1.85 mg, 1.9 mg, 1.95 mg, 2 mg, 2.05 mg, 2.1 mg, 2.15 mg, 2.2 mg, 2.25 mg, 2.3 mg, 2.35 mg, 2.4 mg, 2.45 mg, 2.5 mg, 2.55 mg, 2.6 mg, 2.65 mg, 2.7 mg, 2.75 mg, 2.8 mg, 2.85 mg, 2.9 mg, 2.95 mg, 3 mg, 3.05 mg, 3.1 mg, 3.15 mg, 3.2 mg, 3.25 mg, 3.3 mg, 3.35 mg, 3.4 mg, 3.45 mg, 3.5 mg, 3.55 mg, 3.6 mg, 3.65 mg, 3.7 mg, 3.75 mg, 3.8 mg, 3.85 mg, 3.9 mg, 3.95 mg, 4 mg, 4.025 mg, 4.05 mg, 4.075 mg, 4.1 mg, 4.125 mg, 4.15 mg, 4.175 mg, 4.2 mg, 4.225 mg, 4.25 mg, 4.275 mg, 4.3 mg, 4.325 mg, 4.35 mg, 4.375 mg, 4.4 mg, 4.425 mg, 4.45 mg, 4.475 mg, 4.5 mg, 4.525 mg, 4.55 mg, 4.575 mg, 4.6 mg, 4.625 mg, 4.65 mg, 4.675 mg, 4.7 mg, 4.725 mg, 4.75 mg, 4.775 mg, 4.8 mg, 4.825 mg, 4.85 mg, 4.875 mg, 4.9 mg, 4.925 mg, 4.95 mg, 4.975 mg, 5 mg, 5.05 mg, 5.1 mg, 5.15 mg, 5.2 mg, 5.25 mg, 5.3 mg, 5.35 mg, 5.4 mg, 5.45 mg, 5.5 mg, 5.55 mg, 5.6 mg, 5.65 mg, 5.7 mg, 5.75 mg, 5.8 mg, 5.85 mg, 5.9 mg, 5.95 mg, 6 mg, 6.05 mg, 6.1 mg, 6.15 mg, 6.2 mg, 6.25 mg, 6.3 mg, 6.35 mg, 6.4 mg, 6.45 mg, 6.5 mg, 6.55 mg, 6.6 mg, 6.65 mg, 6.7 mg, 6.75 mg, 6.8 mg, 6.85 mg, 6.9 mg, 6.95 mg, 7 mg, 7.05 mg, 7.1 mg, 7.15 mg, 7.2 mg, 7.25 mg, 7.3 mg, 7.35 mg, 7.4 mg, 7.45 mg, 7.5 mg, 7.55 mg, 7.6 mg, 7.65 mg, 7.7 mg, 7.75 mg, 7.8 mg, 7.85 mg, 7.9 mg, 7.95 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, 10 mg, 10.1 mg, 10.2 mg, 10.3 mg, 10.4 mg, 10.5 mg, 10.6 mg, 10.7 mg, 10.8 mg, 10.9 mg, 11 mg, 11.1 mg, 11.2 mg, 11.3 mg, 11.4 mg, 11.5 mg, 11.6 mg, 11.7 mg, 11.8 mg, 11.9 mg, 12 mg, 12.1 mg, 12.2 mg, 12.3 mg, 12.4 mg, 12.5 mg, 12.6 mg, 12.7 mg, 12.8 mg, 12.9 mg, 13 mg, 13.1 mg, 13.2 mg, 13.3 mg, 13.4 mg, 13.5 mg, 13.6 mg, 13.7 mg, 13.8 mg, 13.9 mg, 14 mg, 14.1 mg, 14.2 mg, 14.3 mg, 14.4 mg, 14.5 mg, 14.6 mg, 14.7 mg, 14.8 mg, 14.9 mg, 15 mg, 15.1 mg, 15.2 mg, 15.3 mg, 15.4 mg, 15.5 mg, 15.6 mg, 15.7 mg, 15.8 mg, 15.9 mg, 16 mg, 16.1 mg, 16.2 mg, 16.3 mg, 16.4 mg, 16.5 mg, 16.6 mg, 16.7 mg, 16.8 mg, 16.9 mg, 17 mg, 17.1 mg, 17.2 mg, 17.3 mg, 17.4 mg, 17.5 mg, 17.6 mg, 17.7 mg, 17.8 mg, 17.9 mg, 18 mg, 18.1 mg, 18.2 mg, 18.3 mg, 18.4 mg, 18.5 mg, 18.6 mg, 18.7 mg, 18.8 mg, 18.9 mg, 19 mg, 19.1 mg, 19.2 mg, 19.3 mg, 19.4 mg, 19.5 mg, 19.6 mg, 19.7 mg, 19.8 mg, 19.9 mg and 20 mg, of 2-(diethylamino)ethyl 2-(4-isobutylphenyl)propionate and/or pharmaceutically acceptable salts thereof.

In one embodiment, a composition including 2-(diethylamino)ethyl 2-(4-isobutylphenyl)propionate is presented as the table below.

| | Unit Formula (mg) | | | | |
|---|---|---|---|---|---|
| | (4.5 mg of 2-(diethylamino)ethyl | | | | |
| | 2-(4-isobutylphenyl)propionate | | | | |
| | hydrochloride per spray strengths) | | | Quality | |
| Components | Amount | | Percentage | Reference | Function |
| 2-(diethylamino)ethyl | 1400 | mg | 7% in 25% ethanol | In-house | Active |
| 2-(4-isobutylphenyl)propionate | | | | | ingredient |
| hydrochloride | | | | | |
| ethanol (25% in water, v/v) | 20 | mL | 25% Ethanol in water | In-house | Diluent |
| | | | | | (excipient) |

In one embodiment, a subject will spray one spray (4.375 mg 2-(diethylamino)ethyl 2-(4-isobutylphenyl)propionate in 25% ethanol) of the drug solution to the medial surface or the lateral surface, or the front surface or the back surface of the knee, once per day until no pain on the knee.

In one embodiment, a subject will spray one spray (4.375 mg 2-(diethylamino)ethyl 2-(4-isobutylphenyl)propionate in 25% ethanol) of the drug solution to the medial surface or the lateral surface, or the front surface or the back surface of the knee, BID, until no pain on the knee.

In one embodiment, a subject will spray one spray (4.375 mg 2-(diethylamino)ethyl 2-(4-isobutylphenyl)propionate in 25% ethanol) of the drug solution to the surface around the ankle joint, once per day until no pain on the ankle.

In one embodiment, a subject will spray one spray (4.375 mg 2-(diethylamino)ethyl 2-(4-isobutylphenyl)propionate in 25% ethanol) of the drug solution to the surface around the ankle joint, BID, until no pain on the ankle.

In one embodiment, a subject will spray one spray (4.375 mg 2-(diethylamino)ethyl 2-(4-isobutylphenyl)propionate in 25% ethanol) of the drug solution to the surface around the elbow joint, once per day until no pain on the elbow.

In one embodiment, a subject will spray one spray (4.375 mg 2-(diethylamino)ethyl 2-(4-isobutylphenyl)propionate in 25% ethanol) of the drug solution to the surface around the elbow joint, BID until no pain on the elbow.

In one embodiment, a subject will spray one spray (4.375 mg 2-(diethylamino)ethyl 2-(4-isobutylphenyl)propionate in 25% ethanol) of the drug solution to the surface around the wrist joint, once per day until no pain on the wrist.

In one embodiment, a subject will spray one spray (4.375 mg 2-(diethylamino)ethyl 2-(4-isobutylphenyl)propionate in 25% ethanol) of the drug solution to the surface around the wrist joint, BID until no pain on the wrist.

In one embodiment, a subject will spray one spray (4.375 mg 2-(diethylamino)ethyl 2-(4-isobutylphenyl)propionate in 25% ethanol) of the drug solution to the surface around the shoulder joint, once per day until no pain on the shoulder.

In one embodiment, a subject will spray one spray (4.375 mg 2-(diethylamino)ethyl 2-(4-isobutylphenyl)propionate in 25% ethanol) of the drug solution to the surface around the shoulder joint, BID until no pain on the shoulder.

In one embodiment, a subject will spray one spray (4.375 mg 2-(diethylamino)ethyl 2-(4-isobutylphenyl)propionate in 25% ethanol) of the drug solution to the surface around the hip joint, once per day until no pain on the hip joint.

In one embodiment, a subject will spray one spray (4.375 mg 2-(diethylamino)ethyl 2-(4-isobutylphenyl)propionate in 25% ethanol) of the drug solution to the surface around the hip joint, BID until no pain on the hip.

In one embodiment, a subject will spray one spray (4.375 mg 2-(diethylamino)ethyl 2-(4-isobutylphenyl)propionate in 25% ethanol) of the drug solution to the surface around the finger joints, once per day until no pain on the fingers.

In one embodiment, a subject will spray one spray (4.375 mg 2-(diethylamino)ethyl 2-(4-isobutylphenyl)propionate in 25% ethanol) of the drug solution to the surface around the finger joints, BID until no pain on the fingers In one embodiment, a subject will spray one spray (4.375 mg 2-(diethylamino)ethyl 2-(4-isobutylphenyl)propionate in 25% ethanol) of the drug solution to the surface around the toe joints, once per day until no pain on the toes.

In one embodiment, a subject will spray one spray (4.375 mg 2-(diethylamino)ethyl 2-(4-isobutylphenyl)propionate in 25% ethanol) of the drug solution to the surface around the toe joints, BID until no pain on the toes.

In one embodiment, a subject will spray two sprays (8.75 mg 2-(diethylamino)ethyl 2-(4-isobutylphenyl)propionate in 25% ethanol) of the drug solution to the knee: one spray to the medial surface and one spray to the lateral surface of the knee, once per day until no pain on the knee.

In one embodiment, a subject will spray two sprays (8.75 mg 2-(diethylamino)ethyl 2-(4-isobutylphenyl)propionate in 25% ethanol) of the drug solution to the knee: one spray to the medial surface and one spray to the lateral surface of the knee, BID, until no pain on the knee.

In one embodiment, a subject will spray two sprays (8.75 mg 2-(diethylamino)ethyl 2-(4-isobutylphenyl)propionate in 25% ethanol) of the drug solution to the surface around the ankle joint, each spray to a different skin area, once per day until no pain on the ankle.

In one embodiment, a subject will spray two sprays (8.75 mg 2-(diethylamino)ethyl 2-(4-isobutylphenyl)propionate in 25% ethanol) of the drug solution to the surface around the ankle joint, BID, each spray to a different skin area, until no pain on the ankle.

In one embodiment, a subject will spray two sprays (8.75 mg 2-(diethylamino)ethyl 2-(4-isobutylphenyl)propionate in 25% ethanol) of the drug solution to the surface around the elbow joint, each spray to a different skin area, once per day until no pain on the elbow.

In one embodiment, a subject will spray two sprays (8.75 mg 2-(diethylamino)ethyl 2-(4-isobutylphenyl)propionate in 25% ethanol) of the drug solution to the surface around the elbow joint, each spray to a different skin area, BID until no pain on the elbow.

In one embodiment, a subject will spray two sprays (8.75 mg 2-(diethylamino)ethyl 2-(4-isobutylphenyl)propionate in 25% ethanol) of the drug solution to the surface around the wrist joint, once per day until no pain on the wrist.

In one embodiment, a subject will spray two sprays (8.75 mg 2-(diethylamino)ethyl 2-(4-isobutylphenyl)propionate in 25% ethanol) of the drug solution to the surface around the wrist joint, each spray to a different skin area, BID until no pain on the wrist.

In one embodiment, a subject will spray two sprays (8.75 mg 2-(diethylamino)ethyl 2-(4-isobutylphenyl)propionate in 25% ethanol) of the drug solution to the surface around the shoulder joint, each spray to a different skin area, once per day until no pain on the shoulder.

In one embodiment, a subject will spray one spray (8.75 mg 2-(diethylamino)ethyl 2-(4-isobutylphenyl)propionate in 25% ethanol) of the drug solution to the surface around the shoulder joint, each spray to a different skin area, BID until no pain on the shoulder.

In one embodiment, a subject will spray two sprays (8.75 mg 2-(diethylamino)ethyl 2-(4-isobutylphenyl)propionate in 25% ethanol) of the drug solution to the surface around the hip joint, each spray to a different skin area, once per day until no pain on the hip joint.

In one embodiment, a subject will spray two sprays (8.75 mg 2-(diethylamino)ethyl 2-(4-isobutylphenyl)propionate in 25% ethanol) of the drug solution to the surface around the hip joint, each spray to a different skin area, BID until no pain on the hip.

In one embodiment, a subject will spray two sprays (8.75 mg 2-(diethylamino)ethyl 2-(4-isobutylphenyl)propionate in 25% ethanol) of the drug solution to the surface around the finger joints, each spray to a different skin area, once per day until no pain on the fingers.

In one embodiment, a subject will spray two sprays (8.75 mg 2-(diethylamino)ethyl 2-(4-isobutylphenyl)propionate in 25% ethanol) of the drug solution to the surface around the finger joints, BID until no pain on the fingers In one embodiment, a subject will spray two sprays (8.75 mg 2-(diethylamino)ethyl 2-(4-isobutylphenyl)propionate in 25% ethanol) of the drug solution to the surface around the toe joints, each spray to a different skin area, once per day until no pain on the toes.

In one embodiment, a subject will spray two sprays (8.75 mg 2-(diethylamino)ethyl 2-(4-isobutylphenyl)propionate in 25% ethanol) of the drug solution to the surface around the toe joints, each spray to a different skin area, BID until no pain on the toes.

In one embodiment, a subject will spray four sprays (17.5 mg 2-(diethylamino)ethyl 2-(4-isobutylphenyl)propionate in 25% ethanol) of the drug solution to around of the knee: one spray to the medial surface, one spray to the lateral surface, one spray the front surface, and one spray the back surface of the knee, once per day until no pain on the knee.

In one embodiment, a subject will spray four sprays (17.5 mg 2-(diethylamino)ethyl 2-(4-isobutylphenyl)propionate in 25% ethanol) of the drug solution to around of the knee: one spray to the medial surface, one spray to the lateral surface, one spray the front surface, and one spray the back surface of the knee, BID, until no pain on the knee.

In one embodiment, a subject will spray four sprays (17.5 mg 2-(diethylamino)ethyl 2-(4-isobutylphenyl)propionate in 25% ethanol) of the drug solution to the surface around the ankle joint, each spray to a different skin area, once per day until no pain on the ankle.

In one embodiment, a subject will spray four sprays (17.5 mg 2-(diethylamino)ethyl 2-(4-isobutylphenyl)propionate in 25% ethanol) of the drug solution to the surface around the ankle joint, each spray to a different skin area, BID, until no pain on the ankle.

In one embodiment, a subject will spray four sprays (17.5 mg 2-(diethylamino)ethyl 2-(4-isobutylphenyl)propionate in 25% ethanol) of the drug solution to the surface around the elbow joint, each spray to a different skin area, once per day until no pain on the elbow.

In one embodiment, a subject will spray four sprays (17.5 mg 2-(diethylamino)ethyl 2-(4-isobutylphenyl)propionate in 25% ethanol) of the drug solution to the surface around the elbow joint, each spray to a different skin area, BID until no pain on the elbow.

In one embodiment, a subject will spray four sprays (17.5 mg 2-(diethylamino)ethyl 2-(4-isobutylphenyl)propionate in 25% ethanol) of the drug solution to the surface around the wrist joint, each spray to a different skin area, once per day until no pain on the wrist.

In one embodiment, a subject will spray four sprays (17.5 mg 2-(diethylamino)ethyl 2-(4-isobutylphenyl)propionate in 25% ethanol) of the drug solution to the surface around the wrist joint, each spray to a different skin area, BID until no pain on the wrist.

In one embodiment, a subject will spray four sprays (17.5 mg 2-(diethylamino)ethyl 2-(4-isobutylphenyl)propionate in 25% ethanol) of the drug solution to the surface around the shoulder joint, each spray to a different skin area, once per day until no pain on the shoulder.

In one embodiment, a subject will spray four sprays (17.5 mg 2-(diethylamino)ethyl 2-(4-isobutylphenyl)propionate in 25% ethanol) of the drug solution to the surface around the shoulder joint, each spray to a different skin area, BID until no pain on the shoulder.

In one embodiment, a subject will spray four sprays (17.5 mg 2-(diethylamino)ethyl 2-(4-isobutylphenyl)propionate in 25% ethanol) of the drug solution to the surface around the hip joint, once per day until no pain on the hip joint.

In one embodiment, a subject will spray four sprays (17.5 mg 2-(diethylamino)ethyl 2-(4-isobutylphenyl)propionate in 25% ethanol) of the drug solution to the surface around the hip joint, each spray to a different skin area, BID until no pain on the hip.

In one embodiment, a subject will spray four sprays (17.5 mg 2-(diethylamino)ethyl 2-(4-isobutylphenyl)propionate in 25% ethanol) of the drug solution to the surface around the finger joints, each spray to a different skin area, once per day until no pain on the fingers.

In one embodiment, a subject will spray four sprays (17.5 mg 2-(diethylamino)ethyl 2-(4-isobutylphenyl)propionate in 25% ethanol) of the drug solution to the surface around the finger joints, each spray to a different skin area, BID until no pain on the fingers.

In one embodiment, a subject will spray four sprays (17.5 mg 2-(diethylamino)ethyl 2-(4-isobutylphenyl)propionate in 25% ethanol) of the drug solution to the surface around the toe joints, each spray to a different skin area, once per day until no pain on the toes.

In one embodiment, a subject will spray four sprays (17.5 mg 2-(diethylamino)ethyl 2-(4-isobutylphenyl)propionate in 25% ethanol) of the drug solution to the surface around the toe joints, each spray to a different skin area, BID until no pain on the toes.

In one embodiment, a subject will spray eight sprays (35 mg 2-(diethylamino)ethyl 2-(4-isobutylphenyl)propionate in 25% ethanol) of the drug solution to the surface around the shoulder joint, once per day until no pain on the shoulder.

In one embodiment, a subject will spray eight sprays (35 mg 2-(diethylamino)ethyl 2-(4-isobutylphenyl)propionate in 25% ethanol) of the drug solution to the surface around the shoulder joint, BID until no pain on the shoulder.

In one embodiment, a subject will spray eight sprays (35 mg 2-(diethylamino)ethyl 2-(4-isobutylphenyl)propionate in 25% ethanol) of the drug solution to the surface around the hip joint, once per day until no pain on the hip.

In one embodiment, a subject will spray eight sprays (35 mg 2-(diethylamino)ethyl 2-(4-isobutylphenyl)propionate in 25% ethanol) of the drug solution to the surface around the hip joint, BID until no pain on the hip.

In one embodiment, a subject will spray eight sprays (35 mg 2-(diethylamino)ethyl 2-(4-isobutylphenyl)propionate in 25% ethanol) of the drug solution to the surface around the knee, once per day until no pain on the knee.

In one embodiment, a subject will spray eight sprays (35 mg 2-(diethylamino)ethyl 2-(4-isobutylphenyl)propionate in 25% ethanol) of the drug solution to the surface around the knee, BID until no pain on the knee.

Detailed examples are shown as follows.

1. Nonclinical Pharmacology and Toxicology

A battery of pharmacology and toxicology studies including a GLP acute dermal maximum tolerated dose study in rats, a GLP acute dermal maximum tolerated dose study in Beagle dogs, a non-GLP 14-day repeated dermal dose toxicity study in rats, a non-GLP 14-day repeated dermal dose toxicity study in Beagle dogs, a 28-day GLP repeated dermal dose toxicity and toxicokinetics study in rats with 14-day recovery, a 28-day GLP repeated dermal dose toxicity and toxicokinetics study in Beagle dogs with 14-day recovery, a GLP bacterial reverse mutation assay (Ames), a GLP in vitro chromosome aberration assay in CHO-WBL Cells, a GLP in vivo bone marrow micronucleus assay in rats, a behavioral effects in rats using the functional observational battery, a rat respiratory safety pharmacology study, a cardiovascular telemetry study in the unrestrained conscious non-naïve dog, a skin irritation study in rabbits, a study on embryo-fetal development in rats, a 39-week GLP repeated dermal dose toxicity and toxicokinetics study in mini pigs with 4-week recovery, a 26-week GLP repeated dermal dose toxicity and toxicokinetics study in rats with 4-week recovery, a study on fertility and early embryonic development to implantation in rats, a study on embryo-fetal development in rabbits, and a sensitization test in guinea pigs were conducted. The study results show 2-(diethylamino)ethyl 2-(4-isobutylphenyl)propionate appeared to be safe and were generally well tolerated.

1.1. Primary Nonclinical Pharmacology

The study results show 2-(diethylamino)ethyl 2-(4-isobutylphenyl)propionate appeared to be safe and were generally well tolerated. A list of pharmacology studies and study results of 2-(diethylamino)ethyl 2-(4-isobutylphenyl)propionate (the Testing Compound) and results are shown in Table 1.

TABLE 1
Pharmacology Studies of 2-(diethylamino)ethyl 2-(4-isobutylphenyl)propionate

| | | Route of | |
|---|---|---|---|
| Study Type | Species | Administration | Results Summary |
| Adjuvant- induced arthritis | Lewis | Transdermal | Treatment with 2-(diethylamino)ethyl 2-(4-isobutylphenyl)propionate at |
| | rats | | 10 mg/kg, 30 mg/kg and 90 mg/kg daily by topical administration |
| | | | significantly inhibited arthritis. |
| Collagen II induced arthritis | Lewis | Transdermal | Treatment with 2-(diethylamino)ethyl 2-(4-isobutylphenyl)propionate at |
| | rats | | 10 mg/kg, 30 mg/kg and 90 mg/kg daily by topical administration showed |
| | | | an inhibitory effect measured by incidence and mean severity, and |
| | | | postponed the onset of the disease. |
| Osteoarthritis Induced by | SD Rats | Transdermal | Treatment with 2-(diethylamino)ethyl 2-(4-isobutylphenyl)propionate |
| Transection of the Anterior | | | showed recognizable reduction in the severity of osteOArthritic |
| Cruciate Ligament (ACL) (I) | | | degeneration but did not reach statistical significance. |
| Treatment started 4 weeks | | | The test compound did exert biological effects to reduce the OA severity, |
| post-surgery and ended 10 weeks | | | judging by histopathology evaluation, although the effects did not reach |
| post-surgery | | | statistical significance. |
| Osteoarthritis Induced by | SD Rats | Transdermal | The experiment on testing compounds that might exert effects on reducing |
| Transection of the Anterior | | | the development of osteOArthritic changes in the rat ALCT model did as |
| Cruciate Ligament (ACL) (II) | | | expected. All test compound treated groups, 2-(diethylamino)ethyl |
| Treatment started 1 week | | | 2-(4-isobutylphenyl)propionate 60 mg/kg or 30 mg/kg had lower disease |
| post-surgery and ended 10 weeks | | | modality scores. The treatment started at Week 2 seemed to be a good |
| post-surgery | | | initiation time for the therapeutic effects. |
| LPS induced Fever Model | SD Rats | Transdermal | LPS induced fever was attenuated by middle (30 mg/kg) and high |
| | | | (90 mg/kg) doses at both the 4 and 6 hour time points, indicating that at |
| | | | these two doses, 2-(diethylamino)ethyl 2-(4-isobutylphenyl)propionate |
| | | | was effective in inhibiting fever. 10 mg/kg 2-(diethylamino)ethyl |
| | | | 2-(4-isobutylphenyl)propionate slightly inhibited fever development, even |
| | | | though no significance was reached. |
| | | | The results clearly indicated that 2-(diethylamino)ethyl |
| | | | 2-(4-isobutylphenyl)propionate when applied topically can attenuate fever. |
| Carrageenan-induced Paw Edema | Wistar | Transdermal | Difference was observed with regard to the parameter tested between |
| in Wistar Rats | Rats | | control group and 2-(diethylamino)ethyl 2-(4-isobutylphenyl)propionate |
| | | | treated groups at various doses. The dosing regimen of test article |
| | | | 2-(diethylamino)ethyl 2-(4-isobutylphenyl)propionate at 90 mg/kg and |
| | | | 30 mg/kg both reduced the paw edema significantly. It showed dose |
| | | | dependent effect when 2-(diethylamino)ethyl |
| | | | 2-(4-isobutylphenyl)propionate concentration decreased. |
| Xylene-Induced Ear Edema in | ICR | Transdermal | The dosing regimen of test article 2-(diethylamino)ethyl |
| Mice | mice | | 2-(4-isobutylphenyl)propionate at 90 mg/kg reduced the ear edema |
| | | | significantly. Both the medium and low dose of 2-(diethylamino)ethyl |
| | | | 2-(4-isobutylphenyl)propionate showed statistical inhibitory effect in this |
| | | | study. |
| Acetic Acid Induced Pain in Mice | ICR | Transdermal | The dosing regimen of test article 2-(diethylamino)ethyl |
| | mice | | 2-(4-isobutylphenyl)propionate (90 and 30 mg/kg) reduced the number of |
| | | | writhes in a dose-dependent manner. High dose of 2-(diethylamino)ethyl |
| | | | 2-(4-isobutylphenyl)propionate showed significant inhibitory effect |
| | | | compared with Vehicle. However, low dose of 2-(diethylamino)ethyl |
| | | | 2-(4-isobutylphenyl)propionate did not show any inhibitory effect in this |
| | | | study. |

1.2. Safety Pharmacology

A battery of safety pharmacology studies including behavioral effects in rats using the functional observational battery, a rat respiratory safety pharmacology study, a cardiovascular telemetry study in the unrestrained conscious non-naïve dogs were completed. The results of the safety pharmacology studies conducted are shown in Table 2. The study results show 2-(diethylamino)ethyl 2-(4-isobutylphenyl)propionate appeared to be safe and were generally well tolerated.

TABLE 2
Safety Pharmacology Studies of 2-(diethylamino)ethyl 2-(4-isobutylphenyl)propionate

| | | Route of | GLP | |
|---|---|---|---|---|
| Study Type | Species | Administration | Compliance | Results Summary |
| Behavioral effects in | Rats | Transdermal | Yes | The single dose dermal administration of 2-(diethylamino)ethyl |
| rats using the | | | | 2-(4-isobutylphenyl)propionate to Sprague Dawley rats at dose levels of 0, 50, |
| functional | | | | 250, and 750 mg/kg of ibuprofenamine were not associated with any |
| observational battery | | | | neurobehavioral changes as assessed by the FOB test. |
| Respiratory safety | Rats | Transdermal | Yes | The single dose dermal administration of 2-(diethylamino)ethyl |
| pharmacology study | | | | 2-(4-isobutylphenyl)propionate to Sprague Dawley rats at dose levels of 0, 50, |
| | | | | 250, and 750 mg/kg of ibuprofenamine were not associated with respiratory |
| | | | | changes. |
| Cardiovascular | Dogs | Transdermal | Yes | A bOArd-certified veterinary cardiologist conducted a qualitative review of the |
| telemetry study in | | | | electrocardiograms obtained twice prior to each dose (at least 30 minutes apart) |
| the unrestrained | | | | and at 4, 8, 12, 24, 36 and 48 hours following the dermal administration of 10, |
| conscious non-naïve | | | | 25, and 50 mg/kg of ibuprofenamine and vehicle in dogs. There were no effects |
| dog | | | | of the dermal administration of 2-(diethylamino)ethyl |
| | | | | 2-(4-isobutylphenyl)propionate on qualitative ECG parameters. |

1.3. Nonclinical Toxicology Summary

1.3.1. Summary of General Toxicity Studies

The study results show 2-(diethylamino)ethyl 2-(4-isobutylphenyl)propionate appeared to be safe and were generally well tolerated. A listing of general toxicology studies of 2-(diethylamino)ethyl 2-(4-isobutylphenyl)propionate are shown in the following Table 3.

TABLE 3
Listing of General Toxicology Studies of 2-(diethylamino)ethyl 2-(4-isobutylphenyl)propionate

| | | Route of | GLP | |
|---|---|---|---|---|
| Study Type | Species | Administration | Compliance | Results Summary |
| Acute dermal maximum | Rats | Transdermal | Yes | The single dose dermal administration of 2-(diethylamino)ethyl |
| tolerated dose study | | | | 2-(4-isobutylphenyl)propionate to rats at 1000 mg/kg was well tolerated |
| | | | | and resulted in slight skin reaction, slightly lower body weight gains |
| | | | | when compared to the low group, and red discoloration of subcutaneous |
| | | | | in the dosing area. Under the conditions of this study, the MTD for |
| | | | | 2-(diethylamino)ethyl 2-(4-isobutylphenyl)propionate in male and female |
| | | | | rats when administered once by dermal application was considered to |
| | | | | be >1000 mg/kg. |
| Acute maximum tolerated | Beagle | Transdermal | Yes | The maximum tolerated dose was at least 50 mg/kg (Ibuprofen average |
| dose study | dogs | | | values of Cₘₐₓ 5875 ng/mL, and AUC_{0-24 hr} 88525 h · ng/mL) following |
| | | | | dermal application under the current study condition. |
| Acute maximum tolerated | Beagle | Subcutaneous | Yes | The maximum tolerated dose of 2-(diethylamino)ethyl |
| dose study | dogs | injection | | 2-(4-isobutylphenyl)propionate by subcutaneous injection was 500 mg/kg |
| | | | | under the current study condition. |
| 14-day repeated dermal | Rats | Transdermal | No | The no observed adverse effect level (NOAEL) of 2-(diethylamino)ethyl |
| dose toxicity study | | | | 2-(4-isobutylphenyl)propionate was considered to be 250 mg/kg/day. |
| 14-day repeated dermal | Beagle | Transdermal | No | The no-observed-adverse-effect (NOAEL) was considered to be 50 |
| dose toxicity study | dogs | | | mg/kg/day in the current study condition. |
| 28-day GLP repeated | Rats | Transdermal | Yes | The systemic No Observed-Adverse-Effect-Level (NOAEL) of |
| dermal dose toxicity and | | | | 2-(diethylamino)ethyl 2-(4-isobutylphenyl)propionate was considered to |
| toxicokinetics study | | | | be 100 mg/kg/day for both male and female rats in this study, and the |
| | | | | AUC_{0-24 h} and Cₘₐₓ of 2-(diethylamino)ethyl |
| | | | | 2-(4-isobutylphenyl)propionate were 165000 h · ng/mL and 47400 ng/mL |
| | | | | for the males, and 422000 h · ng/mL and 46800 ng/mL for the females on |
| | | | | Day 28, respectively. Additionally, the local NOAEL was considered to be |
| | | | | 25 mg/kg/day based upon ulcers at the dermal application site of the rats |
| | | | | given ≥100 mg/kg/day. |
| 28-day GLP repeated | Beagle | Transdermal | Yes | The systemic NOAEL (tissues other than the dermal application site) was |
| dermal dose toxicity and | dogs | | | 80→ 50 mg/kg/day or 24.8 mg/kg/day, based upon the absence of any test |
| toxicokinetics study | | | | article-related organ weight changes, or macroscopic or microscopic |
| | | | | findings. The No-Observed-Adverse-Effect-Level (NOAEL) was not |
| | | | | attained in this study, based upon ulcers at the dermal application site at the |
| | | | | lowest administered dose (12.4 mg/kg/day). |
| A 39-week GLP repeated | Mini | Transdermal | Yes | A 39-week GLP repeated dermal dose toxicity and toxicokinetics study in |
| dermal dose toxicity and | pigs | | | mini pigs with 4-week recovery was done and no test article-related local |
| toxicokinetics study in mini | | | | or systemic adverse effects were found at all dosages 5, 12.5 and 25 mg/kg. |
| pigs with 4-week recovery | | | | |

2. Phase 1 Clinical Study

2.1. Methodology

It is a single center, randomized, double-blind, placebo-controlled dose-escalation study to evaluate the safety, tolerability and pharmacokinetics of single and multiple doses of 2-(diethylamino)ethyl 2-(4-isobutylphenyl)propionate following escalating single and multiple doses administered as a topical application.

The study was conducted in six cohorts. In each cohort of 10 subjects, eight subjects were randomized to receive active drug and two subjects were randomized to receive matching placebo. Dose levels of 17.5, 35, 70, 140 and 280 mg of topical application and 70 mg as spray application were evaluated.

The study design incorporated staggered durations of single doses followed by 7 days of twice daily (b.i.d.) doses of study drug for evaluation of safety and pharmacokinetics as overlapping dose cohorts. There were 2 parts to the study.

For Part 1, subjects were admitted to the clinical research unit (CRU) on the evening prior to dosing and remained sequestered at the CRU until after the 24 hour post-dose blood sample collection on Day 2. Subjects returned to the CRU for 2 out-patient-visits (OPVs) at approximately 0900 hours f 1 hour on Days 3 and 4 for collection of the 48 and 72 hour post-dose blood samples.

For Part 2, subjects were admitted to the CRU the morning of Day 5 (96 hour post-dose blood sample collected) and remained sequestered at the CRU for 8 overnight stays until Day 13, approximately 24 hours after administration of the final Day 12 dose. Subjects returned for 4 OPVs at approximately 0800 hours f 1 hour on Days 14-17 for collection of the 48, 72, 96, and 120 hours post-dose blood samples. Final safety assessments were performed on Day 17 or at early termination, and subjects were discharged from the study. All procedures remained the same for all cohorts.

In Part 1 of the study, each cohort began with single dose administration of drug on Day 1 and serial blood sample collection for evaluation of 2-(diethylamino)ethyl 2-(4-isobutylphenyl)propionate and its metabolite, ibuprofen, concentrations and pharmacokinetic (PK) analysis over the 120-hour post-dose period. Following a 5-day washout period, 2-(diethylamino)ethyl 2-(4-isobutylphenyl)propionate was administered b.i.d. during Days 6-11, and a morning dose only was administered to Day 12. Blood samples were collected for PK analysis over the 120-hour post-dose period following the Day 12 dose. Additionally, pre-dose blood samples were collected on Day 7 through Day 11 to evaluate steady-state status.

A review of safety data was performed prior to initiation of multiple dosing at each dose level and dose escalation to the next higher single dose. In the absence of both dose-limiting adverse events and laboratory toxicities, administration of single dose or multiple doses at the next level was to be initiated.

In Part 2 of the study, a separate cohort of ten subjects (eight subjects randomized to receive active drug and two subjects to receive placebo) received a 70 mg dose as spray application (70 mg is equivalent to 16 sprays). The same study procedures were followed as described in Part 1.

For single dose PK, serial blood samples were collected on Day 1 dose at the following time points: 0 (pre-dose), 0.25, 0.5, 0.75, 1, 1.5, 2, 2.5, 3, 4, 5, 6, 8, 10, 12, 18, 24, 48, 72, 96 and 120 hours post-dose.

For multiple-dose PK, serial blood samples were collected on Day 12 at the following time points: 0 (pre-dose), 0.25, 0.5, 0.75, 1, 1.5, 2, 2.5, 3, 4, 5, 6, 8, 10, 12, 18, 24, 48, 72, 96 and 120 hours post-dose.

Safety assessments included monitoring of adverse events (AEs), vital signs (blood pressure, pulse rate, respiratory rate and oral temperature), clinical laboratory findings, resting 12-lead electrocardiogram (ECG) results, skin irritation assessments and physical examination findings.

The duration of treatment for each subject was up to approximately 6 weeks, including a 21-day screening period and a 17-day treatment period.

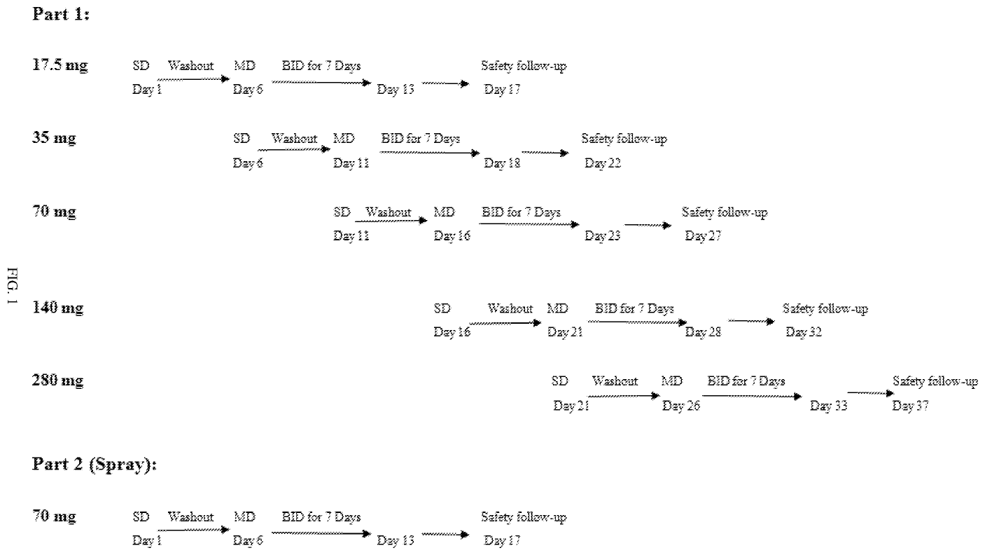

The study schema of the Phase 1 clinical study is shown is FIG. 1.

2.2. Statistical Methods Statistical Methods Planned in the Protocol and Determination of Sample Size

2.2.1. Statistical and Analytical Plans

The planned analysis for this study is described in detail in the Statistical Analysis Plan dated 28 Mar. 2013 (Appendix 16.1.9), which was generated by Frontage and approved by the sponsor prior to database lock and is briefly described below.

The primary objective of this study was to evaluate the safety and tolerability of escalating single and multiple doses of 2-(diethylamino)ethyl 2-(4-isobutylphenyl)propionate administered as a topical application.

The secondary objectives of this study were to characterize the single and steady-state pharmacokinetics of escalating doses of 2-(diethylamino)ethyl 2-(4-isobutylphenyl)propionate and ibuprofen as a topical application and to evaluate relative bioavailability of spray application vs. topical application of 2-(diethylamino)ethyl 2-(4-isobutylphenyl)propionate.

2.2.2. Populations

There are two analysis populations:
Safety Population: all subjects who receive at least one dose of study drug and have at least one post-dose safety assessment.
PK Population: all subjects who receive all planed doses of active study drug, have no major protocol deviations, and have sufficient pharmacokinetic data to obtain reliable estimates of the key pharmacokinetic variables.

Protocol deviations were identified prior to database lock and may have included but were not limited to significant violations of inclusion/exclusion criteria, noncompliance of the trial treatment taken, use of prohibited medications and not following clinical trial protocol procedures.

2.2.3. Pharmacokinetic Evaluations

Pharmacokinetic variables were calculated for 2-(diethylamino)ethyl 2-(4-isobutylphenyl)propionate and ibuprofen from the plasma concentration data using standard, non-compartmental methods using WinNonlin (version 6.2.1) and actual elapsed times of blood collection.

Pharmacokinetic variables determined are displayed in Table 4. Pharmacokinetic parameters are listed by subjects and summarized by treatment using descriptive statistics.

TABLE 4
Pharmacokinetic Parameters

| Parameter | Description |
|---|---|
| AR_{Cmax} | Accumulation ratios of maximum plasma concentration (Day 12 vs. Day 1), calculated by the following: AR_{Cmax} = |
| | Cₘₐₓ on Day 12/Cₘₐₓ on Day 1 |
| AR_{Cmax0-12 hr} | Accumulation ratios of maximum plasma concentration within dosing interval (Day 12 vs. Day 6), calculated by the |
| | following: AR_{Cmax0-12 hr} = Cₘₐₓ within 0-12 on Day 12/Cₘₐₓ within 0-12 on Day 6 |
| AR_{AUCinf} | Accumulation ratios of the area under the concentration versus time curve from time zero to infinity (Day 12 vs. Day |
| | 1), calculated by the following: AR_{AUCinf} = AUC_{inf} on Day 12/AUC_{inf} on Day 1 |
| AR_{AUC0-12 hr} | Accumulation ratios of area under the concentration versus time curve during dosing interval (Day 12 vs. Day 6), |
| | calculated by the following: AR_{AUC0-12 hr} = AUC_{0-12 hr} on Day 12/AUC_{0-12 hr} on Day 6 |
| AUCₗₐₛₜ | The area under the plasma concentration versus time curve, from time zero (0) to the time of the last measurable |
| | plasma concentration (Tₗₐₛₜ) as calculated by the linear trapezoidal method |
| AUCₜₐᵤ | Area under the curve during dosing interval. In this study AUCₜₐᵤ is based on a 12 h interval. |
| AUC_{inf} | The area under the concentration versus time curve from time zero (0) to infinity |
| Cₘₐₓ | Maximum plasma concentration, obtained directly from the observed concentration versus time data |
| Cₘᵢₙ | Minimum plasma concentration, obtained directly from the observed concentration versus time data, associated with |
| | the concentration at the end of the dosing interval, the pre-dose or trough value. |
| Tₘₐₓ | Time of the maximum measured plasma concentration |
| Kₑₗ or λz | Terminal elimination rate constant (absolute value of the slope of the linear regression of the natural logarithm of |
| | concentration vs. time during the terminal phase of the concentration-time curve) |
| Half-life (t_{1/2}) | Half-life, calculated by the following: t_{1/2} = ln(2)/Kₑₗ |

Actual elapsed times of blood collection times were used for all pharmacokinetic analyses. Nominal blood collection times were used to calculate mean plasma concentrations for graphical displays and tabulated group summaries

Accumulation ratios of Cₘₐₓ, C_{max0-12 hr} and AUCs (AUC_{0-12 hr} and AUC_{inf}) (Day 12 vs. Day 1 or Day 12 vs. Day 6) were calculated for subjects in each cohort in Part 1 and for subjects in Part 2 for both 2-(diethylamino)ethyl 2-(4-isobutylphenyl)propionate and ibuprofen, and summarized by treatment using descriptive statistics.

The relative bioavailability of the single dose 70 mg topical application vs. 70 mg spray application was determined based on AUCₜₐᵤ, AUC_{inf} and Cₘₐₓ. The 90% confidence intervals (CIs) on the ratio of topical application vs. spray application were calculated.

Dose proportionality after single doses of 2-(diethylamino)ethyl 2-(4-isobutylphenyl)propionate was assessed by fitting the estimates of natural log transformed parameters AUCₗₐₛₜ, AUC_{inf}, AUCₜₐᵤ and Cₘₐₓ. A linear relationship between the ln transformed PK parameters of 2-(diethylamino)ethyl 2-(4-isobutylphenyl)propionate and the ln-transformed dose was fitted by using the power model:

ln(Y)=β₀+β ln(Dose)+e
where Y represents the PK parameter Cₘₐₓ, AUCₗₐₛₜ, AUC_{inf}, AUC_{inf} following single 2-(diethylamino)ethyl 2-(4-isobutylphenyl)propionate administration and e represents the error term. Slope @ and its 90% CIs were calculated to assess the dose proportionality. If the 90% CIs of 3 was entirely within the critical region [1+ln(θ_{L})/ln(R), 1+ln(θ_{H})/ln(R)], where θ_{L}=0.8 and θ_{H}=1.25, and R is the ratio of the highest dose vs. the lowest dose, then dose proportionality was to be declared.

2.2.4. Safety Evaluations

Safety endpoints consist of all AEs, clinical laboratory test results, ECG, physical examination and vital sign assessments and skin irritation assessments.

Safety summaries are provided for all subjects in the safety population. Data from subjects in each cohort who received placebo treatment were pooled. AE data, clinical laboratory test results, ECG, skin irritation and vital signs data are presented descriptively using summary tables and listings. Change from baseline in vital signs, ECG and clinical laboratory results are presented descriptively using summary tables and listings. Physical examination findings are listed by subject.

2.2.5. Determination of Sample Size

This was an early development study, and therefore no statistical considerations were involved in the sample size determination. It was expected that the sample size of 10 subjects (eight subjects receiving active drug and two subjects receiving placebo) in each cohort should be adequate for evaluation of tolerability and pharmacokinetic parameters in this single and multiple ascending dose study.

2.3. Selection of Study Population

2.3.1. Inclusion Criteria

For inclusion into the trial, subjects were required to fulfill all of the following criteria:
1. Are capable of giving informed consent and complying with study procedures;
2. Are between the ages of 18 and 45 years, inclusive;
3. Female subjects have a negative urine pregnancy test result prior to enrollment if they are of child-bearing potential and must agree to use a medically acceptable form of birth control from screening through to study completion: 3 months previously on hormonal contraceptives (e.g., oral or patch contraceptives), intrauterine device, Depo-Provera®, or a double barrier method (condom with spermicide, diaphragm with spermicide), or meet the following criteria defined as:
a. Surgically sterile for at least 3 months prior to screening by one of the following means:
   Bilateral tubal ligation
   Salpingectomy (with or without oophorectomy)
   Surgical hysterectomy
   Bilateral oophorectomy (with or without hysterectomy)
b. Postmenopausal, defined as:
   Last menstrual period greater than 12 months prior to screening, and confirmed by FSH.
4. Considered healthy by the Principal Investigator, based on a detailed medical history, full physical examination, clinical laboratory tests, 12-lead ECG and vital signs;
5. Nonsmoker, defined as not having smoked or used any form of tobacco in more than 6 months before screening;
6. Body mass index (BMI) of 19 to 30 kg/m² inclusive and body weight not less than 50 kg;
7. Willing and able to adhere to study restrictions and to be confined at the clinical research center.

2.3.2. Exclusion Criteria

Any of the following was regarded as a criterion for exclusion from the trial:
1. Clinically significant history of gastrointestinal, cardiovascular, musculoskeletal, endocrine, hematologic, psychiatric, renal, hepatic, bronchopulmonary, neurologic, immunologic, lipid metabolism disorders, or drug hypersensitivity;
2. A history of gastrointestinal bleeding or peptic ulcers, hypersensitivity to aspirin or other NSAIDs, or a history of asthma or other allergic-type reactions after taking aspirin or other NSAIDs;
3. Any visible skin disease, damage or condition at the application sites which, in the opinion of the investigator, could compromise subject safety and/or interfere with the evaluation of the test site reaction;
4. Known or suspected malignancy;
5. Positive blood screen for human immunodeficiency virus (HIV), hepatitis B surface antigen (HBsAg), or hepatitis C antibody;
6. Positive pregnancy test result, or plan to be pregnant if female;
7. A hospital admission or major surgery within 30 days prior to screening;
8. Participation in any other investigational drug trial within 30 days prior to screening;
9. A history of prescription drug abuse, or illicit drug use within 6 months prior to screening;
10. A history of alcohol abuse according to medical history within 6 months prior to screening;
11. A positive screen for alcohol, drugs of abuse;
12. An unwillingness or inability to comply with food and beverage restrictions during study participation;
13. Donation or blood collection of more than 1 unit (approximate 450 mL) of blood (or blood products) or acute loss of blood during the 90 days prior to screening;
14. Use of prescription or over-the-counter (OTC) medications, and herbal (including St John's Wort, herbal teas, garlic extracts) within 14 days prior to dosing (Note: Use of acetaminophen at <3 g/day is permitted until 24 hours prior to dosing);
15. A history of intolerance or hypersensitivity to ibuprofenamine hydrochloride or any excipients or to the diluent ethanol;
16. An unwillingness of male participants to use appropriate contraceptive measures if engaging in sexual intercourse with a female partner of childbearing potential. Appropriate measures include use of a condom and spermicide and, for female partners, use of an intrauterine device (IUD), diaphragm with spermicide, oral contraceptives, injectable progesterone, progesterone subdermal implants, or a tubal ligation. Sexual intercourse with pregnant or lactating women is prohibited.

2.4. Treatments Administered

Subjects within each cohort were randomly assigned to receive 17.5 mg, 35 mg, 70 mg, 140 mg, and 280 mg of 2-(diethylamino)ethyl 2-(4-isobutylphenyl)propionate or matching placebo administered as a topical application (Part 1, Cohorts 1-5) or 70 mg of 2-(diethylamino)ethyl 2-(4-isobutylphenyl)propionat or placebo administered as a spray (Part 2, Cohort 6). Within each cohort, subjects were assigned to receive active treatment or placebo at a 4:1 ratio. Treatments for each cohort are displayed in Table 5.

TABLE 5
Treatment Assignment

| Cohort | Single Dose | Multiple Dose | Subjects |
|---|---|---|---|
| 1 | 17.5 mg × 1 | 17.5 mg b.i.d. × 7 | 10 (8 active + 2 placebo) |
| 2 | 35 mg × 1 | 35 mg b.i.d. × 7 | 10 (8 active + 2 placebo) |
| 3 | 70 mg × 1 | 70 mg b.i.d. × 7 | 10 (8 active + 2 placebo) |
| 4 | 140 mg × 1 | 140 mg b.i.d. × 7 | 10 (8 active + 2 placebo) |
| 5 | 280 mg × 1 | 280 mg b.i.d. × 7 | 10 (8 active + 2 placebo) |
| 6 | 70 mg × 1 | 70 mg b.i.d. × 7 | 10 (8 active + 2 placebo) |

Doses were administered to Day 1 at approximately 0800 hours±1 hour, on Day 6 through Day 11 at approximately 0800 hours±1 hour and 2000 hours±1 hour, and on Day 12 at approximately 0800 hours±1 hour. All doses were administered by CRU personnel according to a Dosing Manual prepared prior to the start of the study and approved by the sponsor.

2.5. Pharmacokinetics Results

2.5.1. Analysis of Pharmacokinetics

2.5.1.1. Plasma Concentrations

The determination of plasma 2-(diethylamino)ethyl 2-(4-isobutylphenyl)propionate and ibuprofen concentrations was performed in accordance with applicable Good Laboratory Practice regulations (21 CRF 58) and FDA's May 2001 Guidance for Industry, Bioanalytical Method Validation.

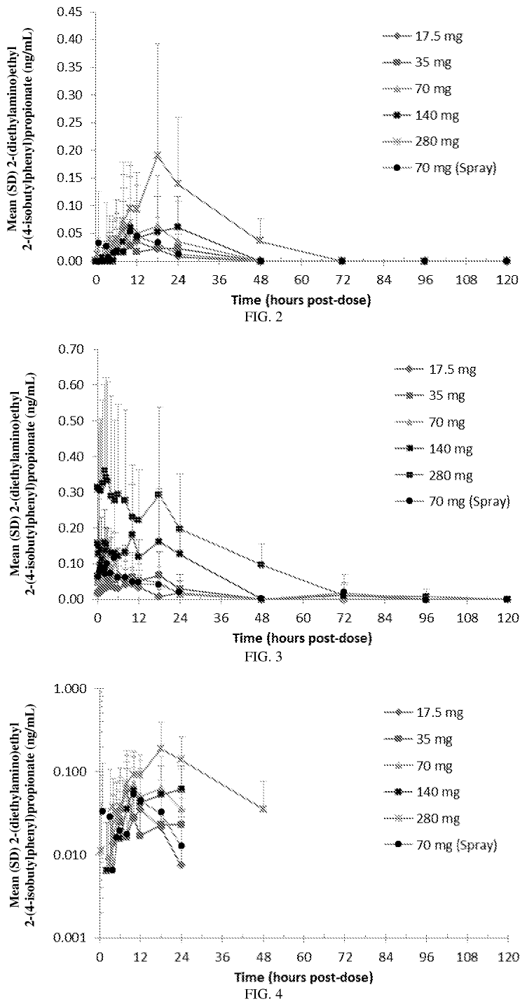

The mean concentration versus time profiles on Day 1 and Day 12 from time 0 to 120 hours post-dose for all 2-(diethylamino)ethyl 2-(4-isobutylphenyl)propionate treatment groups are displayed by treatment on a linear scale in FIG. 2 and FIG. 3, respectively, and on Day 1 and Day 12 on a semi-log scale in FIG. 4 and FIG. 5, respectively.

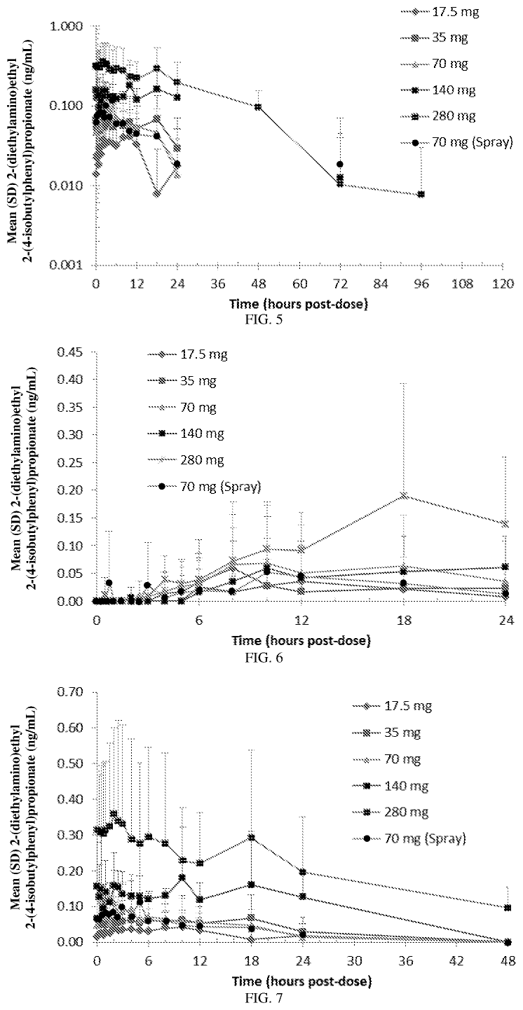

In order to better differentiate the 2-(diethylamino)ethyl 2-(4-isobutylphenyl)propionate concentration versus time profiles by treatment group, this data is displayed for Day 1 from time 0 to 24 hours post-dose in FIG. 6, and from time 0 to 48 hours post-dose for Day 12 in FIG. 7.

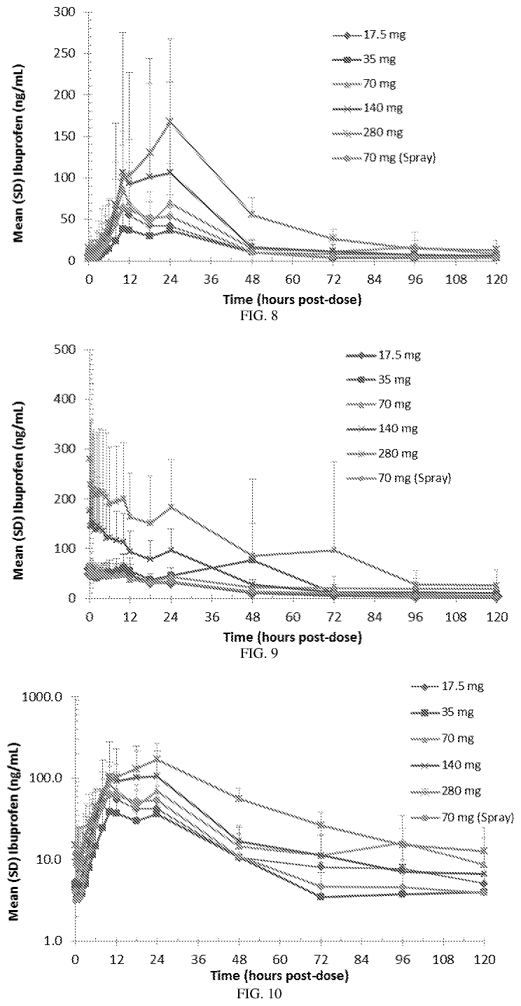

The mean concentration versus time profiles on Day 1 and Day 12 from time 0 to 120 hours post-dose for ibuprofen are displayed by treatment on a linear scale in FIG. 8 and FIG. 9, respectively, and on a semi-log scale in FIG. 10 and FIG. 11, respectively.

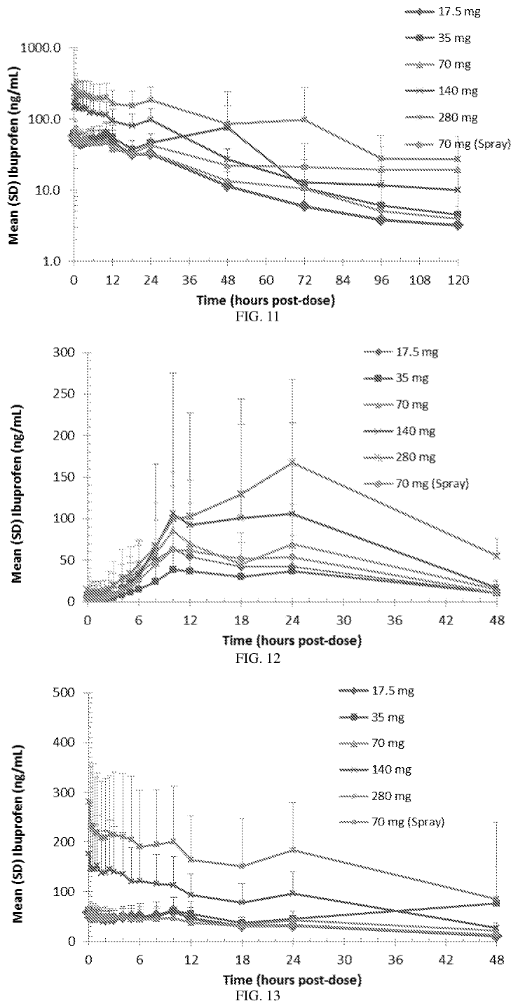

In order to better differentiate the ibuprofen concentration versus time profiles by treatment group, this data is displayed for Day 1 from time 0 to 48 hours post-dose in FIG. 12, and from time 0 to 48 hours post-dose for Day 12 in FIG. 13.

2.5.1.2. Pharmacokinetics Parameters

2-(diethylamino)ethyl 2-(4-isobutylphenyl)propionate and ibuprofen PK parameters are summarized in Table 6 and Table 7 (Day 1) and in Table 8 and Table 9 (Day 12).

TABLE 6
2-(diethylamino)ethyl 2-(4-isobutylphenyl)propionate Pharmacokinetic Parameters (Day 1)—PK population

| | 2-(diethylamino) | 2-(diethylamino) | 2-(diethylamino) | 2-(diethylamino) | 2-(diethylamino) | 2-(diethylamino) |
|---|---|---|---|---|---|---|
| | ethyl 2-(4- | ethyl 2-(4- | ethyl 2-(4- | ethyl 2-(4- | ethyl 2-(4- | ethyl 2-(4- |
| PK | isobutylphenyl) | isobutylphenyl) | isobutylphenyl) | isobutylphenyl) | isobutylphenyl) | isobutylphenyl) |
| Parameter | propionate | propionate | propionate | propionate | propionate | propionate |
| (unit) | 17.5 mg N = 3 | 35 mg N = 4 | 70 mg N = 4 | 140 mg N = 5 | 280 mg N = 7 | 70 mg spray N = 5 |
| Cₘₐₓ (ng/mL) | 0.1583 (0.0959) | 0.0862 (0.0428) | 0.1602 (0.1047) | 0.1162 (0.0768) | 0.2333 (0.1960) | 0.1187 (0.0825) |
| AUCₗₐₛₜ | 1.450 (1.212) | 0.8639 (0.4389) | 1.993 (2.079) | 1.452 (1.105) | 4.479 (4.012) | 0.9466 (1.207) |
| (ng · hr/mL) | | | | | | |
| AUC₀₋₁₂ | 0.8372 (0.5323) | 0.3400 (0.2132) | 0.8360 (0.8404) | 0.4782 (0.4589) | 0.6748 (0.4766) | 0.5136 (0.6656) |
| (ng · hr/mL) | | | | | | |
| Tₘₐₓ (h) | 8.0 (8.0-8.0) | 11.0 (6.0-24.0) | 10.0 (8.0-18.0) | 18.0 (10.0-24.0) | 18.0 (10.0-24.0) | 10.0 (0.75-18.0) |

Values displayed are Mean (SD) except for Tₘₐₓ, which is displayed as median (range).
N/A: Nor Available;
NR: Not Reported.
AUC_{inf}, t_{1/2} or Kₑₗ not reported if Rsq adjusted < 0.8 or t1/2 > one half sampling interval.
AUC_{inf}, t_{1/2} and Kₑₗ data not available for more than one subject in a dose group; summary data not displayed.

TABLE 7
Ibuprofen Pharmacokinetic Parameters (Day 1)—PK population

| | 2-(diethylamino) | 2-(diethylamino) | 2-(diethylamino) | 2-(diethylamino) | 2-(diethylamino) | 2-(diethylamino) |
|---|---|---|---|---|---|---|
| | ethyl 2-(4- | ethyl 2-(4- | ethyl 2-(4- | ethyl 2-(4- | ethyl 2-(4- | ethyl 2-(4- |
| PK | isobutylphenyl) | isobutylphenyl) | isobutylphenyl) | isobutylphenyl) | isobutylphenyl) | isobutylphenyl) |
| Parameter | propionate | propionate | propionate | propionate | propionate | propionate |
| (unit) | 17.5 mg N = 8 | 35 mg N = 8 | 70 mg N = 8 | 140 mg N = 7 | 280 mg N = 8 | 70 mg spray N = 8 |
| Cₘₐₓ (ng/mL) | 73.18 (56.60) | 50.90 (28.01) | 92.41 (65.55) | 133.9 (172.5) | 171.5 (98.04) | 70.86 (39.08) |
| AUCₗₐₛₜ | 2122 (1255) | 1526 (932.9) | 3167 (2433) | 4033 (4546) | 6682 (2505) | 2212 (986.7) |
| (ng · hr/mL) | | | | | | |
| AUC₀₋₁₂ | 391.0 (403.7) | 217.1 (158.4) | 515.2 (481.0) | 572.8 (853.8) | 615.2 (250.9) | 363.0 (229.6) |
| (ng · hr/mL) | | | | | | |
| AUC_{inf} | 1765 (1092) | 1031 (450.7) | 2578 (2010) | NR | 6839 (2189) | 2003 (976.3) |
| (ng · hr/mL) | | | | | | |
| Tₘₐₓ (h) | 21.0 (10.0-24.0) | 14.0 (10.0-48.37) | 18.0 (10.0-24.0) | 24.0 (10.0-24.0) | 24.0 (12.0-24.0) | 11.0 (10.0-24.0) |
| t_{1/2} (h) | 17.27 (5.287) | 13.67 (5.245) | 24.77 (7.360) | 92.47 (N/A) | 24.30 (7.445) | 69.84 (73.26) |
| Kₑₗ (1/h) | 0.0421 (0.0129) | 0.0566 (0.0195) | 0.0303 (0.0105) | 0.0075 (N/A) | 0.0310 (0.0110) | 0.0250 (0.0242) |

Values displayed are Mean (SD) except for Tₘₐₓ, which is displayed as median (range).
AUC_{inf}, t_{1/2} or Kₑₗ not reported if Rsq adjusted < 0.8 or t_{1/2} > one half sampling interval.

TABLE 8
2-(diethylamino)ethyl 2-(4-isobutylphenyl)propionate Pharmacokinetic Parameters (Day 12)—PK Population

| | 2-(diethylamino) | 2-(diethylamino) | 2-(diethylamino) | 2-(diethylamino) | 2-(diethylamino) | 2-(diethylamino) |
|---|---|---|---|---|---|---|
| | ethyl 2-(4- | ethyl 2-(4- | ethyl 2-(4- | ethyl 2-(4- | ethyl 2-(4- | ethyl 2-(4- |
| PK | isobutylphenyl) | isobutylphenyl) | isobutylphenyl) | isobutylphenyl) | isobutylphenyl) | isobutylphenyl) |
| Parameter | propionate | propionate | propionate | propionate | propionate | propionate |
| (unit) | 17.5 mg N = 3 | 35 mg N = 4 | 70 mg N = 4 | 140 mg N = 5 | 280 mg N = 7 | 70 mg spray N = 5 |
| Cₘₐₓ (ng/mL) | 0.0920 (0.0269) | 0.1533 (0.0479) | 0.1623 (0.0753) | 0.3050 (0.1570) | 0.5051 (0.2725) | 0.1852 (0.1410) |
| AUCₗₐₛₜ | 1.308 (0.5898) | 2.290 (0.8101) | 1.488 (1.030) | 3.598 (1.673) | 11.63 (5.337) | 2.619 (2.811) |
| (ng · hr/mL) | | | | | | |
| AUC₀₋₁₂ | 0.8088 (0.1567) | 1.210 (0.3715) | 1.105 (0.6039) | 1.818 (0.5263) | 3.714 (2.515) | 1.065 (0.2206) |
| (ng · hr/mL) | | | | | | |
| AUC_{inf} | NR | NR | NR | NR | 18.11 (4.364) | NR |
| (ng · hr/mL) | | | | | | |
| Tₘₐₓ (h) | 8.0 (8.0-10.0) | 5.125 (0-18.0) | 3.5 (2.0-8.0) | 2.5 (0-18.0) | 0.25 (0-3.0) | 3.0 (0-8.0) |
| t_{1/2} (h) | 28.77 (N/A) | NR | 10.57 (N/A) | 17.40 (N/A) | 37.80 (31.20) | NR |
| Kₑₗ (1/h) | 0.0241 (N/A) | NR | 0.0656 (N/A) | 0.0398 (N/A) | 0.0374 (0.0310) | NR |

Values displayed are Mean (SD) except for Tₘₐₓ, which is displayed as median (range).
N/A: Nor Available;
NR: Not Reported.
AUC_{inf}, t_{1/2} or Kₑₗ not reported if Rsq adjusted < 0.8 or t_{1/2} > one half sampling interval.

TABLE 9
Ibuprofen Pharmacokinetic Parameters (Day 12)—PK Population

| | 2-(diethylamino) | 2-(diethylamino) | 2-(diethylamino) | 2-(diethylamino) | 2-(diethylamino) | 2-(diethylamino) |
|---|---|---|---|---|---|---|
| | ethyl 2-(4- | ethyl 2-(4- | ethyl 2-(4- | ethyl 2-(4- | ethyl 2-(4- | ethyl 2-(4- |
| PK | isobutylphenyl) | isobutylphenyl) | isobutylphenyl) | isobutylphenyl) | isobutylphenyl) | isobutylphenyl) |
| Parameter | propionate | propionate | propionate | propionate | propionate | propionate |
| (unit) | 17.5 mg N = 8 | 35 mg N = 8 | 70 mg N = 8 | 140 mg N = 7 | 280 mg N = 8 | 70 mg spray N = 8 |
| Cₘₐₓ (ng/mL) | 63.40 (27.37) | 115.9 (136.0) | 61.84 (24.33) | 180.2 (100.7) | 304.7 (165.0) | 67.69 (25.26) |
| AUCₗₐₛₜ | 1909 (560.8) | 3753 (4317) | 3155 (1751) | 5028 (2223) | 11855 (6883) | 2087 (882.6) |
| (ng · hr/mL) | | | | | | |
| AUC₀₋₁₂ | 609.1 (273.2) | 625.2 (220.3) | 552.0 (169.9) | 1496 (826.6) | 2414 (1362) | 565.1 (221.0) |
| (ng · hr/mL) | | | | | | |
| AUC_{inf} | 2029 (706.3) | 2038 (632.0) | 1989 (639.1) | 5466 (2632) | 9632 (5094) | 2006 (833.5) |
| (ng · hr/mL) | | | | | | |
| Tₘₐₓ (h) | 5.0 (0-10.0) | 10.0 (0-47.33) | 7.0 (0-71.88) | 0 (0-24.0) | 0 (0-71.88) | 0 (0-2.0) |
| t_{1/2} (h) | 29.74 (7.205) | 29.05 (19.96) | 42.85 (38.65) | 24.00 (7.914) | 22.39 (5.511) | 32.56 (19.88) |
| Kₑₗ (1/h) | 0.0247 (0.0073) | 0.0359 (0.0258) | 0.0244 (0.0126) | 0.0318 (0.0107) | 0.0323 (0.0068) | 0.0256 (0.0092) |

Values displayed are Mean (SD) except for Tₘₐₓ, which is displayed as median (range).
AUC_{inf}, t_{1/2} or Kₑₗ not reported if Rsq adjusted < 0.8 or t_{1/2} > one half sampling interval.

All subjects who received study drug as a topical application had measurable plasma levels of 2-(diethylamino)ethyl 2-(4-isobutylphenyl)propionate by 3 hours post-dose, whereas the active metabolite, ibuprofen, had measurable plasma levels, relatively instantly. Thus upon absorption, the prodrug 2-(diethylamino)ethyl 2-(4-isobutylphenyl)propionate, is rapidly converted (>99% of absorbed prodrug) to its active metabolite, ibuprofen.

Following single and multiple applications of 2-(diethylamino)ethyl 2-(4-isobutylphenyl)propionate topically, mean maximum 2-(diethylamino)ethyl 2-(4-isobutylphenyl)propionate and ibuprofen concentrations (Cₘₐₓ) and AUC₀₋ₗₐₛₜ did not increase in a dose-proportional manner as the 2-(diethylamino)ethyl 2-(4-isobutylphenyl)propionate dose increased from 17.5 mg to 280 mg.

Mean pharmacokinetic parameters could not be reliably calculated for most subjects on Day 1 or Day 12 for 2-(diethylamino)ethyl 2-(4-isobutylphenyl)propionate.

Following 7 consecutive days of b.i.d. dosing of 2-(diethylamino)ethyl 2-(4-isobutylphenyl)propionate at 17.5 mg, 35 mg, 70 mg, 140 mg and 280 mg, mean 2-(diethylamino)ethyl 2-(4-isobutylphenyl)propionate Cₘₐₓ was not significantly different following the 17.5 mg, 35 mg and 70 mg doses but was about 2-fold higher following application of the 140 mg and 280 mg doses. Mean ibuprofen Cₘₐₓ was variable and was about 1.5-2 fold higher on Day 12 compared to Day 1 following the 35 mg, 140 mg and 280 mg doses, while comparable following the 17.5 mg and 70 mg doses. A similar profile was observed for AUCs.

The shapes of the mean plasma concentration-time profiles for ibuprofen were similar for all dose groups. Estimates for many of the reported plasma PK parameters exhibited great inter-subject variability within a dose level.

Mean (SD) Cₘₐₓ of ibuprofen following single topical application of 2-(diethylamino)ethyl 2-(4-isobutylphenyl)propionate at doses of 17.5 mg, 35 mg, 70 mg, 140 mg and 280 mg were 73.2 (56.6), 50.9 (28.1), 92.4 (65.6), 133.9 (172.5) and 171.5 (98.0) ng/mL, respectively.

Mean (SD) Cₘₐₓ of ibuprofen following multiple topical application of 2-(diethylamino)ethyl 2-(4-isobutylphenyl)propionate for 7 days at doses of 17.5 mg, 35 mg, 70 mg, 140 mg and 280 mg were 63.4 (27.4), 115.9 (136.0), 61.8 (24.3), 180.2 (100.7) and 304.7 (165.0) ng/mL, respectively.

Median time to Cₘₐₓ (Tₘₐₓ) for 2-(diethylamino)ethyl 2-(4-isobutylphenyl)propionate and ibuprofen displayed a great deal of variability among subjects within each dose group and did not display a clear relationship to study drug dose.

Mean (SD) AUCₗₐₛₜ of ibuprofen following single topical application of 2-(diethylamino)ethyl 2-(4-isobutylphenyl)propionate at doses of 17.5 mg, 35 mg, 70 mg, 140 mg and 280 mg were 2122 (1255), 1526 (932.9), 3167 (2433), 4033 (4546) and 6682 (2505) ng·hr/mL, respectively.

Mean (SD) AUCₗₐₛₜ of ibuprofen following multiple topical application of 2-(diethylamino)ethyl 2-(4-isobutylphenyl)propionate for 7 days at doses of 17.5 mg, 35 mg, 70 mg, 140 mg and 280 mg were 1909 (560.8), 3753 (4317), 3155 (1751), 5028 (2223) and 11855 (6883) ng·hr/mL, respectively.

The shape of the mean plasma concentration-time profile for 2-(diethylamino)ethyl 2-(4-isobutylphenyl)propionate and ibuprofen when given as a 70 mg spray of 2-(diethylamino)ethyl 2-(4-isobutylphenyl)propionate was consistent with that observed for topical application. Estimates for the PK parameters exhibited a similar greater inter-subject variability.

To determine if steady-state was achieved, trough (pre-dose) levels of plasma 2-(diethylamino)ethyl 2-(4-isobutylphenyl)propionate and ibuprofen were measured prior to administration of study drug on Days 7-12. These values are summarized by treatment as Cₘᵢₙ in Table 10.

TABLE 10
Mean (SD) 2-(diethylamino)ethyl 2-(4-isobutylphenyl)propionate and
Ibuprofen Cₘᵢₙ on Days 7-12—PK population

| | | | | | | |
|---|---|---|---|---|---|---|
| | 2-(diethylamino) | 2-(diethylamino) | 2-(diethylamino) | 2-(diethylamino) | 2-(diethylamino) | 2-(diethylamino) |
| | ethyl 2-(4- | ethyl 2-(4- | ethyl 2-(4- | ethyl 2-(4- | ethyl 2-(4- | ethyl 2-(4- |
| | isobutylphenyl) | isobutylphenyl) | isobutylphenyl) | isobutylphenyl) | isobutylphenyl) | isobutylphenyl) |
| Cₘᵢₙ | propionate | propionate | propionate | propionate | propionate | propionate |
| (ng/mL) | 17.5 mg | 35 mg | 70 mg | 140 mg | 280 mg | 70 mg spray |

2-(diethylamino)ethyl 2-(4-isobutylphenyl)propionate

| | N = 3 | N = 4 | N = 4 | N = 5 | N = 7 | N = 5 |
|---|---|---|---|---|---|---|
| Day 7 | 0.0190 (0.0329) | 0.0636 (0.0442) | 0.0593 (0.0684) | 0.0868 (0.0245) | 0.1098 (0.0974) | 0.0171 (0.0383) |
| Day 8 | 0.0439 (0.0393) | 0.0611 (0.0053) | 0.0352 (0.0413) | 0.1528 (0.0771) | 0.1708 (0.0836) | 0.0522 (0.0538) |
| Day 9 | 0.0245 (0.0425) | 0.1278 (0.0221) | 0.0445 (0.0517) | 0.1509 (0.0479) | 0.2276 (0.1381) | 0.0932 (0.0831) |
| Day 10 | 0.0485 (0.0441) | 0.1305 (0.0112) | 0.0495 (0.0333) | 0.1614 (0.0485) | 0.2127 (0.0939) | 0.0587 (0.0608) |
| Day 11 | 0.0431 (0.0394) | 0.1036 (0.0186) | 0.0507 (0.0616) | 0.1439 (0.0437) | 0.2257 (0.1262) | 0.0849 (0.0421) |
| Day 12 | 0.0371 (0.0322) | 0.1180 (0.0252) | 0.0350 (0.0423) | 0.2038 (0.1128) | 0.3353 (0.1663) | 0.0863 (0.0500) |

Ibuprofen

| | N = 8 | N = 8 | N = 8 | N = 7 | N = 8 | N = 8 |
|---|---|---|---|---|---|---|
| Day 7 | 42.03 (24.13) | 43.51 (18.92) | 54.40 (26.06) | 109.3 (108.3) | 137.6 (89.98) | 44.49 (21.86) |
| Day 8 | 54.08 (27.18) | 49.96 (17.99) | 56.06 (9.294) | 143.8 (94.75) | 211.4 (108.9) | 46.03 (17.30) |
| Day 9 | 38.43 (8.887) | 74.13 (24.40) | 61.78 (34.62) | 160.5 (99.95) | 231.0 (127.6) | 81.55 (36.20) |
| Day 10 | 40.48 (20.56) | 72.58 (27.19) | 51.53 (17.19) | 159.0 (95.94) | 236.6 (135.8) | 54.06 (25.13) |
| Day 11 | 56.80 (25.09) | 64.81 (21.48) | 70.63 (26.68) | 140.9 (88.53) | 249.1 (134.0) | 63.20 (33.45) |
| Day 12 | 57.81 (22.08) | 62.26 (17.77) | 54.90 (26.59) | 175.9 (102.6) | 280.3 (152.1) | 66.99 (25.94) |

Accumulation ratios of Cₘₐₓ, C_{max0-12 hr} and AUCs (AUC_{0-12 hr} and AUC_{inf}) (Day 12 vs. Day or Day 12 vs. Day 6) were calculated for 2-(diethylamino)ethyl 2-(4-isobutylphenyl)propionate and ibuprofen

Based on observed Cₘᵢₙ and accumulation index for ibuprofen (approximately 3-fold, which showed a great deal of variability among subjects within each dose group), it can be concluded that steady-state was reached on Day 10 (5^{th} day of b.i.d. dosing) of the application. Because PK parameters could not be reliably calculated for 2-(diethylamino)ethyl 2-(4-isobutylphenyl)propionate, data from 2-(diethylamino)ethyl 2-(4-isobutylphenyl)propionate were not used to make this assessment Data from subjects in the post-hoc analysis population were consistent with data from subjects in the PK population.

The relative bioavailability of the single and multiple dose 70 mg topical application vs. 70 mg spray application was determined based on geometric means of AUCₜₐᵤ, AUC_{inf} and Cₘₐₓ, and the 90% CIs on the ratio of topical application vs. spray application were calculated. Results for the PK population are summarized in Table 11.

Relative bioavailability of 2-(diethylamino)ethyl 2-(4-isobutylphenyl)propionate based on measured plasma ibuprofen concentrations when given as spray was lower when compared to the topical application following single application, but comparable at steady-state. Data from subjects in the post-hoc analysis population were consistent with data from subjects in the PK population.

TABLE 11
Relative bioavailability of 70 mg topical application vs. 70 mg spray - pk population

| | | Geometric means* | | Ratio | 90% CIs |
|---|---|---|---|---|---|
| Analyte | PK Parameter (unit) | Topical | Spray | Topical/Spray | Topical/Spray |

Day 1

| | | | | | |
|---|---|---|---|---|---|
| 2-(diethylamino)ethyl | Cₘₐₓ (ng/mL) | 0.1375 | 0.1023 | 1.34 | (0.63-2.87) |
| 2-(4-isobutylphenyl) | AUC_{inf} (ng · h/mL) | ND | ND | ND | ND |
| propionate hydrochloride | AUC₀₋₁₂ (ng · h/mL) | 0.9132 | 0.2786 | 3.28 | (0.67-16.00) |
| Ibuprofen | Cₘₐₓ (ng/mL) | 73.67 | 60.33 | 1.22 | (0.66-2.25) |
| | AUC_{inf} (ng · h/mL) | 2053 | 1816 | 1.13 | (0.38-3.35) |
| | AUC₀₋₁₂ (ng · h/mL) | 340.3 | 277.6 | 1.23 | (0.52-2.90) |

Day 12

| | | | | | |
|---|---|---|---|---|---|
| 2-(diethylamino)ethyl | Cₘₐₓ (ng/mL) | 0.1434 | 0.1549 | 0.93 | (0.42-2.06) |
| 2-(4-isobutylphenyl) | AUC_{inf} (ng · h/mL) | ND | ND | ND | ND |
| propionate hydrochloride | AUC₀₋₁₂ (ng · h/mL) | 0.9532 | 1.048 | 0.91 | (0.43-1.93) |
| Ibuprofen | Cₘₐₓ (ng/mL) | 58.33 | 63.82 | 0.91 | (0.67-1.26) |
| | AUC_{inf} (ng · h/mL) | 1900 | 1827 | 1.04 | (0.61-1.76) |
| | AUC₀₋₁₂ (ng · h/mL) | 532.1 | 533.1 | 1.00 | (0.75-1.33) |

*Geometric means are least square means derived from mixed models.

Overall, the results after excluding subjects with >15% of their own Cₘₐₓ (post-hoc analysis) were consistent with the results observed for the pre-specified analysis, suggesting that the pre-dose circulating levels of ibuprofen did not impact the kinetic behavior of the drug 2-(diethylamino)ethyl 2-(4-isobutylphenyl)propionate, nor did it affect overall data interpretation.

2.5.1.3. Pharmacokinetics Results Summary:
Overall, 2-(diethylamino)ethyl 2-(4-isobutylphenyl)propionate and ibuprofen average maximum plasma concentration and exposure increased as 2-(diethylamino)ethyl 2-(4-isobutylphenyl)propionate doses increased from 17.5 mg to 280 mg, but not in a dose-proportional manner.
Following single topical application of 17.5 mg, 35 mg, 70 mg, 140 mg and 280 mg of 2-(diethylamino)ethyl 2-(4-isobutylphenyl)propionate to normal healthy subjects, absorption of 2-(diethylamino)ethyl 2-(4-isobutylphenyl)propionate was rapid and the absorbed 2-(diethylamino)ethyl 2-(4-isobutylphenyl)propionate was rapidly converted (>99%) to ibuprofen.
Following b.i.d. topical application of 2-(diethylamino)ethyl 2-(4-isobutylphenyl)propionate for 7 consecutive days, mean 2-(diethylamino)ethyl 2-(4-isobutylphenyl)propionate Cₘₐₓ was not significantly different at 17.5 mg, 35 mg and 70 mg doses but was about 2-fold higher following 140 mg and 280 mg doses, whereas mean ibuprofen Cₘₐₓ was variable and was about 1.5-2 fold higher on Day 12 compared to Day 1 following the 35 mg, 140 mg and 280 mg doses, and comparable following the 17.5 mg and 70 mg doses.
Dose proportionality was not formally demonstrated as topical doses of 2-(diethylamino)ethyl 2-(4-isobutylphenyl)propionate increased from 17.5 mg to 280 mg.
Based on observed Cₘᵢₙ and accumulation index of ibuprofen, it can be concluded that steady-state was reached following 5 days of b.i.d. dosing.
Relative bioavailability of 2-(diethylamino)ethyl 2-(4-isobutylphenyl)propionate based on measured plasma ibuprofen concentrations when given as spray was lower when compared to the topical application following single application, but was comparable at steady-state.
Post-hoc analysis results were consistent with those observed for the PK population, indicating that the pre-dose circulating plasma levels of ibuprofen did not impact study drug kinetics or overall data interpretation.

2.6. Safety Evaluation

The safety and tolerability of escalating single and multiple ascending oral doses of 2-(diethylamino)ethyl 2-(4-isobutylphenyl)propionate were assessed by evaluation of AEs, vital sign assessments, resting 12-lead ECGs and physical examination findings and skin irritation assessments.

2.6.1. Extent of Exposure

eight subjects each were exposed to single and multiple doses of 17.5 mg, 35 mg, 70 mg, 140 mg, and 280 mg 2-(diethylamino)ethyl 2-(4-isobutylphenyl)propionate as a topical application and 70 mg 2-(diethylamino)ethyl 2-(4-isobutylphenyl)propionate as a spray application, and 12 subjects were exposed to single and multiple doses of placebo.

2.6.2. Adverse Events

2.6.2.1. Brief Summary of Adverse Events

A total of 1 (12.5%), 3 (37.5%), 1 (12.5%), 1 (12.5%), 1 (12.5%) and 2 (25.0%) subjects experienced at least one treatment-emergent AE (TEAE) following a single dose and multiple doses of 17.5 mg, 35 mg, 70 mg, 140 mg, and 280 mg 2-(diethylamino)ethyl 2-(4-isobutylphenyl)propionate as a topical application and 70 mg 2-(diethylamino)ethyl 2-(4-isobutylphenyl)propionate as a spray application, respectively, and 2 (16.7%) subjects experienced at least one TEAE following a single and multiple doses of placebo.

All AEs were considered by the investigator to be mild or moderate in intensity. No SAEs were reported and no subjects discontinued study treatment due to an AE.

An overview of adverse events reported during this study is presented by treatment group and overall in Table 12.

TABLE 12
Overview of Adverse Events—Safety Population

| | Treatment | | | | | | | |
|---|---|---|---|---|---|---|---|---|
| | 2-(diethylamino) | 2-(diethylamino) | 2-(diethylamino) | 2-(diethylamino) | 2-(diethylamino) | 2-(diethylamino) | | |
| | ethyl 2-(4- | ethyl 2-(4- | ethyl 2-(4- | ethyl 2-(4- | ethyl 2-(4- | ethyl 2-(4- | | |
| | isobutylphenyl) | isobutylphenyl) | isobutylphenyl) | isobutylphenyl) | isobutylphenyl) | isobutylphenyl) | | |
| | propionate | propionate | propionate | propionate | propionate | propionate | | |
| | 17.5 mg | 35 mg | 70 mg | 140 mg | 280 mg | 70 mg Spray | Placebo | Overall |
| Category* | N = 8 | N= 8 | N = 8 | N = 8 | N = 8 | N = 8 | N = 12 | (N = 60) |
| Subjects with | 1 (12.5) | 3 (37.5) | 1 (12.5) | 1 (12.5) | 1 (12.5) | 2 (25.0) | 2 (16.7) | 11 (18.3) |
| TEAEs | | | | | | | | |
| Subjects with | 0 | 0 | 0 | 0 | 0 | 0 | 0 | 0 |
| SAEs | | | | | | | | |
| Subjects who | 0 | 0 | 0 | 0 | 0 | 0 | 0 | 0 |
| discontinued | | | | | | | | |
| due to an AE | | | | | | | | |

*Subjects may fall into more than one category

2.6.2.2. Analysis of Adverse Events

The frequency of subjects who experienced at least one TEAE event during this study, regardless of relatedness to study drug, is presented by System Organ Classification, Preferred Term and by treatment in Table 13.

A total of 1 (12.5%), 3 (37.5%), 1 (12.5%), 1 (12.5%), 1 (12.5%) and 2 (25.0%) subjects experienced at least one TEAE following a single dose and multiple doses of 17.5 mg, 35 mg, 70 mg, 140 mg, and 280 mg 2-(diethylamino)ethyl 2-(4-isobutylphenyl)propionate as a topical application and 70 mg 2-(diethylamino)ethyl 2-(4-isobutylphenyl)propionate as a spray application, respectively, and 2 (16.7%) subjects experienced at least one TEAE following a single and multiple doses of placebo.

Most AEs were considered by the investigator to be mild in intensity. One AE of headache was considered moderate in intensity. No AEs were considered to be severe. No SAEs were reported and no subjects discontinued study treatment due to an AE.

Headache, reported by 2 (25.0%), 1 (12.5%) and 1 (8.3%) subjects after receiving 35 mg 2-(diethylamino)ethyl 2-(4-isobutylphenyl)propionate, 140 mg 2-(diethylamino)ethyl 2-(4-isobutylphenyl)propionate and placebo, respectively, was the only AE reported by more than one subject in a dose group. All other AEs were reported by no more than one subject in each dose group.

The most frequent AE reported (reported by more than two subjects) was headache, as noted above, and alanine aminotransferase (ALT) increased, reported for 1 (12.5%) subject each after receiving 70 mg 2-(diethylamino)ethyl 2-(4-isobutylphenyl)propionate, 280 mg 2-(diethylamino)ethyl 2-(4-isobutylphenyl)propionate and 70 mg 2-(diethylamino)ethyl 2-(4-isobutylphenyl)propionate spray. aspartate aminotransferase (ast) increased was reported for 1 (12.5%) subject each after receiving 70 mg 2-(diethylamino)ethyl 2-(4-isobutylphenyl)propionate and 70 mg 2-(diethylamino)ethyl 2-(4-isobutylphenyl)propionate spray.

TABLE 13
Frequency of Subjects Experiencing TEAEs by System Organ Class and Preferred Term—Safety Population

| | | | | | | 70 g | |
|---|---|---|---|---|---|---|---|
| | 17.5 mg | 35 mg | 70 mg | 140 mg | 280 mg | Spray | Placebo |
| | N = 8 | N = 8 | N = 8 | N = 8 | N =8 | N = 8 | N = 12 |
| System organ class/Preferred term* | n (%) | n (%) | n (%) | n (%) | n (%) | n (%) | n (%) |
| Subjects with at least one TEAE | 1 (12.5) | 3 (37.5) | 1 (12.5) | 1 (12.5) | 1 (12.5) | 2 (25.0) | 2 (16.7) |
| Gastrointestinal disorders | 1 (12.5) | 1 (12.5) | 0 | 0 | 0 | 0 | 1 (8.3) |
| Constipation | 0 | 1 (12.5) | 0 | 0 | 0 | 0 | 0 |
| Nausea | 1 (12.5) | 0 | 0 | 0 | 0 | 0 | 1 (8.3) |
| Vomiting | 0 | 0 | 0 | 0 | 0 | 0 | 1 (8.3) |
| General disorders and administration site conditions | 0 | 3 (37.5) | 0 | 0 | 0 | 0 | 0 |
| Chest discomfort | 0 | 1 (12.5) | 0 | 0 | 0 | 0 | 0 |
| Pyrexia | 0 | 1 (12.5) | 0 | 0 | 0 | 0 | 0 |
| Vessel puncture site parasthesia | 0 | 1 (12.5) | 0 | 0 | 0 | 0 | 0 |
| Investigations | 0 | 0 | 1 (12.5) | 0 | 1 (12.5) | 1 (12.5) | 0 |
| Alanine aminotransferase increased | 0 | 0 | 1 (12.5) | 0 | 1 (12.5) | 1 (12.5) | 0 |
| Aspartate aminotransferase increased | 0 | 0 | 1 (12.5) | 0 | 0 | 1 (12.5) | 0 |
| Musculoskeletal and connective tissue disorders | 0 | 1 (12.5) | 0 | 0 | 0 | 1 (12.5) | 1 (8.3) |
| Back pain | 0 | 1 (12.5) | 0 | 0 | 0 | 0 | 1 (8.3) |
| Trigger finger | 0 | 0 | 0 | 0 | 0 | 1 (12.5) | 0 |
| Nervous system disorders | 0 | 2 (25.0) | 0 | 1 (12.5) | 0 | 0 | 1 (8.3) |
| Dizziness | 0 | 0 | 0 | 0 | 0 | 0 | 1 (8.3) |
| Headache | 0 | 2 (25.0) | 0 | 1 (12.5) | 0 | 0 | 1 (8.3) |
| Skin and subcutaneous tissue disorders | 0 | 1 (12.5) | 0 | 0 | 1 (12.5) | 0 | 0 |
| Erythema | 0 | 1 (12.5) | 0 | 0 | 0 | 0 | 0 |
| Rash erythematous | 0 | 0 | 0 | 0 | 1 (12.5) | 0 | 0 |

*Subjects are counted once for each system organ class and once for each preferred term

2.6.2.3. Deaths, Other Serious Adverse Events, and Other Significant Adverse Events

2.6.2.3.1. Deaths

No subjects died during this study.

2.6.2.3.2. Other Serious Adverse Events

No subjects experienced an SAE during this study.

2.6.2.3.3. Other Significant Adverse Events

There were no significant AEs during this study.

2.6.2.3.4. Narratives of Deaths, Other Serious Adverse Events, and Certain Other Significant Adverse Events

There were no events that required narratives during this study.

2.6.2.3.5. Analysis and Discussion of Deaths, Other Serious Adverse Events, and Other Significant Adverse Events

There were no deaths, other SAEs or other significant AEs during this study.

2.6.2.4. Clinical Laboratory Evaluation

Listing of Individual Laboratory Measurements by Subject and Each Abnormal Laboratory Value

Samples for clinical laboratory safety tests (chemistry, hematology, urinalysis) were obtained at the screening visit, Day −1, Day 4, Day 13 and prior to discharge from the study on Day 17 or at early termination. Any clinical laboratory findings considered to be clinically significant were recorded as AEs.

Laboratory parameters collected include:
Chemistry: Blood urea nitrogen (BUN), creatinine, total bilirubin, glucose, albumin, total protein, aspartate transaminase (AST), alanine transaminase (ALT), gamma-glutamyltransferase (GGT), lactate dehydrogenase (LDH), alkaline phosphatase, CO2, phosphate, sodium, potassium, chloride, calcium, total cholesterol, uric acid.
Hematology: hemoglobin, hematocrit, red blood cell (RBC) count, platelet count and white blood cell (WBC) count with differential.
Urinalysis: pH, specific gravity, protein, glucose, ketones, bilirubin, blood, nitrites, leukocytes, urobilinogen and microscopy.

Other:
Urine pregnancy test (females only) at screening, and admission on Day −1 and Day 5, on Day 13 and prior to discharge from the study on Day 17 or at early termination.
Alcohol and drugs of abuse screen (amphetamines, barbiturates, cocaine metabolites, benzodiazepines, cannabinoids, opiates, ethyl alcohol) at screening and admission on Day −1 and Day 5.
Hepatitis B surface antibodies (HBsAb) and hepatitis C antibodies, anti-HIV antibodies at screening.

All subjects had normal clinical laboratory test results at screening, or results that were outside normal reference ranges provided by the laboratory but considered not clinically significant by the Principal Investigator.

Three subjects had laboratory values that were outside normal reference ranges, considered clinically significant by the Principal Investigator, and were recorded as adverse events.

One subject, a 23 year-old male, received the first dose of 70 mg 2-(diethylamino)ethyl 2-(4-isobutylphenyl)propionate on 29 May 2013, began b.i.d. dosing on 3 Jun. 2013, and had increased ALT and increased AST recorded as an AE on 10 Jun. 2013 (Day 13). Both AEs were considered by the investigator as mild in intensity and probably related to study drug. The AE of increased AST was resolved 14 Jun. 2013 (Day 17), at which time the AE of increased ALT was ongoing. At study termination this AE of elevated ALT was still ongoing and the subject not yet recovered.

One subject, a 31 year-old male, received the first dose of 280 mg 2-(diethylamino)ethyl 2-(4-isobutylphenyl)propionate on 8 Jun. 2013, began b.i.d. dosing on 13 Jun. 2013, and had increased ALT recorded as an AE on 20 Jun. 2013 (Day 13). This AE was considered mild in intensity, probably related to study drug, and was ongoing when the subject was discharged from the study on Day 17. At study termination this AE of elevated ALT was still ongoing and the subject not yet recovered.

One subject, a 30 year-old male, received the first dose of 70 mg 2-(diethylamino)ethyl 2-(4-isobutylphenyl)propionate as a spray on 29 May 2013, began b.i.d. dosing on 3 Jun. 2013, and had increased ALT and increased AST recorded as an AE on 10 Jun. 2013 (Day 13). Both AEs were considered by the investigator as mild in intensity, related to study drug, and these AEs resolved on 18 Jun. 2013 (ALT) and 14 Jun. 2013 (AST).

Overall, no apparent trends or dose relationship of elevated laboratory findings and study drug dose were observed.

2.6.2.5. Vital Signs, Physical Findings, and Other Observations Related to Safety

2.6.2.5.1. Vital Signs

Systolic and diastolic blood pressure (mmHg) and pulse rate (beats per minute), respiratory rate (breaths per minute) and oral temperature (° C.) were measured after at least 5 minutes at rest in the seated position at the screening visit, at admission on Day −1, Day 5 and Day 13, and prior to and 1 hour following each dose administration.

Height (cm) and weight (kg) were recorded at screening and BMI was calculated.

There were no clinically significant vital sign values noted for any subject. There were no clinically relevant trends or changes in vital signs noted during this study.

2.6.2.5.2. Physical Examinations

A physical examination was performed at the screening visit and on Day 13, or at early termination, and included assessment of the following body systems: general appearance, dermatological, head, eyes, ears, nose and throat, neck, lymph nodes, lungs, heart, abdomen, neurological and musculoskeletal.

Most physical examination results were normal for all subjects, or were present at screening and considered not clinically significant.

One subject, who received the first dose of 35 mg 2-(diethylamino)ethyl 2-(4-isobutylphenyl)propionate on 22 May 2013 and began b.i.d. dosing on 27 May 2013, had an AE of erythema (left leg) opened on 1 Jun. 2013 (Day 11). This AE was considered mild in intensity, probably related to study drug, and resolved 5 Jun. 2013. Faint erythema on the left leg was noted as a physical examination finding for this subject on 3 Jun. 2013 (Day 13).

One subject, who received the first dose of 280 mg 2-(diethylamino)ethyl 2-(4-isobutylphenyl)propionate on 8 Jun. 2013 and began b.i.d. dosing on 13 Jun. 2013, had an AE of rash erythematous (both knees) opened on 18 Jun. 2013 (Day 11). This AE was considered mild in intensity, related to study drug, and resolved 8 Jul. 2013. Bilateral erythema papule was noted as a clinically significant physical examination finding on 20 Jun. 2013 (Day 13).

One subject received the first dose of 70 mg 2-(diethylamino)ethyl 2-(4-isobutylphenyl)propionate as a spray on 29 May 2013 and began b.i.d. dosing on 3 Jun. 2013, had an AE of trigger finger opened on 3 Jun. 2013 (Day 6). This AE was considered mild in intensity, not related to study drug, and unresolved at the time of discharge from the study. Erythema was noted as a clinically significant physical examination finding on 10 Jun. 2013 (Day 13).

2.6.2.5.3. Electrocardiograms

A resting 12-lead ECG was recorded after at least 5 minutes of rest at the screening visit and on Day 13, or at early termination. The ECG parameters recorded include ventricular rate (bpm), PR interval (msec), QRS duration (msec), QT interval (msec), and QTc interval (msec). QTc interval was calculated at the clinic using a formula internal to the ECG equipment (Mortara).

2.6.2.6. ECG Parameters

There were no clinically significant ECG parameter values noted for any subject. There were no clinically relevant trends or changes in ECG parameters noted during this study.

2.6.2.7. Skin Irritation Assessments

Skin irritation due to study drug application was measured by a dermal response score of 0-7 assigned for each study drug application site prior to and 30 min following each dose administration. A score of 6 or 7 was considered a severe reaction. Pre-dose assessments during the multiple dosing served as the 12 hour post-dose assessment of the prior dose.

Most subjects had all skin irritation scores recorded as 0 (no evidence of irritation). Occasional scores of 1 (minimal erythema, barely perceptible) or 2 (definite erythema, readily visible; minimal edema or minimal popular response) were noted for one subject receiving 35 mg 2-(diethylamino)ethyl 2-(4-isobutylphenyl)propionate, for three subjects receiving 70 mg 2-(diethylamino)ethyl 2-(4-isobutylphenyl)propionate (topical application), and for one subject each receiving 280 mg 2-(diethylamino)ethyl 2-(4-isobutylphenyl)propionate and 70 mg 2-(diethylamino)ethyl 2-(4-isobutylphenyl)propionate as a spray, one subject receiving 70 mg 2-(diethylamino)ethyl 2-(4-isobutylphenyl)propionate as a topical application had a single skin irritation score of 3 (erythema and papules). No subject had a skin irritation score>3.

2.6.2.8. Safety Conclusions

Eight subjects each were exposed to single and multiple doses of 17.5 mg, 35 mg, 70 mg, 140 mg, and 280 mg 2-(diethylamino)ethyl 2-(4-isobutylphenyl)propionate as a topical application and 70 mg 2-(diethylamino)ethyl 2-(4-isobutylphenyl)propionate as a spray application, and 12 subjects were exposed to single and multiple doses of placebo. Safety conclusions for this study are:
Single and multiple doses 17.5 mg, 35 mg, 70 mg, 140 mg, and 280 mg 2-(diethylamino)ethyl 2-(4-isobutylphenyl)propionate as a topical application and 70 mg 2-(diethylamino)ethyl 2-(4-isobutylphenyl)propionate as a spray application were safe and well-tolerated by this population of healthy male and female volunteers.
No dose escalation stopping criteria were met during this study; all dose escalations from single to multiple dose application and from one dose level to the next dose level occurred.
A total of 1 (12.5%), 3 (37.5%), 1 (12.5%), 1 (12.5%), 1 (12.5%) and 2 (25.0%) subjects experienced at least one TEAE following single and multiple doses of 17.5 mg, 35 mg, 70 mg, 140 mg, and 280 mg 2-(diethylamino)ethyl 2-(4-isobutylphenyl)propionate as a topical application and 70 mg 2-(diethylamino)ethyl 2-(4-isobutylphenyl)propionate as a spray application, respectively, and 2 (16.7%) subjects experienced at least one TEAE following a single and multiple doses of placebo.
All AEs were considered by the investigator to be mild or moderate in intensity. No subjects discontinued due to an AE and no SAEs were reported.
There were no clinically meaningful trends noted based on safety laboratory assessments, physical examinations or vital sign measurements during this study.
Skin irritation responses were generally mild and transient in nature. No severe skin irritation responses were observed for any subject in this study.

2.6.2.9. Discussion and Overall Conclusions

This was a single center, randomized, double-blind, placebo-controlled dose-escalation study to evaluate the safety, tolerability and pharmacokinetics of single and multiple doses of 2-(diethylamino)ethyl 2-(4-isobutylphenyl)propionate following escalating single and multiple doses administered as a topical application.

The study was conducted in six cohorts. In each cohort of 10 subjects, eight subjects were randomized to receive active drug and two subjects were randomized to receive matching placebo. Dose levels of 17.5, 35, 70, 140 and 280 mg of topical application and 70 mg as spray application were evaluated.

The study design incorporated staggered durations of single doses followed by 7 days of b.i.d. doses of study drug for evaluation of safety and pharmacokinetics as overlapping dose cohorts.

A review of safety data was performed prior to initiation of multiple dosing at each dose level and dose escalation to the next higher single dose.

For single dose PK, serial blood samples were collected on Day 1 dose at the following time points: 0 (pre-dose), 0.25, 0.5, 0.75, 1, 1.5, 2, 2.5, 3, 4, 5, 6, 8, 10, 12, 18, 24, 48, 72, 96 and 120 hours post-dose.

For multiple-dose PK, serial blood samples were collected on Day 12 at the following time points: 0 (pre-dose), 0.25, 0.5, 0.75, 1, 1.5, 2, 2.5, 3, 4, 5, 6, 8, 10, 12, 18, 24, 48, 72, 96 and 120 hours post-dose.

Safety assessments included monitoring of AEs, vital sign assessments, resting 12-lead ECGs and physical examination findings, and skin irritation assessments.

Pharmacokinetics
Overall, 2-(diethylamino)ethyl 2-(4-isobutylphenyl)propionate and ibuprofen average maximum plasma concentration and exposure increased as 2-(diethylamino)ethyl 2-(4-isobutylphenyl)propionate doses increased from 17.5 mg to 280 mg, but not in a dose-proportional manner.
Following single topical application of 17.5 mg, 35 mg, 70 mg, 140 mg and 280 mg of 2-(diethylamino)ethyl 2-(4-isobutylphenyl)propionate to normal healthy subjects, absorption of 2-(diethylamino)ethyl 2-(4-isobutylphenyl)propionate was rapid and the absorbed 2-(diethylamino)ethyl 2-(4-isobutylphenyl)propionate was rapidly converted to ibuprofen.
Following b.i.d. topical application of 2-(diethylamino)ethyl 2-(4-isobutylphenyl)propionate for 7 consecutive days, mean 2-(diethylamino)ethyl 2-(4-isobutylphenyl)propionate Cₘₐₓ was not significantly different at 17.5 mg, 35 mg and 70 mg doses but was about 2-fold higher following 140 mg and 280 mg doses, whereas mean ibuprofen Cₘₐₓ was variable and was about 1.5-2 fold higher on Day 12 compared to Day 1 following the 35 mg, 140 mg and 280 mg doses, and comparable following the 17.5 mg and 70 mg doses.
Dose proportionality was not formally demonstrated as topical doses of 2-(diethylamino)ethyl 2-(4-isobutylphenyl)propionate increased from 17.5 mg to 280 mg.
Based on observed Cₘᵢₙ and accumulation index of ibuprofen, it can be concluded that steady-state was reached following 5 days of b.i.d. dosing.
Relative bioavailability of 2-(diethylamino)ethyl 2-(4-isobutylphenyl)propionate based on measured plasma ibuprofen concentrations when given as spray was lower when compared to the topical application following single application, but was comparable at steady-state.
Post-hoc analysis results were consistent with those observed for the PK population, indicating that the pre-dose circulating plasma levels of ibuprofen did not impact study drug kinetics or overall data interpretation.

Safety
Single and multiple doses 17.5 mg, 35 mg, 70 mg, 140 mg, and 280 mg 2-(diethylamino)ethyl 2-(4-isobutylphenyl)propionate as a topical application and 70 mg 2-(diethylamino)ethyl 2-(4-isobutylphenyl)propionate as a spray application were safe and well-tolerated by this population of healthy male and female volunteers.
No dose escalation stopping criteria were met during this study; all dose escalations from single to multiple dose application and from one dose level to the next dose level occurred.
A total of 1 (12.5%), 3 (37.5%), 1 (12.5%), 1 (12.5%), 1 (12.5%) and 2 (25.0%) subjects experienced at least one TEAE following single and multiple doses of 17.5 mg, 35 mg, 70 mg, 140 mg, and 280 mg 2-(diethylamino)ethyl 2-(4-isobutylphenyl)propionate as a topical application and 70 mg 2-(diethylamino)ethyl 2-(4-isobutylphenyl)propionate as a spray application, respectively, and 2 (16.7%) subjects experienced at least one TEAE following a single and multiple doses of placebo.
All AEs were considered by the investigator to be mild or moderate in intensity. No subjects discontinued due to an AE and no SAEs were reported.
There were no clinically meaningful trends noted based on safety laboratory assessments, physical examinations or vital sign measurements during this study.
Skin irritation responses were generally mild and transient in nature. No severe skin irritation responses were observed for any subject in this study.

Single and multiple doses of 2-(diethylamino)ethyl 2-(4-isobutylphenyl)propionate ranging from 17.5 mg to 280 mg as a topical application, and as a 70 mg spray, were safe and well-tolerated by this population of healthy male and female volunteers.

The 2-(diethylamino)ethyl 2-(4-isobutylphenyl)propionate prodrug was rapidly absorbed following topical application, and was also rapidly converted to its active metabolite, ibuprofen, upon absorption. Steady-state was achieved following 5 days of b.i.d. dosing. Dose proportionality could not be formally demonstrated as topical doses of 2-(diethylamino)ethyl 2-(4-isobutylphenyl)propionate increased from 17.5 mg to 280 mg. Relative bioavailability of 2-(diethylamino)ethyl 2-(4-isobutylphenyl)propionate when given as spray was lower when compared to the topical application following single application, but was comparable at steady-state. From the PK profile, the drug concentration after the transdermal administration of 2-(diethylamino)ethyl 2-(4-isobutylphenyl)propionate are very constant during 24 hours and the drug can be detected after 5 days, that means that one drug administration per day will work fine, but some people may take twice shows every day; they may rush to work in morning and do not wait 3-5 minutes let the solution dry out before wearing clothing which will absorb the drug solution, or they may exercise and sweat too much to wash away the drug, so twice (morning and after) administrations per day is recommended.

3. Phase 2 Clinical Study

A Phase 2, Multicenter, Randomized, Double Blind (Within Dose), Placebo Controlled, Parallel Group, Dose Range Finding Study to Evaluate the Efficacy, Safety, and Pharmacokinetics of 2-(diethylamino)ethyl 2-(4-isobutylphenyl)propionate hydrochloride spray versus Placebo in Subjects with Mild to Moderate Osteoarthritis of the Knee.

3.1. Methodology/Study Design

This is a Phase 2, multicenter, randomized, double blind (within dose), placebo controlled, parallel group, proof of concept, and dose range finding study to evaluate the efficacy, safety, and PK of 2-(diethylamino)ethyl 2-(4-isobutylphenyl)propionate hydrochloride spray in adult subjects with clinically symptomatic mild to moderate OA of the knee. For subjects with bilateral knee pain, both knees will be treated, but the most symptomatic knee (i.e., the most painful knee as measured by the Western Ontario and McMaster University Osteoarthritis Index [WOMAC® 3.1] pain subscale score at Screening) will be designated as the target knee for efficacy analyses. For subjects with unilateral knee pain, only the symptomatic (target) knee will be treated.

Subjects taking nonsteroidal anti-inflammatory drugs (NSAID) or other analgesics may enroll in the trial, but will discontinue any analgesic therapy for the duration of the study, starting at least 4 days (or 5 half lives, whichever is longer) prior to administration of the first dose of study medication (i.e., for an analgesic washout period before Day 1). Subjects will be allowed to take rescue medication (up to six 325 mg tablets [total of 1950 mg] of acetaminophen per day; provided by the Sponsor) for residual knee or other body pain starting 4 days (or 5 half lives, whichever is longer) prior to administration of the first dose of study medication except during the 24 hours prior to Baseline (Day 1), Week 2, Week 4, Week 8, Week 12/end of study (EOS), and Follow up assessments.

After a Screening Period of up to 3 weeks and radiographic evaluation of the target knee joint space, a subject will be randomly assigned to 1 of 3 treatment groups in a 1:1:1 ratio with a 2:1 ratio of active:placebo within each treatment group (ie, 2 subjects to active treatment and 1 subject to placebo):
Group A: 8.75 mg 2-(diethylamino)ethyl 2-(4-isobutylphenyl)propionate/knee (2 sprays/knee, one spray to the medial surface and one spray to the lateral surface of the knee), twice daily (BID, approximately every 12 hours; n=50) or placebo (2 sprays/knee), BID (approximately every 12 hours; n=25) for a total of 4 sprays per knee per day;
Group B: 17.5 mg 2-(diethylamino)ethyl 2-(4-isobutylphenyl)propionate/knee (4 sprays/knee, one spray to the medial surface and one spray to the lateral surface of the knee, and one spray to front and one spray to the back of the knee), BID (approximately every 12 hours; n=50) or placebo (4 sprays/knee), BID (approximately every 12 hours; n=25) for a total of 8 sprays per knee per day;
Group C: 35 mg 2-(diethylamino)ethyl 2-(4-isobutylphenyl)propionate/knee (8 sprays/knee, each spray is to be applied to a different non-overlapping area around the knee, and the areas are to be uniformly distributed around the knee), BID (approximately every 12 hours; n=50) or placebo (8 sprays/knee), BID (approximately every 12 hours; n=25) for a total of 16 sprays per knee per day.

Each subject will receive study treatment for 12 weeks starting on Day 1. Qualified site personnel will contact subjects 1 week after the first administration of study medication to check if there are any issues with the spray bottles, administration of study medication, or adverse events (AEs). Subjects will return to the site at 2, 4, 8, and 12 weeks of treatment for efficacy and safety assessments as indicated in the study flow chart. The Week 12 visit will be the EOS visit. On the final day of dose administration (Week 12/EOS visit), subjects will receive 1 dose of study medication in the morning only; radiographs will be performed to assess changes in the target knee joint space. Subjects will have a Follow up visit approximately 7 days after the Week 12/EOS visit.

Subjects will record the following information in a Daily Diary starting at Screening: the amount of knee pain in the target knee while walking during the preceding 24 hours (using a 100 mm visual analog scale [VAS]), the times and number of sprays for each administration of study medication, the times when the subject washes his/her knees and/or takes a shower, the number of tablets of rescue medication taken that day and the times taken, the reason for taking the rescue medication (e.g., knee pain, headache, low back pain), any other concomitant medications taken that day, and any AEs occurring that day.

The VAS version of the WOMAC will be used for the Primary and Secondary Efficacy Endpoints. Efficacy endpoints will be assessed at Screening and Baseline (Day 1) and at the Week 2, Week 4, Week 8, and Week 12/EOS visits.

To assess the systemic multiple dose PK profile of 2-(diethylamino)ethyl 2-(4-isobutylphenyl)propionate and ibuprofen, subjects from each treatment group will be assigned to the PK subgroup. All subjects enrolled at designated sites that have the capability to keep subjects overnight will be included in the PK subgroup until PK samples for 12 to 18 subjects per treatment group have been collected. Blood samples for determining trough levels of 2-(diethylamino)ethyl 2-(4-isobutylphenyl)propionate and ibuprofen will be collected at the Week 2 visit and at a PK subgroup only visit during Week 3; these PK blood samples will be obtained prior to the morning dose of study medication. At the Week 4 visit PK blood samples will be collected predose and at 1, 3, 6, 8, 10, and 12 hours postdose. At the Week 12/EOS visit, subjects in the PK subgroup will be confined to the study site for approximately 36 hours after the last dose of study medication so that PK blood samples can be collected predose and at 1, 3, 6, 8, 10, 12, 18, 24, and 36 hours postdose, then return on the next 2 days for 48 and 72 hour postdose PK blood samples. Subjects will only receive the morning dose at the Week 12/EOS visit.

Safety assessments will be performed at each visit and will include assessment of AEs, vital signs (blood pressure, pulse rate, and oral temperature), clinical laboratories, physical examination, skin irritation, and electrocardiograms (ECGs) as indicated in the study flow chart, Table 14.

TABLE 14
Study Flow Chart

| | Screening | | | | | | | | Follow-up |
|---|---|---|---|---|---|---|---|---|---|
| | Period | Treatment Period | | | | | | | Period |
| | Visit | | | | | | | | |
| | | Day | Check-in | Week | Week | Week | Week | Week 12/EOS^{a} | Follow-up |
| | Screening | 1^{a} | Phone Call | 2^{a} | 3^{a,b} | 4^{a} | 8^{a} | (Early Term^{c}) | Visit |
| | Study Day | | | | | | | | |
| | Up to 3 | | | | | | | | |
| | weeks | 1 | 7 ± 2 | 14 ± 2 | 21 ± 2 | 28 ± 2 | 56 ± 2 | 84 ± 2 | 91 ± 2 |
| Informed consent | X | | | | | | | | |
| Inclusion/exclusion criteria | X | X | | | | | | | |
| Demographics | X | | | | | | | | |
| Medical/surgical history | X | | | | | | | | |
| Previous medications | X | | | | | | | | |
| Concomitant medications | X | X | | X | | X | X | X | X |
| Screening/Day 1 Pain Assessment (VAS) | X | X | | | | | | | |
| Body weight/height/BMI | X | | | | | | | | |
| Physical exam | X | | | | | | | X | |
| Vital signs^{d} | X | X | | X | | X | X | X | X |
| 12-Lead electrocardiogram | X | X | | | | | | X | |
| Clinical chemistry panel and CBC | X | X | | X | | X | X | X | |
| FSH^{e} | X | | | | | | | | |
| Pregnancy test (serum β-hCG)^{f} | X | | | | | | | X | |
| Urine pregnancy test^{f} | | X | | | | | | | |
| Urinalysis | X | | | | | | | | |
| Fecal occult blood test | X | X | | X | | X | X | X | |
| Hepatitis & HIV screen | X | | | | | | | | |
| Drug screen | X | | | | | | | | |
| Randomization | | X | | | | | | | |
| WOMAC 3.1 | X | X | | X | | X | X | X | X |
| Subject's global assessment of disease status | X | X | | X | | X | X | X | X |
| Subject's global assessment of response to | | | | X | | X | X | X | X |
| therapy | | | | | | | | | |
| Investigator's global assessment of disease | X | X | | X | | X | X | X | X |
| status | | | | | | | | | |
| Investigator's global assessment of response to | | | | X | | X | X | X | X |
| therapy | | | | | | | | | |
| X-rays and radiographic evaluation of knee | X | | | | | | | X | |
| joint space^{g} | | | | | | | | | |
| PK blood samples (PK-subgroup-only)^{h} | | | | X | X | X | | X | |
| Diary trainingⁱ/review^{j} | Xⁱ | X^{j,k} | | X^{j,k} | | X | X | X^{j} | |
| Check-in phone call | | | X^{l} | | | | | | |
| Adverse events | X | X | X | X | | X | X | X | X |
| Skin irritation evaluation (knee)^{m} | X | X^{m} | | X^{m} | | X^{m} | X^{m} | X^{m} | X |
| Drug administrationⁿ | | X^{o} | | X^{p} | X^{p} | X^{p} | X^{p} | X^{p} | |
| Study Drug Dispensing, Return, and | | X | | | | X | X | X | |
| Accountability | | | | | | | | | |
| Rescue Medication Dispensing, Return, and | X | | | X | | X | X | X | |
| Accountability^{q} | | | | | | | | | |

β hCG = human chorion gonadotropin β subunit;
AE = adverse event;
BMI = body mass index;
CBC = complete blood cell count;
Early Term = early termination;
EOS = end of study;
FSH = follicle stimulating hormone;
HIV = human immunodeficiency virus;
PK = pharmacokinetics;
VAS = visual analog scale;
WOMAC = Western Ontario and McMaster Osteoarthritis Index
Safety and efficacy assessments and/or PK blood samples should be completed prior to dose administration at each site visit.
Only subjects assigned to the PK subgroup will return to the clinic for the Week 3 visit for PK blood sampling (see footnote “h” below).
Week 12/EOS visit assessments (except PK blood samples) should be done at Early Termination visits.
Vital signs include sitting blood pressures, pulse rate, and oral temperature.
Only required for women who have had continuous amenorrhea for at least 12 months.
Serum and urine pregnancy tests are required for all women of childbearing potential.
Kellgren-Lawrence grading will be done by the Investigator or a local radiologist.
A total of 12 to 18 subjects from each treatment group will be selected for PK sampling. PK blood samples at Week 2 and Week 3 visits will be obtained prior to the morning dose of study medication. At the Week 4 visit, PK samples will be collected predose and at 1, 3, 6, 8, 10, and 12 hours postdose from subjects in the PK subgroup. At the Week 12/EOS visit, subjects in the PK subgroup will be confined to the study site for approximately 36 hours so that PK blood samples can be collected predose and at 1, 3, 6, 8, 10, 12, 18, 24, and 36 hours postdose, then return on the next 2 days for 48 and 72 hours postdose PK blood samples.
At the Screening visit, trained site personnel will give eligible subjects a Daily Diary and train them to record the amount of pain in the target knee while walking during the preceding 24 hours, the time and number of sprays for each administration of study medication, the times when the subject washes his/her knees and/or takes a shower, the number of tablets of rescue medication taken that day and the times taken, the reason for taking the rescue medication, any other concomitant medications taken that day, and any AEs occurring that day in the Daily Diary. Each subject will be instructed to complete the diary each day before going to bed starting 14 days before the Day 1 visit and to bring the diary to each visit.
Trained site personnel should review the diary for completeness and consistency including the amounts of study and rescue medications returned by the subject. The subject should be asked about any missing data and the reasons for missing data should be noted in the Daily Diary.
At the Day 1 and Week 2, Week 4, and Week 8 visits, subjects will be given a new Daily Diary and instructed to complete the diary each day before going to bed and to bring the diary to the next visit.
Qualified site personnel will contact subjects 1 week after the first administration of study medication to check if there are any issues with the spray bottles, administration of study medication, or AEs.
Skin irritation assessments will be performed prior to and 30 (±5) minutes after the morning dose of study medication at the Day 1, Week 2, Week 4, Week 8, and Week 12/EOS visits.
Subjects will be randomized to receive 2-(diethylamino)ethyl 2-(4-isobutylphenyl)propionate or placebo within Group A (2 sprays/knee), Group B (4 sprays per knee), or Group C (8 sprays/knee) twice daily for 12 weeks according to the randomization schedule. On the final day of dose administration (the Week 12/EOS visit), subjects will receive one dose of study medication in the morning only.
On Day 1, qualified site personnel will train the subject to clean their knees with a wet towel to remove any dirt or residual skin care products and thoroughly dry them just prior to administering study medication, to self administer study medication according to the Subject's randomly assigned treatment group. Prior to administration of the first dose of study medication on Day 1, the Subject's knees should be washed with soap and water and thoroughly dried to remove any dirt or residual skin care products.
On the mornings of the Week 2, Week 4, Week 8, and Week 12/EOS visits (and the Week 3 PK subgroup only visit for subjects in the PK subgroup), subjects are not to self administer the morning dose of study medication prior to returning to the site. On these days, subjects will self administer the morning dose of study medication only after safety and efficacy assessments and PK blood collections have been completed.
Subjects should not take any rescue medication during the 24 hour period prior to Baseline (Day 1), Week 2, Week 4, Week 8, and final (Week 12/EOS) assessments.

3.2. Diagnosis and Main Criteria for Inclusion:

Adult subjects with a diagnosis of primary OA of the knee will be enrolled in this study. The following are the key inclusion criteria:
1. A subject must be a male or female between 35 and 85 years of age, inclusive.
2. A subject must have a body mass index between 18.5 and 39.9 kg/m2, inclusive.
3. A subject must have a diagnosis of idiopathic OA according to the American College of Rheumatology clinical and radiographic criteria (knee pain, osteophytes, and at least one of the following: >50 years of age, morning stiffness lasting<30 minutes after getting up in the morning, or crepitus).
4. A subject must have a Kellgren Lawrence Grade of 1 or 2 as determined by the Investigator or a local radiologist at Screening.
5. A subject must have a history of clinically symptomatic mild to moderate OA of the knee for >6 months.
6. A subject must have had knee pain while standing, walking, and/or on motion for at least 14 days during the month prior to Screening.
7. A subject must have a knee pain score≥40 mm and <90 mm on a 100 mm VAS (with or without analgesic medication) on at least 10 of the 14 days prior to randomization.
8. A subject must be willing to discontinue any NSAIDs or other analgesic (e.g., aspirin, acetaminophen) or potentially confounding concomitant treatments (e.g., physiotherapy, acupuncture) starting 4 days (or 5 half lives, whichever is longer) before the administration of the first dose of study medication until completing participation in the study. (The use of ≤325 mg acetylsalicylic acid per day as cardiac prophylaxis is permitted.) The subject will be allowed to take rescue medication (acetaminophen) for pain during the study except during the 24 hours prior to Baseline (Day 1), Week 2, Week 4, Week 8, Week 12/EOS, and Follow-up assessments.
9. A subject must be willing to discontinue applying any topical preparations containing Vitamin A acids (including all trans retinoic acid (tretinoin), 13 cis retinoic acid [isotretinoin], 9 cis retinoic acid [alitretinoin], vitamin A [retinol], retinal, and their derivatives) to the lower limbs starting on Day 1 until completing participation in the study. (Topical preparations containing Vitamin A acids or retinol may be applied to areas of the skin above the waist, but should not be applied to areas of the skin exposed to study medication.)
10. A subject must be willing to avoid unaccustomed physical activity (e.g., starting a new weight lifting routine) for the duration of the study.
11. With the exception of OA of the knee, the subject must be in good general health with no clinically significant findings from medical history, vital signs, physical examination, ECG, and routine laboratory tests that could interfere with subject safety, or pain and functional assessments, as determined by the Investigator.

3.3. Criteria for Exclusion

The following are the main exclusion criteria:
1. A subject who has secondary OA of the knee or OA of lower limb joints other than the knee that, in the opinion of the Investigator, could interfere with pain and functional assessments related to the knee;
2. A subject who has OA of the knee with a Kellgren Lawrence Grade≥3 as determined by the Investigator or a local radiologist at Screening;
3. A subject who has a history of total or partial knee replacement, arthroplasty, or other knee surgery on either knee;
4. A subject who has had significant injury, as judged by the Investigator, involving the target knee within the 6 months before Screening.
5. A subject who has skin lesions or wounds on or near the knees to be treated at Screening or on Day 1 prior to the first administration of study medication;
6. A subject who has used opiates or corticosteroids within 30 days before Screening or who requires treatment with chronic opiates or corticosteroids;
7. A subject who has had intra articular injections of corticosteroids, hyaluronic acid, or viscosupplements (e.g., Synvisc®) to a knee to be treated within the 3 months before Screening.
8. A subject who has a history of significant hypersensitivity, intolerance, or allergy to ibuprofen, any NSAIDs, aspirin, or acetaminophen;
9. A subject who has had an active peptic ulceration in the 12 months prior to Screening or a history of gastrointestinal (GI) bleeding within 5 years of Screening;
10. A subject who has used an anticoagulant (except aspirin up to 325 mg/day for cardiac prophylaxis) in the month prior to Screening;
11. A subject who has positive results on fecal occult blood testing at Screening or on Day 1 prior to the first administration of study medication;
12. A subject who has a history of chronic inflammatory disease (such as rheumatoid arthritis, psoriatic arthritis, gouty arthritis), fibromyalgia, conditions that may affect the target joint (e.g., osteonecrosis, chondrocalcinosis), or asthma.

3.4. Study Medications:

Test Product, Dose, Dosage Form, and Mode of Administration:

The test product is a 7% solution of 2-(diethylamino)ethyl 2-(4-isobutylphenyl)propionate hydrochloride in 25% ethanol, which will be administered topically as a spray, the 7% topical spray solution consists of 700 mg 2-(diethylamino)ethyl 2-(4-isobutylphenyl)propionate hydrochloride (equivalent to 625 mg 2-(diethylamino)ethyl 2-(4-isobutylphenyl)propionate free base) in 10 mL 25% ethanol (v/v), the spray bottle deposits 70 mg of spray solution and 4.375 mg 2-(diethylamino)ethyl 2-(4-isobutylphenyl)propionate free base per spray on the skin. Subjects are to apply each spray to a different skin area around the knee (e.g., lateral, medial, front, and back surfaces of the knee) based on the randomized dose level assigned.

Reference Therapy, Dose, Dosage Form, and Mode of Administration:

The reference therapy is placebo, which will be administered topically as a spray. The spray bottle deposits 70 mg of spray solution per spray on the skin. Subjects are to apply each spray to a different skin area around the knee (e.g., lateral, medial, front, and back surfaces of the knee) based on the randomized dose level assigned.

Subjects will not take a shower or wash their knees until at least 8 hours after administration of study medication.

3.5. Dose and Regimen:

Subjects will receive the following treatments BID for 12 weeks. On the last day of dose administration (at the Week 12/EOS) visit, subjects will receive 1 dose of study medication in the morning only. The following treatments will be randomly assigned:
Group A: 8.75 mg 2-(diethylamino)ethyl 2-(4-isobutylphenyl)propionate/knee (2 sprays/knee, one spray to the medial surface and one spray to the lateral surface of the knee), BID (approximately every 12 hours; n=50) or placebo (2 sprays/knee), BID (approximately every 12 hours; n=25) for a total of 4 sprays per knee per day;
Group B: 17.5 mg 2-(diethylamino)ethyl 2-(4-isobutylphenyl)propionate/knee (4 sprays/knee, one spray to the medial surface and one spray to the lateral surface of the knee, and one spray to front and one spray to the back of the knee), BID (approximately every 12 hours; n=50) or placebo (4 sprays/knee), BID (approximately every 12 hours; n=25) for a total of 8 sprays per knee per day;
Group C: 35 mg 2-(diethylamino)ethyl 2-(4-isobutylphenyl)propionate/knee (8 sprays/knee, each spray is to be applied to a different non overlapping area around the knee, and the areas are to be uniformly distributed around the knee), BID (approximately every 12 hours; n=50) or placebo (8 sprays/knee), BID (approximately every 12 hours; n=25) for a total of 16 sprays per knee per day.

3.6. Number of Investigators and Study Centers:
20 sites in the United States

3.7. Duration of Subject Participation in Study:
Screening Period: up to 3 weeks
Treatment Period: 12 weeks
Length of Each Confinement: 36 hours after the last dose of study medication, only subjects in the PK subgroup will be confined during the study. These subjects will check into the study site at the Week 12/EOS visit and remain at the site until the 36 hour postdose PK blood collection has been completed, then return on the next 2 days for 48 and 72 hours postdose PK blood samples.
Follow-up Period: 7 days

3.8. Study Populations:

Safety Analysis Set (SAS): The SAS is defined as all subjects who were administered study medication and have at least 1 postdose safety assessment.

Full Analysis Set (FAS): The FAS is defined as all subjects who were administered study medication and have at least 1 postdose efficacy assessment.

Pharmacokinetics Analysis Set (PKAS): The PKAS is defined as all subjects who were administered 2-(diethylamino)ethyl 2-(4-isobutylphenyl)propionate and have at least 1 evaluable postdose 2-(diethylamino)ethyl 2-(4-isobutylphenyl)propionate and/or ibuprofen plasma concentration.

3.9. Evaluation: Efficacy:

3.9.1. Primary Efficacy Endpoint:

The Primary Efficacy Endpoint is the change from Baseline in the WOMAC (VAS) pain subscale score for the target knee at 12 weeks of treatment.

3.9.2. Secondary Efficacy Endpoints:
1. Change from Baseline in the WOMAC (VAS) pain subscale score for the target knee at 2, 4, and 8 weeks of treatment;
2. Change from Baseline in the WOMAC (VAS) stiffness subscale scores for the target knee and WOMAC (VAS) functional ability subscale scores at 2, 4, 8, and 12 weeks of treatment;
3. Change from Baseline in the overall WOMAC (VAS) score at 2, 4, 8, and 12 weeks of treatment.

3.9.3. Exploratory Efficacy Endpoints:
1. Subject's global assessment of disease status of the target knee at 2, 4, 8, and 12 weeks of treatment;
2. Investigator's global assessment of disease status of the target knee at 2, 4, 8, and 12 weeks of treatment;
3. Subject's global assessment of response to therapy of the target knee at 2, 4, 8, and 12 weeks of treatment;
4. Investigator's global assessment of response to therapy of the target knee at 2, 4, 8, and 12 weeks of treatment;
5. Change from Baseline over time in VAS pain scores for the target knee from Daily Diary data;
6. Amount of rescue medication (acetaminophen) consumed per day for target knee pain.

3.10. Evaluation: Safety

Safety assessments will include AEs, vital signs (blood pressures, pulse rate, and oral temperature), clinical laboratories, physical examination, skin irritation, and ECGs at various time points during the study as indicated in the study flow chart.

Adverse events of interest include: local skin reactions around the treated knee(s), upper stomach pain, GI bleeding, serious cardiovascular side effects (e.g., thrombotic events, myocardial infarction, or stroke), jaundice, elevated liver function tests, and nausea.

3.11. Statistical Methods:

Efficacy analyses will be conducted on the FAS.

3.11.1. Primary Efficacy Analysis:

The Primary Efficacy Endpoint is change from Baseline in the WOMAC (VAS) pain subscale score for the target knee at 4 weeks of treatment, and will be analyzed using an analysis of covariance (ANCOVA). Treatment will be included as a fixed class effect and WOMAC Baseline pain subscale score as covariates. The primary comparisons of interest will be the difference between active Group A (8.75 mg 2-(diethylamino)ethyl 2-(4-isobutylphenyl)propionate/knee) and combined placebo, active Group B (17.5 mg 2-(diethylamino)ethyl 2-(4-isobutylphenyl)propionate/knee) and combined placebo, and active Group C (35 mg 2-(diethylamino)ethyl 2-(4-isobutylphenyl)propionate/knee) and combined placebo.

3.11.2. Secondary Efficacy Analyses:

A sensitivity analysis will also be conducted on the Primary Efficacy Endpoint using an ANCOVA with treatment as a fixed class effect and WOMAC Baseline pain subscale score as covariates, but the comparisons of interest will be the difference between the active and placebo subjects within each treatment group.

The Secondary Efficacy Endpoints, change from Baseline in the WOMAC subscale scores for pain, stiffness, and functional ability, and overall WOMAC score at 2, 4, 8, and 12 weeks of treatment, will be analyzed using the same methods as for the Primary Efficacy Endpoint.

3.11.3. Exploratory Efficacy Endpoints:

Data for the exploratory efficacy endpoints will be summarized using descriptive statistics.

3.11.4. Safety Analyses:

Safety analyses will be conducted on the SAS. Safety parameters will be listed and summarized using standard descriptive statistics, as appropriate. No formal statistical analyses are planned.

3.12. Clinical Results

The blinded individual clinical data show the efficacy of 2-(diethylamino)ethyl 2-(4-isobutylphenyl)propionate spray are excellent. Especially, in the middle and high dose cohorts, many patients are almost pain-free at Week 12 (see italicized and bold numbers). The WOMAC Pain Subscale Scores, WOMAC Joint Stiffness Subscale Scores, and WOMAC Difficulty Performing Daily Activities Subscale Scores are showing in Table 15 through Table 23.

TABLE 15
Blinded WOMAC Pain Subscale Scores of All Patients Who
Finished 12 Weeks Treatment and
Follow-up Visit at Low Dose (2 spray/knee, 8.75 mg/knee) Cohort

| Patient | | Week | Week | Week | Week | Follow- |
|---|---|---|---|---|---|---|
| No. | Day 1 | 2 | 4 | 8 | 12 | up |
| 1002 | 66.2 | 51.2 | 23.2 | 27.8 | 12 | 24.8 |
| 1004 | 26.8 | 19.6 | 19.4 | 10.2 | 14.8 | 15.2 |
| 1012 | 78.6 | 74.6 | 56.2 | 60.6 | 65.4 | 74 |
| 1014 | 32.2 | 5.2 | 10.8 | 9.2 | 3.8 | 5 |
| 1018 | 58.2 | 42.2 | 49.6 | 30.6 | 74.8 | 73.4 |
| 1019 | 35.2 | 43.8 | 17.6 | 26 | 14.8 | 18.4 |
| 1023 | 59.4 | 47.4 | 53.8 | 57.4 | 58.4 | 58.2 |
| 1026 | 58.2 | 42.8 | 52.6 | 44.8 | 48.8 | 32.6 |
| 1029 | 78.8 | 75.6 | 72.6 | 71.4 | 73.6 | 73.8 |
| 1036 | 53.6 | 48 | 23 | 27.8 | 19.4 | 16 |
| 1039 | 40.4 | 12.8 | 11.6 | 11 | 8.4 | 7.8 |
| 1040 | 53.6 | 35.6 | 40 | 36.2 | 28.4 | 31.4 |
| 1057 | 58.8 | 58.6 | 36 | 25.2 | 48.4 | 65 |
| 1063 | 27.2 | 29.2 | 22 | 23.4 | 26.2 | 24.6 |
| 1065 | 29.6 | 26.8 | 15.6 | 11.6 | 13.2 | 9 |
| 1068 | 74.8 | 66.6 | 63 | 14 | 49.4 | 33.2 |
| 1451 | 69.4 | 57.8 | 59.8 | 56.6 | 56.2 | 51.6 |
| 1454 | 83.4 | 63.8 | 9 | 6.6 | 1.6 | 3.4 |
| 1457 | 71 | 61 | 61.2 | 59.4 | 65 | 79.2 |
| 1460 | 30 | 31.8 | 12.6 | 6.8 | 15.4 | 23.8 |
| 1462 | 58 | 59.8 | 55.8 | 44.8 | 61.2 | 60 |
| 1467 | 79.4 | 69.8 | 59.6 | 59 | 55.4 | 50.6 |
| 1471 | 55.2 | 54.6 | 37.2 | 31 | 61 | 64.6 |
| 1475 | 54.4 | 28.4 | 31 | 26.8 | 12.8 | 26.6 |
| 1477 | 86.4 | 86.8 | 85.4 | 83.4 | 89.2 | 86 |
| 1479 | 79.6 | 74.6 | 63 | 33.8 | 19 | 15.8 |
| 1482 | 50.6 | 47.4 | 47.6 | 47.6 | 58.2 | 43 |
| 1483 | 81 | 75.4 | 64.4 | 52.2 | 47.6 | 57 |
| 1487 | 65.6 | 61.4 | 62.6 | 64.6 | 54 | 59.6 |
| 1488 | 50.6 | 58 | 53.2 | 42.6 | 8 | 16.6 |
| 1492 | 61.2 | 50 | 58.6 | 53.4 | 54 | 59.2 |
| 1499 | 55.4 | 46.8 | 21.6 | 25.8 | 16.8 | 36.2 |
| 1504 | 34 | 72.8 | 85.2 | 78.6 | 71.8 | 77.8 |
| 1516 | 48.6 | 62.6 | 69 | 69.8 | 66.2 | 65.8 |
| 1517 | 81.8 | 85.6 | 87.4 | 93.4 | 91.4 | 94.8 |
| 1521 | 58.2 | 43 | 34.6 | 40 | 49.8 | 52.4 |
| 1525 | 55.6 | 53.6 | 33.2 | 53.8 | 27.8 | 44 |
| 1528 | 75.2 | 2.2 | 3 | 6 | 5.8 | 38 |
| 1531 | 75.2 | 62.8 | 47.2 | 54.6 | 49.2 | 53.4 |
| 1533 | 52.8 | 39.6 | 19.6 | 23 | 29.4 | 22.4 |
| 1538 | 61.2 | 62.6 | 58.6 | 48.4 | 40 | 41 |
| 1544 | 78 | 67 | 75.8 | 66 | 74 | 49 |
| 1547 | 54.2 | 57.6 | 42.4 | 41 | 41.2 | 46.6 |
| 9001 | 67.6 | 44.2 | 17.8 | 10.6 | 5.2 | 4.4 |
| 9002 | 51 | 22 | 23.6 | 28 | 13.2 | 12.2 |
| 9005 | 40.6 | 7 | 9.6 | 8.2 | 7.2 | 7.8 |
| 9012 | 46.8 | 24.4 | 35.8 | 35.8 | 29.6 | 10 |
| 9014 | 58.2 | 62.8 | 57 | 50 | 49.2 | 60.8 |
| 9015 | 56.2 | 43.8 | 55 | 53.6 | 68.6 | 69 |
| 9020 | 68.8 | 23.6 | 52 | 34.2 | 45.4 | 31.8 |
| 9021 | 57.4 | 68.8 | 68.2 | 66 | 63 | 62.6 |
| 9023 | 65.6 | 51.4 | 67.8 | 54.6 | 59.4 | 62.2 |
| 9025 | 88.4 | 54.2 | 43.4 | 23.8 | 22.4 | 64.8 |
| 9028 | 43.8 | 24.6 | 19.8 | 18 | 18.2 | 10.4 |
| 9030 | 72.4 | 55.6 | 67.8 | 55.6 | 16.4 | 4.6 |
| 9035 | 73.8 | 71.6 | 54 | 61.4 | 48.6 | 59.8 |
| 9452 | 66.8 | 1 | 0.2 | 0.2 | 1 | 0.6 |
| 9453 | 66 | 21.2 | 11.8 | 9.4 | 21.4 | 17 |
| 9458 | 57.2 | 58.2 | 65.8 | 53.4 | 52.2 | 58.4 |
| 9463 | 72.2 | 57 | 10 | 7 | 7 | 4.6 |
| 9464 | 76.2 | 68.6 | 53.8 | 35.6 | 34 | 14.4 |
| 9466 | 43.6 | 36 | 36 | 21.2 | 25.4 | 22.6 |
| Avg. | 59.8 | 48.6 | 42.8 | 38.4 | 38.2 | 39.7 |

TABLE 16
Blinded WOMAC Joint Stiffness Subscale Scores of All Patients Who
Finished 12 Weeks Treatment and
Follow-up Visit at Low Dose (2 spray/knee, 8.75 mg/knee) Cohort

| Patient | | Week | Week | Week | Week | Follow- |
|---|---|---|---|---|---|---|
| No. | Day 1 | 2 | 4 | 8 | 12 | up |
| 1002 | 70.5 | 55.5 | 23.5 | 28.5 | 20 | 16.5 |
| 1004 | 75 | 40 | 62 | 60 | 44 | 46 |
| 1012 | 83.5 | 83 | 76.5 | 75.5 | 80 | 56 |
| 1014 | 0.5 | 5.5 | 5.5 | 4.5 | 2.5 | 3.5 |
| 1018 | 84 | 51.5 | 53 | 30 | 56.5 | 71 |
| 1019 | 12.5 | 14 | 0 | 1.5 | 19.5 | 10.5 |
| 1023 | 54.4 | 49.5 | 56.5 | 62 | 55 | 56.5 |
| 1026 | 54 | 56 | 59.5 | 29 | 50.5 | 30 |
| 1029 | 76.5 | 75 | 72.5 | 74 | 73 | 69 |
| 1036 | 35 | 22.5 | 13 | 22 | 8 | 9.5 |
| 1039 | 72.5 | 25 | 25.5 | 34.5 | 17.5 | 19.5 |
| 1040 | 60.5 | 38 | 57 | 49.5 | 42.5 | 49.5 |
| 1057 | 47.5 | 75 | 87 | 59 | 71.5 | 75 |
| 1063 | 34 | 38.5 | 25.5 | 21.5 | 36.5 | 38.5 |
| 1065 | 34.5 | 37.5 | 15.5 | 10.5 | 12.5 | 8 |
| 1068 | 75 | 69.5 | 41 | 12 | 33 | 39 |
| 1451 | 62 | 49.5 | 47.5 | 61 | 67.5 | 69.5 |
| 1454 | 91.5 | 50 | 6.5 | 11.5 | 2.5 | 14.5 |
| 1457 | 75.5 | 71 | 79 | 79 | 83.5 | 77 |
| 1460 | 48.5 | 34 | 9.5 | 12.5 | 21.5 | 32.5 |
| 1462 | 68.5 | 63.5 | 62 | 50.5 | 70.5 | 53.5 |
| 1467 | 91 | 83.5 | 66.5 | 75.5 | 72.5 | 61 |
| 1471 | 75.5 | 63.5 | 62 | 50.5 | 70.5 | 53.5 |
| 1475 | 75.5 | 52.5 | 53 | 42.5 | 35.5 | 15 |
| 1477 | 82.5 | 85 | 85 | 83.5 | 89 | 90 |
| 1479 | 87.5 | 87.5 | 64.5 | 41 | 27 | 10.5 |
| 1482 | 51.5 | 45 | 44.5 | 50 | 59.5 | 51.5 |
| 1483 | 82 | 72 | 62 | 56.5 | 44 | 58 |
| 1487 | 63 | 54.5 | 39 | 55.5 | 38.5 | 28.5 |
| 1488 | 44.5 | 52 | 51.5 | 44.5 | 8.5 | 16.5 |
| 1492 | 62.5 | 59 | 63.5 | 64.5 | 55.5 | 58 |
| 1499 | 59 | 50.5 | 33.5 | 37.5 | 42 | 58.5 |
| 1504 | 77.5 | 93 | 85.5 | 77.5 | 78.5 | 69.5 |
| 1516 | 64 | 64 | 66 | 73 | 70 | 69.5 |
| 1517 | 87.5 | 86 | 91.5 | 97 | 95 | 98 |
| 1521 | 54 | 46.5 | 35.5 | 44 | 49.5 | 49.5 |
| 1525 | 49 | 68 | 38 | 66.5 | 61 | 37.5 |
| 1528 | 79.5 | 19.5 | 22 | 7 | 9.5 | 39.5 |
| 1531 | 76.5 | 70.5 | 56 | 54.5 | 54 | 52.5 |
| 1533 | 82.5 | 53.5 | 6.5 | 13 | 29.5 | 14.5 |
| 1538 | 75 | 59 | 46.5 | 57 | 21 | 33 |
| 1544 | 76 | 72.5 | 73 | 58.5 | 74 | 54 |
| 1547 | 66.5 | 57 | 44.5 | 41.5 | 37.5 | 52 |
| 9001 | 60.5 | 44.5 | 12.5 | 9 | 7 | 6.5 |
| 9002 | 92.5 | 48 | 59.5 | 26.5 | 48 | 13 |
| 9005 | 43.5 | 25.5 | 8.5 | 11.5 | 5.5 | 7 |
| 9012 | 37.5 | 42 | 23 | 70 | 14 | 10 |
| 9014 | 58 | 58.5 | 55.5 | 50 | 53.5 | 70 |
| 9015 | 54.5 | 44 | 52 | 40.5 | 64.5 | 59 |
| 9020 | 74.5 | 44.5 | 63.5 | 47 | 57 | 66 |
| 9021 | 64.5 | 66.5 | 74 | 68 | 69.5 | 67 |
| 9023 | 63 | 46 | 65.5 | 48 | 53.5 | 66.5 |
| 9025 | 86 | 56 | 58 | 38.5 | 47.5 | 74 |
| 9028 | 51 | 44.5 | 20.5 | 14.5 | 14.5 | 9.5 |
| 9030 | 69 | 25 | 65.5 | 64.5 | 11 | 4 |
| 9035 | 64 | 49.5 | 50 | 64 | 53 | 58 |
| 9452 | 80.5 | 0 | 0 | 0 | 0.5 | 1 |
| 9453 | 71 | 47 | 17.5 | 15 | 10.5 | 17 |
| 9458 | 30 | 40.5 | 61.5 | 47 | 47.5 | 62.5 |
| 9463 | 85 | 65.5 | 15 | 8.5 | 11 | 7.5 |
| 9464 | 67 | 63 | 42.5 | 10 | 15.5 | 8 |
| 9466 | 50.5 | 47 | 39 | 30 | 17.5 | 28.5 |
| Avg. | 64.2 | 52.5 | 45.7 | 42.3 | 41.8 | 41.4 |

TABLE 17
Blinded WOMAC Difficulty Performing Daily Activities Subscale
Scores of All Patients Who Finished 12 Weeks Treatment and
Follow-up Visit at Low Dose (2 spray/knee, 8.75 mg/knee) Cohort

| Patient | | Week | Week | Week | Week | Follow- |
|---|---|---|---|---|---|---|
| No. | Day 1 | 2 | 4 | 8 | 12 | up |
| 1002 | 73.8 | 40.4 | 24.4 | 24.0 | 14.5 | 29.8 |
| 1004 | 37.9 | 27.8 | 24.2 | 23.2 | 25.8 | 20 |
| 1012 | 84.2 | 80.9 | 65.9 | 66.1 | 69.1 | 62.6 |
| 1014 | 9.6 | 5.1 | 6.9 | 6.4 | 2.4 | 4.8 |
| 1018 | 57.6 | 48.4 | 52.4 | 22.7 | 71.2 | 57.5 |
| 1019 | 30.2 | 24.8 | 4.0 | 7.5 | 22.7 | 12.9 |
| 1023 | 65.9 | 48.7 | 57.1 | 63.1 | 60.2 | 55.8 |
| 1026 | 54.4 | 56.7 | 60.1 | 48.6 | 44.7 | 46.4 |
| 1029 | 74.9 | 76.1 | 71.1 | 73.2 | 71.6 | 75.8 |
| 1036 | 62.6 | 39.9 | 25.2 | 25.9 | 16.3 | 19.1 |
| 1039 | 25.0 | 16.5 | 13.8 | 13.7 | 14.7 | 8.2 |
| 1040 | 57.0 | 44.8 | 46.0 | 43.6 | 41.3 | 43.3 |
| 1057 | 31.5 | 62.4 | 53.9 | 42.8 | 42.5 | 58.3 |
| 1063 | 41.6 | 37.6 | 34.1 | 38.5 | 34.7 | 35.9 |
| 1065 | 35.2 | 29.9 | 13.3 | 10.9 | 13.5 | 9.6 |
| 1068 | 73.8 | 67.1 | 41.8 | 13.3 | 36.9 | 22.1 |
| 1451 | 52.8 | 39.2 | 47.4 | 61.8 | 65.1 | 63.9 |
| 1454 | 91.7 | 65.2 | 14.4 | 13.8 | 8.7 | 9.5 |
| 1457 | 58.3 | 61.4 | 64.2 | 66.2 | 68.9 | 64.8 |
| 1460 | 30.5 | 34.4 | 13.4 | 14.4 | 14.4 | 21.2 |
| 1462 | 63.8 | 56.6 | 62.8 | 50.5 | 59.9 | 59.4 |
| 1467 | 75.1 | 76.6 | 58.5 | 63.8 | 60.6 | 60.5 |
| 1471 | 69.4 | 47.6 | 32.6 | 31.1 | 67.3 | 67.2 |
| 1475 | 63.4 | 40.4 | 30.0 | 29.1 | 20.5 | 16.5 |
| 1477 | 84.4 | 86.2 | 85.1 | 83.5 | 89.5 | 86.7 |
| 1479 | 75.2 | 71.1 | 49.9 | 29.8 | 19.1 | 11.0 |
| 1482 | 49.1 | 47.8 | 47.7 | 47.2 | 57.7 | 52.9 |
| 1483 | 79.6 | 76.1 | 64.4 | 54.3 | 44.5 | 54.2 |
| 1487 | 62.8 | 48.4 | 59.5 | 59.2 | 55.7 | 54.5 |
| 1488 | 56.1 | 60.6 | 54.8 | 49.1 | 9.1 | 20.7 |
| 1492 | 57.9 | 49.8 | 59.2 | 51.8 | 48.6 | 57.0 |
| 1499 | 62.5 | 57.6 | 38.8 | 39.4 | 28.1 | 46.6 |
| 1504 | 64.9 | 74.9 | 75.1 | 81.4 | 73.2 | 66.9 |
| 1516 | 53.8 | 65.1 | 65.2 | 74.8 | 67.8 | 69.1 |
| 1517 | 94.4 | 87.9 | 89.9 | 94.8 | 97.3 | 97.7 |
| 1521 | 54.2 | 45.4 | 47.9 | 50.9 | 50.7 | 53.7 |
| 1525 | 37.6 | 61.8 | 49.4 | 53.5 | 35.7 | 42.6 |
| 1528 | 80.9 | 2.4 | 3.1 | 9.7 | 5.6 | 17.5 |
| 1531 | 76.8 | 69.6 | 56.8 | 55.4 | 53.9 | 51.2 |
| 1533 | 67.4 | 44.7 | 27.9 | 22.4 | 22.9 | 24.9 |
| 1538 | 65.5 | 52.0 | 49.2 | 51.0 | 36.5 | 36.9 |
| 1544 | 78.0 | 71.8 | 74.2 | 63.9 | 72.7 | 50.5 |
| 1547 | 50.4 | 51.5 | 43.8 | 48.4 | 45.2 | 50.0 |
| 9001 | 64.5 | 33.9 | 11.9 | 11.3 | 4.2 | 4.1 |
| 9002 | 76.9 | 41.2 | 34.3 | 56.8 | 61.6 | 32.9 |
| 9005 | 45.7 | 18.9 | 9.3 | 8.4 | 8.1 | 7.8 |
| 9012 | 37.4 | 32.3 | 44.0 | 32.3 | 18.0 | 13.0 |
| 9014 | 56.7 | 62.8 | 53.9 | 50.9 | 54.6 | 67.5 |
| 9015 | 63.7 | 49.4 | 52.1 | 45.4 | 65.9 | 65.3 |
| 9020 | 67.6 | 33.9 | 63.4 | 54.2 | 55.3 | 58.3 |
| 9021 | 61.9 | 67.1 | 73.4 | 69.7 | 68.8 | 65.8 |
| 9023 | 49.2 | 45.5 | 63.3 | 51.8 | 50.6 | 57.6 |
| 9025 | 82.3 | 55.4 | 52.6 | 35.4 | 30.9 | 69.3 |
| 9028 | 57.1 | 39.7 | 23.9 | 22.0 | 14.4 | 11.1 |
| 9030 | 73.4 | 46.1 | 66.4 | 47.2 | 8.4 | 3.8 |
| 9035 | 71.7 | 64.6 | 57.5 | 57.1 | 47.6 | 49.0 |
| 9452 | 86.8 | 0.5 | 0.4 | 0.2 | 0.2 | 0.5 |
| 9453 | 45.9 | 19.3 | 15.5 | 9.2 | 19.6 | 18.9 |
| 9458 | 48.5 | 58.3 | 57.6 | 50.4 | 50.8 | 54.9 |
| 9463 | 75.8 | 69.5 | 15.4 | 7.8 | 7.5 | 6.7 |
| 9464 | 69.8 | 66.9 | 49.6 | 39.6 | 19.2 | 8.9 |
| 9466 | 38.8 | 27.1 | 22.3 | 21.9 | 19.5 | 18.0 |
| Avg. | 60.4 | 49.8 | 44.0 | 41.1 | 39.8 | 40.1 |

TABLE 18
Blinded WOMAC Pain Subscale Scores of All Patients
Who Finished 12 Weeks Treatment and Follow-up
Visit at Middle Dose (4 spray/knee, 17.5 mg/knee) Cohort

| Patient | | Week | Week | Week | Week | Follow- |
|---|---|---|---|---|---|---|
| No. | Day 1 | 2 | 4 | 8 | 12 | up |
| 1001 | 67.2 | 65.4 | 67.8 | 69.8 | 70.2 | 75.4 |
| 1008 | 52.4 | 3.2 | 1.8 | 5.0 | 2.0 | 12.0 |
| 1011 | 86.2 | 78.2 | 73.6 | 68.2 | 73.4 | 85.8 |
| 1017 | 34.4 | 12.4 | 3 | 15.8 | 39.6 | 12.4 |
| 1020 | 76.4 | 38.4 | 11.2 | 5.4 | 11.6 | 11.6 |
| 1024 | 72.2 | 76.4 | 78.6 | 83.4 | 80.8 | 70.2 |
| 1025 | 20.8 | 11.6 | 11.4 | 21.4 | 21.0 | 13.8 |
| 1028 | 34.0 | 20.0 | 8.8 | 6.4 | 5.2 | 7.0 |
| 1030 | 67.6 | 64.4 | 51.0 | 18.8 | 1.0 | 0.0 |
| 1032 | 43.2 | 43.2 | 15.0 | 32.0 | 10.4 | 23.0 |
| 1038 | 93.2 | 86.6 | 85.6 | 86.4 | 77.4 | 82.8 |
| 1043 | 76.4 | 23.8 | 14.4 | 26.4 | 17.8 | 19.4 |
| 1048 | 41 | 19.6 | 6.8 | 5.4 | 3.2 | 15.2 |
| 1049 | 63.6 | 64.6 | 48.8 | 61.6 | 25.4 | 45 |
| 1052 | 80.2 | 60.4 | 78.0 | 73.0 | 71.6 | 79.8 |
| 1058 | 56.2 | 2.4 | 3.6 | 5.0 | 4.6 | 81.4 |
| 1061 | 75.0 | 55.4 | 73.8 | 40.0 | 29.6 | 8.6 |
| 1062 | 29.6 | 25 | 28.2 | 14.8 | 20.0 | 18.6 |
| 1064 | 32.6 | 21.6 | 1.8 | 1 | 0 | 1.6 |
| 1453 | 53.0 | 32.8 | 31.8 | 34.8 | 31.0 | 39.0 |
| 1456 | 62.0 | 24.8 | 54.0 | 17.8 | 26.0 | 34.2 |
| 1458 | 59.4 | 37.4 | 51.6 | 23.0 | 18.2 | 19.8 |
| 1465 | 63.6 | 32.6 | 17.4 | 17.0 | 7.4 | 11.4 |
| 1470 | 64.6 | 54.8 | 35.8 | 33.2 | 22 | 73.4 |
| 1473 | 52.8 | 29.2 | 25.2 | 5.2 | 3.8 | 7.8 |
| 1476 | 81.2 | 85.0 | 80.4 | 84.4 | 82.2 | 82.6 |
| 1480 | 69.2 | 58.0 | 47.2 | 51.2 | 55.6 | 46.8 |
| 1484 | 64.2 | 68.0 | 61.8 | 61.0 | 62.4 | 64.6 |
| 1486 | 44.8 | 38.2 | 44.6 | 36.2 | 60.0 | 55.0 |
| 1490 | 90.8 | 83.8 | 77.2 | 81.6 | 54.6 | 78.2 |
| 1494 | 50.6 | 42.0 | 41.8 | 45.0 | 52.0 | 46.4 |
| 1495 | 69.4 | 61.0 | 44.2 | 23.8 | 16.8 | 30.6 |
| 1500 | 61.6 | 36.4 | 32.0 | 16.8 | 35.0 | 13.8 |
| 1502 | 66.8 | 69.4 | 48.4 | 42.0 | 53.0 | 31.0 |
| 1507 | 81.8 | 80.2 | 68.6 | 75.8 | 77.4 | 76.2 |
| 1510 | 43.2 | 41.6 | 45.0 | 47.4 | 48.0 | 54.6 |
| 1518 | 64.8 | 64.2 | 61.8 | 67.6 | 49.2 | 63.0 |
| 1519 | 80.0 | 57.0 | 61.4 | 48.0 | 69.6 | 74.2 |
| 1523 | 90.4 | 86.0 | 88.0 | 93.6 | 90.0 | 95.2 |
| 1526 | 57.2 | 14.4 | 6.2 | 8.2 | 3.0 | 3.8 |
| 1529 | 77.4 | 71.4 | 8.0 | 30.4 | 27.2 | 16.0 |
| 1532 | 55.6 | 41.6 | 43.8 | 20.4 | 7.2 | 7.2 |
| 1536 | 53.4 | 50.4 | 55.2 | 52.8 | 43.0 | 45.6 |
| 1540 | 33.4 | 27.6 | 15.2 | 8.0 | 7.8 | 18.0 |
| 1541 | 44.2 | 43.6 | 10.2 | 6.2 | 2.0 | 2.0 |
| 1545 | 46.6 | 41.8 | 49.4 | 52.8 | 60.2 | 68.6 |
| 9001 | 67.6 | 44.2 | 17.8 | 10.6 | 5.2 | 4.4 |
| 9004 | 44.6 | 12.4 | 11.4 | 5.6 | 3.6 | 20.0 |
| 9007 | 46.4 | 47.6 | 47.2 | 38.2 | 37.4 | 44 |
| 9011 | 58.6 | 43.8 | 34.6 | 29.6 | 26.0 | 32.8 |
| 9013 | 52.2 | 52.2 | 47.0 | 22.2 | 36.2 | 33.4 |
| 9018 | 51.0 | 22.8 | 6.8 | 7.6 | 0.6 | 1.2 |
| 9022 | 51.8 | 47.0 | 41.2 | 62.6 | 48.2 | 36.6 |
| 9024 | 81.6 | 12.0 | 6.2 | 3.6 | 4.0 | 53.2 |
| 9025 | 88.4 | 54.2 | 43.4 | 23.8 | 22.4 | 64.8 |
| 9026 | 60.4 | 56.8 | 51.2 | 44.2 | 25.8 | 25.8 |
| 9029 | 68.0 | 68.8 | 72.0 | 64.8 | 59.2 | 70.6 |
| 9034 | 85.0 | 61.8 | 66.0 | 42.6 | 37.4 | 30.6 |
| 9036 | 76.0 | 57.8 | 80.0 | 21.0 | 75.2 | 23.0 |
| 9451 | 74.0 | 37.6 | 58.4 | 61.6 | 63.2 | 65.6 |
| 9455 | 45.6 | 32.2 | 13.2 | 5.8 | 17.0 | 10.8 |
| 9456 | 81.6 | 77.6 | 74.6 | 78.0 | 79.2 | 76.8 |
| 9460 | 43.4 | 45.2 | 35.6 | 39.0 | 28.6 | 27.4 |
| 9461 | 43.8 | 22.8 | 18.0 | 9.6 | 11.8 | 45.6 |
| Avg. | 61.0 | 46.0 | 40.5 | 35.9 | 34.5 | 38.9 |

TABLE 19
Blinded WOMAC Joint Stiffness Subscale Scores of All
Patients Who Finished 12 Weeks Treatment and Follow-up
Visit at Middle Dose (4 spray/knee, 17.5 mg/knee) Cohort

| Patient | | Week | Week | Week | Week | Follow- |
|---|---|---|---|---|---|---|
| No. | Day 1 | 2 | 4 | 8 | 12 | up |
| 1001 | 72.0 | 57.0 | 71.0 | 71.5 | 69.0 | 80.0 |
| 1008 | 61.5 | 10.5 | 1.5 | 3.0 | 5.5 | 11.5 |
| 1011 | 87.5 | 74.0 | 67.0 | 75.0 | 65.5 | 84.5 |
| 1017 | 45.5 | 9.5 | 5.5 | 41.5 | 33.0 | 21.0 |
| 1020 | 70.0 | 50.0 | 22.5 | 3.5 | 14.0 | 10.5 |
| 1024 | 84.5 | 84.0 | 86.5 | 86.5 | 87.0 | 80.0 |
| 1025 | 60.0 | 14.5 | 29.0 | 24.0 | 24.5 | 20.5 |
| 1028 | 61.5 | 18.5 | 8.0 | 7.5 | 5.5 | 6.5 |
| 1030 | 75.5 | 78.0 | 79.5 | 59.0 | 2.0 | 0.5 |
| 1032 | 52.0 | 13.0 | 19.0 | 55.5 | 24.0 | 29.0 |
| 1038 | 88.5 | 78.5 | 85.5 | 86.0 | 85.5 | 77.0 |
| 1043 | 87.0 | 24.0 | 16.0 | 48.5 | 11.5 | 19.0 |
| 1048 | 69.0 | 24.5 | 8.5 | 7.5 | 3.0 | 12.5 |
| 1049 | 57.0 | 65.5 | 61.5 | 42.0 | 23.0 | 42.0 |
| 1052 | 80.0 | 60.5 | 81.0 | 74.0 | 78.5 | 81.0 |
| 1058 | 56.0 | 2.5 | 3.5 | 3.5 | 4.5 | 81.0 |
| 1061 | 92.5 | 83.5 | 87.5 | 86.0 | 54.5 | 19.5 |
| 1062 | 45.0 | 59.0 | 39.0 | 27.5 | 27.0 | 29.5 |
| 1064 | 66.5 | 20.5 | 12.5 | 0.0 | 1.0 | 1.0 |
| 1453 | 54.5 | 39.5 | 30.5 | 31.5 | 46.0 | 50.5 |
| 1456 | 65.5 | 16.5 | 28.5 | 11.0 | 7.5 | 7.5 |
| 1458 | 85.5 | 82.5 | 75.5 | 37.5 | 47.0 | 28.0 |
| 1465 | 69.5 | 35.0 | 19.5 | 17.5 | 8.0 | 7.5 |
| 1470 | 70.0 | 64.5 | 59.0 | 47.5 | 33.5 | 85.0 |
| 1473 | 64.5 | 49.5 | 36.0 | 4.0 | 4.5 | 6.5 |
| 1476 | 89.5 | 77.0 | 80.0 | 73.5 | 85.0 | 87.5 |
| 1480 | 88.0 | 59.0 | 80.0 | 66.5 | 71.0 | 57.0 |
| 1484 | 58.5 | 46.0 | 56.0 | 57.5 | 61.5 | 64.0 |
| 1486 | 73.5 | 52.5 | 64.5 | 53.0 | 53.0 | 59.5 |
| 1490 | 91.5 | 87.5 | 82.5 | 66.0 | 89.0 | 75.5 |
| 1494 | 41.5 | 29.0 | 37.0 | 43.0 | 47.5 | 40.0 |
| 1495 | 75.5 | 63.0 | 49.0 | 35.5 | 17.0 | 44.0 |
| 1500 | 60.0 | 41.0 | 29.5 | 19.5 | 43.0 | 12.5 |
| 1502 | 68.5 | 68.0 | 55.0 | 51.0 | 53.0 | 29.0 |
| 1507 | 91.5 | 88.5 | 67.5 | 82.0 | 84.0 | 92.5 |
| 1510 | 49.5 | 41.5 | 41.5 | 47.5 | 41.5 | 51.5 |
| 1518 | 68.5 | 69.0 | 63.5 | 74.5 | 72.0 | 67.0 |
| 1519 | 66.5 | 57.0 | 76.5 | 67.0 | 83.0 | 88.0 |
| 1523 | 86.5 | 92.5 | 93.5 | 92.0 | 93.5 | 95.0 |
| 1526 | 61.5 | 21.5 | 5.5 | 6.0 | 2.5 | 3.0 |
| 1529 | 80.5 | 74.0 | 13.0 | 44.5 | 37.0 | 31.5 |
| 1532 | 71.5 | 62.0 | 40.5 | 45.0 | 5.0 | 2.0 |
| 1536 | 51.0 | 42.0 | 49.0 | 47.5 | 46.0 | 42.5 |
| 1540 | 61.5 | 17.5 | 12.0 | 11.5 | 7.0 | 21.0 |
| 1541 | 66.0 | 48.0 | 21.5 | 1.5 | 2.0 | 2.0 |
| 1545 | 55.0 | 43.5 | 54.5 | 55.5 | 62.0 | 63.5 |
| 9001 | 60.5 | 44.5 | 12.5 | 9.0 | 7.0 | 6.5 |
| 9004 | 64.5 | 10.0 | 14.5 | 5.0 | 3.5 | 28.0 |
| 9007 | 55.5 | 53.5 | 45.0 | 41.0 | 42.0 | 44.0 |
| 9011 | 45.0 | 39.0 | 26.5 | 29.5 | 27.0 | 31.0 |
| 9013 | 51.5 | 44.0 | 43.0 | 18.5 | 35.5 | 28.5 |
| 9018 | 57.0 | 31.0 | 12.5 | 11.0 | 1.0 | 1.0 |
| 9022 | 29.5 | 52.5 | 35.0 | 60.5 | 45.0 | 18.0 |
| 9024 | 70.5 | 36.5 | 17.0 | 24.0 | 22.0 | 84.0 |
| 9025 | 86.0 | 56.0 | 58.0 | 38.5 | 47.5 | 74.0 |
| 9026 | 66.0 | 62.0 | 56.5 | 47.5 | 30.0 | 27.5 |
| 9029 | 83.5 | 79.5 | 76.5 | 71.0 | 72.0 | 78.5 |
| 9034 | 89.0 | 63.5 | 69.5 | 42.0 | 38.5 | 36.0 |
| 9036 | 89.0 | 73.0 | 89.0 | 63.5 | 60.5 | 50.0 |
| 9451 | 78.0 | 38.5 | 50.0 | 55.0 | 63.0 | 75.5 |
| 9455 | 83.5 | 30.5 | 28.0 | 16.0 | 13.0 | 19.5 |
| 9456 | 82.5 | 75.5 | 76.0 | 85.0 | 84.0 | 83.5 |
| 9460 | 40.0 | 43.5 | 27.5 | 42.5 | 25.0 | 27.0 |
| 9461 | 42.0 | 20.5 | 16.0 | 8.0 | 8.0 | 51.0 |
| Avg. | 68.0 | 49.2 | 44.7 | 41.5 | 38.2 | 41.9 |

TABLE 20
Blinded WOMAC Difficulty Performing Daily Activities Subscale
Scores of All Patients Who Finished 12 Weeks Treatment and
Follow-up Visit at Middle Dose (4 spray/knee, 17.5 mg/knee) Cohort

| Patient | | Week | Week | Week | Week | Follow- |
|---|---|---|---|---|---|---|
| No. | Day 1 | 2 | 4 | 8 | 12 | up |
| 1001 | 61.8 | 64.2 | 63.4 | 73.1 | 73.2 | 70.3 |
| 1008 | 53.3 | 4.9 | 2.1 | 4.3 | 2.6 | 12.1 |
| 1011 | 82.9 | 73.8 | 71.8 | 72.7 | 63.4 | 81.2 |
| 1017 | 38.7 | 7.7 | 6.1 | 23.1 | 34.1 | 13.4 |
| 1020 | 77.9 | 37.0 | 9.4 | 4.5 | 11.6 | 11.6 |
| 1024 | 54.8 | 60.7 | 62.9 | 64.8 | 60.5 | 58.9 |
| 1025 | 32.4 | 18.1 | 17.4 | 34.8 | 29.4 | 23.8 |
| 1028 | 37.9 | 17.8 | 10.0 | 7.8 | 6.8 | 6.9 |
| 1030 | 75.4 | 68.6 | 70.3 | 54.2 | 0.2 | 0.6 |
| 1032 | 43.0 | 9.7 | 12.7 | 22.6 | 21.6 | 17.1 |
| 1038 | 89.4 | 88.0 | 85.0 | 86.2 | 85.1 | 77.9 |
| 1043 | 76.2 | 25.7 | 13.4 | 33.4 | 17.5 | 18.9 |
| 1048 | 63.3 | 18.5 | 7.4 | 6.6 | 3.5 | 16.6 |
| 1049 | 64.6 | 69.7 | 66.8 | 63.7 | 39.9 | 52.1 |
| 1052 | 78.5 | 49.4 | 77.3 | 74.9 | 73.2 | 80.5 |
| 1058 | 49.2 | 2.5 | 3.5 | 3.2 | 4.4 | 59.7 |
| 1061 | 84.4 | 70.8 | 76.2 | 67.3 | 36.2 | 15.6 |
| 1062 | 42.1 | 47.6 | 38.9 | 21.9 | 23.9 | 19.5 |
| 1064 | 33.6 | 24.4 | 1.1 | 0.5 | 0.6 | 0.4 |
| 1453 | 54.4 | 37.6 | 40.6 | 39.5 | 43.2 | 44.6 |
| 1456 | 69.0 | 19.8 | 45.1 | 16.1 | 26.6 | 28.6 |
| 1458 | 72.3 | 57.1 | 54.2 | 30.6 | 21.2 | 24.0 |
| 1465 | 65.8 | 27.2 | 18.6 | 16.4 | 6.6 | 7.4 |
| 1470 | 72.0 | 61.2 | 41.9 | 46.9 | 30.6 | 76.7 |
| 1473 | 57.9 | 48.4 | 39.3 | 6.2 | 3.9 | 7.5 |
| 1476 | 80.3 | 84.2 | 82.4 | 85.0 | 79.4 | 91.2 |
| 1480 | 75.9 | 50.4 | 62.9 | 61.4 | 63.4 | 53.8 |
| 1484 | 62.1 | 68.1 | 65.1 | 57.4 | 64.1 | 65.4 |
| 1486 | 44.5 | 47.1 | 38.2 | 41.6 | 48.6 | 44.8 |
| 1490 | 87.6 | 81.0 | 80.5 | 79.5 | 49.6 | 77.2 |
| 1494 | 41.4 | 37.9 | 46.1 | 44.4 | 51.0 | 44.5 |
| 1495 | 67.1 | 64.5 | 52.6 | 24.4 | 18.1 | 41.1 |
| 1500 | 61.9 | 46.8 | 29.8 | 24.4 | 47.6 | 12.3 |
| 1502 | 63.8 | 65.4 | 43.9 | 48.9 | 52.4 | 26.9 |
| 1507 | 77.0 | 73.7 | 66.8 | 73.8 | 75.5 | 77.9 |
| 1510 | 45.1 | 42.3 | 42.9 | 45.4 | 46.9 | 57.1 |
| 1518 | 67.9 | 68.6 | 48.8 | 57.1 | 55.6 | 60.3 |
| 1519 | 73.8 | 63.9 | 72.1 | 70.1 | 81.6 | 82.1 |
| 1523 | 92.7 | 93.7 | 93.6 | 93.9 | 96.8 | 96.4 |
| 1526 | 61.1 | 14.7 | 6.9 | 7.5 | 2.8 | 5.0 |
| 1529 | 81.1 | 75.0 | 9.9 | 24.0 | 28.6 | 21.5 |
| 1532 | 58.8 | 31.3 | 26.8 | 17.8 | 3.2 | 5.2 |
| 1536 | 51.5 | 47.9 | 53.7 | 50.4 | 44.5 | 44.5 |
| 1540 | 42.0 | 36.5 | 16.2 | 9.2 | 8.9 | 26.1 |
| 1541 | 63.8 | 67.5 | 21.5 | 8.6 | 2.2 | 2.4 |
| 1545 | 53.2 | 51.6 | 53.5 | 57.5 | 61.8 | 67.4 |
| 9001 | 64.5 | 33.9 | 11.9 | 11.3 | 4.2 | 4.1 |
| 9004 | 51.1 | 9.0 | 11.2 | 4.6 | 3.4 | 24.3 |
| 9007 | 53.1 | 47.9 | 48.7 | 41.6 | 37.9 | 47.3 |
| 9011 | 57.4 | 47.2 | 43.8 | 36.1 | 40.5 | 42.2 |
| 9013 | 61.9 | 52.8 | 47.8 | 37.5 | 43.4 | 37.9 |
| 9018 | 60.4 | 24.7 | 7.5 | 10.8 | 0.2 | 2.6 |
| 9022 | 58.6 | 53.4 | 50.1 | 70.4 | 56.8 | 52.1 |
| 9024 | 76.0 | 35.8 | 13.4 | 10.5 | 5.6 | 39.8 |
| 9025 | 82.3 | 55.4 | 52.6 | 35.4 | 30.9 | 69.3 |
| 9026 | 58.8 | 56.9 | 55.6 | 47.6 | 31.2 | 25.4 |
| 9029 | 73.4 | 78.4 | 72.2 | 74.3 | 68.3 | 73.8 |
| 9034 | 80.6 | 60.3 | 61.9 | 48.2 | 42.4 | 35.6 |
| 9036 | 79.9 | 77.4 | 84.2 | 26.4 | 72.7 | 55.5 |
| 9451 | 70.7 | 40.8 | 50.4 | 58.1 | 61.6 | 73.5 |
| 9455 | 64.5 | 33.2 | 21.1 | 11.6 | 14.3 | 18.0 |
| 9456 | 83.4 | 78.7 | 74.8 | 81.2 | 78.9 | 79.3 |
| 9460 | 45.5 | 45.0 | 33.9 | 41.6 | 26.5 | 23.6 |
| 9461 | 48.8 | 26.2 | 18.5 | 10.1 | 12.3 | 46.3 |
| Avg. | 63.4 | 48.1 | 42.3 | 39.3 | 36.5 | 40.3 |

TABLE 21
Blinded WOMAC Pain Subscale Scores of All Patients
Who Finished 12 Weeks Treatment and Follow-up
Visit at High Dose (8 spray/knee, 35 mg/knee) Cohort

| Patient | | Week | Week | Week | Week | Follow- |
|---|---|---|---|---|---|---|
| No. | Day 1 | 2 | 4 | 8 | 12 | up |
| 1003 | 51.2 | 27.0 | 15.8 | 9.2 | 8.0 | 10.8 |
| 1005 | 61.8 | 39.8 | 39.4 | 56.4 | 27.2 | 55.6 |
| 1006 | 85.6 | 49.8 | 66.4 | 33.4 | 53.4 | 60.0 |
| 1010 | 37.8 | 50.4 | 33.4 | 38.6 | 46.6 | 67.0 |
| 1013 | 41.6 | 34.0 | 33.6 | 26.6 | 27.4 | 22.8 |
| 1015 | 40.0 | 40.6 | 31.4 | 43.6 | 31.8 | 34.2 |
| 1021 | 71.6 | 53.8 | 66.6 | 63.0 | 62.2 | 56.4 |
| 1027 | 48.6 | 17.4 | 13.2 | 11.0 | 16.6 | 13.2 |
| 1032 | 43.2 | 43.2 | 15.0 | 32.0 | 10.4 | 23.0 |
| 1033 | 69.0 | 87.0 | 60.2 | 33.2 | 19.0 | 23.6 |
| 1034 | 77.0 | 31.0 | 14.6 | 7.6 | 4.0 | 6.0 |
| 1035 | 64.0 | 49.0 | 19.6 | 39.2 | 16.4 | 12.2 |
| 1042 | 47.4 | 38.0 | 33.4 | 26.4 | 23.2 | 48.6 |
| 1044 | 59.4 | 40.4 | 38.6 | 43.6 | 38.2 | 35.2 |
| 1045 | 71.2 | 55.4 | 42.2 | 25.0 | 28.0 | 20.8 |
| 1046 | 38.0 | 39.2 | 29.2 | 40.8 | 51.8 | 38.8 |
| 1047 | 37.4 | 31.0 | 8.6 | 6.8 | 7.6 | 8.6 |
| 1054 | 57.6 | 54.2 | 27.6 | 22.2 | 25.6 | 36.8 |
| 1055 | 56.8 | 45.8 | 28.0 | 14.2 | 13.4 | 18.6 |
| 1056 | 51.8 | 31.0 | 22.6 | 28.0 | 12.0 | 10.0 |
| 1066 | 57.0 | 56.0 | 28.8 | 21.4 | 19.4 | 23.8 |
| 1067 | 80.0 | 62.6 | 70.4 | 27.6 | 35.2 | 48.0 |
| 1455 | 76.0 | 73.8 | 41.6 | 33.0 | 23.6 | 24.4 |
| 1457 | 71.0 | 61.0 | 61.2 | 59.4 | 65.0 | 79.2 |
| 1459 | 79.0 | 74.2 | 69.0 | 76.2 | 73.0 | 64.6 |
| 1464 | 68.6 | 11.6 | 46.4 | 50.8 | 61.4 | 53.6 |
| 1468 | 87.8 | 69.2 | 70.0 | 68.6 | 67.6 | 67.0 |
| 1469 | 60.0 | 66.2 | 70.4 | 58.4 | 20.2 | 7.2 |
| 1472 | 76.0 | 76.2 | 67.8 | 84.2 | 49.4 | 55.6 |
| 1478 | 69.8 | 43.0 | 33.4 | 30.6 | 49.0 | 44.0 |
| 1481 | 59.4 | 18.2 | 52.0 | 47.2 | 44.2 | 51.4 |
| 1485 | 51.6 | 47.0 | 50.4 | 47.6 | 60.4 | 55.6 |
| 1489 | 52.0 | 35.8 | 18.8 | 8.2 | 8.0 | 14.0 |
| 1491 | 87.8 | 83.0 | 84.6 | 68.2 | 47.4 | 38.4 |
| 1493 | 65.2 | 59.8 | 47.6 | 56.8 | 53.2 | 64.8 |
| 1497 | 52.0 | 53.0 | 49.0 | 44.4 | 37.2 | 40.2 |
| 1503 | 82.6 | 86.0 | 85.6 | 87.8 | 89.6 | 90.2 |
| 1506 | 79.8 | 22.2 | 2.8 | 1.6 | 4.6 | 4.0 |
| 1511 | 80.8 | 82.4 | 35.2 | 43.4 | 15.2 | 14.2 |
| 1512 | 46.0 | 44.4 | 40.2 | 41.8 | 41.4 | 27.4 |
| 1520 | 56.8 | 35.6 | 21.8 | 26.2 | 40.0 | 36.8 |
| 1524 | 62.2 | 65.4 | 65.4 | 53.4 | 7.0 | 4.2 |
| 1527 | 53.6 | 18.2 | 13.6 | 7.8 | 9.2 | 8.2 |
| 1530 | 68.8 | 72.0 | 64.8 | 52.4 | 41.2 | 55.4 |
| 1534 | 54.4 | 60.4 | 43.8 | 45.8 | 48.2 | 42.4 |
| 1537 | 71.6 | 72.2 | 73.4 | 77.4 | 79.8 | 80.8 |
| 1542 | 72.8 | 45.8 | 66.8 | 43.0 | 35.4 | 19.2 |
| 1543 | 49.0 | 33.0 | 33.0 | 45.8 | 29.8 | 40.2 |
| 9003 | 58.2 | 83.6 | 88.2 | 87.8 | 79.4 | 79.0 |
| 9006 | 37.0 | 5.6 | 3.0 | 4.0 | 1.6 | 3.6 |
| 9009 | 80.6 | 68.0 | 70.0 | 12.2 | 14.2 | 43.8 |
| 9010 | 61.6 | 28.2 | 41.0 | 28.6 | 19.4 | 12.0 |
| 9013 | 52.2 | 52.2 | 47.0 | 22.2 | 36.2 | 33.4 |
| 9017 | 75.2 | 20.6 | 16.0 | 16.0 | 15.8 | 40.6 |
| 9019 | 31.2 | 11.6 | 34.6 | 46.6 | 26.8 | 27.4 |
| 9031 | 5.2 | 8.0 | 6.2 | 6.2 | 5.0 | 4.2 |
| 9032 | 16.6 | 11.2 | 6.2 | 4.4 | 3.2 | 3.0 |
| 9033 | 51.0 | 49.4 | 50.4 | 28.2 | 39.2 | 21.8 |
| 9454 | 57.8 | 44.2 | 24.4 | 9.2 | 17.4 | 16.8 |
| 9467 | 87.2 | 65.6 | 59.6 | 67.4 | 69.8 | 67.6 |
| 9459 | 27.8 | 20.2 | 11.8 | 12.0 | 7.0 | 7.6 |
| 9462 | 36.6 | 46.2 | 56.4 | 49.8 | 56.2 | 56.2 |
| 9465 | 47.2 | 30.0 | 44.8 | 32.6 | 35.6 | 39.2 |
| 1474 | 80.4 | 52.2 | 50.4 | 33.2 | 58.8 | 39.0 |
| Avg. | 59.2 | 46.6 | 41.5 | 37.0 | 33.8 | 35.2 |

TABLE 22
Blinded WOMAC Joint Stiffness Subscale Scores of All
Patients Who Finished 12 Weeks Treatment and Follow-up
Visit at High Dose (8 spray/knee, 35 mg/knee) Cohort

| Patient | | Week | Week | Week | Week | Follow- |
|---|---|---|---|---|---|---|
| No. | Day 1 | 2 | 4 | 8 | 12 | up |
| 1003 | 67.5 | 41.0 | 26.0 | 18.0 | 17.0 | 41.5 |
| 1005 | 58.0 | 37.0 | 33.5 | 60.0 | 21.0 | 55.0 |
| 1006 | 81.5 | 45.5 | 66.5 | 54.0 | 40.0 | 61.5 |
| 1010 | 81.5 | 80.5 | 70.0 | 68.0 | 73.5 | 70.0 |
| 1013 | 43.5 | 32.5 | 28.5 | 19.5 | 19.0 | 24.0 |
| 1015 | 37.0 | 35.0 | 48.5 | 41.0 | 34.5 | 37.0 |
| 1021 | 80.0 | 57.5 | 58.5 | 65.0 | 53.0 | 55.5 |
| 1027 | 55.5 | 43.5 | 19.0 | 19.0 | 28.0 | 17.5 |
| 1032 | 52.0 | 13.0 | 19.0 | 55.5 | 24.0 | 29.0 |
| 1033 | 92.0 | 96.5 | 92.0 | 16.0 | 59.0 | 71.0 |
| 1034 | 87.0 | 52.0 | 16.0 | 12.5 | 13.0 | 14.5 |
| 1035 | 66.0 | 49.0 | 21.5 | 28.5 | 8.0 | 8.5 |
| 1042 | 54.5 | 48.5 | 39.0 | 30.5 | 25.0 | 46.0 |
| 1044 | 50.5 | 35.0 | 35.5 | 58.0 | 31.0 | 42.5 |
| 1045 | 53.0 | 47.5 | 56.5 | 23.0 | 23.0 | 26.5 |
| 1046 | 42.5 | 47.0 | 37.0 | 48.5 | 61.5 | 41.5 |
| 1047 | 24.5 | 46.5 | 12.5 | 8.5 | 5.0 | 13.5 |
| 1054 | 68.0 | 53.0 | 41.5 | 46.0 | 48.0 | 57.0 |
| 1055 | 59.5 | 54.5 | 29.5 | 12.0 | 16.0 | 10.0 |
| 1056 | 48.5 | 45.0 | 44.5 | 43.5 | 23.5 | 17.0 |
| 1066 | 67.5 | 68.0 | 42.0 | 29.0 | 17.5 | 22.5 |
| 1067 | 89.0 | 81.5 | 85.5 | 34.0 | 49.0 | 59.0 |
| 1455 | 74.5 | 68.5 | 52.0 | 31.5 | 23.0 | 25.0 |
| 1457 | 75.5 | 71.0 | 79.0 | 79.0 | 83.5 | 77.0 |
| 1459 | 81.0 | 79.5 | 76.0 | 76.0 | 61.0 | 63.0 |
| 1464 | 65.5 | 14.0 | 54.5 | 67.5 | 68.5 | 59.0 |
| 1468 | 84.0 | 66.5 | 70.5 | 72.5 | 76.5 | 64.5 |
| 1469 | 61.0 | 66.5 | 70.0 | 66.0 | 19.0 | 5.5 |
| 1472 | 86.5 | 81.5 | 82.5 | 89.0 | 68.5 | 41.0 |
| 1478 | 76.5 | 48.5 | 54.0 | 45.5 | 73.5 | 65.0 |
| 1481 | 58.5 | 25.5 | 53.0 | 54.5 | 56.0 | 52.5 |
| 1485 | 57.0 | 45.5 | 43.5 | 53.0 | 57.0 | 50.5 |
| 1489 | 55.5 | 43.0 | 17.0 | 9.5 | 7.0 | 14.0 |
| 1491 | 86.5 | 87.0 | 86.0 | 86.5 | 75.0 | 72.5 |
| 1493 | 64.5 | 60.0 | 53.0 | 67.0 | 62.5 | 55.0 |
| 1497 | 4.0 | 7.5 | 0.0 | 0.0 | 0.0 | 0.0 |
| 1503 | 91.0 | 87.5 | 89.0 | 93.0 | 89.5 | 90.0 |
| 1506 | 83.0 | 17.5 | 3.5 | 15.5 | 17.0 | 11.5 |
| 1511 | 78.0 | 90.5 | 29.5 | 44.5 | 13.5 | 13.5 |
| 1512 | 54.5 | 54.5 | 44.0 | 45.5 | 50.0 | 21.0 |
| 1520 | 64.0 | 36.0 | 25.0 | 27.5 | 38.0 | 39.5 |
| 1524 | 84.5 | 71.0 | 63.0 | 15.0 | 4.0 | 3.5 |
| 1527 | 57.0 | 20.5 | 16.0 | 14.5 | 10.5 | 8.0 |
| 1530 | 73.5 | 65.5 | 63.5 | 70.5 | 39.0 | 41.5 |
| 1534 | 57.0 | 61.5 | 53.0 | 54.5 | 46.5 | 50.5 |
| 1537 | 73.5 | 75.0 | 78.0 | 80.0 | 80.0 | 81.5 |
| 1542 | 78.0 | 75.0 | 52.0 | 55.5 | 42.0 | 55.5 |
| 1543 | 39.6 | 45.0 | 28.5 | 35.5 | 30.5 | 34.5 |
| 9003 | 60.0 | 85.5 | 89.0 | 86.0 | 81.0 | 84.5 |
| 9006 | 60.5 | 4.0 | 2.0 | 2.5 | 1.0 | 3.5 |
| 9009 | 87.0 | 67.0 | 46.0 | 17.0 | 15.5 | 52.0 |
| 9010 | 58.0 | 41.0 | 39.0 | 29.0 | 40.5 | 23.0 |
| 9013 | 51.5 | 44.0 | 43.0 | 18.5 | 35.5 | 28.5 |
| 9017 | 72.0 | 31.5 | 13.5 | 11.5 | 15.0 | 34.0 |
| 9019 | 51.5 | 53.5 | 49.5 | 43.0 | 50.5 | 27.0 |
| 9031 | 2.5 | 2.0 | 5.0 | 2.0 | 2.5 | 3.0 |
| 9032 | 14.5 | 9.5 | 5.0 | 3.0 | 6.0 | 9.5 |
| 9033 | 39.0 | 67.5 | 48.5 | 27.5 | 38.5 | 25.5 |
| 9454 | 52.5 | 42.0 | 31.5 | 17.0 | 6.5 | 19.0 |
| 9467 | 92.0 | 72.5 | 60.0 | 72.5 | 77.0 | 63.5 |
| 9459 | 27.5 | 32.5 | 14.5 | 6.5 | 6.5 | 8.0 |
| 9462 | 44.5 | 62.5 | 65.5 | 73.0 | 71.0 | 65.5 |
| 9465 | 55.0 | 47.0 | 52.0 | 42.0 | 43.0 | 44.5 |
| 1474 | 84.0 | 14.0 | 25.5 | 11.5 | 65.5 | 7.5 |
| Avg. | 62.1 | 50.8 | 44.5 | 40.6 | 38.1 | 37.7 |

TABLE 23
Blinded WOMAC Difficulty Performing Daily Activities Subscale
Scores of All Patients Who Finished 12 Weeks Treatment and
Follow-up Visit at High Dose (8 spray/knee, 35 mg/knee) Cohort

| Patient | | Week | Week | Week | Week | Follow- |
|---|---|---|---|---|---|---|
| No. | Day 1 | 2 | 4 | 8 | 12 | up |
| 1003 | 54.9 | 29.5 | 13.9 | 10.2 | 12.6 | 24.2 |
| 1005 | 59.2 | 44.8 | 39.6 | 58.4 | 29.8 | 62.1 |
| 1006 | 89.2 | 59.8 | 68.1 | 63.9 | 67.0 | 74.8 |
| 1010 | 64.5 | 64.4 | 68.7 | 46.5 | 61.8 | 62.7 |
| 1013 | 47.1 | 34.0 | 31.8 | 27.1 | 26.3 | 25.8 |
| 1015 | 33.5 | 39.9 | 32.2 | 39.4 | 34.1 | 37.6 |
| 1021 | 68.4 | 50.2 | 64.3 | 61.1 | 51.6 | 59.9 |
| 1027 | 36.2 | 30.2 | 11.6 | 8.9 | 12.4 | 23.2 |
| 1032 | 43.0 | 9.7 | 12.7 | 22.6 | 21.6 | 17.1 |
| 1033 | 54.4 | 72.5 | 56.9 | 14.6 | 9.9 | 16.5 |
| 1034 | 64.2 | 30.7 | 14.2 | 8.5 | 5.5 | 6.7 |
| 1035 | 64.2 | 49.5 | 26.8 | 36.2 | 13.2 | 12.4 |
| 1042 | 39.4 | 41.7 | 28.0 | 23.8 | 28.3 | 19.6 |
| 1044 | 43.9 | 44.0 | 45.7 | 48.1 | 44.1 | 38.4 |
| 1045 | 54.4 | 46.2 | 47.2 | 20.8 | 22.3 | 26.4 |
| 1046 | 42.2 | 43.7 | 33.8 | 43.5 | 49.4 | 41.1 |
| 1047 | 44.6 | 29.7 | 17.0 | 9.7 | 6.1 | 10.3 |
| 1054 | 56.2 | 51.4 | 35.5 | 35.1 | 38.8 | 44.6 |
| 1055 | 49.4 | 52.6 | 33.9 | 15.6 | 13.0 | 16.6 |
| 1056 | 39.6 | 28.9 | 24.3 | 25.2 | 18.1 | 11.7 |
| 1066 | 67.9 | 59.5 | 28.8 | 24.4 | 20.4 | 28.8 |
| 1067 | 89.0 | 78.0 | 78.9 | 36.9 | 44.4 | 57.5 |
| 1455 | 75.4 | 72.1 | 39.2 | 32.6 | 21.4 | 24.5 |
| 1457 | 58.3 | 61.4 | 64.2 | 66.2 | 68.9 | 64.8 |
| 1459 | 84.7 | 81.9 | 75.0 | 77.5 | 76.8 | 77.4 |
| 1464 | 68.4 | 15.0 | 40.5 | 54.9 | 51.5 | 49.7 |
| 1468 | 71.9 | 71.8 | 71.1 | 67.8 | 75.9 | 72.4 |
| 1469 | 60.3 | 63.4 | 64.4 | 65.5 | 20.9 | 7.6 |
| 1472 | 83.4 | 80.4 | 78.6 | 87.4 | 57.8 | 49.3 |
| 1478 | 68.9 | 51.5 | 46.6 | 46.4 | 60.3 | 59.6 |
| 1481 | 62.4 | 22.0 | 56.7 | 55.6 | 55.3 | 50.8 |
| 1485 | 54.5 | 49.4 | 51.8 | 54.9 | 59.6 | 61.6 |
| 1489 | 55.8 | 34.9 | 17.9 | 8.1 | 6.6 | 14.2 |
| 1491 | 90.2 | 84.2 | 85.6 | 73.2 | 67.2 | 60.2 |
| 1493 | 65.1 | 64.9 | 55.3 | 61.2 | 62.8 | 54.9 |
| 1497 | 24.4 | 29.6 | 45.2 | 13.9 | 28.2 | 25.1 |
| 1503 | 91.1 | 86.1 | 88.2 | 92.3 | 90.3 | 90.6 |
| 1506 | 33.1 | 15.9 | 3.2 | 4.2 | 10.7 | 6.2 |
| 1511 | 74.0 | 87.2 | 29.0 | 43.3 | 15.3 | 15.2 |
| 1512 | 48.5 | 48.4 | 43.4 | 44.4 | 42.2 | 25.9 |
| 1520 | 62.9 | 38.7 | 30.4 | 30.8 | 34.5 | 40.2 |
| 1524 | 63.1 | 64.2 | 64.1 | 22.3 | 4.8 | 3.4 |
| 1527 | 39.9 | 15.6 | 11.6 | 8.3 | 8.1 | 7.9 |
| 1530 | 76.4 | 67.4 | 60.4 | 54.7 | 48.7 | 56.8 |
| 1534 | 57.9 | 62.1 | 44.6 | 49.8 | 46.9 | 50.5 |
| 1537 | 74.5 | 77.5 | 78.7 | 79.5 | 83.5 | 85.6 |
| 1542 | 76.9 | 56.6 | 53.4 | 46.8 | 43.4 | 31.4 |
| 1543 | 54.1 | 43.1 | 33.9 | 37.9 | 33.4 | 32.2 |
| 9003 | 60.1 | 88.7 | 90.4 | 86.2 | 79.2 | 79.5 |
| 9006 | 31.2 | 3.5 | 2.6 | 1.6 | 1.8 | 4.2 |
| 9009 | 81.2 | 64.2 | 68.0 | 16.2 | 19.3 | 52.0 |
| 9010 | 48.6 | 26.3 | 33.2 | 30.1 | 24.9 | 12.6 |
| 9013 | 61.9 | 52.8 | 47.8 | 37.5 | 43.4 | 37.9 |
| 9017 | 67.6 | 23.1 | 16.6 | 21.4 | 17.5 | 25.0 |
| 9019 | 38.8 | 25.2 | 51.2 | 49.2 | 49.3 | 33.4 |
| 9031 | 2.8 | 1.9 | 3.2 | 3.7 | 5.2 | 3.2 |
| 9032 | 16.4 | 6.0 | 7.1 | 3.4 | 2.5 | 2.8 |
| 9033 | 53.9 | 54.5 | 60.2 | 26.7 | 44.7 | 28.1 |
| 9454 | 44.6 | 39.7 | 20.9 | 7.8 | 13.4 | 20.1 |
| 9467 | 84.4 | 67.2 | 63.5 | 67.9 | 72.4 | 70.8 |
| 9459 | 25.8 | 21.7 | 13.1 | 10.8 | 7.6 | 8.2 |
| 9462 | 49.9 | 57.9 | 63.2 | 65.4 | 63.3 | 64.8 |
| 9465 | 52.4 | 40.5 | 47.0 | 44.2 | 38.8 | 42.6 |
| 1474 | 73.7 | 64.2 | 80.5 | 62.4 | 45.5 | 49.2 |
| Avg. | 57.4 | 48.0 | 44.0 | 39.0 | 36.5 | 37.0 |

However, after the database lock, when we reviewed the top-line study results from the CRO, we noticed a number of abnormalities in the datasets as follows:
1. PK Data: Some patients assigned to receive the placebo had comparable high plasma ibuprofen concentrations as those of patients treated with active drug; and vice versa some patients on active drug had no plasma ibuprofen concentrations more than the blank background (see Tables 24 and 25).
2. Efficacy Endpoints: For the change from baseline in the WOMAC (VAS) pain subscale score for the target knee of treatment, in group A (8.75 mg/knee) patients that received placebo apparently had much better response than patients on active drug at 2, 4, 8 and 12 (EOS) weeks, respectively (Table 26), but in group C(35 mg/knee), patients that received active drug apparently had much better response than patients on placebo at 2, 4, 8 and 12 (EOS) weeks (Table 27). Normally, the placebo effects should be similar in all groups, or high dose group has higher placebo effects, but the data shown the low group has much higher placebo effects (more than double in the first 4 weeks).
3. Placebo Effects: For OA trials, typically placebo effects reach the maximum at week 2-4 then the placebo effects diminish after 4 weeks. However in this trial, placebo effects increased from week 2 to week 12.

TABLE 24
Plasma Ibuprofen Concentrations of
Some Patients Treated with Placebo

| | Week 2 | Week 3 | Cₘₐₓ | Cₘₐₓ |
|---|---|---|---|---|
| | (hour 0) | (hour 0) | (week 4) | (week 12) |
| Subjects | (ng/mL) | (ng/mL) | (ng/mL) | (ng/mL) |
| 9001 | No blood drawn | No blood drawn | <0.500 | <0.500 |
| 9002 | No blood drawn | No blood drawn | 1.47 | 1.65 |
| 9008 | 129 | 38.8 | Drop out | Drop out |
| 9009 | 1.02 | 0.695 | 1.63 | 0.951 |
| 9010 | 23.2 | 34.0 | 27.6 | 21.6 |
| 9012 | <0.500 | 0.582 | <0.500 | <0.500 |
| 9013 | 5.87 | 5.78 | 8.85 | 9.29 |
| 9019 | 32.1 | 2.82 | 9.82 | 24.1 |
| 9021 | 751 | 34.6 | 10.8 | 68.9 |
| 9025 | 4.07 | 2.87 | 1.61 | 2.51 |
| 9028 | 2.42 | 1.37 | 6.4 | 4.99 |
| 9033 | 1.95 | 9.41 | 4.63 | 11.3 |
| 9036 | <0.500 | <0.500 | <0.500 | <0.500 |
| 9453 | 15.6 | 15.2 | 15.1 | 8.31 |
| 9455 | 13.9 | 54.0 | 24.5 | 13.7 |
| 9459 | 0.657 | <0.500 | <0.500 | 0.719 |
| 9461 | 0.621 | 1.59 | 3.12 | 3.42 |
| 9462 | 3.52 | 3.85 | 19.6 | 4.44 |

TABLE 25
Plasma Ibuprofen Concentrations of Some Patients Treated
with 2-(diethylamino)ethyl 2-(4-isobutylphenyl)propionate

| | Week 2 | Week 3 | | |
|---|---|---|---|---|
| | (hour 0) | (hour 0) | Cₘₐₓ(week 4) | Cₘₐₓ(week 12) |
| Subjects | (ng/mL) | (ng/mL) | (ng/mL) | (ng/mL) |
| 9004 | 3.87 | 5.59 | 8.72 | 4.93 |
| 9014 | 8.78 | 8.42 | 8.42 | 16.1 |
| 9016 | No result | 8.63 | 14.3 | Drop out |
| 9018 | 5.91 | 6.34 | 10.4 | 43.8 |
| 9020 | 4.43 | 5.69 | 5.69 | 13.3 |
| 9023 | 4.47 | 1.66 | 11.2 | 9.61 |
| 9451 | 7.46 | 3.38 | 11.8 | 12.0 |
| 9460 | 377 | 3.81 | 3.77 | 6.05 |

TABLE 26
Efficacy Analysis of Change from Baseline in the WOMAC Pain Subscale Scores (Group: A)

| | | | | | difference of | | |
|---|---|---|---|---|---|---|---|
| | LS mean^{b} (95% ci)^{c} | | | | LS mean | (95% CI) for | |
| Visit | n^{a} | 8.75 mg/knee, bid | n^{a} | placebo, bid | 8.75 mg-control) | the Difference^{d} | P-value^{e} |
| Week 2^{f} | 33 | −7.5 (−13.3, −1.7) | 20 | −14.2 (−21.8, −6.6) | 6.7 | (−2.8, 16.2) | 0.1654 |
| Week 4^{f} | 38 | −10.6 (−17.1, −4.1) | 18 | −21.0 (−29.9, −12.1) | 10.5 | (−0.6, 21.5) | 0.0635 |
| Week 8^{f} | 37 | −15.7 (−22.5, −8.8) | 19 | −23.6 (−32.9, −14.3) | 7.9 | (−3.6, 19.5) | 0.1721 |
| Week 12/ | 35 | −16.1 (−23.6, −8.6) | 19 | −24.7 (−34.8, −14.5) | 8.6 | (−4.0, 21.2) | 0.1771 |
| EOS^{f} | | | | | | | |

EOS = End of study,
BID = twice a day;
WOMAC = Western Ontario and McMaster Universities
Model: Change from baseline in score = Baseline score + Visit + Treatment + Visit * Treatment + Subject + Random Error
^{a}n was the number of subjects.
^{b}Least squares means from ANCOVA.
^{c}95% confidence interval for the least squares means.
^{d}95% confidence interval for the difference of least squares means.
^{e}P-value from two-tailed t-test obtained from repeated measures analyses by using mixed model.
^{f}Scale ranged from 0 to 100 mm with negative change indicating improvement

TABLE 27
Efficacy Analysis of Change from Baseline in the WOMAC Pain Subscale Scores (Group: C)

| | | | | | | | difference of LS | (95% CI) | |
|---|---|---|---|---|---|---|---|---|---|
| | | LS mean^{b} (95% ci)^{c} | | | | | mean testing | for the | |
| Visit | n^{a} | 35 mg/knee, bid | | n^{a} | placebo, bid | | Drug-control) | Difference^{d} | P-value^{e} |
| Week 2^{f} | 39 | −15.6 | (−20.8, −10.5) | 14 | −4.1 | (−12.6, 4.3) | −11.5 | (−21.4, −1.6) | 0.0238 |
| Week 4^{f} | 40 | −21.2 | (−26.9, −15.5) | 13 | −8.4 | (−17.8, 1.1) | −12.8 | (−23.8, −1.7) | 0.0240 |
| Week 8^{f} | 39 | −24.0 | (−30.7, −17.4) | 14 | −18.1 | (−28.9, −7.2) | −6.0 | (−18.7, 6.7) | 0.3496 |
| Week 12/ | 39 | −26.9 | (−33.8, −20.0) | 16 | −22.8 | (−33.7, −11.8) | −4.1 | (−17.1, 5.5) | 0.5274 |
| EOS^{f} | | | | | | | | | |

EOS = End of study, BID = twice a day; WOMAC = Western Ontario and McMaster Universities
Model: Change from baseline in score = Baseline score + Treatment + Visit*Treatment + Subject + Random Error
^{a}n was the number of subjects.
^{b}Least squares means from ANOVA.
^{c}95% confidence interval for the least squares means.
^{d}95% confidence interval for the difference of least squares means.
^{e}P-value from two-tailed t-test obtained from repeated measured analyses by using mixed model.
^{f}Scale ranged from 0 to 100 mm with negative change indicating improvement

Patient Baseline Characteristics are shown in Table 28.

TABLE 28
Patient Baseline Characteristics

| | 2 Sprays/knee | 4 Sprays/knee | 8 Sprays/knee |
|---|---|---|---|
| | (8.75 mg/knee, | (17.5 mg/knee, | (35 mg/knee, |
| Characteristics | BID) (n = 62) | BID) (n = 64) | BID) (n = 64) |
| Age, y, Mean ± SD | 64.2 ± 8.7 | 65.2 ± 9.3 | 64.9 ± 9.5 |
| Female % | 77.3% | 79.1% | 78.1% |
| WOMAC Pain Subscales Scores* (±SD) | 59.8(±15.6) | 61.0(±17.1) | 59.2(±17.7) |
| WOMAC Joint Stiffness Subscales | 64.2(±19.0) | 68.0(±15.3) | 62.1(±20.5) |
| Scores* (±SD) | | | |
| WOMAC Difficulty Performing Daily | 60.4(±17.5) | 63.4(±14.8) | 57.4(±18.6) |
| Activities Subscales Scores* (±SD) | | | |
| Mean ± SEM WOMAC Osteoarthritis | 59.5(±16.3) | 64.2(±14.1) | 59.5(±18.0) |
| Index Composite Score Change | | | |
| from Baselines* (±SD) | | | |
| Subject's Global Assessment of | 59.5(±18.8) | 58.0(±20.6) | 56.0(±20.5) |
| Disease Status* (±SD) | | | |

*Scale ranged from 0-100 with lower score as better

Taking together the aforementioned abnormalities of the top-line PK and efficacy data, it is very unlikely that the placebo effects could have such markedly and sustained efficacy even better than the active drug. Therefore we suspect that the active drug (2-(diethylamino)ethyl 2-(4-isobutylphenyl)propionate) and placebo drug were mixed up in some manner.

Despite the mixing up active drug and placebo in some patients on this study, the robustness of efficacy of 2-(diethylamino)ethyl 2-(4-isobutylphenyl)propionate was clearly demonstrated in this Phase 2 study in OA patients in a dose-response manner.

To determine this, we assumed that all patients received 2-(diethylamino)ethyl 2-(4-isobutylphenyl)propionate, active drug treatment (i.e. all patients who received 2-(diethylamino)ethyl 2-(4-isobutylphenyl)propionate or placebo treatment for 12 weeks or mixed up treatment during the 12-week treatment, as 2-(diethylamino)ethyl 2-(4-isobutylphenyl)propionate-treated). Based on this assumption, that is, to treat this trial as an open-label study and that all patients received active treatment, we conducted efficacy analysis using top-line efficacy datasets prepared by CRO and the re-analysis results are presented in the following tables (Tables 29-47). The re-analysis of the efficacy data demonstrated dose-response and was better to the efficacy of currently marketed drugs such as naproxen or celecoxib (See Table 29 for Celecoxib and Naproxen data). (William G. Bensen, Justus J. Fiechtner, James 1. McMillen, et. al. Treatment of Osteoarthritis with Celecoxib. Mayo Clin Proc, November 1999. Vol 74, 1095-1105.)

TABLE 29
Effect of Treatment on Sign and Symptoms of Osteoarthritis
with placebo, Celecoxib and Naproxen orally at 12 Weeks

| | | Celecoxib | Celecoxib | Celecoxib | Naproxen |
|---|---|---|---|---|---|
| | Placebo | (50 mg, BID) | (100 mg, BID) | (200 mg, BID) | (500 mg, BID) |
| | (n = 203) | (n = 203) | (n = 197) | (n = 202) | (n = 198) |
| Mean ± SEM WOMAC | −6.1 ± 1.09 | −9.5 ± 1.11 | −13.3 ± 1.17 | −12.0 ± 1.22 | −11.9 ± 1.29 |
| Osteoarthritis Index | | | | | |
| Composite Score Change | | | | | |
| from Baselines* | | | | | |

*Scale ranged from 0 to 96 with negative change indicating improvement.

As shown in Table 29, the difference from placebo for Celecoxib (50 mg, BID), Celecoxib (100 mg, BID), Celecoxib (200 mg, BID), and Naproxen (500 mg, BID) are only −3.4, −7.2, −5.9 and −5.8 at Week 12, the best result is −7.2 for Celecoxib (100 mg, BID).

TABLE 30
Effect of Treatment on Sign and Symptoms of Osteoarthritis with 2-(diethylamino)ethyl
2-(4-isobutylphenyl)propionate Transdermally at 12 Weeks

| Mean ± SEM WOMAC Osteoarthritis | 2 sprays/knee | 4 sprays/knee | 8 sprays/knee |
|---|---|---|---|
| Index Composite Score Change from | (8.75 mg, BID) | (17.5 mg, BID) | (35 mg, BID) |
| Baselines* | (n = 54) | (n = 57) | (n = 55) |
| Week 2 | −11.1 ± 2.50 | −15.1 ± 2.89 | −11.7 ± 2.88 |
| Week 4 | −18.0 ± 2.50 | −21.3 ± 3.32 | −17.0 ± 2.96 |
| Week 8 | −18.1 ± 2.92 | −24.8 ± 3.26 | −21.7 ± 3.28 |
| Week 12 | −19.5 ± 3.18 | −28.9 ± 3.38 | −23.0 ± 3.27 |
| Follow-up (Week13) | −18.4 ± 3.20 | −23.4 ± 3.52 | −22.1 ± 3.23 |

*Scale ranged from 0 to 100 with negative change indicating improvement.

As shown in Table 30, if there is same placebo effect as the oral Celebrex −6.1, then the difference from placebo for transdermal 2-(diethylamino)ethyl 2-(4-isobutylphenyl)propionate (8.75 mg, BID), 2-(diethylamino)ethyl 2-(4-isobutylphenyl)propionate (17.5 mg, BID), and 2-(diethylamino)ethyl 2-(4-isobutylphenyl)propionate (35 mg, BID) is −13.4, −22.8, and −16.9. The best result for 2-(diethylamino)ethyl 2-(4-isobutylphenyl)propionate is −22.8 (17.5 mg, BID) and 3 times of Celecoxib (100 mg, BID).

In this clinical study, the ratio of 2-(diethylamino)ethyl 2-(4-isobutylphenyl)propionate:placebo is 2800:2000=58.3:41.7. If the data are adjusted by 58.3% of the testing drug. The adjusted data is shown in Table 31.

TABLE 31
Effect of Treatment on Sign and Symptoms of Osteoarthritis with
2-(diethylamino)ethyl 2-(4-isobutylphenyl)propionate Transdermally
at 12 Weeks (adjusted by 58.3% of the testing drug amount)

| Mean ± SEM WOMAC Osteoarthritis | 2 sprays/knee | 4 sprays/knee | 8 sprays/knee |
|---|---|---|---|
| Index Composite Score Change from | (8.75 mg, BID) | (17.5 mg, BID) | (35 mg, BID) |
| Baselines* | (n = 54) | (n = 57) | (n = 55) |
| Week 2 | −19.0 ± 4.29 | −25.9 ± 4.96 | −20.1 ± 4.94 |
| Week 4 | −30.9 ± 4.29 | −36.5 ± 5.69 | −29.2 ± 5.08 |
| Week 8 | −31.0 ± 5.01 | −42.5 ± 5.59 | −37.2 ± 5.63 |
| Week 12 | −33.4 ± 5.45 | −49.6 ± 5.80 | −39.5 ± 5.61 |
| Follow-up (Week 13) | −31.6 ± 5.49 | −40.1 ± 6.04 | −37.9 ± 5.54 |

*Scale ranged from 0 to 100 with negative change indicating improvement.

The best result for 2-(diethylamino)ethyl 2-(4-isobutylphenyl)propionate is −43.5 (17.5 mg, BID) and 6 times of Celecoxib (100 mg, BID).

Changes in WOMAC pain subscale scores and percentage of improvement from baseline are respectively shown in FIG. 14 and FIG. 15, and are shown in Table 32.

TABLE 32
Efficacy Analysis of Change from Baseline in the WOMAC* Pain Subscale Scores

| | | 8.75 mg/knee | 17.5 mg/knee | 35 mg/knee |
|---|---|---|---|---|
| Characteristic | Time | (BID*) | (BID) | (BID) |
| Change of | Week 2 (day 14) | −11.2(±2.39) | −15.0(±2.18) | −12.6(±2.33) |
| WOMAC pain | Week 4 (day 28) | −17.0(±2.79) | −20.5(±2.79) | −17.7(±2.54) |
| score from | Week 8 (day 56) | −21.4(±2.81) | −25.1(±2.88) | −22.2(±2.97) |
| baseline | Week 12 (day 84) | −21.6(±3.13) | −26.5(±2.94) | −25.4(±3.04) |
| (±SEM)*** | Follow up (week 13, day 91, 7 days | −20.1(±3.25) | −22.1(±3.01) | −24.0(±3.09) |
| (mm) | after the treatment was stopped) | | | |
| Percentage of | Week 2 (day 14) | −18.7% | −24.6% | −21.3% |
| improvement | Week 4 (day 28) | −28.4% | −33.6% | −29.9% |
| from baseline | Week 8 (day 56) | −35.8% | −41.1% | −37.5% |
| | Week 12 (day 84) | −36.1% | −43.4% | −42.9% |
| | Follow up (Week 13, day 91, 7 days | −33.6% | −36.2% | −40.5% |
| | after the treatment was stopped) | | | |

*BID = twice a day; WOMAC = Western Ontario and McMaster Universities.
**Scale ranged from 0 to 100 mm with lower score as better.
***Negative numbers mean improved.

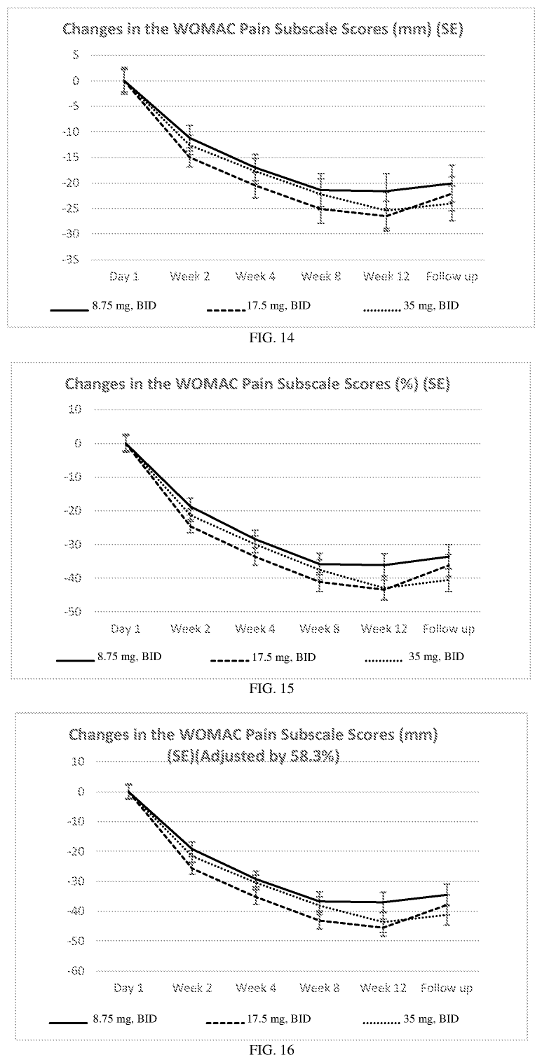

Changes in WOMAC pain subscale scores and percentage of improvement from baseline, both of which are adjusted by 58.3%, are respectively shown in FIG. 16 and FIG. 17, and are shown in Table 33.

TABLE 33
Efficacy Analysis of Change from Baseline in the WOMAC* Pain Subscale
Scores (adjusted by 58.3% of the Testing drug amount)

| | | 8.75 mg/knee | 17.5 mg/knee | 35 mg/knee |
|---|---|---|---|---|
| Characteristic | Time | (BID*) | (BID) | (BID) |
| Change of | Week 2 (day 14) | −19.2(±4.1) | −25.7(±3.7) | −21.6(±4.0) |
| WOMAC pain | Week 4 (day 28) | −29.2(±4.8) | −35.2(±4.8) | −30.4(±4.4) |
| score from | Week 8 (day 56) | −36.7(±4.8) | −43.1(±4.9) | −38.1(±5.1) |
| baseline | Week 12 (day 84) | −37.0(±5.4) | −45.5(±5.0) | −43.6(±5.2) |
| (±SEM)*** | Follow up (week 13, day 91, 7 days | −34.5(±5.6) | −37.9(±5.2) | −41.2(±5.3) |
| | after the treatment was stopped) | | | |
| Percentage of | Week 2 (day 14) | −32.1% | −42.2% | −36.5% |
| improvement | Week 4 (day 28) | −48.7% | −57.6% | −51.2% |
| from baseline | Week 8 (day 56) | −61.4% | −70.5% | −64.3% |
| | Week 12 (day 84) | −61.9% | −74.4% | −73.6% |
| | Follow up (week 13, day 91, 7 days | −57.6% | −62.1% | −69.5% |
| | after the treatment was stopped) | | | |

*BID = twice a day; WOMAC = Western Ontario and McMaster Universities.
**Scale ranged from 0 to 100 mm with lower score as better.
***Negative numbers mean improved.

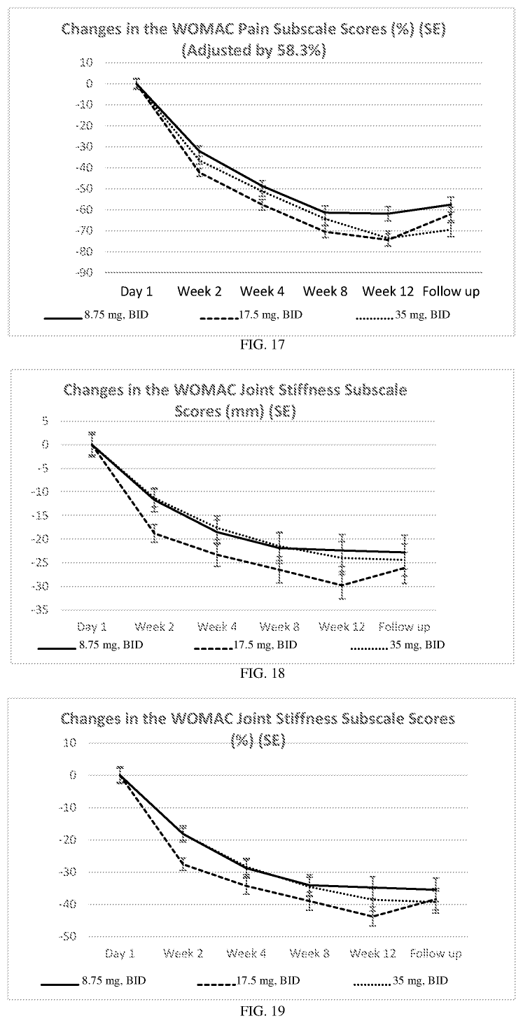

Changes in WOMAC joint stiffness subscale scores and percentage of improvement from baseline, are respectively shown in FIG. 18 and FIG. 19, and are shown in Table 34.

TABLE 34
Efficacy Analysis of Change from Baseline in the WOMAC* Joint Stiffness Subscale Scores

| | | 8.75 mg/knee | 17.5 mg/knee | 35 mg/knee |
|---|---|---|---|---|
| Characteristic | Time | (BID*) | (BID) | (BID) |
| Change of | Week 2 (day 14) | −11.7(±2.5) | −18.8(±2.5) | −11.3(±2.7) |
| WOMAC | Week 4 (day 28) | −18.5(±3.3) | −23.3(±3.0) | −17.6(±2.9) |
| Joint Stiffness | Week 8 (day 56) | −21.9(±3.5) | −26.5(±3.1) | −21.5(±3.4) |
| score from | Week 12 (day 84) | −22.4(±3.1) | −29.8(±3.4) | −24.0(±3.3) |
| baseline | Follow up (week 13, day 91, 7 days | −22.8(±3.7) | −26.1(±3.6) | −24.4(±3.1) |
| (±SEM)*** | after the treatment was stopped) | | | |
| Percentage of | Week 2 (day 14) | −18.2% | −27.6% | −18.2% |
| improvement | Week 4 (day 28) | −28.8% | −34.3% | −28.3% |
| from baseline | Week 8 (day 56) | −34.1% | −39.0% | −34.6% |
| | Week 12 (day 84) | −34.8% | −43.8% | −38.6% |
| | Follow up (week 13, day 91, 7 days | −35.5% | −38.4% | −39.3% |
| | after the treatment was stopped) | | | |

*BID = twice a day; WOMAC = Western Ontario and McMaster Universities.
**Scale ranged from 0 to 100 mm with lower score as better.
***Negative numbers mean improved.

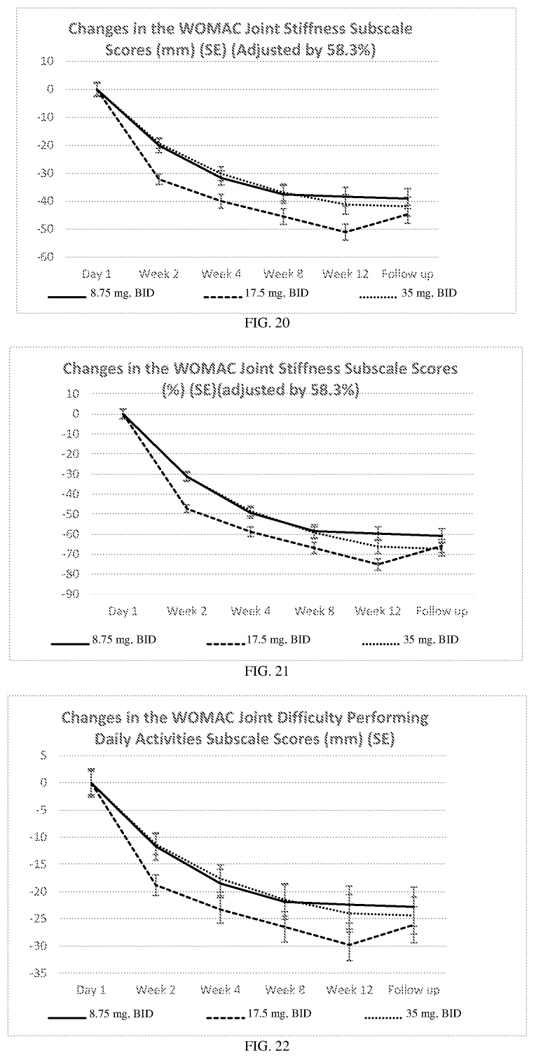

Changes in WOMAC joint stiffness subscale scores and percentage of improvement from baseline, both of which are adjusted by 58.3%, are respectively shown in FIG. 20 and FIG. 21, and are shown in Table 35.

TABLE 35
Efficacy Analysis of Change from Baseline in the WOMAC* Joint Stiffness
Subscale Scores adjusted by 58.3% of the Testing Drug Amount.

| | | 8.75 mg/knee | 17.5 mg/knee | 35 mg/knee |
|---|---|---|---|---|
| Characteristic | Time | (BID*) | (BID) | (BID) |
| Change of | Week 2 (day 14) | −20.1(±4.3) | −32.2(±4.3) | −19.3(±4.6) |
| WOMAC | Week 4 (day 28) | −31.7(±5.7) | −40.0(±5.1) | −30.2(±5.0) |
| Joint Stiffness | Week 8 (day 56) | −37.6(±6.0) | −45.5(±5.3) | −36.9(±5.8) |
| score from | Week 12 (day 84) | −38.4(±5.3) | −51.1(±5.8) | −41.2(±5.7) |
| baseline | Follow up (week 13, day 91, 7 days | −39.1(±5.1) | −44.7(±6.2) | −41.9(±5.3) |
| (±SEM)*** | after the treatment was stopped) | | | |
| Percentage of | Week 2 (day 14) | −31.2% | −47.3% | −31.2% |
| improvement | Week 4 (day 28) | −49.4% | −58.8% | −48.5% |
| from baseline | Week 8 (day 56) | −58.5% | −66.9% | −59.3% |
| | Week 12 (day 84) | −59.7% | −75.1% | −66.2% |
| | Follow up (week 13, day 91, 7 days | −60.9% | −65.9% | −67.4% |
| | after the treatment was stopped) | | | |

*BID = twice a day; WOMAC = Western Ontario and McMaster Universities.
**Scale ranged from 0 to 100 mm with lower score as better.
***Negative numbers mean improved.

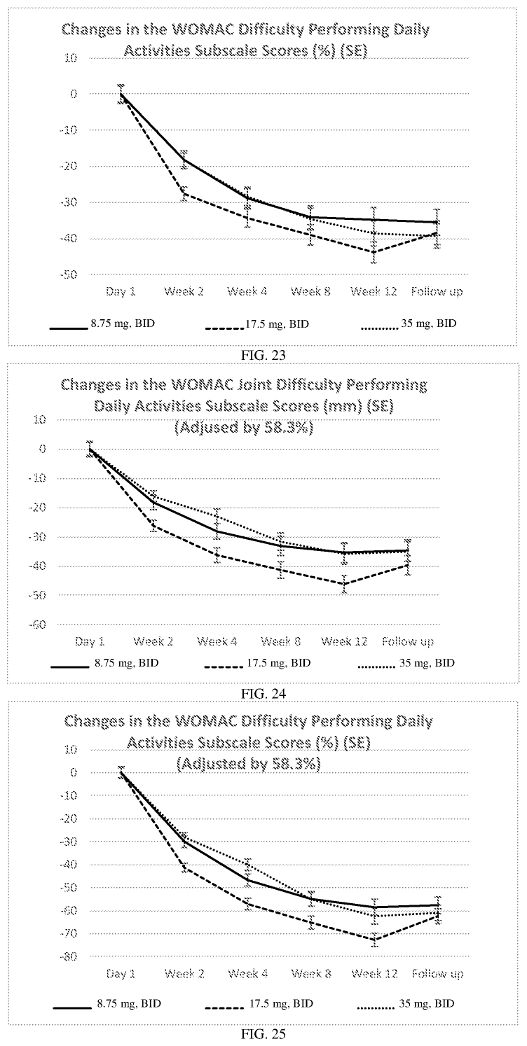

Changes in WOMAC difficulty performing daily activities subscale scores and percentage of improvement from baseline, are respectively shown in FIG. 22 and FIG. 23, and are shown in Table 36.

TABLE 36
Efficacy Analysis of Change from Baseline in the WOMAC*
Difficulty Performing Daily Activities Subscale Scores

| | | 8.75 mg/knee | 17.5 mg/knee | 35 mg/knee |
|---|---|---|---|---|
| Characteristic | Time | (BID) | (BID) | (BID) |
| Change of WOMAC | Week 2 (day 14) | −10.6(±2.6) | −15.3(±2.4) | −9.4(±1.9) |
| Difficulty | Week 4 (day 28) | −16.4(±3.0) | −21.1(±2.8) | −13.4(±2.3) |
| performing daily | Week 8 (day 56) | −19.3(±3.1) | −24.1(±3.0) | −18.4(±2.6) |
| activities score | Week 12 (day 84) | −20.6(±3.4) | −26.9(±3.2) | −20.9(±2.8) |
| from baseline | Follow up (week 13, day 91, 7 days | −20.3(±3.4) | −23.1(±3.2) | −20.4(±2.6) |
| (±SEM)*** | after the treatment was stopped) | | | |
| Percentage of | Week 2 (day 14) | −17.5% | −24.1% | −16.4% |
| improvement | Week 4 (day 28) | −27.2% | −33.3% | −23.3% |
| from baseline | Week 8 (day 56) | −32.0% | −38.0% | −32.1% |
| | Week 12 (day 84) | −34.1% | −42.4% | −36.4% |
| | Follow up (week 13, day 91, 7 days | −33.6% | −36.4% | −35.5% |
| | after the treatment was stopped) | | | |

*BID = twice a day; WOMAC = Western Ontario and McMaster Universities.
**Scale ranged from 0 to 100 mm with lower score as better.
***Negative numbers mean improved.

Changes in WOMAC difficulty performing daily activities subscale scores and percentage of improvement from baseline, both of which are adjusted by 58.3%, are respectively shown in FIG. 24 and FIG. 25, and are shown in Table 37.

TABLE 37
Efficacy Analysis of Change from Baseline in the WOMAC* Difficulty Performing
Daily Activities Subscale Scores adjusted by 58.3% of the Testing Drug Amount.

| | | 8.75 mg/knee | 17.5 mg/knee | 35 mg/knee |
|---|---|---|---|---|
| Characteristic | Time | (BID) | (BID) | (BID) |
| Change of WOMAC | Week 2 (day 14) | −18.2(±4.4) | −26.2(±4.1) | −16.1(±3.3) |
| Difficulty | Week 4 (day 28) | −28.1(±5.1) | −36.2(±4.8) | −23.0(±3.9) |
| performing daily | Week 8 (day 56) | −33.1(±5.3) | −41.3(±5.1) | −31.6(±4.5) |
| activities score | Week 12 (day 84) | −35.3(±5.8) | −46.1(±5.5) | −35.8(±4.8) |
| from baseline | Follow up (week 13, day 91, 7 days | −34.6(±5.8) | −39.6(±5.5) | −35.0(±4.5) |
| (±SEM)*** | after the treatment was stopped) | | | |
| Percentage of | Week 2 (day 14) | −30.0% | −41.3% | −28.1% |
| improvement | Week 4 (day 28) | −46.7% | −57.1% | −40.0% |
| from baseline | Week 8 (day 56) | −54.9% | −65.2% | −55.1% |
| | Week 12 (day 84) | −58.5% | −72.7% | −62.4% |
| | Follow up (week 13, day 91, 7 days | −57.6% | −62.4% | −60.9% |
| | after the treatment was stopped) | | | |

*BID = twice a day; WOMAC = Western Ontario and McMaster Universities.
**Scale ranged from 0 to 100 mm with lower score as better.
***Negative numbers mean improved.

TABLE 38
Efficacy Analysis of Change from Baseline in the
Physician's Global Assessment of Disease Status

| | | 8.75 | 17.5 | 35 |
|---|---|---|---|---|
| | | mg/knee | mg/knee | mg/knee |
| | | (BID) | (BID) | (BID) |
| Physician's | Improved* | 10.5% | 35.6% | 30.9% |
| Global | Worsened** | 1.8% | 1.7% | 0% |
| Assessment | | | | |

Scale ranged from 0 (very well), 1 (well), 2 (fair), 3 (poor) and 4 (very poor).
*Percentage of patients who improved by 2 grades or more from baseline for score 2 to 4 or changed in grade from a score of 1 to 0 in patient's global assessment.
**Percentage of patients who worsened by 2 grades or more from baseline for score 0 to 3 or changed in grade from a score of 3 to 4 in patient's global assessment.

As shown in Table 38, from the disclosed clinical trial data, the percentage of patients who improved by 2 grades or more from baseline is very similar for patients using placebo or active drugs such as naproxen and celecoxib, 21% for placebo, 33% for naproxen (500 mg, BID), 30% for Celecoxib (50 mg, BID), 36% for Celecoxib (100 mg, BID), and 32% for Celecoxib (200 mg, BID), respectively. From this Phase 2 trial, we see that there was a clear dose response. The results for 17.5 mg/knee and 35 mg/knee (BID) groups were comparable to naproxen and celecoxib, even when patients had a 41.7% chance to use placebo. However, there was only 10.5% for 8.75 mg/knee (BID) group; thus it was very unlikely from placebo effect. If it is from placebo effects, the changes in all groups should be similar, but the data show the changes of the middle and high dose groups are 2 times higher than that the change of the low dose.

TABLE 39
Efficacy Analysis of Change from Baseline in the Subject and Physician’s Global Assessment of
Response to Therapy

| | 8.75 mg/knee (BID) (%) | | | | | 17.5 mg/knee (BID) (%) | | | | | 35 mg/knee (BID) (%) | | | | |
|---|---|---|---|---|---|---|---|---|---|---|---|---|---|---|---|
| | Excellent | good | fair | poor | none | Excellent | good | fair | poor | none | Excellent | good | fair | poor | none |
| Subject’s global | 7.8 | 43.8 | 29.7 | 9.4 | 9.4 | 20.3 | 50.0 | 17.2 | 4.7 | 7.8 | 18.0 | 49.2 | 18.3 | 4.9 | 9.8 |
| assessment of | | | | | | | | | | | | | | | |
| response to therapy | 51.6 | | | | | 70.3 | | | | | 67.2 | | | | |
| Physician’s global | 11.1 | 38.1 | 36.5 | 6.3 | 7.9 | 23.2 | 43.5 | 18.8 | 8.7 | 5.8 | 18.8 | 42.2 | 17.2 | 14.1 | 7.8 |
| of assessment | | | | | | | | | | | | | | | |

| | | | |
|---|---|---|---|
| response to therapy | 49.2 | 66.7 | 60.9 |

As shown in Table 39, the results of 17.5 mg/knee and 35 mg/knee (BID) groups were better than the result of 8.75 mg/knee (BID) group. The total percentages of excellent and good were comparable to published results even when the patients have a 41.7% chance to use placebo.

Zeidler thought that paracetamol as rescue medication in nearly all OA trials may be a missing link to explain at least partially the large placebo response in OA trials. (Henning Zeidler, Paracetamol and the Placebo Effect in Osteoarthritis Trials: A Missing Link? Pain Research and Treatment, Volume 2011, 1-6.) From published data, the difference of the used amount of paracetamol is very small between the placebo and active drug cohorts. Sawitzke et al. reported that 645 mg/day/patient of paracetamol was used in placebo group and 465 mg/day/patient of paracetamol was used in the celecoxib group, a 28% decrease. (A. D. Sawitzke, H. Shi, M. F. Finco et al., “Clinical efficacy and safety of glucosamine, chondroitin sulphate, their combination, celecoxib or placebo taken to treat osteoarthritis of the knee: 2-year results from GAIT.” Annals of the Rheumatic Diseases, vol. 69, no. 8, pp. 1459-1464, 2010.) Schnitzer et al. reported that average daily use in the placebo group of 885 mg versus 665 mg to 715 mg in the naproxcinod groups and 670 mg in the naproxen group, a 19.2% to 24.9% decrease. (T. J. Schnitzer, A. Kivitz, H. Frayssinet, and B. Duquesroix, “Efficacy and safety of naproxcinod in the treatment of patients with osteoarthritis of the knee: a 13-week prospective, randomized, multicenter study.” Osteoarthritis and Cartilage, vol. 18, no. 5, pp. 629-639, 2010.) In this trial, NSAIDs or any analgesic therapy was discontinued for the duration of the study, starting at least 14 days (or 5 half lives, whichever is longer) prior to administration of the first dose of study medication (ie, for an analgesic washout period before Day 1). Subjects were allowed to take rescue medication (up to six 325 mg tablets [total of 1950 mg] of acetaminophen per day; provided by the Sponsor) for residual knee or other body pain starting at least 4 days (or 5 half lives, whichever is longer) prior to administration of the first dose of study medication except during the 24 hours prior to Baseline (Day 1), Week 2, Week 4, Week 8, Week 12/end of study (EOS), and Follow-up assessments. There was a maximum 84.9% decrease in the use of rescue medication, paracetamol, the results of which are shown in Table 40-42.

TABLE 40
Efficacy Analysis of Change from Baseline in the use of rescue
medication (paracetamol, mg/day/patient) only for knee pain

| | | | Daily amount, | Daily amount, | Daily amount, | Daily amount, |
|---|---|---|---|---|---|---|
| | | Daily amount, | mg/day/patient | mg/day/patient | mg/day/patient | mg/day/patient |
| | | mg/day/patient | (% of decrease | (% of decrease | (% of decrease | (% of decrease |
| | | Day −2 to | from baseline*) | from baseline*) | from baseline*) | from baseline*) |
| | | day −5 | Week 2 | Week 4 | Week 8, | Week 12, |
| 8.75 | mg/knee (BID) | 349 | 216(38.1%) | 167(52.1%) | 166(52.4%) | 159(54.4%) |
| 17.5 | mg/knee (BID) | 243 | 86(64.6%) | 67(72.4%) | 54(77.8) | 56(77.0%) |
| 35 | mg/knee (BID) | 291 | 98(66.3%) | 88(69.8%) | 54(81.4%) | 44(84.9%) |

*The baseline is the average daily use of paracetamol during Day −2 to day −5.

TABLE 41
Efficacy Analysis of Change from Baseline in the percentage
of patients using rescue medication only for knee pain

| | | Percentage | | | | |
|---|---|---|---|---|---|---|
| | | of patients | Percentage | Percentage | Percentage | Percentage |
| | | using rescue | of patients | of patients | of patients | of patients |
| | | medication | using rescue | using rescue | using rescue | using rescue |
| | | Day −2 to | medication | medication | medication | medication |
| | | day −5 | Week 2 | Week 4 | Week 8 | Week 12 |
| 8.75 | mg/knee (BID) | 35.3% | 23.6% | 18.4% | 17.1% | 16.3% |
| 17.5 | mg/knee (BID) | 24.0% | 11.3% | 8.4% | 7.2% | 6.5% |
| 35 | mg/knee (BID) | 34.8% | 12.4% | 11.0% | 7.6% | 5.8% |

* The baseline is the average daily use of paracetamol during Day −2 to day −5

There was a dose response and the use of rescue medication was getting less from pre-treatment, week 2, week 4, week 8 to week 12 and was up to 85% less in this study. At the end of the study, only 17.3%, 6.5% and 5.8% of patients used rescue medication for 8.75 mg/knee, 17.5 mg/knee and 35 mg/knee, respectively, which was much lower than the published studies.

TABLE 42
Efficacy Analysis of Change from Baseline in the use of rescue medication
(paracetamol, mg/day/patient) for all pain and other conditions

| | | | Daily amount, | Daily amount, | Daily amount, | Daily amount, |
|---|---|---|---|---|---|---|
| | | Daily amount, | mg/day/patient | mg/day/patient | mg/day/patient | mg/day/patient |
| | | mg/day/patient | (% of decrease | (% of decrease | (% of decrease | (% of decrease |
| | | Day −2 to | from baseline*) | from baseline*) | from baseline*) | from baseline*) |
| | | day −5 | Week 2 | Week 4 | Week 8 | Week 12 |
| 8.75 | mg/knee (BID) | 364 | 263(27.7%) | 208(42.9%) | 210(42.3%) | 209(42.6%) |
| 17.5 | mg/knee (BID) | 278 | 136(51.1%) | 115(58.6%) | 87(68.7) | 69(75.2%) |
| 35 | mg/knee (BID) | 310 | 171(44.8%) | 158(49.0%) | 111(64.2%) | 94(69.7%) |

*The baseline is the average daily use of paracetamol during Day −2 to day −5.

As shown in Table 43, the use of rescue medication for all pain and other conditions was low (less than 100 mg/day/patient for 17.5 mg/knee group and 35 mg/knee group at week 12) during the treatment, especially for a locally used dreg. The maximum decrease from pre-treatment to week 12 was up to 75% and the decrease was much larger than the published data.

TABLE 43
The use of rescue medication (paracetamol, mg/day/patient) for other conditions excluding knee pain

| | Day −2 to day −5 | Week 2 | Week 4 | Week 8 | Week 12 |
|---|---|---|---|---|---|
| | Daily amount, | Daily amount, | Daily amount, | Daily amount, | Daily amount, |
| | mg/day/patient | mg/day/patient | mg/day/patient | mg/day/patient | mg/day/patient |
| 8.75 mg/knee (BID) | 15 | 47 | 41 | 44 | 50 |
| 17.5 mg/knee (BID) | 35 | 50 | 48 | 33 | 13 |
| 35 mg/knee (BID) | 19 | 73 | 70 | 57 | 50 |

*The baseline is the average daily use of paracetamol during Day −2 to day −5.

The use of rescue medication for other conditions excluding knee pain was increased during the treatment than pre-treatment period. This may indicate the less use of rescue medication during the treatment was not from placebo effect. The use of rescue medication for other condition excluding knee pain was less during pre-treatment because other conditions were improved by more often use of rescue medication for knee pain during pre-treatment.

In the middle of this study, there was a drug shortage issue. A small second batch of testing drug were manufactured and 5 patients used these active drug kits for at least 8 weeks. WOMAC pain, joint stiffness, and difficulty performing daily activities subscale score for all patients were reduced dramatically and are shown in the following Tables 44-46.

TABLE 44
Change from Baseline in the WOMAC* Pain Subscale Scores

| | WOMAC VAS | WOMAC VAS | WOMAC VAS | WOMAC VAS | WOMAC VAS | WOMAC VAS |
|---|---|---|---|---|---|---|
| Patient | Score(mm) | Score(mm) | Score(mm) | Score(mm) | Score(mm) | Score(mm) |
| Number | Day 1 | Week 2 | Week 4 | Week 8 | Week 12 | Follow Up |
| 1056 | 51.8 | 31.0 | 22.6 | 28.0 | 12.0 | 10.0 |
| 1064 | 32.6 | 21.6 | 1.8 | 1.0 | 0 | 1.6 |
| 1535 | 40.4 | 2.8 | 2.6 | 4.0 | 0.6 | 0.0 |
| 1539 | 63.0 | 47.6 | 48.2 | 1.8 | 9.4 | 5.0 |
| 9032 | 16.6 | 11.2 | 6.2 | 4.4 | 3.2 | 3.0 |

The reduction of pain WOMAC from baseline to week 12 was between 81-98% and the pain WOMAC scores at the follow up visit were almost same as those at week 12.

TABLE 45
Change from Baseline in the WOMAC* Joint Stiffness Subscale Scores

| | WOMAC VAS | WOMAC VAS | WOMAC VAS | WOMAC VAS | WOMAC VAS | WOMAC VAS |
|---|---|---|---|---|---|---|
| Patient | Score(mm) | Score(mm) | Score(mm) | Score(mm) | Score(mm) | Score(mm) |
| Number | Day 1 | Week 2 | Week 4 | Week 8 | Week 12 | Follow Up |
| 1056 | 48.5 | 45.0 | 44.5 | 43.5 | 23.5 | 17.0 |
| 1064 | 66.5 | 20.5 | 12.5 | 0.0 | 1.0 | 1.0 |
| 1535 | 55.0 | 3.5 | 3.0 | 3.0 | 0.0 | 0.0 |
| 1539 | 70.0 | 54.5 | 44.0 | 8.0 | 3.0 | 2.0 |
| 9032 | 14.5 | 9.5 | 5.0 | 3.0 | 6.0 | 9.5 |

The reduction of Stiffness WOMAC from baseline to week 12 was between 58.6-100% and the Stiffness WOMAC scores at the follow up visit were almost same as those at week 12.

TABLE 46
Change from Baseline in the WOMAC* Difficulty Performing Daily Activities Subscale Scores

| | WOMAC VAS | WOMAC VAS | WOMAC VAS | WOMAC VAS | WOMAC VAS | WOMAC VAS |
|---|---|---|---|---|---|---|
| Patient | Score(mm) | Score(mm) | Score(mm) | Score(mm) | Score(mm) | Score(mm) |
| Number | Day 1 | Week 2 | Week 4 | Week 8 | Week 12 | Follow Up |
| 1056 | 39.6 | 27.1 | 24.4 | 25.2 | 18.1 | 11.6 |
| 1064 | 33.6 | 24.4 | 1.1 | 0.5 | 0.6 | 0.4 |
| 1535 | 51.6 | 3.3 | 2.8 | 3.5 | 0.2 | 0.2 |
| 1539 | 73.0 | 47.8 | 43 | 5.5 | 8.4 | 4.4 |
| 9032 | 16.4 | 6.0 | 7.1 | 3.4 | 2.5 | 2.8 |

The reduction of difficulty performing daily activities WOMAC from baseline to week 12 was between 65.1-100% and the difficulty performing daily activities WOMAC scores at the follow up visit were almost same as that of week 12.

As shown by the reduction in pain, stiffness, and functional ability severity in the patient's assessment in WOMAC (VAS) pain subscale scores, WOMAC (VAS) stiffness subscale scores, WOMAC (VAS) functional ability subscale scores, and use of rescue medication, by the percentage of patients who reported good or excellent in Subject's and investigator's global assessment of response to therapy, 2-(diethylamino)ethyl 2-(4-isobutylphenyl)propionate can reduce the signs and symptoms of osteoarthritis significantly in a dose response manner. The efficacy of 2-(diethylamino)ethyl 2-(4-isobutylphenyl)propionate in the relief of the signs and symptoms of osteoarthritis is found to be superior to the marketed common NSAIDs, such as Celebcoxib and Naproxen.

3.13. Safety Summary

All three doses of 2-(diethylamino)ethyl 2-(4-isobutylphenyl)propionate appeared to be safe and were generally well tolerated. As stated previously, Gastrointestinal disorders are the major problem of all NSAIDs, but there were only 12 very mild incidences in this study (3 constipation, 4 diarrhea, 1 gastroesophageal reflux disorder, 1 abdominal discomfort, 1 abdominal pain, 1 abdominal pain upper and 1 nausea) and none of them appeared to be drug related. The incidence rates were generally similar across all three treatment groups. No notable upper GI tract ulcer complication (ie, bleeding episode, perforation, or gastric outlet obstruction) occurred during the study. Mean and median blood pressures remained unchanged. Even the skin irritation (a common AE of topical drugs) incidence rates were very low (total 8 incidences) and mild due to the simple formulation and fact that 2-(diethylamino)ethyl 2-(4-isobutylphenyl)propionate is a biological inactive prodrug when it is on outside of the body.

3.14. PK Summary:

Following topical applications of 8.75 mg, 17 mg, and 35 mg of 2-(diethylamino)ethyl 2-(4-isobutylphenyl)propionate to OA subjects, absorption of 2-(diethylamino)ethyl 2-(4-isobutylphenyl)propionate was rapid and the absorbed 2-(diethylamino)ethyl 2-(4-isobutylphenyl)propionate was rapidly converted to ibuprofen. The ratio of ibuprofen and 2-(diethylamino)ethyl 2-(4-isobutylphenyl)propionate was greater than 99:1, overall, ibuprofen and 2-(diethylamino)ethyl 2-(4-isobutylphenyl)propionate mean maximum plasma concentration and AUC increased as 2-(diethylamino)ethyl 2-(4-isobutylphenyl)propionate doses increased from 8.75 mg to 35 mg but dose proportionality was not formally demonstrated as topical doses of 2-(diethylamino)ethyl 2-(4-isobutylphenyl)propionate increased from 8.75 mg to 35 mg. Based on observed Cmin and accumulation index of ibuprofen, it can be concluded that steady-state was reached following 5 days of b.i.d. dosing. Relative bioavailability of 2-(diethylamino)ethyl 2-(4-isobutylphenyl)propionate based on measured plasma ibuprofen concentrations when given as spray was lower when compared to the topical application following single application, but was comparable at steady-state.

4. The Second Phase 2 Clinical Study

A Phase 2, Multicenter, Randomized, Double Blind (Within Dose), Ibuprofen and Placebo Controlled, Parallel Group, Dose Range Finding Study to Evaluate the Efficacy, Safety, and Pharmacokinetics of 2-(diethylamino)ethyl 2-(4-isobutylphenyl)propionate spray versus Placebo and oral ibuprofen (3×400 mg/day) in Subjects with Osteoarthritis of the Knee.

4.1. Methodology/Study Design

This is a Phase 2, multicenter, randomized, double blind (within dose), Ibuprofen-placebo controlled, parallel group, dose range finding study to evaluate the efficacy, safety, and PK of 2-(diethylamino)ethyl 2-(4-isobutylphenyl)propionate spray in adult subjects with clinically symptomatic OA of the knee. For subjects with bilateral knee pain, both knees will be treated, but the most symptomatic knee (ie, the most painful knee as measured by the Western Ontario and McMaster University Osteoarthritis Index [WOMAC® 3.1] pain subscale score at Screening) will be designated as the target knee for efficacy analyses. For subjects with unilateral knee pain, only the symptomatic (target) knee will be treated.

Subjects taking nonsteroidal anti inflammatory drugs (NSAID) or other analgesics may enroll in the trial, but will discontinue any analgesic therapy for the duration of the study, starting at least 4 days (or 5 half lives, whichever is longer) prior to administration of the first dose of study medication (i.e., for an analgesic washout period before Day 1). Subjects will be allowed to take rescue medication (up to six 500 mg tablets [total of 3000 mg] of acetaminophen per day; provided by the Sponsor) for residual knee or other body pain starting 4 days (or 5 half lives, whichever is longer) prior to administration of the first dose of study medication except during the 24 hours prior to Baseline (Day 1), Week 2, Week 4, Week 8, Week 12/end of study (EOS), and Follow up assessments.

After a Screening Period of up to 3 weeks, a subject will be randomly assigned to 1 of 3 treatment groups in a 1:1:1 ratio with a 3:1:1 ratio of the Testing drug:placebo:oral ibuprofen within each treatment group (i.e., 3 subjects to the testing drug treatment, 1 subject to placebo, and 1 subject to oral ibuprofen):
Group A: 4.375 mg 2-(diethylamino)ethyl 2-(4-isobutylphenyl)propionate/knee (1 sprays/knee, one spray to the medial surface or the lateral surface of the knee), twice daily (BID, approximately every 12 hours; n=72) for a total of 2 sprays per knee per day and oral placebo, thrice daily (approximately every 8 hours), placebo spray (1 sprays/knee), BID (approximately every 12 hours; n=24) for a total of 2 sprays per knee per day and oral placebo, thrice daily (approximately every 8 hours), and oral ibuprofen, thrice daily (approximately every 8 hours; n=24) and placebo spray (1 sprays/knee), BID (approximately every 12 hours; n=24) for a total of 2 sprays per knee per day;
Group B: 8.75 mg 2-(diethylamino)ethyl 2-(4-isobutylphenyl)propionate/knee (2 sprays/knee, one spray to the medial surface and one spray to the lateral surface of the knee), twice daily (BID, approximately every 12 hours; n=72) for a total of 4 sprays per knee per day and oral placebo, thrice daily (approximately every 8 hours), placebo spray (2 sprays/knee), BID (approximately every 12 hours; n=24) for a total of 4 sprays per knee per day and oral placebo, thrice daily (approximately every 8 hours), and oral ibuprofen, thrice daily (approximately every 8 hours; n=24) and placebo spray (2 sprays/knee), BID (approximately every 12 hours; n=24) for a total of 4 sprays per knee per day;
Group C: 17.5 mg 2-(diethylamino)ethyl 2-(4-isobutylphenyl)propionate/knee (4 sprays/knee, one spray to the medial surface and one spray to the lateral surface of the knee, and one spray to front and one spray to the back of the knee), twice daily (BID, approximately every 12 hours; n=72) for a total of 4 sprays per knee per day and oral placebo, thrice daily (approximately every 8 hours), placebo spray (4 sprays/knee), BID (approximately every 12 hours; n=24) for a total of 8 sprays per knee per day and oral placebo, thrice daily (approximately every 8 hours), and oral ibuprofen, thrice daily (approximately every 8 hours; n=24) and placebo spray (4 sprays/knee), BID (approximately every 12 hours; n=24) for a total of 8 sprays per knee per day.

Each subject will receive study treatment for 12 weeks starting on Day 1. Qualified site personnel will contact subjects 1 week after the first administration of study medication to check if there are any issues with the spray bottles, administration of study medication, or adverse events (AEs). Subjects will return to the site at 2, 4, 8, and 12 weeks of treatment for efficacy and safety assessments as indicated in the study flow chart. The Week 12 visit will be the EOS visit. On the final day of dose administration (Week 12/EOS visit), subjects will receive 1 dose of study medication in the morning only; Subjects will have a Follow up visit approximately 14 days after the Week 12/EOS visit.

Subjects will record the following information in a Daily Diary starting at Screening: the amount of knee pain in the target knee while walking during the preceding 24 hours (using a 100 mm visual analog scale [VAS]), the times and number of sprays for each administration of study medication, the times when the subject washes his/her knees and/or takes a shower, the number of tablets of rescue medication taken that day and the times taken, the reason for taking the rescue medication (e.g., knee pain, headache, low back pain), any other concomitant medications taken that day, and any AEs occurring that day.

The VAS version of the WOMAC will be used for the Primary and Secondary Efficacy Endpoints. Efficacy endpoints will be assessed at Screening and Baseline (Day 1) and at the Week 2, Week 4, Week 8, and Week 12/EOS visits.

To assess the systemic multiple dose PK profile of 2-(diethylamino)ethyl 2-(4-isobutylphenyl)propionate and ibuprofen, subjects from each treatment group will be assigned to the PK subgroup. All subjects enrolled at designated sites that have the capability to keep subjects overnight will be included in the PK subgroup until PK samples for 40 subjects per treatment group have been collected. Blood samples for determining trough levels of 2-(diethylamino)ethyl 2-(4-isobutylphenyl)propionate and ibuprofen will be collected at the Week 4 visit, PK blood samples will be collected predose and at 1, 3, and 5 hours postdose. At the Week 12/EOS visit, PK blood samples can be collected predose and at 2, 4, and 6 hours postdose. Subjects will only receive the morning dose at the Week 12/EOS visit.

Safety assessments will be performed at each visit and will include assessment of AEs, vital signs (blood pressure, pulse rate, and oral temperature), clinical laboratories, physical examination, skin irritation, and electrocardiograms (ECGs) as indicated in the study flow chart, Table 47.

TABLE 47
Study Flow Chart

| | Screening | | | | | | | | Follow-up |
|---|---|---|---|---|---|---|---|---|---|
| | Period | Treatment Period | | | | | | | Period |
| | Visit | | | | | | | | |
| | | Day | Check-in | Week | Week | Week | Week | Week 12/EOS^{a} | Follow-up |
| | Screening | 1^{a} | Phone Call | 2^{a} | 3^{a,b} | 4^{a} | 8^{a} | (Early Term^{c}) | Visit |
| | Study Day | | | | | | | | |
| | Up to 3 weeks | 1 | 7 ± 2 | 14 ± 2 | 21 ± 2 | 28 ± 2 | 56 ± 2 | 84 ± 2 | 91 ± 2 |
| Informed consent | X | | | | | | | | |
| Inclusion/exclusion | X | X | | | | | | | |
| criteria | | | | | | | | | |
| Demographics | X | | | | | | | | |
| Medical/surgical | X | | | | | | | | |
| history | | | | | | | | | |
| Previous | X | | | | | | | | |
| medications | | | | | | | | | |
| Concomitant | X | X | | X | | X | X | X | X |
| medications | | | | | | | | | |
| Screening/Day 1 | X | X | | | | | | | |
| Pain Assessment | | | | | | | | | |
| (VAS) | | | | | | | | | |
| Body weight/ | X | | | | | | | | |
| height/BMI | | | | | | | | | |
| Physical exam | X | | | | | | | X | |
| Vital signs^{d} | X | X | | X | | X | X | X | X |
| 12-Lead electro- | x | X | | | | | | X | |
| cardiogram | | | | | | | | | |
| Clinical chemistry | X | X | | X | | X | X | X | |
| panel and CBC | | | | | | | | | |
| FSH^{e} | X | | | | | | | | |
| Pregnancy test | X | | | | | | | X | |
| (serum β-hCG)^{f} | | | | | | | | | |
| Urine pregnancy | | X | | | | | | | |
| test^{f} | | | | | | | | | |
| Urinalysis | X | | | | | | | | |
| Fecal occult | | | | | | | | | |
| blood test | X | X | | X | | X | X | X | |
| Hepatitis & HIV | X | | | | | | | | |
| screen | | | | | | | | | |
| Drug screen | X | | | | | | | | |
| Randomization | | X | | | | | | | |
| WOMAC 3.1 | X | X | | X | | X | X | X | X |
| Subject’s global | X | X | | X | | X | X | X | X |
| assessment of | | | | | | | | | |
| disease status | | | | | | | | | |
| Subject’s global | | | | X | | X | X | X | X |
| assessment of | | | | | | | | | |
| response to therapy | | | | | | | | | |
| Investigator’s | X | X | | X | | X | X | X | X |
| global assessment | | | | | | | | | |
| of disease status | | | | | | | | | |
| Investigator’s | | | | X | | X | X | X | X |
| global assessment | | | | | | | | | |
| of response to | | | | | | | | | |
| therapy | | | | | | | | | |
| PK blood samples | | | | | | | X | X | |
| (PK-subgroup- | | | | | | | | | |
| only)^{h} | | | | | | | | | |
| Diary trainingⁱ/ | Xⁱ | X^{j,k} | | X^{j,k} | | X | X | X^{j} | |
| review^{j} | | | | | | | | | |
| Check-in phone | | | X^{l} | | | | | | |
| call | | | | | | | | | |
| Adverse events | X | X | X | X | | X | X | X | X |
| Skin irritation | X | X^{m} | | X^{m} | | X^{m} | X^{m} | X^{m} | X |
| evaluation (knee)^{m} | | | | | | | | | |
| Drug | | X^{o} | | X^{p} | X^{p} | X^{p} | X^{p} | X^{p} | |
| administrationⁿ | | | | | | | | | |
| Study Drug, | | | | | | | | | |
| Dispensing, Return, | | X | | | | X | X | X | |
| And Accountability | | | | | | | | | |
| Rescue Medication | | | | | | | | | |
| Dispensing, Return, | X | | | X | | X | X | X | |
| and Accountability^{q} | | | | | | | | | |

β hCG = human chorion gonadotropin β subunit;
AE = adverse event;
BMI = body mass index;
CBC = complete blood cell count;
Early Term = early termination;
EOS = end of study;
FSH = follicle stimulating hormone;
HIV = human immunodeficiency virus;
PK = pharmacokinetics;
VAS = visual analog scale;
WOMAC = Western Ontario and McMAster Osteoarthritis Index
Safety and efficacy assessments and/or blood samples should be completed prior to dose administration at each site visit.
Only subjects assigned to the PK subgroup will return to the clinic for the Week 3 visit for PK blood sampling (see footnote “h” below).
Week 12/EOS visit assessments (except PK blood samples) should be done at Early Termination visits.
Vital signs include sitting blood pressures, pulse rate, and oral temperature.
Only required for woman who have had continuous ammenorrhea for at least 12 months.
Serum and urine pregnancy tests are required for all women of childbearing potential.
A total of 40 subjects from each temperature group will be selected for PK sampling. PK blood samples at the Week 8 visit, PK samples will be collected predose and at 1, 3, and 5 hours postdose in the PK subgroup. At the Week 12/EOS visit, PK blood samples can be collected predose and at 2, 4, amd 6 hours postdose.
At the Screening visit, trained site personnel will give eligible subjects a Daily Diary and train them to record the amount of pain in the target kneee while walking during the preceding 24 hours, the time and number of sprays for each administration of study medication, the times when the subject washes his/her knees and/or takes a shower, the number of tablets of rescue medication taken that day and the times taken, the reason for taking the rescue medication, any other concomitant medications taken that day, and any AEs occurring that day in the Daily Diary. Each subject will be instructed to complete the diary each day before going to bed starting 14 days before the Day 1 visit and to bring the diary to each visit.
Trained site personnel should review the diary for completeness and consistency including the amounts of study and rescue medications returned by the subject. The subject should be asked about any missing data and the reasons for missing data should be noted in the Daily Diary.
At the Day 1, and Week 2, Week 4, and Week 8 visits, subjects will be given a new Daily Diary and instructed to complete the diary each day before going to bed and to bring the diary to the next visit.
Qualified site personnel will contact subjects 1 week after the first administration of study medication to check if there are any issues with the spray bottles, administration of study medication, or AEs.
Skin irritation assessments will be performed prior to and 30(±5) minutes after the morning dose of study medication at the Day 1, Week 2, Week 4, Week 8, and Week 12/EOS visits.
Subjects will randomized to receive 2-(diethylamino)ethyl 2-(4-isobutylphenyl)propionate or placebo within Group A (1 sprays/knee), Group B (2 sprays per knee), or Group C (4 sprays/knee) twice daily for 12 weeks according to the randomization schedule. On the final day of dose administration (the Week 12/EOS visit), subjects will receive one dose of study medication in the morning only.
On Day 1, qualified site personnel will train the subject to clean their knees with a wet towel to remove any dirt or residual skin care products and thoroughly dry them just prior to administering study medication, to self sdminister study medication according to the Subject’s knees should be washed with soap and water and thoroughly dried to remove an dirt or residual skin care products.
On the mornings of Week 2, Week 4, Week 8, and Week 12/EOS visits subjects are not to self administer the morning dose of study medication prior to returning to the site. On these days, subjects will self administer the morning dose of study medication only after safety and efficacy assessments and PK blood collections have been completed.
Subjects should not take any rescue medication during the 24 hour period prior to Baseline (Day 1), Week 2, Week 4, Week 8, and final (Week 12/EOS) assessments.

4.2. Diagnosis and Main Criteria for Inclusion:

Adult subjects with a diagnosis of primary OA of the knee will be enrolled in this study. The following are the key inclusion criteria:
1. A subject must be a male or female between 40 and 75 years of age, inclusive.
2. A subject must have a diagnosis of idiopathic OA according to the American College of Rheumatology clinical and radiographic criteria (knee pain, osteophytes, and at least one of the following: >50 years of age, morning stiffness lasting<30 minutes after getting up in the morning, or crepitus).
3. A subject must have a history of clinically symptomatic mild to moderate OA of the knee for >6 months.
4. A subject must have had knee pain while standing, walking, and/or on motion for at least 14 days during the month prior to Screening.
5. A subject must have a knee pain score≥40 mm and <90 mm on a 100 mm VAS (with or without analgesic medication) on at least 10 of the 14 days prior to randomization.
6. A subject must be willing to discontinue any NSAIDs or other analgesic (e.g., aspirin, acetaminophen) or potentially confounding concomitant treatments (e.g., physiotherapy, acupuncture) starting 4 days (or 5 half lives, whichever is longer) before the administration of the first dose of study medication until completing participation in the study. (The use of ≤325 mg acetylsalicylic acid per day as cardiac prophylaxis is permitted.) The subject will be allowed to take rescue medication (acetaminophen) for pain during the study except during the 24 hours prior to Baseline (Day 1), Week 2, Week 4, Week 8, Week 12/EOS, and Follow-up assessments.
7. A subject must be willing to discontinue applying any topical preparations containing Vitamin A acids (including all trans retinoic acid (tretinoin), 13 cis retinoic acid [isotretinoin], 9 cis retinoic acid [alitretinoin], vitamin A [retinol], retinal, and their derivatives) to the lower limbs starting on Day 1 until completing participation in the study. (Topical preparations containing Vitamin A acids or retinol may be applied to areas of the skin above the waist, but should not be applied to areas of the skin exposed to study medication.)
8. A subject must be willing to avoid unaccustomed physical activity (e.g., starting a new weight lifting routine) for the duration of the study.
9. With the exception of OA of the knee, the subject must be in good general health with no clinically significant findings from medical history, vital signs, physical examination, ECG, and routine laboratory tests that could interfere with subject safety, or pain and functional assessments, as determined by the Investigator.

4.3. Criteria for Exclusion

The following are the main exclusion criteria:
1. A subject who has secondary OA of the knee or OA of lower limb joints other than the knee that, in the opinion of the Investigator, could interfere with pain and functional assessments related to the knee;
2. A subject who has a history of total or partial knee replacement, arthroplasty, or other knee surgery on either knee;
3. A subject who has had significant injury, as judged by the Investigator, involving the target knee within the 6 months before Screening.
4. A subject who has skin lesions or wounds on or near the knees to be treated at Screening or on Day 1 prior to the first administration of study medication;
5. A subject who has used opiates or corticosteroids within 30 days before Screening or who requires treatment with chronic opiates or corticosteroids;
6. A subject who has had intra articular injections of corticosteroids, hyaluronic acid, or viscosupplements (e.g., Synvisc®) to a knee to be treated within the 3 months before Screening.
7. A subject who has a history of significant hypersensitivity, intolerance, or allergy to ibuprofen, any NSAIDs, aspirin, or acetaminophen;
8. A subject who has had an active peptic ulceration in the 12 months prior to Screening or a history of gastrointestinal (GI) bleeding within 5 years of Screening;
9. A subject who has used an anticoagulant (except aspirin up to 325 mg/day for cardiac prophylaxis) in the month prior to Screening;
10. A subject who has positive results on fecal occult blood testing at Screening or on Day 1 prior to the first administration of study medication;
11. A subject who has a history of chronic inflammatory disease (such as rheumatoid arthritis, psoriatic arthritis, gouty arthritis), fibromyalgia, conditions that may affect the target joint (e.g., osteonecrosis, chondrocalcinosis), or asthma.

4.4. Study Medications:

Test Product, Dose, Dosage Form, and Mode of Administration:

The test product is a 7% solution of 2-(diethylamino)ethyl 2-(4-isobutylphenyl)propionate hydrochloride in 25% ethanol, which will be administered topically as a spray, the 7% topical spray solution consists of 700 mg 2-(diethylamino)ethyl 2-(4-isobutylphenyl)propionate hydrochloride (equivalent to 625 mg 2-(diethylamino)ethyl 2-(4-isobutylphenyl)propionate free base) in 10 mL 25% ethanol (v/v), the spray bottle deposits 70 mg of spray solution and 4.375 mg 2-(diethylamino)ethyl 2-(4-isobutylphenyl)propionate (free base) per spray on the skin. Subjects are to apply each spray to a different skin area around the knee (e.g., lateral, medial, front, and back surfaces of the knee) based on the randomized dose level assigned.

Reference Therapy, Dose, Dosage Form, and Mode of Administration:

The reference therapy is placebo, which will be administered topically as a spray. The spray bottle deposits 70 mg of spray solution per spray on the skin. Subjects are to apply each spray to a different skin area around the knee (e.g., lateral, medial, front, and back surfaces of the knee) based on the randomized dose level assigned.

Subjects will not take a shower or wash their knees until at least 8 hours after administration of study medication.

4.5. Dose and Regimen:

Subjects will receive the following treatments BID for 12 weeks. On the last day of dose administration (at the Week 12/EOS) visit, subjects will receive 1 dose of study medication in the morning only. The following treatments will be randomly assigned:
Group A: 4.375 mg 2-(diethylamino)ethyl 2-(4-isobutylphenyl)propionate/knee (1 sprays/knee, one spray to the medial surface or the lateral surface of the knee), twice daily (BID, approximately every 12 hours; n=72) for a total of 2 sprays per knee per day and oral placebo, thrice daily (approximately every 8 hours), placebo spray (1 sprays/knee), BID (approximately every 12 hours; n=24) for a total of 2 sprays per knee per day and oral placebo, thrice daily (approximately every 8 hours), and oral ibuprofen, thrice daily (approximately every 8 hours; n=24) and placebo spray (1 sprays/knee), BID (approximately every 12 hours; n=24) for a total of 2 sprays per knee per day;
Group B: 8.75 mg 2-(diethylamino)ethyl 2-(4-isobutylphenyl)propionate/knee (2 sprays/knee, one spray to the medial surface and one spray to the lateral surface of the knee), twice daily (BID, approximately every 12 hours; n=72) for a total of 4 sprays per knee per day and oral placebo, thrice daily (approximately every 8 hours), placebo spray (2 sprays/knee), BID (approximately every 12 hours; n=24) for a total of 4 sprays per knee per day and oral placebo, thrice daily (approximately every 8 hours), and oral ibuprofen, thrice daily (approximately every 8 hours; n=24) and placebo spray (2 sprays/knee), BID (approximately every 12 hours; n=24) for a total of 4 sprays per knee per day;
Group C: 17.5 mg 2-(diethylamino)ethyl 2-(4-isobutylphenyl)propionate/knee (4 sprays/knee, one spray to the medial surface and one spray to the lateral surface of the knee, and one spray to front and one spray to the back of the knee), twice daily (BID, approximately every 12 hours; n=72) for a total of 4 sprays per knee per day and oral placebo, thrice daily (approximately every 8 hours), placebo spray (4 sprays/knee), BID (approximately every 12 hours; n=24) for a total of 8 sprays per knee per day and oral placebo, thrice daily (approximately every 8 hours), and oral ibuprofen, thrice daily (approximately every 8 hours; n=24) and placebo spray (4 sprays/knee), BID (approximately every 12 hours; n=24) for a total of 8 sprays per knee per day.

4.6. Number of Investigators and Study Centers:
11 sites in China

4.7. Duration of Subject Participation in Study:
Screening Period: up to 3 weeks
Treatment Period: 12 weeks.
Follow-up Period: 14 days

4.8. Study Populations:

Safety Analysis Set (SAS): The SAS is defined as all subjects who were administered study medication and have at least 1 postdose safety assessment.

Full Analysis Set (FAS): The FAS is defined as all subjects who were administered study medication and have at least 1 postdose efficacy assessment.

Pharmacokinetics Analysis Set (PKAS): The PKAS is defined as all subjects who were administered 2-(diethylamino)ethyl 2-(4-isobutylphenyl)propionate and have at least 1 evaluable postdose 2-(diethylamino)ethyl 2-(4-isobutylphenyl)propionate and/or ibuprofen plasma concentration.

4.9. Evaluation: Efficacy:

4.9.1. Primary Efficacy Endpoint:

The Primary Efficacy Endpoint is the change from Baseline in the WOMAC (VAS) pain subscale score for the target knee at 12 weeks of treatment.

4.9.2. Secondary Efficacy Endpoints:
1. Change from Baseline in the WOMAC (VAS) pain subscale score for the target knee at 2, 8, and 12 weeks of treatment;
2. Change from Baseline in the WOMAC (VAS) stiffness subscale scores for the target knee and WOMAC (VAS) functional ability subscale scores at 2, 4, 8, and 12 weeks of treatment;
3. Change from Baseline in the overall WOMAC (VAS) score at 2, 4, 8, and 12 weeks of treatment;
4. Subject's global assessment of disease status of the target knee at 2, 4, 8, and 12 weeks of treatment;
5. Subject's global assessment of response to therapy of the target knee at 2, 4, 8, and 12 weeks of treatment;
6. Amount of rescue medication (acetaminophen) consumed per day for target knee pain.

4.9.3. Exploratory Efficacy Endpoints:
1. Investigator's global assessment of response to therapy of the target knee at 2, 4, 8, and 12 weeks of treatment;
2. Change from Baseline over time in overall WOMAC (VAS) score for the target knee after the treatment is stopped for 2 weeks.

4.10. Evaluation: Safety

Safety assessments will include AEs, vital signs (blood pressures, pulse rate, and oral temperature), clinical laboratories, physical examination, skin irritation, and ECGs at various time points during the study as indicated in the study flow chart.

Adverse events of interest include: local skin reactions around the treated knee(s), upper stomach pain, GI bleeding, serious cardiovascular side effects (e.g., thrombotic events, myocardial infarction, or stroke), jaundice, elevated liver function tests, and nausea.

4.11. Statistical Methods:

Efficacy analyses will be conducted on the FAS.

4.11.1. Primary Efficacy Analysis:

The Primary Efficacy Endpoint is change from Baseline in the WOMAC (VAS) pain subscale score for the target knee at 4 weeks of treatment, and will be analyzed using an analysis of covariance (ANCOVA). Treatment will be included as a fixed class effect and WOMAC Baseline pain subscale score as covariates. The primary comparisons of interest will be the difference between active Group A (4.375 mg 2-(diethylamino)ethyl 2-(4-isobutylphenyl)propionate/knee) and combined placebo, active Group B (8.75 mg 2-(diethylamino)ethyl 2-(4-isobutylphenyl)propionate/knee) and combined placebo, and active Group C (17.5 mg 2-(diethylamino)ethyl 2-(4-isobutylphenyl)propionate/knee) and combined placebo.

4.11.2. Secondary Efficacy Analyses:

A sensitivity analysis will also be conducted on the Primary Efficacy Endpoint using an ANCOVA with treatment as a fixed class effect and WOMAC Baseline pain subscale score as covariates, but the comparisons of interest will be the difference between the active and placebo subjects within each treatment group.

The Secondary Efficacy Endpoints, change from Baseline in the WOMAC subscale scores for pain, stiffness, and functional ability, and overall WOMAC score at 2, 4, 8, and 12 weeks of treatment, will be analyzed using the same methods as for the Primary Efficacy Endpoint.

4.11.3. Exploratory Efficacy Endpoints:

Data for the exploratory efficacy endpoints will be summarized using descriptive statistics.

4.11.4. Safety Analyses:

Safety analyses will be conducted on the SAS. Safety parameters will be listed and summarized using standard descriptive statistics, as appropriate. No formal statistical analyses are planned.

When the first 130 subjects were enrolled in 11 sites, a clinical trial patient recruitment company was hired to accelerate patient recruiting.

Tables 48-61 show the clinical data of the first 130 subjects (from Randomization number 001-130).

TABLE 48
Patient Demographic and Baseline Characteristics

| | 4.375 | 8.75 | 17.5 | Oral | |
|---|---|---|---|---|---|
| | mg/knee | mg/knee | mg/knee, | Ibuprofen | |
| | BID, | BID, | BID, | (3 × 400 mg) | Placebo |
| Characteristic | (n = 23) | (n = 24) | (n = 19) | (n = 20) | (n = 25) |
| Mean (rang) age(y) | 57.2 (43-68) | 60.8 (51-70) | 57.4 (43-71) | 60.5 (54-72) | 56.7 (46-72) |
| Female sex (%) | 19 (82.6%) | 20 (83.3%) | 15 (78.9%) | 15 (75.0%) | 19 (76.0%) |
| Male sex (%) | 4 (17.4%) | 4 (16.7%) | 4 (21.1%) | 5 (25.0%) | 6 (24.0%) |
| Asian | 100% | 100% | 100% | 100% | 100% |
| Mean ± SD | 60.0 (±11.9) | 60.7 (±10.3) | 64.1 (±12.0) | 67.0 (±11.3) | 63.7 (±14.1) |
| WOMAC pain | | | | | |
| Mean ± SD | 50.2 (±19.6) | 52.8 (±15.4) | 57.6 (±15.9) | 56.4 (±21.5) | 53.4 (±18.2) |
| WOMAC stiffness | | | | | |
| Mean ± SD | 60.2 (±14.0) | 61.4 (±9.4) | 63.9 (±11.5) | 65.5 (±12.6) | 61.0 (±16.1) |
| WOMAC difficulty | | | | | |
| Mean ± SD | 64.8 (±15.1) | 67.0 (±10.2) | 69.3 (±10.6) | 75.2 (±11.1) | 66.3 (±13.7) |
| WOMAC Patient's | | | | | |
| global assessment | | | | | |

TABLE 49
Efficacy Analysis of Change from Baseline in the WOMAC* Pain Subscale Scores at Week 12

| | Baseline | Week 12 | Changes from | | | | Different | | | Different | | |
|---|---|---|---|---|---|---|---|---|---|---|---|---|
| | (LS | (LS | Baseline | % of improvement from | | | from placebo | | | oral ibuprofen | | |
| | Mean & | Mean & | (LS Mean & | | | Oral | (LS Mean & | | P | (LS Mean & | | P |
| Cohort | 95% CI) | 95% CI) | 95% CI) | baseline | Placebo | Ibuprofen | 95% CI) | | value | 95% CI) | | value |
| Placebo | 63.7 | 42.6 | −21.1 | −33.1% | — | −0.9% | — | | — | — | | — |
| (n = 25) | (58.2, | (35.0, | (−28.6, | | | | | | | | | |
| | 69.2) | 50.2) | −13.6) | | | | | | | | | |
| Oral | 67.0 | 45.4 | −21.6 | −32.2% | 0.9% | — | −0.5 | (011.5, 10.5) | 0.9287 | — | | — |
| Ibuprofen | (62.0, | (36.8, | (−29.7, | | | | | | | | | |
| (3 × 400 | 72.0) | 54.0) | −13.5) | | | | | | | | | |
| mg) | | | | | | | | | | | | |
| (n = 20) | | | | | | | | | | | | |
| 4.375 mg/ | 60.0 | 34.3 | −25.7 | −41.8% | −8.7% | −9.6% | −4.3 | (−13.6, 5.0) | 0.3644 | −4.0 | (−13.9, 5.9) | 0.4296 |
| knee BID, | (55.1, | (27.6, | (−31.4, | | | | | | | | | |
| (n = 23) | 64.9) | 41.0) | −20.0) | | | | | | | | | |
| 8.75 mg/ | 61.0 | 33.3 | −27.7 | −45.4% | −12.3% | −13.2% | −6.6 | (−17.2, 4.0) | 0.2304 | −6.1 | (−17.1, 4.9) | 0.2867 |
| knee BID, | (56.9, | (27.3, | (−35.2, | | | | | | | | | |
| (n = 24) | 65.1) | 39.3) | −20.2) | | | | | | | | | |
| 17.5 mg/ | 64.1 | 27.9 | −36.2 | −56.5% | −23.4% | −24.3% | −15.0 | (−27.7, −2.3) | 0.0250 | −14.5 | (−27.5, −1.5) | 0.0352 |
| knee, BID, | (59.4, | (23.0, | (−45.1, | | | | | | | | | |
| (n = 19) | 68.8) | 36.8) | −27.3) | | | | | | | | | |

*BID = twice a day; WOMAC = Western Ontario and McMaster Universities.
**Scale ranged from 0 to 100 mm with lower score as better.
***Negative numbers mean improved.

TABLE 50
Efficacy Analysis of Change from Baseline in the WOMAC* Stiffness Subscale Scores at Week 12

| | Baseline | Week 12 | Changes from | | | | Different | | | Different from | | |
|---|---|---|---|---|---|---|---|---|---|---|---|---|
| | (LS | (LS | Baseline | % of improvement from | | | from placebo | | | oral ibuprofen | | |
| | Mean & | Mean & | (LS Mean & | | | Oral | (LS Mean & | | P | (LS Mean & | | P |
| Cohort | 95% CI) | 95% CI) | 95% CI) | baseline | Placebo | Ibuprofen | 95% CI) | | value | 95% CI) | | value |
| Placebo | 53.4 | 39.4 | −14.0 | −26.2% | — | 10.3% | — | | — | — | | — |
| (n = 25) | (46.3, | (31.3, | (−22.4, | | | | | | | | | |
| | 50.5) | 47.5) | −5.6) | | | | | | | | | |
| Oral Ibuprofen | 56.4 | 35.8 | −20.6 | −36.5% | −10.3% | — | −6.6 | (−17.7, 4.5) | 0.2460 | — | | — |
| (3 × 400 mg) | (47.0, | (27.3, | (−28.3, | | | | | | | | | |
| (n = 20) | 65.8) | 44.3) | −12.9) | | | | | | | | | |
| 4.375 mg/knee | 50.2 | 32.2 | −18.0 | −35.9% | −9.7% | 0.6% | −4.0 | (−14.5, 6.5) | 0.4559 | — | | — |
| BID, (n = 23) | (42.2, | (25.0, | (−24.4, | | | | | | | | | |
| | 58.2) | 39.4) | −11.6) | | | | | | | | | |
| 8.75 mg/knee | 52.4 | 32.0 | −20.4 | −38.9% | −12.7% | −2.4% | −6.4 | (−17.6, 4.8) | 0.2667 | 0.2 | (−10.5, 10.9) | 0.9699 |
| BID, (n = 24) | (46.2, | (26.0, | (−27.8, | | | | | | | | | |
| | 58.6) | 37.8) | −13.0) | | | | | | | | | |
| 17.5 mg/knee, | 57.6 | 24.6 | −33.1 | −57.5% | −31.3% | −21.0% | −19.1 | (−31.8, −6.4) | 0.0051 | −12.4 | (−24.4, −0.4) | 0.0220 |

| | | | |
|---|---|---|---|
| BID, (n = 19) | (50.5, | (17.9, | (−42.5, |
| | 64.8) | 31.3) | −23.7) |

*BID = twice a day; WOMAC = Western Ontario and McMaster Universities.
**Scale ranged from 0 to 100 mm with lower score as better.
***Negative numbers mean improved.

TABLE 51
Efficacy Analysis of Change from Baseline in the WOMAC* Difficulty Performing Daily Activities
Subscale Scores at Week 12

| | Baseline | Week 12 | Changes from | | | | Different | | | Different from | | |
|---|---|---|---|---|---|---|---|---|---|---|---|---|
| | (LS | (LS | Baseline (LS | % of improvement from | | | from placebo | | | oral ibuprofen | | |
| | Mean & | Mean & | Mean & | | | Oral | (LS Mean & | | P | (LS Mean & | | P |
| Cohort | 95% CI) | 95% CI) | 95% CI) | baseline | Placebo | Ibuprofen | 95% CI) | | value | 95% CI) | | value |
| Placebo | 61.0 | 42.9 | −18.1 | −29.7% | — | 3.3% | — | | — | — | | — |
| (n = 25) | (54.7, | (35.5, | (−25.9, | | | | | | | | | |
| | 67.3) | 50.3) | −10.3) | | | | | | | | | |
| Oral Ibuprofen | 65.5 | 43.9 | −21.6 | −33.0% | −3.3% | — | −3.5 | (−14.8, 7.8) | 0.5457 | — | | — |
| (3 × 400 mg) | (60.0, | (34.6, | (−29.8, | | | | | | | | | |
| (n = 20) | 71.0) | 53.1) | −13.4) | | | | | | | | | |
| 4.375 mg/knee | 60.2 | 33.8 | −26.4 | −43.9% | −14.2% | −10.9% | −8.3 | (−18.1, 1.5) | 0.1051 | −4.8 | (−14.9, 6.3) | 0.3619 |
| BID, (n = 23) | (54.5, | (27.0, | (−32.4, | | | | | | | | | |
| | 65.9) | 46.6) | −20.4) | | | | | | | | | |
| 8.75 mg/knee | 61.6 | 33.4 | −28.1 | −45.6% | −15.9% | −12.5% | −10.0 | (−21.1, 1.1) | 0.0859 | −6.6 | (−17.9, 4.9)) | 0.2737 |
| BID, (n = 24) | (57.9, | (26.8, | (−35.4, | | | | | | | | | |
| | 65.3) | 40.0) | −20.2) | | | | | | | | | |
| 17.5 mg/knee, | 63.9 | 26.7 | −37.2 | −58.2% | −28.5% | −25.2% | −19.1 | (−31.7, −6.5) | 0.0050 | −15.6 | (−28.2, −3) | 0.0205 |

| | | | |
|---|---|---|---|
| BID, (n = 19) | (58.7, | (18.5, | (−47.1, |
| | 69.1) | 34.9) | −27.3) |

*BID = twice a day; WOMAC = Western Ontario and McMaster Universities.
**Scale ranged from 0 to 100 mm with lower score as better.
***Negative numbers mean improved.

TABLE 52
Efficacy Analysis of Change from Baseline in the WOMAC* Overall Scores at Week 12

| | Baseline | Week 12 | Changes from | | | | Different | | | Different from | | |
|---|---|---|---|---|---|---|---|---|---|---|---|---|
| | (LS | (LS | Baseline (LS | % of improvement from | | | from placebo | | | oral ibuprofen | | |
| | Mean & | Mean & | Mean & | | | Oral | (LS Mean & | | P | (LS Mean & | | P |
| Cohort | 95% CI) | 95% CI) | 95% CI) | baseline | placebo | Ibuprofen | 95% CI) | | value | 95% CI) | | value |
| Placebo | 60.9 | 42.5 | −18.4 | −30.2% | — | 2.9% | — | | — | — | | — |
| (n = 25) | (55.0, | (35.1, | (−26.0, | | | | | | | | | |
| | 66.8) | 49.9) | −10.8) | | | | | | | | | |
| Oral Ibuprofen | 65.0 | 43.5 | −21.5 | −33.1% | −2.9% | — | −3.1 | (−13.9, 7.7) | 0.5845 | — | | — |
| (3 × 400 mg) | (60.0, | (34.6, | (−29.3, | | | | | | | | | |
| (n = 20) | 70.0) | 52.4) | −13.7) | | | | | | | | | |
| 4.37 5 mg/knee | 59.3 | 33.8 | −25.6 | −43.2% | −13.0% | −10.1% | −7.1 | (−16.5, 2.3) | 0.1452 | −4.1 | (−13.6, 5.5) | 0.4069 |
| BID, (n = 23) | (53.9, | (27.1, | (−31.2, | | | | | | | | | |
| | 64.7) | 40.5) | −20.0) | | | | | | | | | |
| 8.75 mg/knee | 60.7 | 33.3 | −27.4 | −45.1% | −14.9% | −11.8% | −9.0 | (−19.8, 1.8) | 0.1053 | −5.9 | (−16.8, 5.0) | 0.2934 |
| BID, (n = 24) | (57.1, | (26.9, | (−35.0, | | | | | | | | | |
| | 64.3) | 39.7) | −19.8) | | | | | | | | | |
| 17.5 mg/knee, | 63.4 | 26.8 | −36.6 | −57.7% | −27.5% | −24.6% | −18.2 | (−30.6, −5.8) | 0.0061 | −15.2 | (−28.2, −3.0) | 0.0224 |

| | | | |
|---|---|---|---|
| BID, (n = 19) | (58.4, | (18.8, | (−46.4, |
| | 68.4) | 34.8) | −26.8) |

*BID = twice a day; WOMAC = Western Ontario and McMaster Universities.
**Scale ranged from 0 to 100 mm with lower score as better.
***Negative numbers mean improved.

TABLE 53
Efficacy Analysis of Change from Baseline in the Subject’s Global Assessment of Disease Status Scores

| | Baseline | Week 12 | Changes from | | | | Different | | | Different from | | |
|---|---|---|---|---|---|---|---|---|---|---|---|---|
| | (LS | (LS | Baseline (LS | % of improvement from | | | from placebo | | | oral ibuprofen | | |
| | Mean & | Mean & | Mean & | | | Oral | (LS Mean & | | P | (LS Mean & | | P |
| Cohort | 95% CI) | 95% CI) | 95% CI) | baseline | placebo | Ibuprofen | 95% CI) | | value | 95% CI) | | value |
| Placebo | 67.0 | 42.6 | −24.4 | −36.4% | — | 2.0% | — | | — | 4.5 | (−8.4, 14.0) | — |
| (n = 25) | (61.7, | (35.9, | (−31.6, | | | | | | | | | |
| | 72.3) | 49.3) | −17.2) | | | | | | | | | |
| Oral Ibuprofen | 75.2 | 46.3 | −28.9 | −38.4% | −2.0% | — | −4.4 | (−13.8, 5.0) | 0.4681 | — | | — |
| (3 × 400 mg) | (70.3, | (37.0, | (−38.3, | | | | | | | | | |
| (n = 20) | 80.1) | 55.6) | −19.5) | | | | | | | | | |
| 4.375 mg/knee | 64.8 | 32.0 | −32.8 | −50.6% | −14.2% | −12.2% | −8.3 | (−18.6, 2.0) | 0.1191 | −3.9 | (−15.8, 8.0) | 0.5225 |
| BID, (n = 23) | (58.6, | (24.9, | (−40.2, | | | | | | | | | |
| | 71.0) | 39.1) | −25.4) | | | | | | | | | |
| 8.75 mg/knee | 67.2 | 33.1 | −34.1 | −50.7% | −14.3% | −11.8% | −9.6 | (−19.9, 0.7) | 0.0733 | −5.2 | (−17.1, 6.7) | 0.3956 |
| BID, (n = 24) | (63.0, | (26.0, | (−41.5, | | | | | | | | | |
| | 71.4) | 40.2) | −26.7) | | | | | | | | | |
| 17.5 mg/knee, | 69.3 | 26.2 | −43.1 | −62.2% | −25.8% | −23.8% | −18.6 | (−30.1, −7.1) | 0.0029 | −14.2 | (−27.2, −1.2) | 0.0388 |
| BID, (n = 19) | (64.5, | (17.8, | (−52.1, | | | | | | | | | |
| | 74.1) | 34.6) | −34.1) | | | | | | | | | |

*BID = twice a day; WOMAC = Western Ontario and McMaster Universities.
**Scale ranged from 0 to 100 mm with lower score as better.
***Negative numbers mean improved.

TABLE 54
Efficacy Analysis of Change from Baseline in the Subject’s pain Assessment Scores at Week 12

| | Baseline | Week 12 | Changes from | | | | Different | | | Different from | | |
|---|---|---|---|---|---|---|---|---|---|---|---|---|
| | (LS | (LS | Baseline (LS | % of improvement from | | | from placebo | | | oral ibuprofen | | |
| | Mean & | Mean & | Mean & | | | Oral | (LS Mean & | | P | (LS Mean & | | P |
| Cohort | 95% CI) | 95% CI) | 95% CI) | baseline | placebo | Ibuprofen | 95% CI) | | value | 95% CI) | | value |
| Placebo | 64.8 | 45.9 | −18.9 | −29.2% | — | 7.0% | — | | — | 6.9 | (−5.2, 16.6) | — |
| (n = 25) | (59.3, | (38.1, | (−26.1, | | | | | | | | | |
| | 70.3) | 53.7) | −11.6) | | | | | | | | | |
| Oral Ibuprofen | 71.2 | 45.4 | −25.8 | −36.2% | −7.0% | — | −6.9 | (−17.7, 4.1) | 0.2254 | — | | — |
| (3 × 400 mg) | (66.8, | (35.8, | (−33.9, | | | | | | | | | |
| (n = 20) | 75.6) | 55.0) | −17.7) | | | | | | | | | |
| 4.375 mg/knee | 59.7 | 36.7 | −23.0 | −38.5% | −9.3% | −2.3% | −4.0 | (−14.5, 5.5) | 0.4103 | 2.8 | (−7.4, 13.0) | — |
| BID, (n = 23) | (54.4, | (30.2, | (−29.1, | | | | | | | | | |
| | 65.0) | 43.2) | −16.9) | | | | | | | | | |
| 8.75 mg/knee | 63.5 | 33.2 | −30.3 | −47.7% | −18.5% | −10.4% | −11.4 | (−22.8, 0.0) | 0.0561 | −3.8 | (−15.6, 8.0) | 0.5301 |
| BID, (n = 24) | (59.0, | (26.8, | (−39.1, | | | | | | | | | |
| | 68.0) | 39.6) | −21.5) | | | | | | | | | |
| 17.5 mg/knee, | 68.6 | 28.7 | −39.9 | −58.2% | −29.0% | −22.0% | −21.0 | (−33.8, −8.0) | 0.0028 | −14.1 | (−27.1, −0.7) | 0.0461 |

| | | | |
|---|---|---|---|
| BID, (n = 19) | (64.1, | (19.9, | (−50.6, |
| | 73.1) | 37.5) | −29.2) |

*BID = twice a day; WOMAC = Western Ontario and McMaster Universities.
**Scale ranged from 0 to 100 mm with lower score as better.
***Negative numbers mean improved.

As shown by the reduction in pain, stiffness, functional ability severity in the patient's assessment in WOMAC (VAS) subscale scores, WOMAC overall scores, the Subject's Global Assessment of Disease Status scores (SGADS), and the Subject's pain Assessment Scores (SPA), 2-(diethylamino)ethyl 2-(4-isobutylphenyl)propionate can reduce the signs and symptoms of osteoarthritis in a dose response manner. As shown in Tables 49-54, the 17.5 mg/knee of 2-(diethylamino)ethyl 2-(4-isobutylphenyl)propionate, BID, has much higher efficacy than oral ibuprofen (3×400 mg/day, −14.5 mm in pain, −12.4 mm in stiffness, −15.6 in functional ability, −15.2 mm in WOMAC overall, −14.2 mm in SGADS, and −14.1 mm in SPA) and placebo (−15.0 mm in pain, −19.1 mm in stiffness, −19.1 in functional ability, −18.2 mm in WOMAC overall, −18.6 mm in SGADS, and −21.0 mm in SPA) significantly. The efficacy (measured by overall of the six scores) of ibuprofen is better than placebo, but not significantly.

TABLE 55
Efficacy Analysis in Subject Global Impression of Change (SGIC), Investigator’s global assessment
of disease status (IGADS), and in Investigator’s assessment of response to therapy (IART) at Week 12

| | | | Oral Ibuprofen | 4.375 mg/knee, | 8.75 mg/knee, | 17.5 mg/knee, |
|---|---|---|---|---|---|---|
| | | Placebo | (3 x 400 mg) | BID, | BID, | BID, |
| Cohort | | (n = 25) | (n = 20) | (n = 23) | (n = 24) | (n = 19) |
| SGIC | Good and Very good | 20.0% | 55.0% | 39.1% | 37.5% | 73.7% |
| | fair | 48.0% | 35.0% | 56.5% | 45.8% | 21.1% |
| | Poor and Very Poor | 32.0% | 10.0% | 4.3% | 16.7% | 5.3% |
| IGADS | Good and Very good | 20.0% | 40.0% | 43.5% | 41.7% | 68.4% |
| | fair | 60.0% | 45.0% | 53.2% | 54.2% | 31.6% |
| | Poor and Very Poor | 20.0% | 15.0% | 4.3% | 4.2% | 0% |
| IART | Good and Very good | 24.0% | 50.0% | 39.1% | 37.5% | 57.9% |
| | fair | 36.0% | 25.0% | 56.5% | 50.0% | 36.8% |
| | Poor and Very Poor | 40.0% | 25.0% | 4.3% | 12.5% | 5.3% |

Interestingly, as shown in Table 55, the efficacy measured by WOMAC VAS scores of oral ibuprofen is better than placebo, but not significantly, however, the SGIC, IGADS, IART scores show clearly that oral ibuprofen is much better than placebo, 17.5 mg/knee of 2-(diethylamino)ethyl 2-(4-isobutylphenyl)propionate is the best one and has a very high Subject's satisfaction rate (73.7%) which is very much better than oral ibuprofen (55.0%) and placebo (20.0%). 2-(diethylamino)ethyl 2-(4-isobutylphenyl)propionate is a prodrug of ibuprofen which can penetrate cartilage, skin, and bone in a very high rate and >99.9% of 2-(diethylamino)ethyl 2-(4-isobutylphenyl)propionate is metabolized to ibuprofen in any human tissue (except skin, the in vitro T1/2 of 2-(diethylamino)ethyl 2-(4-isobutylphenyl)propionate with animal skin is ˜20 hours) in a very short time (2-(diethylamino)ethyl 2-(4-isobutylphenyl)propionate is very difficult to be detected in plasma due to the very short T1/2 (in vitro: ˜4 min. in plasma). 2-(diethylamino)ethyl 2-(4-isobutylphenyl)propionate is designed to minimize the plasma exposure to minimize side effects and maximize the local tissue exposure to maximize the efficacy. When is 2-(diethylamino)ethyl 2-(4-isobutylphenyl)propionate (9 mg/kg, equal to 6.73 mg/kg ibuprofen sodium) administered to mini pig transdermally, the plasma exposure (AUCₗₐₛₜ 1516 h-ng/mL) is only ˜10% of the plasma exposure (AUCₗₐₛₜ 15466 h-ng/mL) of oral ibuprofen sodium and the Cₘₐₓ (38.61 ng/mL) is less than 0.3% Cₘₐₓ(13110.55 ng/mL) of oral ibuprofen, however the cartilage exposure (AUCₗₐₛₜ 23.91 h·μg/mL) and muscle exposure (AUCₗₐₛₜ 35.89 h·μg/mL) are 8-9 time higher than that of oral ibuprofen (cartilage: AUCₗₐₛₜ 3.10 h·μg/mL, muscle: AUCₗₐₛₜ 4.21 h·μg/mL), ibuprofen is one of most used NSAIDs, so high efficacy and safety of 2-(diethylamino)ethyl 2-(4-isobutylphenyl)propionate can be expected reasonably. As shown by the reduction in pain, stiffness, functional ability severity in the patient's assessment in WOMAC (VAS) pain subscale scores, WOMAC overall scores, the Subject's Global Assessment of Disease Status Scores (SGADS), and the Subject's pain Assessment Scores (SPA) in the second Phase 2 clinical trial, 2-(diethylamino)ethyl 2-(4-isobutylphenyl)propionate can reduce the signs and symptoms of osteoarthritis in a dose response manner: the efficacy of 17.5 mg/knee of 2-(diethylamino)ethyl 2-(4-isobutylphenyl)propionate, BID>8.75 mg/knee of 2-(diethylamino)ethyl 2-(4-isobutylphenyl)propionate>4.375 mg/knee of 2-(diethylamino)ethyl 2-(4-isobutylphenyl)propionate>3×400 mg of oral ibuprofen>placebo. The 17.5 mg/knee of 2-(diethylamino)ethyl 2-(4-isobutylphenyl)propionate, BID is better than oral ibuprofen (3×400 mg/day) and placebo significantly, the differences from oral ibuprofen: −14.5 mm in pain, −12.4 mm in stiffness, −15.6 in functional ability, −15.2 mm in WOMAC overall, −14.2 in SGADS, and −14.1 in SPA; and from placebo: −15.0 mm in pain, −19.1 mm in stiffness, −19.1 in functional ability, −18.2 mm in WOMAC overall, −18.6 mm in SGADS, and −21.0 mm in SPA.

The difference between oral ibuprofen (3×400 mg) and placebo is quite small: −0.5 mm in pain, −6.7 mm in stiffness, −3.5 mm in functional ability, −3.0 mm in WOMAC overall, −4.2 mm in the Subject's Global Assessment of Disease Status scores, and 6.1 mm in the Subject's pain Assessment Scores, however, in the “good and very good” rate of the Subject Global Impression of Change is 55% (oral ibuprofen) vs. 20% (placebo) (17.5 mg/knee, BID of 2-(diethylamino)ethyl 2-(4-isobutylphenyl)propionate is 73.7%) and the difference is very clear. For the small difference between oral ibuprofen and placebo, the first reason may be the sample size is small, the second one may be the placebo effects are higher in Asian subjects. Margaret N Essex etc. (4 researchers are all Pfizer employees) reported a small different from placebo in subjects with Asian descent in 31 centers in the US in compliance with the principles of Good Clinical Practice and the Declaration of Helsinki: celecoxib vs. placebo: −5.6 mm (0-20 mm) vs.−4.3 mm (0-20 mm), difference: −1.3 mm (0-20 mm) or −6.5 mm (0-100 mm) in WOMAC pain; −17.3 mm (0-68 mm) vs. −13.9 mm (0-68 mm), difference: 3.4 mm (0-68 mm) or 5.0 mm (0-100 mm) in WOMAC functional ability; −2.0 mm (0-8 mm) vs. −1.6 mm (0-8 mm), difference: 0.4 mm (0-8 mm) or 5.0 mm (0-100 mm) in WOMAC joint stiffness; −24.9 mm (0-96 mm) vs. −19.7 mm (0-96 mm), difference: 5.2 mm (0-96 mm) or 5.4 mm (0-100 mm) in WOMAC total. In Patient's Assessment of Arthritis Pain (VAS 0-100 mm), celecoxib (n=121) vs. placebo (n=58): −37.1 mm vs. −33.6 mm, P value=0.2403; Naproxen (n=107) vs. placebo (n=58): −37.5 mm vs. −33.6 mm, P value=0.2027.

Based on the safety and efficacy results of two Phase 1 and two Phase 2 clinical trials, 2-(diethylamino)ethyl 2-(4-isobutylphenyl)propionate can be the first high effective and safe OA drug for everyday use, and the subjects in high dose group have average scores below 30 mm at Week 12 in all six subscales, that may mean that people with OA will have a normal life with every day use of 2-(diethylamino)ethyl 2-(4-isobutylphenyl)propionate.

The sign and symptoms relief are getting better and better by time and the onset of the sign and symptoms relief is fast as shown in the following Tables 56-61.

TABLE 56
The Change from Baseline in the WOMAC* Pain
Subscale Scores from Week 2 to Week 12

| | Week | Week | Week | Week |
|---|---|---|---|---|
| | 2 (SD) | 4 (SD) | 8 (SD) | 12 (SD) |
| Cohort | in mm | in mm | in mm | in mm |
| Placebo | −6.1 (8.9) | −10.4 (11.6) | −15.6 (12.6) | −21.1 (19.0) |
| (n = 25) | | | | |
| Oral | −8.8 (14.4) | −10.6 (15.9) | −16.0 (18.2) | −21.6 (18.4) |
| Ibuprofen | | | | |
| (3 × 400 mg) | | | | |
| (n = 20) | | | | |
| 4.375 mg/knee, | −11.9 (9.8) | −14.4 (10.9) | −20.2 (12.8) | −25.7 (14.0) |
| BID, (n = 23) | | | | |
| 8.75 mg/knee, | −7.9 (15.7) | −16.5 (18.0) | −21.2 (17.2) | −27.7 (18.8) |
| BID, (n = 24) | | | | |
| 17.5 mg/knee, | −10.4 (9.0) | −19.6 (16.3) | −30.6 (19.3) | −36.2 (22.8) |
| BID, (n = 19) | | | | |

TABLE 57
The Change from Baseline in the WOMAC* Stiffness
Subscale Scores from Week 2 to Week 12

| | Week | Week | Week | Week |
|---|---|---|---|---|
| | 2 (SD) | 4 (SD) | 8 (SD) | 12 (SD) |
| Cohort | in mm | in mm | in mm | in mm |
| Placebo | −2.8 (9.8) | −4.6 (15.7) | −9.8 (16.4) | −14.0 (21.4) |
| (n = 25) | | | | |
| Oral | −8.8 (14.8) | −7.1 (21.6) | −14.1 (18.1) | −20.6 (17.6) |
| Ibuprofen | | | | |
| (3 × 400 mg) | | | | |
| (n = 20) | | | | |
| 4.375 mg/knee, | −5.8 (13.8) | −12.6 (14.4) | −14.6 (16.4) | −18.0 (15.6) |
| BID, (n = 23) | | | | |
| 8.75 mg/knee, | −5.3 (14.2) | −11.1 (18.1) | −16.0 (17.7) | −20.4 (18.5) |
| BID, (n = 24) | | | | |
| 17.5 mg/knee, | −10.9 (11.3) | −16.8 (14.9) | −27.9 (19.6) | −33.1 (21.0) |
| BID, (n = 19) | | | | |

TABLE 58
Change from Baseline in the WOMAC* Difficulty Performing
Daily Activities Subscale Scores from Week 2 to Week 12

| | Week | Week | Week | Week |
|---|---|---|---|---|
| | 2 (SD) | 4 (SD) | 8 (SD) | 12 (SD) |
| Cohort | in mm | in mm | in mm | in mm |
| Placebo | −4.3 (9.4) | −8.6 (13.3) | −13.2 (14.7) | −18.1 (19.9) |
| (n = 25) | | | | |
| Oral | −8.2 (12.6) | −11.1 (13.3) | −16.0 (16.6) | −21.6 (18.7) |
| Ibuprofen | | | | |
| (3 × 400 mg) | | | | |
| (n = 20) | | | | |
| 4.375 mg/knee, | −12.0 (11.6) | −15.7 (12.3) | −21.4 (13.8) | −26.4 (14.6) |
| BID, (n = 23) | | | | |
| 8.75 mg/knee, | −7.4 (15.8) | −17.6 (18.9) | −22.3 (18.6) | −28.1 (19.9) |
| BID, (n = 24) | | | | |
| 17.5 mg/knee, | −12.0 (12.6) | −19.2 (17.5) | −31.9 (19.4) | −37.2 (22.1) |
| BID, (n = 19) | | | | |

TABLE 59
The Change from Baseline in the WOMAC*
Overall Scores from Week 2 to Week 12

| | Week | Week | Week | Week |
|---|---|---|---|---|
| | 2 (SD) | 4 (SD) | 8 (SD) | 12 (SD) |
| Cohort | in mm | in mm | in mm | in mm |
| Placebo | −4.5 (8.7) | −8.6 (12.6) | −13.4 (13.9) | −18.4 (19.4) |
| (n = 25) | | | | |
| Oral | −7.8 (12.2) | −10.5 (13.3) | −15.5 (16.3) | −21.5 (17.7) |
| Ibuprofen | | | | |
| (3 × 400 mg) | | | | |
| (n = 20) | | | | |
| 4.375 mg/knee, | −11.5 (10.8) | −15.2 (11.5) | −20.6 (13.1) | −25.6 (13.8) |
| BID, (n = 23) | | | | |
| 8.75 mg/knee, | −7.4 (15.0) | −16.8 (18.1) | −21.6 (17.7) | −27.4 (19.1) |
| BID, (n = 24) | | | | |
| 17.5 mg/knee, | −11.6 (11.4) | −19.1 (16.5) | −31.3 (18.8) | −36.6 (21.8) |
| BID, (n = 19) | | | | |

TABLE 60
The Change from Baseline in the Subject's Global Assessment
of Disease Status Scores from Week 2 to Week 12

| | Week | Week | Week | Week |
|---|---|---|---|---|
| | 2 (SD) | 4 (SD) | 8 (SD) | 12 (SD) |
| Cohort | in mm | in mm | in mm | in mm |
| Placebo | −7.3 (9.1) | −15.1 (14.1) | −20.1 (13.8) | −24.4 (18.4) |
| (n = 25) | | | | |
| Oral | −10.7 (11.7) | −18.5 (14.0) | −24.8 (20.5) | −28.9 (21.4) |
| Ibuprofen | | | | |
| (3 × 400 mg) | | | | |
| (n = 20) | | | | |
| 4.375 mg/knee, | −14.3 (17.5) | −20.9 (16.1) | −25.9 (18.3) | −32.8 (18.0) |
| BID, (n = 23) | | | | |
| 8.75 mg/knee, | −10.1 (11.9) | −21.0 (16.1) | −25.0 (16.9) | −34.1 (18.4) |
| BID, (n = 24) | | | | |
| 17.5 mg/knee, | −11.5 (11.9) | −24.7 (17.3) | −35.9 (19.1) | −43.1 (20.0) |
| BID, (n = 19) | | | | |

TABLE 61
The Change from Baseline in the Subject's
pain Assessment Scores from Week 2 to Week 12

| | Week | Week | Week | Week |
|---|---|---|---|---|
| | 2 (SD) | 4 (SD) | 8 (SD) | 12 (SD) |
| Cohort | in mm | in mm | in mm | in mm |
| Placebo | −5.0 (7.7) | −10.8 (10.5) | −13.6 (12.5) | −18.9 (18.5) |
| (n = 25) | | | | |
| Oral | −9.8 (10.0) | −12.4 (12.2) | −21.2 (15.9) | −25.8 (18.5) |
| Ibuprofen | | | | |
| (3 × 400 mg) | | | | |
| (n = 20) | | | | |
| 4.375 mg/knee, | −10.0 (13.4) | −12.2 (10.2) | −18.1 (15.0) | −23.0 (15.0) |
| BID, (n = 23) | | | | |
| 8.75 mg/knee, | −9.2 (13.6) | −18.6 (18.6) | −21.3 (18.9) | −30.3 (22.0) |
| BID, (n = 24) | | | | |
| 17.5 mg/knee, | −13.3 (13.7) | −23.1 (20.9) | −33.1 (20.5) | −39.9 (23.8) |
| BID, (n = 19) | | | | |

The clinical data of all subjects, including the volunteer subjects and the recruited subjects by the third party, a clinical trial patient recruitment services company are shown from Tables 62-69.

TABLE 62
Patient Demographic and Baseline Characteristics

| | 4.375 | 8.75 | 17.5 | Oral | |
|---|---|---|---|---|---|
| | mg/knee | mg/knee | mg/knee, | Ibuprofen | |
| | BID, | BID, | BID, | (3 × 400 mg) | Placebo |
| Characteristic | (n = 49) | (n = 54) | (n = 52) | (n = 48) | (n = 48) |
| Mean(rang) age(y) | 56.0 (43-69) | 58.8 (44-71) | 58.8 (43-71) | 59.7 (49-72) | 56.1 (44-69) |
| Female sex (%) | 40 (81.6%) | 43 (79.6%) | 42 (80.8%) | 38 (79.2%) | 41 (85.4%) |
| Male sex (%) | 9 (18.4%) | 11 (20.4%) | 10 (19.2%) | 10 (20.8%) | 7 (14.6%) |
| Asian | 100% | 100% | 100% | 100% | 100% |
| Mean ± SD | 61.1 (±11.5) | 62.0 (±11.2) | 62.2 (±12.0) | 61.6 (±11.7) | 61.6 (±11.5) |
| WOMAC pain | | | | | |
| Mean ± SD | 48.8 (±19.2) | 55.3 (±14.4) | 52.3 (±15.8) | 51.6 (±21.4) | 50.9 (±18.0) |
| WOMAC stiffness | | | | | |
| Mean ± SD | 59.3 (±14.5) | 60.5 (±9.4) | 59.8 (±12.4) | 60.9 (±12.2) | 58.9 (±14.7) |
| WOMAC difficulty | | | | | |
| Mean ± SD | 66.8 (±14.8) | 69.0 (±9.3) | 66.9 (±11.6) | 71.4 (±12.1) | 66.3 (±11.8) |
| WOMAC Patients's | | | | | |
| global assessment | | | | | |

TABLE 63
Efficacy Analysis of Change from Baseline in the WOMAC* Pain Subscale Scores at Week 12

| | Baseline | Week 12 | Changes from | | | | Different | | | Different from | | |
|---|---|---|---|---|---|---|---|---|---|---|---|---|
| | (LS | (LS | Baseline (LS | % of improvement from | | | from placebo | | | oral ibuprofen | | |
| | Mean & | Mean & | Mean & | | | Oral | (LS Mean & | | P | (LS Mean & | | P |
| Cohort | 95% CI) | 95% CI) | 95% CI) | baseline | Placebo | Ibuprofen | 95% CI) | | value | 95% CI) | | value |
| Placebo | 61.6 | 39.0 | −22.6 | −36.7% | — | 4.5% | — | | — | — | | — |
| (n = 48) | (58.4, | (33.6, | (−27.7, | | | | | | | | | |
| | 64.8) | 44.4) | −17.5) | | | | | | | | | |
| Oral Ibuprofen | 61.6 | 36.3 | −25.4 | −41.2% | −4.5% | — | — | | — | — | | — |
| (3 × 400 mg) | (58.3, | (31.3, | (−30.4, | | | | | | | | | |
| (n = 48) | 64.9) | 41.3) | −20.4) | | | | | | | | | |
| 4.375 mg/knee | 61.1 | 35.5 | −25.6 | −41.9% | −5.2% | −0.7% | — | | — | — | | — |
| BID, (n = 49) | (57.9, | (30.5, | (−30.8, | | | | | | | | | |
| | 64.3) | 40.5) | −20.5) | | | | | | | | | |
| 8.75 mg/knee | 62.0 | 33.4 | −28.6 | −46.1% | −9.4% | −4.9% | −6.0 | (−12.9, 0.9) | 0.0915 | −3.2 | (−10.0, 3.6) | 0.3582 |
| BID, (n = 54) | (59.0, | (29.1, | (−33.2, | | | | | | | | | |
| | 65.0) | 37.7) | −24.0) | | | | | | | | | |
| 17.5 mg/knee, | 62.1 | 30.5 | −31.7 | −51.0% | −14.3% | −9.8% | −9.1 | (−16.3, −1.9) | 0.0152 | −6.3 | (−13.4, 0.8) | 0.0868 |

| | | | |
|---|---|---|---|
| BID, (n = 52) | (58.8, | (26.2, | (−36.6, |
| | 65.4) | 34.8) | −26.8) |

*BID = twice a day; WOMAC = Western Ontario and McMaster Universities.
**Scale ranged from 0 to 100 mm with lower score as better.
***Negative numbers mean improved.

TABLE 64
Efficacy Analysis of Change from Baseline in the WOMAC* Stiffness Subscale Scores at Week 12

| | Baseline | Week 12 | Changes from | | | | Different | | Different from | |
|---|---|---|---|---|---|---|---|---|---|---|
| | (LS | (LS | Baseline (LS | % of improvement from | | | from placebo | | oral ibuprofen | |
| | Mean & | Mean & | Mean & | | | Oral | (LS Mean & | P | (LS Mean & | P |
| Cohort | 95% CI) | 95% CI) | 95% CI) | baseline | Placebo | Ibuprofen | 95% CI) | value | 95% CI) | value |
| Placebo | 50.9 | 34.4 | −16.5 | −32.4% | — | 6.9% | — | — | — | — |
| (n = 48) | (47.7, 54.1) | (28.7, 40.1) | (−22.9, −10.1) | | | | | | | |
| Oral Ibuprofen | 51.6 | 31.3 | −20.3 | −39.3% | −6.9% | — | — | — | — | — |
| (3 × 400 mg) | (45.6, 57.6) | (25.7, 36.9) | (−25.8, −14.8) | | | | | | | |
| (n = 48) | | | | | | | | | | |
| 4.375 mg/knee | 48.8 | 31.9 | −16.9 | −34.6% | −2.2% | 4.7% | — | — | — | — |
| BID, (n = 49) | (43.4, 54.2) | (26.7, 37.1) | (−22.6, −11.2) | | | | | | | |
| 8.75 mg/knee | 55.3 | 32.0 | −23.3 | −42.1% | −9.7% | −2.8% | −6.7 | 0.1157 | −3.0 | 0.4461 |
| BID, (n = 54) | (51.5, 59.1) | (27.2, 36.8) | (−28.5, −18.1) | | | | (−15.0, 1.6) | | (−10.6, 4.6) | |
| 17.5 mg/knee, | 53.3 | 28.1 | −25.2 | −47.3% | −14.9% | −8.0% | −8.6 | 0.0587 | −4.9 | 0.2460 |
| BID, (n = 52) | (49.0, 57.6) | (23.8, 32.4) | (−31.2, −19.2) | | | | (−17.4, 0.2) | | (−13.1, 3.3) | |

*BID = twice a day; WOMAC = Western Ontario and McMaster Universities.
**Scale ranged from 0 to 100 mm with lower score as better.
***Negative numbers mean improved.

TABLE 65
Efficacy Analysis of Change from Baseline in the WOMAC* Difficulty Performing Daily Activities
Subscale Scores at Week 12

| | | | Changes from | | | | Different | | Different from | |
|---|---|---|---|---|---|---|---|---|---|---|
| | Baseline | Week 12 | Baseline | % of improvement from | | | from placebo | | oral ibuprofen | |
| | (LS Mean | (LS Mean | (LS Mean | | | Oral | (LS Mean | P | (LS Mean | P |
| Cohort | &95% CI) | &95% CI) | &95% CI) | baseline | Placebo | Ibuprofen | &95% CI) | value | &95% CI) | value |
| Placebo (n = 48) | 58.9 | 36.9 | −21.9 | −37.2% | — | 4.3 % | — | — | — | — |
| | (54.7, 63.1) | (31.4, 42.4) | (−27.4, −16.4) | | | | | | | |
| Oral Ibuprofen | 60.9 | 35.7 | −25.3 | −41.5% | −4.3% | — | — | — | — | — |
| (3 × 400 mg) | (57.4, 64.3) | (30.4, 41.0) | (−30.7, −19.9) | | | | | | | |
| (n = 48) | | | | | | | | | | |
| 4.375 mg/knee | 59.3 | 33.9 | −25.4 | −42.8% | −5.6% | −1.3% | — | — | — | — |
| BID, (n = 49) | (55.2, 63.4) | (29.1, 38.7) | (−30.4, −20.4) | | | | | | | |
| 8.75 mg/knee | 60.5 | 32.6 | −27.9 | −46.1% | −8.9% | −4.6% | −6.0 | 0.1157 | −2.7 | 0.4781 |
| BID, (n = 54) | (58.0, 63.0) | (27.9, 37.3) | (−32.9, −22.9) | | | | (−13.4, 1.4) | | (−10.1, 4.7) | |
| 17.5 mg/knee, | 59.8 | 28.3 | −31.4 | −52.5% | −15.3% | −11.0% | −9.5 | 0.0164 | −6.2 | 0.1126 |
| BID, (n = 52) | (56.4, 63.2) | (24.2, 32.4) | (−36.6, −26.1) | | | | (−17.1, −1.9) | | (−13.8, 1.4) | |

*BID = twice a day; WOMAC = Western Ontario and McMaster Universities.
**Scale ranged from 0 to 100 mm with lower score as better.
***Negative numbers mean improved.

TABLE 66
Efficacy Analysis of Change from Baseline in the WOMAC* Overall Scores at Week 12

| | | | Changes from | | | | Different | | Different from | |
|---|---|---|---|---|---|---|---|---|---|---|
| | Baseline | Week 12 | Baseline | % of improvement from | | | from placebo | | oral ibuprofen | |
| | (LS Mean | (LS Mean | (LS Mean | | | Oral | (LS Mean | P | (LS Mean | P |
| Cohort | &95% CI) | &95% CI) | &95% CI) | baseline | Placebo | Ibuprofen | &95% CI) | value | &95% CI) | value |
| Placebo (n = 48) | 58.8 | 37.5 | −21.3 | −36.2% | — | 5.1% | — | — | — | — |
| | (55.0, 62.6) | (32.1, 42.9) | (−26.7, −15.9) | | | | | | | |
| Oral Ibuprofen | 60.3 | 35.4 | −24.9 | −41.3% | −5.1% | — | — | — | — | — |
| (3 × 400 mg) | (57.2, 63.4) | (30.3, 40.5) | (−30.0, −19.8) | | | | | | | |
| (n = 48) | | | | | | | | | | |
| 4.375 mg/knee | 59.4 | 34.0 | −25.4 | −42.8% | −6.6% | −1.5% | — | — | — | — |
| BID, (n = 49) | (55.7, 63.1) | (29.3, 38.7) | (−30.1, −20.7) | | | | | | | |
| 8.75 mg/knee | 60.4 | 32.7 | −27.7 | −45.9% | −9.7% | −4.6% | −6.4 | 0.0818 | −2.8 | 0.4297 |
| BID, (n = 54) | (57.9, 62.9) | (28.2, 37.2) | (−32.5, −22.9) | | | | (−13.5, 0.7) | | (−9.9, 4.3) | |
| 17.5 mg/knee, | 59.5 | 28.6 | −30.9 | −51.9% | −15.7% | −10.6% | −9.6 | 0.0123 | −6.0 | 0.1095 |
| BID, (n = 52) | (56.4, 62.6) | (24.5, 32.7) | (−36.0, −25.8) | | | | (−17.0, −2.2) | | (−13.2, 1.2) | |

*BID = twice a day; WOMAC = Western Ontario and McMaster Universities.
**Scale ranged from 0 to 100 mm with lower score as better.
***Negative numbers mean improved.

TABLE 67
Efficacy Analysis of Change from Baseline in the Subject's Global Assessment of Disease Status Scores

| | | | Changes from | | | | Different | | Different from | |
|---|---|---|---|---|---|---|---|---|---|---|
| | Baseline | Week 12 | Baseline | % of improvement from | | | from placebo | | oral ibuprofen | |
| | (LS Mean | (LS Mean | (LS Mean | | | Oral | (LS Mean | P | (LS Mean | P |
| Cohort | &95% CI) | &95% CI) | &95% CI) | baseline | Placebo | Ibuprofen | &95% CI) | value | &95% CI) | value |
| Placebo (n = 48) | 66.3 | 35.8 | −30.4 | −45.9% | — | 0.7% | — | — | — | — |
| | (63.0, 69.6) | (30.8, 40.8) | (−35.7, −25.1) | | | | | | | |
| Oral Ibuprofen | 71.4 | 38.1 | −33.3 | −46.6% | −0.7% | — | — | — | — | — |
| (3 × 400 mg) | (68.0, 74.8) | (32.7, 43.5) | (−39.1, −27.5) | | | | | | | |
| (n = 48) | | | | | | | | | | |
| 4.375 mg/knee | 66.8 | 32.1 | −34.7 | −51.9% | −6.0% | −5.3% | — | — | — | — |
| BID, (n = 49) | (62.7, 70.9) | (27.1, 37.1) | (−40.6, −28.8) | | | | | | | |
| 8.75 mg/knee | 69.0 | 32.4 | −36.6 | −53.0% | −7.1% | −6.4% | −6.2 | 0.1072 | −3.4 | 0.3957 |
| BID, (n = 54) | (66.5, 71.5) | (27.5, 37.3) | (−41.7, −31.5) | | | | (−13.6, 1.2) | | (−11.0, 4.4) | |
| 17.5 mg/knee, | 67.0 | 28.7 | −38.3 | −57.2% | −11.3% | −10.6% | −7.9 | 0.0374 | −5.1 | 0.1970 |
| BID, (n = 52) | (63.8, 70.1) | (24.0, 33.4) | (−43.2, −33.2) | | | | (−15.2, −0.6) | | (−12.8, 2.6) | |

*BID = twice a day; WOMAC = Western Ontario and McMaster Universities.
**Scale ranged from 0 to 100 mm with lower score as better.
***Negative numbers mean improved.

TABLE 68
Efficacy Analysis of Change from Baseline in the Subject's pain Assessment Scores at Week 12

| | | | Changes from | | | | Different | | Different from | |
|---|---|---|---|---|---|---|---|---|---|---|
| | Baseline | Week 12 | Baseline | % of improvement from | | | from placebo | | oral ibuprofen | |
| | (LS Mean | (LS Mean | (LS Mean | | | Oral | (LS Mean | P | (LS Mean | P |
| Cohort | &95% CI) | &95% CI) | &95% CI) | baseline | Placebo | Ibuprofen | &95% CI) | value | &95% CI) | value |
| Placebo (n = 48) | 65.1 | 41.0 | −24.1 | −37.0% | — | 8.4% | — | — | — | — |
| | (61.9, 68.3) | (35.4, 46.6) | (−29.6, −18.6) | | | | | | | |
| Oral Ibuprofen | 67.1 | 36.6 | −30.5 | −45.4% | −8.4% | — | — | — | — | — |
| (3 × 400 mg) | (63.7, 70.5) | (31.3, 41.9) | (−35.7, −25.3) | | | | | | | |
| (n = 48) | | | | | | | | | | |
| 4.375 mg/knee | 63.2 | 37.5 | −25.6 | −40.5% | −3.5% | 4.9% | — | — | — | — |
| BID, (n = 49) | (59.6, 66.8) | (32.3, 42.7) | (−30.9, −20.3) | | | | | | | |
| 8.75 mg/knee | 64.9 | 33.5 | −31.4 | −48.4% | −11.4% | −3.0% | −7.2 | 0.0762 | −0.8 | 0.8333 |
| BID, (n = 54) | (61.9, 67.9) | (29.0, 38.0) | (−37.0, −25.8) | | | | (−15.1, −0.7) | | (−8.4, 6.8) | |
| 17.5 mg/knee, | 64.5 | 30.0 | −34.6 | −53.6% | −16.6% | −8.2% | −10.5 | 0.0083 | −4.1 | 0.2788 |
| BID, (n = 52) | (61.3, 67.5) | (25.6, 34.2) | (−39.7, −29.5) | | | | (−18.1, −2.9) | | (−11.4, 3.3) | |

*BID = twice a day; WOMAC = Western Ontario and McMaster Universities.
**Scale ranged from 0 to 100 mm with lower score as better.
***Negative numbers mean improved.

TABLE 69
Efficacy Analysis in Subject Global Impression of Change (SGIC), Investigator's global assessment
of disease status (IGADS), and in Investigator's assessment of response to therapy (IART) at Week 12

| | | Placebo | Oral Ibuprofen | 4.375 mg/knee, | 8.75 mg/knee, | 17.5 mg/knee, |
|---|---|---|---|---|---|---|
| | | (n = 48) | (3 × 400 mg) (n = 48) | BID, (n = 49) | BID, (n = 54) | BID, (n = 52) |
| SGIC | Good and Very good | 29.2% | 47.9% | 32.7% | 46.3% | 61.5% |
| | fair | 43.8% | 43.8% | 57.1% | 37.0% | 32.7% |
| | Poor and Very Poor | 27.1% | 8.3% | 10.2% | 16.7% | 5.8% |
| IGADS | Good and Very good | 31.3% | 39.6% | 40.8% | 48.1% | 63.5% |
| | fair | 54.2% | 56.3% | 51.0% | 44.4% | 34.6% |
| | Poor and Very Poor | 12.5% | 4.1% | 8.2% | 7.4% | 1.9% |
| IART | Good and Very good | 29.2% | 47.9% | 40.8% | 50.0% | 61.5% |
| | fair | 37.5% | 43.8% | 49.0% | 37.0% | 34.6% |
| | Poor and Very Poor | 33.3% | 8.3% | 10.2% | 13.0% | 3.8% |

As shown by the reduction in pain, stiffness, functional ability severity in the patient's assessment in WOMAC (VAS) pain subscale scores, WOMAC overall scores, the Subject's Global Assessment of Disease Status scorns (SGADS), and the Subject's pain Assessment Scores (SPA), 2-(diethylamino)ethyl 2-(4-isobutylphenyl)propionate can reduce the signs and symptoms of osteoarthritis in a dose response manner. The 17.5 mg/knee of 2-(diethylamino)ethyl 2-(4-isobutylphenyl)propionate, BID, has higher efficacy than placebo significantly and to higher efficacy than oral ibuprofen (3×400 mg/day), but not significantly (Table 70).

TABLE 70
The Difference of Six Subscales between 17.5 mg, BID of 2-(diethylamino)ethyl
2-(4-isobutylphenyl)propionate, Oral Ibuprofen (3 × 400 mg), and Placebo

| | | | WOMAC | | | |
|---|---|---|---|---|---|---|
| | | WOMAC | Functional | WOMAC | | |
| Cohort vs. Cohort | WOMAC Pain | Stiffness | Ability | Total | SGADS | SPA |
| Oral Ibuprofen | −2.8 mm | −3.8 mm | −3.4 mm | −3.6 mm | −2.9 mm | −6.4 mm |
| (3 × 400 mg, n = 48) | | | | | | |
| vs. Placebo (n = 48) | | | | | | |
| 17.5 mg/knee, BID | −9.1 mm | −8.6 mm | −9.5 mm | −9.6 mm | −7.9 mm | −10.5 mm |
| 2-(diethylamino)ethyl | (p value = | (p value = | (p value = | (p value = | (p value = | (p value = |
| 2-(4-isobutylphenyl)propionate | 0.0152) | 0.0587) | 0.0164) | 0.0152) | 0.0374) | 0.0083) |
| (n = 52) vs. Placebo (n = 48) | | | | | | |
| 17.5 mg/knee, BID | −6.3 mm | −4.9 mm | −6.1 mm | −6.0 mm | −5.0 mm | −4.1 mm |
| 2-(diethylamino)ethyl | | | | | | |
| 2-(4-isobutylphenyl)propionate | | | | | | |
| (n = 52) vs. Oral Ibuprofen | | | | | | |
| (3 × 400 mg, n = 48) | | | | | | |

The WOMAC scores of oral ibuprofen show some better than placebo, but not significantly, however, the results of the Subject Global Impression of Change (SGIC), Investigator's global assessment of disease status (IGADS), and in Investigator's assessment of response to therapy (IART) show oral ibuprofen is much better than placebo clearly. In overall of Subject Global Impression of Change, Investigator's global assessment of disease status, and in Investigator's assessment of response to therapy at Week 12: 17.5 mg/knee of 2-(diethylamino)ethyl 2-(4-isobutylphenyl)propionate, BID>8.75 mg/knee of 2-(diethylamino)ethyl 2-(4-isobutylphenyl)propionate, BID˜Oral Ibuprofen (3×400 mg)>4.375 mg/knee of 2-(diethylamino)ethyl 2-(4-isobutylphenyl)propionate, BID>Placebo. For elder subjects, VAS WOMAC scoring system may be difficult to use, it may be much easier for them to use “very good, good, fair, poor, or very poor” to judge the efficacy. The reason for the difference between the volunteer subjects and the recruited subjects by the third party is unknown.

In Phase 3 design, all subjects will be treated with placebo for 2-3 weeks during the screening period with subject-blinded method and subjects with a placebo response exceeding 25% improvement in the average Western Ontario and McMaster Osteoarthritis Index [WOMAC] pain subscale score from Screening Visit to Day 1 will be excluded to minimize the placebo effect and get the true efficacy.

Based on above clinical data, 2-(diethylamino)ethyl 2-(4-isobutylphenyl)propionate may be the first transdermal NSAID with higher efficacy than oral NSAIDs and it can be used every day due to low side effects and high efficacy.

The sign and symptoms relief are getting better and better by time and the onset of the sign and symptoms relief is fast as shown in the following table 71-76.

TABLE 71
The Change from Baseline in the WOMAC* Pain
Subscale Scores from Week 2 to Week 12

| | Week | Week | Week | Week |
|---|---|---|---|---|
| | 2 (SD) | 4 (SD) | 8 (SD) | 12 (SD) |
| Cohort | in mm | in mm | in mm | in mm |
| Placebo | −6.7 (10.3) | −12.6 (12.8) | −19.8 (16.4) | −22.6 (18.1) |
| (n = 48) | | | | |
| Oral | −9.1 (13.2) | −12.1 (15.5) | −19.2 (17.2) | −25.4 (17.8) |
| Ibuprofen | | | | |
| (3 × 400 mg) | | | | |
| (n = 48) | | | | |
| 4.375 mg/knee, | −8.7 (10.7) | −13.1 (13.4) | −19.2 (14.7) | −25.6 (18.4) |
| BID, (n = 49) | | | | |
| 8.75 mg/knee, | −9.5 (13.2) | −15.4 (14.4) | −21.7 (15.9) | −28.6 (17.2) |
| BID, (n = 54) | | | | |
| 17.5 mg/knee, | −10.2 (11.4) | −17.3 (14.3) | −27.0 (17.5) | −31.7 (18.6) |
| BID, (n = 52) | | | | |

TABLE 72
The Change from Baseline in the WOMAC* Stiffness
Subscale Scores from Week 2 to Week 12

| | Week | Week | Week | Week |
|---|---|---|---|---|
| | 2 (SD) | 4 (SD) | 8 (SD) | 12 (SD) |
| Cohort | in mm | in mm | in mm | in mm |
| Placebo | −4.1 (11.0) | −7.4 (16.5) | −13.8 (19.2) | −16.5 (22.8) |
| (n = 48) | | | | |
| Oral | −9.3 (15.8) | −11.1 (17.0) | −15.9 (18.9) | −20.3 (19.4) |
| Ibuprofen | | | | |
| (3 × 400 mg) | | | | |
| (n = 48) | | | | |
| 4.375 mg/knee, | −3.3 (13.0) | −8.8 (14.7) | −12.5 (18.5) | −16.9 (20.3) |
| BID, (n = 49) | | | | |
| 8.75 mg/knee, | −9.2 (11.5) | −12.8 (15.4) | −18.1 (17.5) | −23.3 (19.6) |
| BID, (n = 54) | | | | |
| 17.5 mg/knee, | −9.2 (15.2) | −12.5 (19.0) | −21.1 (21.7) | −25.2 (22.2) |
| BID, (n = 52) | | | | |

TABLE 73
Change from Baseline in the WOMAC* Difficulty Performing
Daily Activities Subscale Scores from Week 2 to Week 12

| | Week | Week | Week | Week |
|---|---|---|---|---|
| | 2 (SD) | 4 (SD) | 8 (SD) | 12 (SD) |
| Cohort | in mm | in mm | in mm | in mm |
| Placebo | −6.2 (10.3) | −11.0 (14.2) | −18.5 (18.4) | −21.9 (19.5) |
| (n = 48) | | | | |
| Oral | −10.2 (15.0) | −12.5 (16.3) | −18.8 (18.2) | −25.3 (19.2) |
| Ibuprofen | | | | |
| (3 × 400 mg) | | | | |
| (n = 48) | | | | |
| 4.375 mg/knee, | −7.3 (13.8) | −13.2 (13.5) | −20.0 (15.5) | −25.4 (17.7) |
| BID, (n = 49) | | | | |
| 8.75 mg/knee, | −9.0 (12.6) | −16.0 (14.7) | −22.4 (16.4) | −27.9 (18.6) |
| BID, (n = 54) | | | | |
| 17.5 mg/knee, | −9.5 (11.6) | −15.3 (15.7) | −25.2 (18.3) | −31.4 (19.3) |
| BID, (n = 52) | | | | |

TABLE 74
The Change from Baseline in the WOMAC*
Overall Scores from Week 2 to Week 12

| | Week | Week | Week | Week |
|---|---|---|---|---|
| | 2 (SD) | 4 (SD) | 8 (SD) | 12 (SD) |
| Cohort | in mm | in mm | in mm | in mm |
| Placebo | −6.1 (9.8) | −11.1 (13.60) | −18.4 (17.7) | −21.3 (19.2) |
| (n = 48) | | | | |
| Oral | −9.9 (14.0) | −12.3 (15.5) | −18.6 (17.3) | −24.9 (18.1) |
| Ibuprofen | | | | |
| (3 × 400 mg) | | | | |
| (n = 48) | | | | |
| 4.375 mg/knee, | −7.9 (12.1) | −13.5 (12.4) | −20.0 (15.3) | −25.4 (16.9) |
| BID, (n = 49) | | | | |
| 8.75 mg/knee, | −9.1 (12.0) | −15.6 (14.0) | −21.8 (15.6) | −27.7 (17.8) |
| BID, (n = 54) | | | | |
| 17.5 mg/knee, | −9.5 (11.1) | −15.4 (15.0) | −25.1 (17.8) | −30.9 (18.7) |
| BID, (n = 52) | | | | |

TABLE 75
The Change from Baseline in the Subject's Global Assessment
of Disease Status Scores from Week 2 to Week 12

| | Week | Week | Week | Week |
|---|---|---|---|---|
| | 2 (SD) | 4 (SD) | 8 (SD) | 12 (SD) |
| Cohort | in mm | in mm | in mm | in mm |
| Placebo | −9.0 (14.6) | −17.6 (17.0) | −25.3 (17.6) | −30.4 (18.8) |
| (n = 48) | | | | |
| Oral | −13.9 (16.3) | −20.0 (16.6) | −26.8 (19.0) | −33.3 (20.4) |
| Ibuprofen | | | | |
| (3 × 400 mg) | | | | |
| (n = 48) | | | | |
| 4.375 mg/knee, | −10.6 (16.3) | −19.7 (17.1) | −27.8 (19.7) | −34.7 (21.1) |
| BID, (n = 49) | | | | |
| 8.75 mg/knee, | −10.8 (10.8) | −19.8 (14.3) | −28.4 (17.2) | −36.6 (19.0) |
| BID, (n = 54) | | | | |
| 17.5 mg/knee, | −10.4 (14.2) | −22.1 (18.1) | −30.6 (18.9) | −38.3 (18.6) |
| BID, (n = 52) | | | | |

TABLE 76
The Change from Baseline in the Subject's
pain Assessment Scores from Week 2 to Week 12

| | Week | Week | Week | Week |
|---|---|---|---|---|
| | 2 (SD) | 4 (SD) | 8 (SD) | 12 (SD) |
| Cohort | in mm | in mm | in mm | in mm |
| Placebo | −9.2 (13.2) | −13.9 (15.1) | −20.2 (17.0) | −24.1 (19.7) |
| (n = 48) | | | | |
| Oral | −11.5 (15.2) | −14.2 (16.6) | −25.0 (16.9) | −30.5 (18.3) |
| Ibuprofen | | | | |
| (3 × 400 mg) | | | | |
| (n = 48) | | | | |
| 4.375 mg/knee, | −9.6 (13.3) | −13.8 (13.0) | −19.3 (17.5) | −25.6 (18.9) |
| BID, (n = 49) | | | | |
| 8.75 mg/knee, | −10.4 (11.8) | −15.9 (16.0) | −22.3 (16.8) | −31.4 (21.0) |
| BID, (n = 54) | | | | |
| 17.5 mg/knee, | −11.4 (12.3) | −19.6 (17.0) | −27.8 (18.9) | −34.6 (19.1) |
| BID, (n = 52) | | | | |

4.12. Safety Summary

The treatment-emergent adverse events are shown in Table 77. The frequency of musculoskeletal and connective tissue disorders, nervous system disorders, respiratory, thoracic and mediastinal disorders, infections and infestations, injury, poisoning and procedural complications, investigations, skin and subcutaneous tissue disorders, psychiatric disorders, metabolism and nutrition disorders, eye disorders, ear and labyrinth disorders, reproductive system and breast disorders, vascular disorders, blood and lymphatic system disorders, gastrointestinal disorders, positive fecal occult blood test, administration site conditions, treatment-emergent adverse events, and moderate adverse events are respectively shown in Tables 78-97. Several serious treatment-emergent adverse events are shown in Tables 97-100.

TABLE 77
Treatment-emergent Adverse Events (All Causalities)

| | | | Oral Ibuprofen | 4.375 | 8.75 | 17.5 | |
|---|---|---|---|---|---|---|---|
| | | Placebo | (3 × 400 mg) | mg/knee, | mg/knee, | mg/knee, | Total |
| | | (n = 69) | (n = 71) | BID, (n = 71) | BID, (n = 70) | BID, (n = 70) | (n = 351) |
| Treatment-emergent | Events | 60 | 87 | 68 | 71 | 52 | 338 |
| adverse events | Subjects | 41 (59.4%) | 42 (59.2%) | 41 (57.7%) | 39 (55.7%) | 30 (42.9%) | 193 (55.0%) |
| Severe treatment-emergent | Events | 1 | 1 | 1 | 1 | 0 | 4 |
| adverse events | Subjects | 1 (1.4%) | 1 (1.4%) | 1 (1.4%) | 1 (1.4%) | 0 | 4 (1.1%) |
| Serious treatment-emergent | Events | 1 | 1 | 1 | 1 | 0 | 4 |
| adverse events | Subjects | 1 (1.4%) | 1 (1.4%) | 1 (1.4) | 1 (1.4) | 0 | 4 (1.1%) |
| To be discontinued from the treatment | Events | 1 | 0 | 1 | 1 | 0 | 3 |
| temporarily due to adverse events | Subjects | 1 (1.4%) | 0 | 1 (1.4%) | 1 (1.4%) | 0 | 3 (0.9%) |
| To be discontinued from the study | Events | 0 | 0 | 1 | 0 | 1 | 2 |
| due to adverse events | Subjects | 0 | 0 | 1 (1.4%) | 0 | 1 (1.4%) | 2 (0.6%) |
| Results in death | | 0 | 0 | 1 (1.4%) | 0 | 0 | 1 (0.3%) |
| Requires hospitalization | Events | 1 | 1 | 0 | 1 | 0 | 3 |
| | Subjects | 1 (1.4%) | 1 (1.4%) | 0 | 1 (1.4%) | 0 | 3 (0.9%) |
| Adverse events of special interest | Events | 20 | 17 | 16 | 12 | 14 | 79 |
| | Subjects | 20 (29.0%) | 14 (19.7%) | 11 (15.5%) | 10 (14.3) | 11 (15.7) | 66 (18.8%) |
| Local skin reactions | Events | 2 | 0 | 1 | 1 | 5 | 9 |
| around the treated knees | Subjects | 2 (2.9%) | 0 | 1 (1.4%) | 1 (1.4%) | 4 (5.7%) | 8 (2.3%) |

TABLE 78
Frequency of Musculoskeletal and Connective Tissue Disorders

| | | | | 4.375 mg | 8.75 mg | 17.5 mg | |
|---|---|---|---|---|---|---|---|
| | | | Oral | 2-(diethylamino)ethyl | 2-(diethylamino)ethyl | 2-(diethylamino)ethyl | |
| | | Placebo, | Ibuprofen | 2-(4-isobutylphenyl) | 2-(4-isobutylphenyl) | 2-(4-isobutylphenyl) | |
| System Organ Class | | BID, | (3 × 400 mg) | propionate/knee, | propionate/knee, | propionate/knee, | Overall |
| Preferred Term | | (N = 69) | (N = 71) | BID (N = 71) | BID (N = 70) | BID(N = 70) | (N = 351) |
| Overall Total | | 3(4.3%)[3] | 7(9.9%)[9] | 3(4.2%)[3] | 7(10.0%)[7] | 6(8.6%)[8] | 26(7.4%)[29] |
| | Back Pain | 1(%)[1] | 2(2.8%)[2] | 1(%)[1] | 2(%)[2] | 1(%)[1] | 7(2.0%)[7] |
| | Pain in Extremity | 1(%)[1] | 3(4.2%)[5] | — | 1(%)[1] | 1(%)[1] | 6(1.7%)[8] |
| | Arthralgia | — | — | — | — | — | — |
| | Neck Pain | 1(%)[1] | — | 1(%)[1] | 3(%)[3] | 1(%)[1] | 6(1.7%)[6] |
| | Myalgia | — | — | — | — | — | — |
| | Musculoskeletal Pain | — | — | 1(%)[1] | — | 1(%)[2] | 2(0.6%)[3] |
| | Joint Swelling | — | — | — | — | — | — |
| | Musculoskeletal Stiffness | — | — | — | — | — | — |
| | Groin Pain | — | — | — | — | — | — |
| | Joint Stiffness | — | 1(1.4%)[1] | — | 1(%)[1] | — | 2(0.6%)[2] |
| | Muscle Spasms | — | 1(1.4%)[1] | — | — | 2(%)[3] | 3(0.9%)[4] |
| | Musculoskeletal Chest Pain | — | — | — | — | — | — |

N = Number of subjects studied
( ) = Percentage of subjects with adverse events
[ ] = Number of adverse events

TABLE 79
Frequency of Nervous System Disorders

| | | | | 4.375 mg | 8.75 mg | 17.5 mg | |
|---|---|---|---|---|---|---|---|
| | | | Oral | 2-(diethylamino)ethyl | 2-(diethylamino)ethyl | 2-(diethylamino)ethyl | |
| | | Placebo, | Ibuprofen | 2-(4-isobutylphenyl) | 2-(4-isobutylphenyl) | 2-(4-isobutylphenyl) | |
| System Organ Class | | BID, | (3 × 400 mg) | propionate/knee, | propionate/knee, | propionate/knee, | Overall |
| Preferred Term | | (N = 69) | (N = 71) | BID (N = 71) | BID (N = 70) | BID (N = 70) | (N = 351) |
| Nervous System Disorders | | 4(5.8%)[5] | 5(7.0%)[6] | 1(1.4%)[1] | 7(10.0%)[8] | 2(2.9%)[2] | 19(5.4%)[22] |
| | Headache | 2(2.9%)[3] | 4(5.6%)[5] | 1(1.4%)[1] | 4(5.7%)[6] | 1(1.4%)[1] | 12(3.4%)[16] |
| | Dizziness | 2(2.9%)[2] | 1(1.4%)[1] | — | 2(2.9%)[2] | 1(1.4%)[1] | 6(1.7%)[6] |
| | Hypoaesthesia | — | — | — | 1(1.4%)[1] | — | 1(0.3%)[1] |
| | Sciatica | — | — | — | — | — | — |
| | Migraine | — | — | — | — | — | — |
| | Paraesthesia | — | — | — | — | — | — |
| | Sinus Headache | — | — | — | — | — | — |

N = Number of subjects studied
( ) = Percentage of subjects with adverse events
[ ] = Number of adverse events

TABLE 80
Frequency of Respiratory, Thoracic and Mediastinal Disorders

| | | | | 4.375 mg | 8.75 mg | 17.5 mg | |
|---|---|---|---|---|---|---|---|
| | | | | 2-(diethylamino) | 2-(diethylamino) | 2-(diethylamino) | |
| | | | Oral | ethyl | ethyl | ethyl | |
| | | Placebo, | Ibuprofen | 2-(4-isobutyl- | 2-(4-isobutyl- | 2-(4-isobutyl- | |
| System Organ Class | | BID, | (3 × 400 mg) | phenyl)propionate/ | phenyl)propionate/ | phenyl)propionate/ | Overall |
| Preferred Term | | (N = 69) | (N = 71) | knee, BID (N = 71) | knee, BID (N = 70) | knee, BID (N = 70) | (N = 351) |
| Respiratory, Thoracic and | | 3(4.3%)[3] | 5(7.9%)[5] | 6(8.5%)[6] | 3(4.3%)[3] | 2(2.9%)[2] | 19(5.4%)[19] |
| Mediastinal Disorders | | | | | | | |
| | Oropharyngeal Pain | 1(1.4%)[1] | 1(1.4%)[1] | 2(1.4%)[2] | 2(1.4%)[2] | — | 6(1.7%)[6] |
| | Pulmonary Congestion | | | | | | |
| | Cough | 1(1.4%)[1] | 1(1.4%)[1] | 1(1.4%)[1] | — | 2(2.9%)[2] | 5(1.4%)[5] |
| | Rhinorrhoea | — | — | 1(1.4%)[1] | — | — | 1(0.3%)[1] |
| | Throat Irritation | 1(1.4%)[1] | 1(1.4%)[1] | 2(2.8%)[2] | 1(1.4%)[1] | — | 5(1.4%)[5] |
| | Chronic Obstructive | — | — | — | — | — | — |
| | Pulmonary Disease | | | | | | |
| | Pharyngeal Erythema | — | — | — | — | — | — |
| | Rhinitis Seasonal | — | 1(1.4%)[1] | — | — | — | 1(0.3%)[1] |
| | Sinus Congestion | | | | | | |
| | Sneezing | — | — | — | — | — | — |
| | Epistaxis | — | — | — | — | — | — |
| | Nasal Congestion | — | 1(1.4%)[1] | — | — | — | 1(0.3%)[1] |

N = Number of subjects studied
( ) = Percentage of subjects with adverse events
[ ] = Number of adverse events

TABLE 81
Frequency of Infections and Infestations

| | | | | 4.375 mg | 8.75 mg | 17.5 mg | |
|---|---|---|---|---|---|---|---|
| | | | | 2-(diethylamino) | 2-(diethylamino) | 2-(diethylamino) | |
| | | | | ethyl | ethyl | ethyl | |
| | | | | 2-(4-isobutyl- | 2-(4-isobutyl- | 2-(4-isobutyl- | |
| | | | Oral | phenyl)- | phenyl)- | phenyl)- | |
| | | | Ibuprofen | propionate/ | propionate/ | propionate/ | |
| System Organ Class | | Placebo, BID, | (3 × 400 mg) | knee, BID | knee, BID | knee, BID | Overall |
| Preferred Term | | (N = 69) | (N = 71) | (N = 71) | (N = 70) | (N = 70) | (N = 351) |
| Infections and Infestations | | 14(20.2%)[14] | 31(43.7%)[32] | 23(32.4%)[24] | 20(28.6%)[20] | 13(18.6%)[14] | 100(28.5%)[105] |
| | Nasopharyngitis | — | — | — | — | — | — |
| | Bronchitis | — | — | — | — | — | — |
| | Influenza | 1(1.4%)[1] | — | 3(4.2%)[3] | 1(1.4%)[1] | — | 5(1.4%)[5] |
| | Sinusitis | — | — | — | — | — | — |
| | Diverticulitis | — | 1(1.4%)[1] | — | — | — | 1(0.3%)[1] |
| | Laryngitis | — | — | — | — | — | — |
| | Onychomycosis | — | — | — | — | — | |
| | Skin Bacterial Infection | — | 1(1.4%)[1] | — | 1(1.4%)[1] | — | 2(0.6%)[2] |
| | Upper Respiratory | 7(10.1%)[7] | 15(21.1%)[16] | 11(1.5%)[12] | 12(15.7%)[12] | 5(7.1%)[5] | 50(14.2%)[52] |
| | Tract Infection | | | | | | |
| | Urinary Tract Infection | 6(8.7%)[6] | 10(14.1%)[11] | 8(11.3%)[8] | 6(8.6%)[6] | 8(11.4%)[9] | 38(10.8%)[40] |
| | Viral Upper Respiratory | — | — | — | — | — | — |
| | Tract Infection | | | | | | |
| | Clostridium Difficile | — | 1(1.4%)[1] | — | — | — | 1(0.3%)[1] |
| | Infection | | | | | | |
| | Pneumonia | — | — | — | — | — | — |
| | Tooth Infection | — | 3(4.2%)[3] | — | 1(1.4%)[1] | — | 4(1.1%)[4] |

N = Number of subjects studied
( ) = Percentage of subjects with adverse events
[ ] = Number of adverse events

TABLE 82
Frequency of Injury, Poisoning and Procedural Complications

| | | | | 4.375 mg | 8.75 mg | 17.5 mg | |
|---|---|---|---|---|---|---|---|
| | | | | 2-(diethylamino) | 2-(diethylamino) | 2-(diethylamino) | |
| | | | Oral | ethyl | ethyl | ethyl | |
| | | Placebo, | Ibuprofen | 2-(4-isobutyl- | 2-(4-isobutyl- | 2-(4-isobutyl- | |
| System Organ Class | | BID, | (3 × 400 mg) | phenyl)propionate/ | phenyl)propionate/ | phenyl)propionate/ | Overall |
| Preferred Term | | (N = 69) | (N = 71) | knee, BID (N = 71) | knee, BID (N = 70) | knee, BID (N = 70) | (N = 351) |
| Injury, Poisoning and | | 2(2.9%)[2] | 3(4.2%)[4] | 3(4.2%)[3] | — | — | 8(2.3%)[9] |
| | Procedural Complications | | | | | | |
| | Fall | 1(1.4%)[1] | — | 1(1.4%)[1] | — | — | 2(0.6%)[2] |
| | Ligament Sprain | — | 1(1.4%)[2] | — | — | — | 1(0.3%)[2] |
| | Contusion | 1(1.4%)[1] | 2(2.8%)[2] | 2(2.8%)[2] | — | — | 5(1.4%)[5] |
| | Arthropod Bite | — | — | — | — | — | — |
| | Epicondylitis | — | — | — | — | — | — |
| | Eye Contusion | — | — | — | — | — | — |
| | Animal Bite | — | — | — | — | — | — |
| | Excoriation Muscle Strain | — | — | — | — | — | — |

N = Number of subjects studied
( ) = Percentage of subjects with adverse events
[ ] = Number of adverse events

TABLE 83
Frequency of Investigations

| | | | | 4.375 mg | 8.75 mg | 17.5 mg | |
|---|---|---|---|---|---|---|---|
| | | | | 2-(diethylamino) | 2-(diethylamino) | 2-(diethylamino) | |
| | | | Oral | ethyl | ethyl | ethyl | |
| | | Placebo, | Ibuprofen | 2-(4-isobutyl- | 2-(4-isobutyl- | 2-(4-isobutyl- | |
| System Organ Class | | BID, | (3 × 400 mg) | phenyl)propionate/ | phenyl)propionate/ | phenyl)propionate/ | Overall |
| Preferred Term | | (N = 69) | (N = 71) | knee, BID (N = 71) | knee, BID (N = 70) | knee, BID (N = 70) | (N = 351) |
| Investigations | | 3(4.3%)[3] | 3(4.2%)[3] | 11(15.5%)[11] | 5(7.1%)[5] | 1(1.4%)[1] | 23(6.6%)[23] |
| | Blood Lactate Dehydrogenase | — | — | — | 1(1.4%)[1] | — | 1(0.3%)[1] |
| | Increased | | | | | | |
| | Gamma-Glutamyltransferase | 1(1.4%)[1] | 1(1.4%)[1] | 2(2.8%)[2] | — | — | 4(1.1%)[4] |
| | Increased | | | | | | |
| | Alanine Aminotransferase | — | 1(1.4%)[1] | 1(1.4%)[1] | 1(1.4%)[1] | — | 3(0.9%)[3] |
| | Increased | | | | | | |
| | Blood Alkaline Phosphatase | — | — | 1(1.4%)[1] | — | — | 1(0.3%)[1] |
| | Increased | | | | | | |
| | Blood Potassium Increased | — | — | — | — | — | — |
| | Eosinophil Count Increased | — | — | — | — | — | — |
| | Transaminases Increased | — | — | 1(1.4%)[1] | 1(1.4%)[1] | — | 2(0.6%)[1] |
| | White Blood Cell Count | 1(1.4%)[1] | — | 2(2.8%)[2] | 2(2.9%)[2] | — | 5(1.4%)[5] |
| | Decreased | | | | | | |
| | Aspartate Aminotransferase | 1(1.4%)[1] | 1(1.4%)[1] | 3(4.2%)[3] | — | 1(1.4%)[1] | 6(1.7%)[6] |
| | Increased | | | | | | |
| | Blood Bilirubin Increased | — | — | 1(1.4%)[1] | — | — | 1(0.3%)[1] |

N = Number of subjects studied
( ) = Percentage of subjects with adverse events
[ ] = Number of adverse events

TABLE 84
Frequency of Skin and Subcutaneous Tissue Disorders

| | | | | 4.375 mg | 8.75 mg | 17.5 mg | |
|---|---|---|---|---|---|---|---|
| | | | | 2-(diethylamino) | 2-(diethylamino) | 2-(diethylamino) | |
| | | | Oral | ethyl | ethyl | ethyl | |
| | | Placebo, | Ibuprofen | 2-(4-isobutyl- | 2-(4-isobutyl- | 2-(4-isobutyl- | |
| System Organ Class | | BID, | (3 × 400 mg) | phenyl)propionate/ | phenyl)propionate/ | phenyl)propionate/ | Overall |
| Preferred Term | | (N = 69) | (N = 71) | knee, BID (N = 71) | knee, BID (N = 70) | knee, BID (N = 70) | (N = 351) |
| Skin and Subcutaneous | | 4(5.8%)[4] | 2(2.8%)[2] | 2(2.8%)[2] | 1(1.4%)[1] | 5(7.1%)[6] | 14(4.0%)[15] |
| | Tissue Disorders | | | | | | |
| | Skin Irritation | 3(1.4%)[3] | 1(1.4%)[1] | 1(1.4%)[1] | 1(1.4%)[1] | 3(1.4%)[4] | 9(2.6%)[10] |
| | Dermatitis Contact | — | — | — | — | — | — |
| | Erythema | — | — | — | — | — | — |
| | Psoriasis | — | — | — | — | — | — |
| | Swelling Face | — | — | — | — | — | — |
| | Rash | 1(1.4%)[1] | 1(1.4%)[1] | 1(1.4%)[1] | — | 2(4.2%) [2] | 5(1.4%)[5] |

N = Number of subjects studied
( ) = Percentage of subjects with adverse events
[ ] = Number of adverse events

TABLE 85
Frequency of Psychiatric Disorders

| | | | 4.375 mg | 8.75 mg | 17.5 mg | |
|---|---|---|---|---|---|---|
| | | | 2-(diethylamino)- | 2-(diethylamino)- | 2-(diethylamino)- | |
| | | Oral | ethyl | ethyl | ethyl | |
| | Placebo, | Ibuprofen | 2-(4-isobutyl- | 2-(4-isobutyl- | 2-(4-isobutyl- | |
| System Organ Class | BID, | (3 × 400 mg) | phenyl)propionate/ | phenyl)propionate/ | phenyl)propionate/ | Overall |
| Preferred Term | (N = 69) | (N = 71) | knee, BID (N = 71) | knee, BID (N = 70) | knee, BID (N = 70) | (N = 351) |
| Psychiatric Disorders | 1(1.4%) [1] | — | — | — | — | 1(0.3%)[1] |
| Insomnia | 1(1.4%) [1] | — | — | — | — | — |
| Depression | — | — | — | — | — | — |
| Emotional Disorder | — | — | — | — | — | — |

N = Number of subjects studied
( ) = Percentage of subjects with adverse events
[ ] = Number of adverse events

TABLE 86
Frequency of Metabolism and Nutrition Disorders

| | | | 4.375 mg | 8.75 mg | 17.5 mg | |
|---|---|---|---|---|---|---|
| | | Oral | 2-(diethylamino) | 2-(diethylamino) | 2-(diethylamino) | |
| | | Ibuprofen | ethyl | ethyl | ethyl | |
| | Placebo, | (3 × 400 | 2-(4-isobutylphenyl) | 2-(4-isobutylphenyl) | 2-(4-isobutylphenyl) | |
| System Organ Class | BID, (N = | mg) (N = | propionate/knee, | propionate/knee, | propionate/knee, | Overall |
| Preferred Term | 69) | 71) | BID (N = 71) | BID (N = 70) | BID (N = 70) | (N = 351) |
| Metabolism and Nutrition Disorders | 8(11.6%)[8] | 8(11.3%)[8] | 6(8.5%)[7] | 8(12.9%)[8] | 9(11.4%)[9] | 39(11.1%)[40] |
| Fluid Retention | — | — | — | — | — | — |
| Gout | — | — | — | — | 1(1.4%)[1] | 1(0.3%)[1] |
| Hypercholesterolaemia | 4(5.8%) [4] | 5(7.0%) [5] | 1(1.4%) [1] | 5(7.1%) [5] | 2(2.9%) [2] | 17(4.8%)[17] |
| Hypertriglyceridemia | 4(5.8%) [4] | 3(4.2%) [3] | 5(7.0%) [6] | 3(4.3%) [3] | 6(8.6%) [6] | 21(6.0%)[22] |

N = Number of subjects studied
( ) = Percentage of subjects with adverse events
[ ] = Number of adverse events

TABLE 87
Frequency of Eye Disorders

| | | | 4.375 mg | 8.75 mg | 17.5 mg | |
|---|---|---|---|---|---|---|
| | | Oral | 2-(diethylamino) | 2-(diethylamino) | 2-(diethylamino) | |
| | | Ibuprofen | ethyl | ethyl | ethyl | |
| | Placebo, | (3 × 400 | 2-(4-isobutylphenyl) | 2-(4-isobutylphenyl) | 2-(4-isobutylphenyl) | |
| System Organ Class | BID, (N = | mg) (N = | propionate/knee, | propionate/knee, | propionate/knee, | Overall |
| Preferred Term | 69) | 71) | BID (N = 71) | BID (N = 70) | BID (N = 70) | (N = 351) |
| Eye Disorders | — | 1(1.4%)[1] | — | — | — | 1(1.4%)[1] |
| Eye Pain | — | — | — | — | — | — |
| Vitreous Degeneration | — | — | — | — | — | — |
| Vitreous Floaters | — | 1(1.4%)[1] | — | — | — | 1(1.4%)[1] |
| Conjunctivitis | — | — | — | — | — | — |
| Erythema of Eyelid | — | — | — | — | — | — |
| Eyelid Oedema | — | — | — | — | — | — |

N = Number of subjects studied
( ) = Percentage of subjects with adverse events
[ ] = Number of adverse events

TABLE 88
Frequency of Ear and Labyrinth Disorders

| | | | 4.375 mg | 8.75 mg | 17.5 mg | |
|---|---|---|---|---|---|---|
| | | Oral | 2-(diethylamino) | 2-(diethylamino) | 2-(diethylamino) | |
| | | Ibuprofen | ethyl | ethyl | ethyl | |
| | Placebo, | (3 × 400 | 2-(4-isobutylphenyl) | 2-(4-isobutylphenyl) | 2-(4-isobutylphenyl) | |
| System Organ Class | BID, (N = | mg) (N = | propionate/knee, | propionate/knee, | propionate/knee, | Overall |
| Preferred Term | 69) | 71) | BID (N = 71) | BID (N = 70) | BID (N = 70) | (N = 351) |
| Ear and Labyrinth Disorders | — | — | 1(1.4%)[1] | 2(2.9%)[2] | — | 3(0.9%) [3] |
| Ear Pain | — | — | 1(%)[1] | — | — | — |
| Vertigo | — | — | — | 2(%)[2] | — | — |
| Cerumen Impaction | — | — | — | — | — | — |

N = Number of subjects studied
( ) = Percentage of subjects with adverse events
[ ] = Number of adverse events

TABLE 89
Frequency of Reproductive System and Breast Disorders

| | | | 4.375 mg | 8.75 mg | 17.5 mg | |
|---|---|---|---|---|---|---|
| | | Oral | 2-(diethylamino) | 2-(diethylamino) | 2-(diethylamino) | |
| | | Ibuprofen | ethyl | ethyl | ethyl | |
| | Placebo, | (3 × 400 | 2-(4-isobutylphenyl) | 2-(4-isobutylphenyl) | 2-(4-isobutylphenyl) | |
| System Organ Class | BID, (N = | mg) (N = | propionate/knee, | propionate/knee, | propionate/knee, | Overall |
| Preferred Term | 69) | 71) | BID (N = 71) | BID (N = 70) | BID (N = 70) | (N = 351) |
| Reproductive System and Breast Disorders | — | — | — | — | — | — |
| Dysmenorrhoea | — | — | — | — | — | — |
| Premenstrual Pain | — | — | — | — | — | — |

N = Number of subjects studied
( ) = Percentage of subjects with adverse events
[ ] = Number of adverse events

TABLE 90
Frequency of Vascular Disorders

| | | | 4.375 mg | 8.75 mg | 17.5 mg | |
|---|---|---|---|---|---|---|
| | | Oral | 2-(diethylamino) | 2-(diethylamino) | 2-(diethylamino) | |
| | | Ibuprofen | ethyl | ethyl | ethyl | |
| | Placebo, | (3 × 400 | 2-(4-isobutylphenyl) | 2-(4-isobutylphenyl) | 2-(4-isobutylphenyl) | |
| System Organ Class | BID, (N = | mg) (N = | propionate/knee, | propionate/knee, | propionate/knee, | Overall |
| Preferred Term | 69) | 71) | BID (N = 71) | BID (N = 70) | BID (N = 70) | (N = 351) |
| Vascular Disorders | — | 1(1.4%)[1] | 1(1.4%)[1] | 2(2.9%)[2] | — | 4(1.1%)[4] |
| Hypertension | — | 1(1.4%)[1] | 1(1.4%)[1] | 1(1.4%)[1] | — | 3(0.9%)[3] |
| Thrombosis | — | — | — | 1(1.4%)[1] | — | 1(0.3%)[1] |

N = Number of subjects studied
( ) = Percentage of subjects with adverse events
[ ] = Number of adverse events

TABLE 91
Frequency of Blood and Lymphatic System Disorders

| | | | 4.375 mg | 8.75 mg | 17.5 mg | |
|---|---|---|---|---|---|---|
| | | Oral | 2-(diethylamino) | 2-(diethylamino) | 2-(diethylamino) | |
| | | Ibuprofen | ethyl | ethyl | ethyl | |
| | Placebo, | (3 × 400 | 2-(4-isobutylphenyl) | 2-(4-isobutylphenyl) | 2-(4-isobutylphenyl) | |
| System Organ Class | BID, (N = | mg) (N = | propionate/knee, | propionate/knee, | propionate/knee, | Overall |
| Preferred Term | 69) | 71) | BID (N = 71) | BID (N = 70) | BID (N = 70) | (N = 351) |
| Blood and Lymphatic System Disorders | — | — | — | — | — | — |
| Neutropenia | — | — | — | — | — | — |

N = Number of subjects studied
( ) = Percentage of subjects with adverse events
[ ] = Number of adverse events

TABLE 92
Frequency of Gastrointestinal Disorders

| | | | 4.375 mg | 8.75 mg | 17.5 mg | |
|---|---|---|---|---|---|---|
| | | Oral | 2-(diethylamino) | 2-(diethylamino) | 2-(diethylamino) | |
| | | Ibuprofen | ethyl | ethyl | ethyl | |
| | Placebo, | (3 × 400 | 2-(4-isobutylphenyl) | 2-(4-isobutylphenyl) | 2-(4-isobutylphenyl) | |
| System Organ Class | BID, (N = | mg) (N = | propionate/knee, | propionate/knee, | propionate/knee, | Overall |
| Preferred Term | 69) | 71) | BID (N = 71) | BID (N = 70) | BID (N = 70) | (N = 351) |
| Gastrointestinal Disorders | 10(14.5%)[10] | 15(21.1%)[17] | 3(4.2%)[3] | 10(14.3%)[11] | 4(5.7%)[5] | 42(12.0%)[46] |
| Toothache | 4(5.8%)[4] | 3( 4.2%) [4] | 2(2.8%)[2] | 1(1.4%)[1] | 1(1.4%)[1] | 11(3.1%)[12] |
| Constipation | — | 1(1.4% )[1] | — | — | 1(1.4%)[1] | 2(0.6%)[2] |
| Diarrhoea | 2(2.9%)[2] | 1(1.4%)[1] | — | 2(2.9%)[2] | — | 5(1.4%)[5] |
| Dental Caries | — | 1(1.4%)[1] | — | — | — | 1(0.3%)[1] |
| Gastrooesophageal Reflux Disease | — | 2(2.8%)[2] | — | — | — | 2(0.6%)[2] |
| Abdominal Discomfort | 2(2.9%)[2] | 3(4.2%)[3] | — | 3(4.3%)[4] | 1(1.4%)[1] | 9(2.6%)[10] |
| Abdominal Pain | — | — | — | — | — | — |
| Abdominal Pain Upper | 1(2.9%)[1] | 4(5.6%)[5] | 1(1.4%)[1] | 1(1.4%)[1] | 1(1.4%)[2] | 8(2.3%)[10] |
| Nausea | 1(2.9%)[1] | — | — | 3(4.3%)[3] | — | 4(1.1%)[4] |

N = Number of subjects studied
( ) = Percentage of subjects with adverse events
[ ] = Number of adverse events

TABLE 93
Frequency of Positive Fecal Occult Blood Test
Oral

| | | | 4.375 mg | 8.75 mg | 17.5 mg | |
|---|---|---|---|---|---|---|
| | | Oral | 2-(diethylamino) | 2-(diethylamino) | 2-(diethylamino) | |
| | | Ibuprofen | ethyl | ethyl | ethyl | |
| | Placebo, | (3 × 400 | 2-(4-isobutylphenyl) | 2-(4-isobutylphenyl) | 2-(4-isobutylphenyl) | |
| | BID, (N = | mg) (N = | propionate/knee, | propionate/knee, | propionate/knee, | Overall |
| | 69) | 71) | BID (N = 71) | BID (N = 70) | BID (N = 70) | (N = 351) |
| Frequency of | 10 | 9 | 6 | 6 | 5 | 36 |
| Positive Fecal | | | | | | |
| Occult Blood | | | | | | |
| Test | | | | | | |
| Subjects with | 10 | 8 (11.3%) | 5 (7.0%) | 5 (7.1%) | 5 (7.1%) | 33 |
| Positive Fecal | (14.5%) | | | | | (9.4%) |
| Occult Blood | | | | | | |
| Test | | | | | | |

TABLE 94
Frequency of Administration Site Conditions

| | | | 4.375 mg | 8.75 mg | 17.5 mg | |
|---|---|---|---|---|---|---|
| | | Oral | 2-(diethylamino) | 2-(diethylamino) | 2-(diethylamino) | |
| | | Ibuprofen | ethyl | ethyl | ethyl | |
| | Placebo, | (3 × 400 | 2-(4-isobutylphenyl) | 2-(4-isobutylphenyl) | 2-(4-isobutylphenyl) | |
| System Organ Class | BID, (N = | mg) (N = | propionate/knee, | propionate/knee, | propionate/knee, | Overall |
| Preferred Term | 69) | 71) | BID (N = 71) | BID (N = 70) | BID (N = 70) | (N = 351) |
| Administration Site Conditions | 2 (2.9%)[2] | — | 1 (1.4%)[1] | 1 (1.4%)[1] | 4 (5.7%)[4] | 8 (2.3%) [8] |
| Pain | — | — | — | — | — | — |
| Application Site Rash | 1 (1.4%)[1] | — | — | — | 3 (4.3%)[3] | 4 (1.1%)[4] |
| Application Site Paraesthesia | — | — | — | — | — | — |
| Application Site Anaesthesia | — | — | — | — | — | — |
| Application Site Pain | — | — | — | — | — | — |
| Application Site Pruritus | — | — | — | — | — | — |
| Fatigue | — | — | — | — | — | — |
| Injection Site Pain | — | — | — | — | — | — |
| Application Site Irritation | 1 (1.4%)[1] | — | 1 (1.4%)[1] | 1 (1.4%)[1] | 1 (1.4%)[1] | 4 (1.1%)[4] |

N = Number of subjects studied
( ) = Percentage of subjects with adverse events
[ ] = Number of adverse events

TABLE 95
Frequency of Treatment-Emergent Adverse Events (Possibly, Probably, or Definitely Related to Study Drug)

| | | | 4.375 mg | 8.75 mg | 17.5 mg | |
|---|---|---|---|---|---|---|
| | | Oral | 2-(diethylamino) | 2-(diethylamino) | 2-(diethylamino) | |
| | | Ibuprofen | ethyl | ethyl | ethyl | |
| | | (3 × 400 | 2-(4-isobutylphenyl) | 2-(4-isobutylphenyl) | 2-(4-isobutylphenyl) | |
| System Organ Class | Placebo, BID, | mg) (N = | propionate/knee, | propionate/knee, | propionate/knee, | Overall |
| Preferred Term | (N = 69) | 71) | BID (N = 71) | BID (N = 70) | BID (N = 70) | (N = 351) |
| Total Adverse Events | 33(47.8%)[34] | 33(46.5%)[36] | 24(33.8%)[26] | 29(41.4%)[34] | 25(35.7%) [25] | 144(41.0%)[155] |
| Administration Site | 2 (2.9%)[2] | — | 1 (1.4%)[1] | 1 (1.4%)[1] | 4 (5.7%)[4] | 8 (2.3%) [8] |
| Conditions | | | | | | |
| Pain | — | — | — | — | — | — |
| Application Site Rash | 1 (1.4%)[1] | — | — | — | 3 (4.3%) [3] | 4 (1.1%)[4] |
| Application Site Paraesthesia | — | — | — | — | — | — |
| Application Site Anaesthesia | — | — | — | — | — | — |
| Application Site Pain | — | — | — | — | — | — |
| Application Site Pruritus | — | — | — | — | — | — |
| Fatigue | — | — | — | — | — | — |
| Injection Site Pain | — | — | — | — | — | — |
| Application Site Irritation | 1 (1.4%)[1] | — | 1 (1.4%)[1] | 1 (1.4%)[1] | 1 (1.4%)[1] | 4 (1.1%)[4] |
| Positive Fecal Occult Blood | 10(14.5%[10] | 8(11.3%)[9] | 5(7.0%)[6] | 5(7.1%)[6] | 5(7.1%)[5] | 33(9.4)[36] |
| Test | | | | | | |
| Abdominal Pain Upper | 1(2.9%)[1] | 4(5.6%)[5] | 1(1.4%)[1] | 1(1.4%)[1] | 1(1.4%)[2] | 8(2.3%)[10] |
| Abdominal Discomfort | 2(2.9%)[2] | 3(4.2%) [3] | — | 3(4.3%)[4] | 1(1.4%)[1] | 9(2.6%)[10] |
| White Blood Cell Count | 1(1.4%)[1] | — | 2(2.8%)[2] | 2(2.9%)[2] | — | 5(1.4%)[5] |
| Decreased | | | | | | |
| Gastrooesophageal Reflux | — | 2(2.8%)[2] | — | — | — | 2(0.6%)[2] |
| Disease | | | | | | |
| Abdominal Discomfort | 2(2.9%)[2] | 3(4.2%)[3] | — | 3(4.3%)[4] | 1(1.4%)[1] | 9(2.6%)[10] |
| Hypertriglyceridemia | 4(5.8%) [4] | 3(4.2%) [3] | 5(7.0%) [6] | 3(4.3%) [3] | 6(8.6%) [6] | 21(6.0%)[22] |
| Gamma-Glutamyltransferase | 1(1.4%)[1] | 1(1.4%)[1] | 2(2.8%)[2] | — | — | 4(1.1%)[4] |
| Increased | — | — | — | — | — | — |
| Alanine Aminotransferase | — | 1(1.4%)[1] | 1(1.4%)[1] | 1(1.4%)[1] | — | 3(0.9%)[3] |
| Increased | | | | | | |
| Aspartate Aminotransferase | 1(1.4%)[1] | 1(1.4%)[1] | 3(4.2%)[3] | — | 1(1.4%)[1] | 6(1.7%)[6] |
| Increased | — | — | — | — | — | — |
| Headache | 2(2.9%)[3] | 4(5.6%)[5] | 1(1.4%)[1] | 4(5.7%)[6] | 1(1.4%)[1] | 12(3.4%)[16] |
| Dizziness | 2(2.9%)[2] | 1(1.4%)[1] | — | 2(2.9%)[2] | 1(1.4%)[1] | 6(1.7%)[6] |
| Throat Irritation | 1(1.4%)[1] | 1(1.4%)[1] | 2(2.8%)[2] | 1(1.4%)[1] | — | 5(1.4%)[5] |
| Diarrhoea | 2(2.9%)[2] | 1(1.4%)[1] | — | 2(2.9%)[2] | — | 5(1.4%)[5] |

N = Number of subjects studied
( ) = Percentage of subjects with adverse events
[ ] = Number of adverse events

TABLE 96
Frequency of Moderate Adverse Events

| | | | 4.375 mg | 8.75 mg | 17.5 mg | |
|---|---|---|---|---|---|---|
| | | Oral | 2-(diethylamino) | 2-(diethylamino) | 2-(diethylamino) | |
| | | Ibuprofen | ethyl | ethyl | ethyl | |
| | Placebo, | (3 × 400 | 2-(4-isobutylphenyl) | 2-(4-isobutylphenyl) | 2-(4-isobutylphenyl) | |
| System Organ Class | BID, (N = | mg) (N = | propionate/knee, | propionate/knee, | propionate/knee, | Overall |
| Preferred Term | 69) | 71) | BID (N = 71) | BID (N = 70) | BID (N = 70) | (N = 351) |
| Total Adverse Events | 3(4.3%)[3] | 6(8.4%)[6] | 3(4.2%)[3] | 4(5.7%)[4] | — | 16(4.6%)[16] |
| Gastroenteritis | — | — | 1 (1.4%)[1] | — | — | 1(0.3%)[1] |
| Upper Respiratory Tract Infection | — | 1 (1.4%)[1] | 1 (1.4%)[1] | 4 (1.4%)[4] | — | 6(1.7%)[6] |
| skin irritation | — | 1 (1.4%)[1] | — | — | — | 1(0.3%)[1] |
| Left shoulder pain | — | 1 (1.4%)[1] | — | — | — | 1(0.3%)[1] |
| Atherosclerosis | — | 1 (1.4%)[1] | — | — | — | 1(0.3%)[1] |
| hyperhomocysteinemia | — | 1 (1.4%)[1] | — | — | — | 1(0.3%)[1] |
| Oral infections | 1 (1.4%)[1] | — | — | — | — | 1(0.3%)[1] |
| Muscle Spasms | — | 1(1.4%)[1] | — | — | — | 1(0.3%)[1] |
| Toothache | 2(1.4%)[2] | — | — | — | — | 2(0.6%)[2] |
| Gastroesophageal reflux disease | — | — | 1(1.4%)[1] | — | — | 1(0.3%)[1] |

N = Number of subjects studied
( ) = Percentage of subjects with adverse events
[ ] = Number of adverse events

TABLE 97
Serious Treatment-emergent Adverse Events (Group/Treatment: 8.75 mg of 2-(diethylamino)ethyl
2-(4-isobutylphenyl)propionate, BID)

| Subject/ | | | | | Severity/ | | Action Taken | Other Action | |
|---|---|---|---|---|---|---|---|---|---|
| Site/ | Adverse Event [Preferred | Start Date | Stop Date | Duration | SAE | Relationship | With Study | Taken With | |
| Sex/Age | Term] (System Organ Class) | [Start Time] | [Start Time] | day:h:min | Criteriaª | to Study Drug | Treatment | Subject | Outcome |
| 279/08/ | Transient Ischemic Attack | 16 May 2019 | 26 May 2019 | Less than | Severe/6 | Possible | discontinued | No | Recovered/ |
| M66 | [Thrombosis] (Vascular | [06:00] | [unknown] | 11 days | | unrelated | for 11 days | | resolved |
| | Disorders) | | | | | | | | |

ª1 = Results in Death; 2 = Is Life Threatening; 3 = Requires subject hospitalization or prolongation of an existing hospitalization; 4 = Results in persistent or significant disability or incapacity; 5 = Results in a congenital anomaly or birth defect; 6 = Results in an important medical event

TABLE 98
Serious Treatment-emergent Adverse Events (Group/Treatment: Placebo)

| Subject/ | | | | | Severity/ | | Action Taken | Other Action | |
|---|---|---|---|---|---|---|---|---|---|
| Site/ | Adverse Event [Preferred | Start Date | Stop Date | Duration | SAE | Relationship | With Study | Taken With | |
| Sex/Age | Term] (System Organ Class) | [Start Time] | [Start Time] | day:h:min | Criteriaª | to Study Drug | Treatment | Subject | Outcome |
| 105/07/ | Hepatotoxicity [Hepatobiliary | 11 Mar. 2019 | 29 Mar. 2019 | 17:23:10 | Severe | Possible | No | No | Getting |
| F64 | disorders] (liver & | [09:30] | [08:40] | | | related | | | better |
| | gallbladder system) | | | | | | | | |

ª1 = Results in Death; 2 = Is Life Threatening; 3 = Requires subject hospitalization or prolongation of an existing hospitalization; 4 = Results in persistent or significant disability or incapacity; 5 = Results in a congenital anomaly or birth defect; 6 = Results in an important medical event

TABLE 99
Serious Treatment-emergent Adverse Events (Group/Treatment: Oral ibuprofen 3 × 400 mg)

| Subject/ | | | | | Severity/ | | Action Taken | Other Action | |
|---|---|---|---|---|---|---|---|---|---|
| Site/ | Adverse Event [Preferred | Start Date | Stop Date | Duration | SAE | Relationship | With Study | Taken With | |
| Sex/Age | Term] (System Organ Class) | [Start Time] | [Start Time] | day:h:min | Criteriaª | to Study Drug | Treatment | Subject | Outcome |
| 128/05/ | Diverticulitis [Diverticulitis] | 6 Jan. 2019 | 15 Mar. 2019 | 67:20:40 | Severe/6 | Unrelated | discontinued for | Surgery | Recovered/ |
| F64 | (Infections and Infestations) | [14:30] | [11:10] | | | | a few of days | | resolved |

ª1 = Results in Death; 2 = Is Life Threatening; 3 = Requires subject hospitalization or prolongation of an existing hospitalization; 4 = Results in persistent or significant disability or incapacity; 5 = Results in a congenital anomaly or birth defect; 6 = Results in an important medical event

TABLE 100
Serious Treatment-emergent Adverse Events (Group/Treatment: 4.375 mg of 2-(diethylamino)ethyl
2-(4-isobutylphenyl)propionate, BID)

| Subject/ | | | | | Severity/ | Relation- | Action Taken | Other Action | |
|---|---|---|---|---|---|---|---|---|---|
| Site/ | Adverse Event [Preferred | Start Date | Stop Date | Duration | SAE | to ship | With Study | Taken With | |
| Sex/Age | Term] (System Organ Class) | [Start Time] | [Start Time] | day:h:min | Criteriaª | Study Drug | Treatment | Subject | Outcome |
| 105/07/ | abdominal aortic aneurysm | 22 Jul. 2019 | 23 Jul. 2019 | 00:03:48 | Severe/1 | Unrelated | discontinued | Hospi- | Results |
| F64 | [aortic aneurysm] (Blood and | [22:40] | [02:200] | | | | for a few of | talization | in |
| | Lymphatic System Disorders) | | | | | | days | | Death |

ª1 = Results in Death; 2 = Is Life Threatening; 3 = Requires subject hospitalization or prolongation of an existing hospitalization; 4 = Results in persistent or significant disability or incapacity; 5 = Results in a congenital anomaly or birth defect; 6 = Results in an important medical event

All three doses of 2-(diethylamino)ethyl 2-(4-isobutylphenyl)propionate appeared to be safe and were generally well tolerated. As stated previously, Gastrointestinal disorders are the major problem of all NSAIDs, there are less GI disorders in 2-(diethylamino)ethyl 2-(4-isobutylphenyl)propionate groups, the incidence rates: 21.1% (oral ibuprofen), 4.2%(4.375 mg of 2-(diethylamino)ethyl 2-(4-isobutylphenyl)propionate), 14.3% (8.75 mg of 2-(diethylamino)ethyl 2-(4-isobutylphenyl)propionate), 5.7%(17.5 mg of 2-(diethylamino)ethyl 2-(4-isobutylphenyl)propionate), and 14.5% (placebo), there are less positive fecal occult blood rests in 2-(diethylamino)ethyl 2-(4-isobutylphenyl)propionate groups, the incidence rates: 11.3% (oral ibuprofen), 7.0% (4.375 mg of 2-(diethylamino)ethyl 2-(4-isobutylphenyl)propionate), 7.1% (8.75 mg of 2-(diethylamino)ethyl 2-(4-isobutylphenyl)propionate), 7.1%(17.5 mg of 2-(diethylamino)ethyl 2-(4-isobutylphenyl)propionate), and 14.5% (placebo), there are less possibly, probably, or definitely related to study drug incidences in 2-(diethylamino)ethyl 2-(4-isobutylphenyl)propionate groups, the incidence rates: 46.5% (oral ibuprofen), 33.8% (4.375 mg of 2-(diethylamino)ethyl 2-(4-isobutylphenyl)propionate), 41.4% (8.75 mg of 2-(diethylamino)ethyl 2-(4-isobutylphenyl)propionate), 35.7% (17.5 mg of 2-(diethylamino)ethyl 2-(4-isobutylphenyl)propionate), and 47.8% (placebo). NSAIDs may increase infection incidences. (Amy E. Bryand, Clifford R. Bayer, Michael J. Aldape, and Dennis L. Stevens. “The roles of injury and nonsteroidal anti-inflammatory drugs in the development and out comes of severe group A streptococcal soft tissue infections.” Curr Opin. Infect Dis. 2015 June: 28(3): 231-239; Guillaume Voiriot, Quentin Philippot, Alexandre Elabbadi, Carole Elbim, Martin Chalumcau, and Muriel Fartoukh. “Risks Related to the Use of Non-Steroidal Anti-Inflarmatory Drugs in Community-Acquired Pneumonia in Adult and Pediatric Patients.” J. Clin. Med. 2019, 8, 786-795.) There are less infections and infestations in 2-(diethylamino)ethyl 2-(4-isobutylphenyl)propionate groups, the incidences rates: 43.7% (oral ibuprofen), 32.4% (4.375 mg of 2-(diethylamino)ethyl 2-(4-isobutylphenyl)propionate), 28.6% (8.75 mg of 2-(diethylamino)ethyl 2-(4-isobutylphenyl)propionate), 18.6% (17.5 mg of 2-(diethylamino)ethyl 2-(4-isobutylphenyl)propionate), and 20.2% (placebo). There are more skin irritation (a common AE of topical drugs) incidences in high dose group than other group which may be related to 2-(diethylamino)ethyl 2-(4-isobutylphenyl)propionate, but the incidence rates were very low (total 9) and all are mild.

Overall safety data show 2-(diethylamino)ethyl 2-(4-isobutylphenyl)propionate is safer than oral ibuprofen.

4.13. PK Summary:

From previous 1 Phase land 2 Phase 2 clinical studies, absorption of 2-(diethylamino)ethyl 2-(4-isobutylphenyl)propionate was rapid and the absorbed 2-(diethylamino)ethyl 2-(4-isobutylphenyl)propionate was rapidly converted to ibuprofen and 2-(diethylamino)ethyl 2-(4-isobutylphenyl)propionate cannot be detected in most of subjects' plasma of low dose levels (below 35 mg/day) of 2-(diethylamino)ethyl 2-(4-isobutylphenyl)propionate. Thus only ibuprofen was analyzed in this trial. Following topical applications of 4.375 mg, 8.75 mg, and 17.5 mg of 2-(diethylamino)ethyl 2-(4-isobutylphenyl)propionate to OA subjects, ibuprofen mean maximum plasma concentration and AUC increased as 2-(diethylamino)ethyl 2-(4-isobutylphenyl)propionate dose increased from 4.375 mg to 17.5 mg.

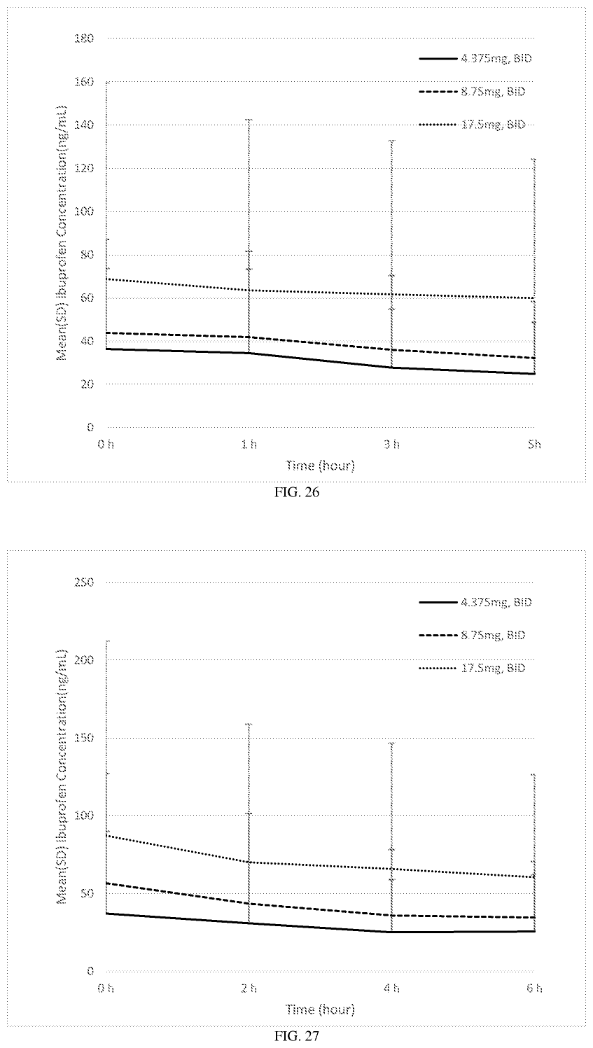

Line charts of mean (SD) Ibuprofen Plasma Concentration vs. Time by Treatment at Week 8 and Week 12 (linear scale) (n=18-20) in the second Phase 2 clinical study are respectively shown in FIG. 26 and FIG. 27.

### Applications of 2-(diethylamino)ethyl 2-(4-isobutylphenyl)propionate

A pharmaceutical composition of 2-(diethylamino)ethyl 2-(4-isobutylphenyl)propionate which capable of penetrating cartilage can be used for treating osteoarthritis of humans and animals.

### Advantage

In certain embodiments, since 2-(diethylamino)ethyl 2-(4-isobutylphenyl)propionate of the present disclosure is capable of crossing one or more biological barriers and can be administered locally (e.g., topically or transdermally) to reach a location where a condition occurs without the necessity of a systematic administration (e.g., oral or parenteral administration) of a large amount of drug.

As shown by the reduction in pain, stiffness, and functional ability severity in the patient's assessment in WOMAC (VAS) pain subscale scores, WOMAC (VAS) stiffness subscale scores, WOMAC (VAS) functional ability subscale scores, and use of rescue medication, by the percentage of patients who reported good or excellent in Subject's and investigator's global assessment of response to therapy, 2-(diethylamino)ethyl 2-(4-isobutylphenyl)propionate can reduce the signs and symptoms of osteoarthritis significantly in a dose response manner. The efficacy of 2-(diethylamino)ethyl 2-(4-isobutylphenyl)propionate in the relief of the signs and symptoms of osteoarthritis is found to be superior to the marketed common NSAIDs, such as Ibuprofen, Celebcoxib and Naproxen.

All 4.375 mg, 8.75 mg, 17.5 mg and 35 mg/knee BID of 2-(diethylamino)ethyl 2-(4-isobutylphenyl)propionate appeared to be safe and were generally well tolerated. Gastrointestinal disorders are the major problem of all NSAIDs, but there were not drug-related gastrointestinal disorders in these studies. No notable upper GI tract ulcer complication (ie, bleeding episode, perforation, or gastric outlet obstruction) occurred during these studies. Mean and median blood pressures remained unchanged. Even the skin irritation (a common AE of topical drugs) incidence rates were very low and mild due to the simple formulation.

All publications cited in this specification are incorporated herein by reference to the same extent as if each individual publication was specifically and individually indicated to be incorporated by reference.

Although certain embodiments are described in detail above, they are only shown illustration and not as limitations of the invention. The principal features of this invention can be employed in various embodiments without departing from the scope of the invention. Those having ordinary skill in the art will clearly understand that many modifications are possible in the claims without departing from the teachings thereof, and will recognize, or be able to ascertain using no more than routine study, numerous equivalents to the specific procedures described herein. All such modifications and equivalents are intended to be encompassed within the claims of the invention and are covered by the claims.

1. (canceled)

2. (canceled)

3. (canceled)

4. A method of treatment of a subject suffering from, at risk of suffering from, or potentially capable of suffering from a pain or inflammation, comprising topically administrating 2-(diethylamino)ethyl 2-(4-isobutylphenyl)propionate, or a pharmaceutically acceptable salt thereof, to one or more sites of the subject, in an amount of about 1 mg to about 80 mg per day per site.

5. The method of claim 4, wherein the amount is about 2 mg to about 40 mg per dose per site.

6. The method of claim 4, wherein the amount is about 5 μg/cm² to about 2 mg/cm² per dose per site.

7. A kit for treatment of a subject, comprising 2-(diethylamino)ethyl 2-(4-isobutylphenyl)propionate, or a pharmaceutically acceptable salt thereof, for topical administration to one or more sites of the subject, in an amount of about 1 mg to about 80 mg per day per site.

8. The kit of claim 7, wherein the amount is about 2 mg to about 40 mg per dose per site.

9. The kit of claim 7, wherein the amount is about 5 μg/cm² to about 2 mg/cm² per dose per site.

10. A dosage form comprising 2-(diethylamino)ethyl 2-(4-isobutylphenyl)propionate, or a pharmaceutically acceptable salt thereof, selected from the group consisting of transdermal patch, cream, foam, gel, lotion, ointment, paste, powder, shake lotion, solid, sponge, tape, tinkture, vapor, drops, rinses, spray, and solution, preferably from transdermal drops, rinses and spray.

11. The dosage form of claim 10, selected from the group consisting of an alcohol solution, an acetone solution, a dimethyl sulfoxide solution, an alcohol-water solution, an acetone-water solution, and a dimethyl sulfoxide-water solution, preferably an ethanol-water solution, preferably a 10% to 50% (v/v) ethanol-water solution, particularly a 25% (v/v) ethanol water solution.

12. The dosage form of claim 10, wherein the 2-(diethylamino)ethyl 2-(4-isobutylphenyl)propionate, or a pharmaceutically acceptable salt thereof, is at a concentration of about 10 mg/mL to about 200 mg/mL, or about 10 mg/g to about 200 mg/g.

13. (canceled)

14. The method of claim 4, wherein 2-(diethylamino)ethyl 2-(4-isobutylphenyl)propionate, or a pharmaceutically acceptable salt thereof, is topically administered in an amount of about 0.5 mg to about 64 mg, particularly about 8 mg to about 16 mg, or particularly about 16 mg to about 32 mg, or particularly about 32 mg to about 64 mg, per day per site.

15. The method of claim 4, wherein 2-(diethylamino)ethyl 2-(4-isobutylphenyl)propionate, or a pharmaceutically acceptable salt thereof, is topically administered in an amount of about 8 mg to about 64 mg, particularly about 4 mg to about 8 mg, or particularly about 8 mg to about 16 mg, or particularly about 16 mg to about 32 mg, per dose per site.

16. The method of claim 4, wherein 2-(diethylamino)ethyl 2-(4-isobutylphenyl)propionate, or a pharmaceutically acceptable salt thereof, is topically administered in an amount of about 30 μg/cm² to about 480 μg/cm², particularly about 30 μg/cm² to about 60 μg/cm², particularly about 60 μg/cm² to about 120 μg/cm², or particularly about 120 μg/cm² to about 240 μg/cm², or particularly about 240 μg/cm² to about 480 μg/cm², per dose per site.

17. (canceled)

18. The method of claim 4, wherein the pain is an arthritis pain, osteoarthritis pain, bone pain, or muscle pain; and wherein the inflammation is osteoarthritis, rheumatoid arthritis, gout, lupus, fibromyalgia, and/or septic arthritis.

19. The method of claim 4, wherein said site of the subject is selected from the group consisting of knees, ankles, elbows, wrists, shoulders, hips, fingers, toes, neck spines, back spines, and tissues, and combinations thereof.

20. The method of claim 4, wherein one of said site of the subject is one knee, one ankle, one elbow, one wrist, one shoulder, one side of hips, one finger, one toe, one neck spine, one back spine, or one area of soft tissues.

21. The method of claim 4, wherein 2-(diethylamino)ethyl 2-(4-isobutylphenyl)propionate, or a pharmaceutically acceptable salt thereof, is topically administered to one or more surfaces of the site, in particular topically administered to the medial surface, the lateral surface, the front surface and/or the back surface of the site.

22. The method of claim 4, wherein 2-(diethylamino)ethyl 2-(4-isobutylphenyl)propionate, or a pharmaceutically acceptable salt thereof, is topically administered by a dosage form selected from transdermal patch, cream, foam, gel, lotion, ointment, paste, powder, shake lotion, solid, sponge, tape, tinkture, vapor, drops, rinses, spray, and solution, in particular from transdermal drops, rinses or spray, in particular spray.

23. The method of claim 4, wherein 2-(diethylamino)ethyl 2-(4-isobutylphenyl)propionate is topically administered in a dosage form selected from the group consisting of an alcohol solution, an acetone solution, a dimethyl sulfoxide solution, an alcohol water solution, an acetone water solution, or a dimethyl sulfoxide water solution, preferably an ethanol water solution, more preferably 10% to 50% (v/v) ethanol-water solution, particularly 25% (v/v) ethanol-water solution.

24. The method of claim 4, wherein the 2-(diethylamino)ethyl 2-(4-isobutylphenyl)propionate, or a pharmaceutically acceptable salt thereof, is administered to the subject as a solution having a concentration of about 10 mg/mL to about 200 mg/mL, preferably about 30 mg/mL to about 100 mg/mL, more preferably about 50 mg/mL to about 80 mg/mL, particularly about 70 mg/mL.

25. The method of claim 4, wherein the 2-(diethylamino)ethyl 2-(4-isobutylphenyl)propionate, or a pharmaceutically acceptable salt thereof, is topically administered to the subject in a unit dose of about 0.01 mL to about 1 mL, particularly about 0.03 mL to about 0.3 mL, particularly about 0.04 mL to about 0.2 mL, particularly about 0.05 mL to about 0.1 mL, particularly about 0.07 mL.

26. The method of claim 4, wherein the 2-(diethylamino)ethyl 2-(4-isobutylphenyl)propionate or pharmaceutically acceptable salt thereof is administered in an amount of about 0.1 mg to about 8 mg, particularly about 2 mg to about 6 mg, particularly about 3 mg to about 5 mg, particularly about 4 mg to about 4.75 mg, particularly about 4.5 mg, per unit dose.

27. The method of claim 4, wherein one or more unit doses comprising a composition comprising 2-(4-isobutylphenyl)propionate, or a pharmaceutically acceptable salt thereof, is topically administered to the subject in a single dose per site; and wherein said one or more unit doses are 1-20 unit doses, particularly 1-10 unit doses, particularly 1-5 unit doses, particularly 2-4 unit doses, particularly 2-3 unit doses, or particularly 1 unit dose, or particularly 2 unit doses, or particularly 4 unit doses, or particularly 8 unit doses.

28. The method of claim 4, wherein the 2-(diethylamino)ethyl 2-(4-isobutylphenyl)propionate, or a pharmaceutically acceptable salt thereof is administered once, twice, three times, four times, five times, six times, seven times or eight times a day, preferably twice a day.

29. The method of claim 4, wherein the 2-(diethylamino)ethyl 2-(4-isobutylphenyl)propionate or pharmaceutically acceptable salt thereof is administered once every 1, 2, 3, 4, 5, 6, 7, 8, 9, 10, 11, 12, 13, 14, 15, 16, 17, 18, 19, 20, 21, 22, 23, or 24 hours, preferably once every 12 hours.

30. The method of claim 4, wherein the 2-(diethylamino)ethyl 2-(4-isobutylphenyl)propionate or pharmaceutically acceptable salt thereof is administered for 1 day to life time, particularly 7 to 365, 14 to 91, 14 to 84, 28 to 84, or 56 to 84 consecutive or non-consecutive days, particularly consecutive days.
